(19) United States
(12) Patent Application Publication
(10) Pub. No.: US 2025/0352639 A1
(43) Pub. Date: Nov. 20, 2025

(54) Bicistronic LAMP Constructs Comprising Immune Response Enhancing Genes and Methods of Use Thereof

(71) Applicant: Immunomic Therapeutics, Inc., Rockville, MD (US)

(72) Inventors: Teri Heiland, New Market, MD (US); Wei Shen, North Potomac, MD (US); Yun-Ting Kao, Germantown, MD (US); Renhuan Xu, North Potomac, MD (US)

(73) Assignee: Immunomic Therapeutics, Inc., Rockville, MD (US)

(21) Appl. No.: 18/854,261

(22) PCT Filed: Apr. 10, 2023
(86) PCT No.: PCT/US2023/065588

§ 371 Date: Oct. 4, 2024

Related U.S. Application Data
(60) Provisional application No. 63/329,463, filed on Apr. 10, 2022.

(51) Int. Cl. A61K 39/215; A61K 9/00; A61K 31/7105; A61K 31/711; A61K 39/00; A61P 31/14; C07K 14/705; C12N 15/62

(52) U.S. Cl. CPC A61K 39/215; A61K 9/0019; A61K 31/7105; A61K 31/711; A61P 31/14; C07K 14/70596; C12N 15/62; A61K 2039/53; A61K 2039/54

(57) ABSTRACT

The present disclosure provides nucleic acid molecules (e.g., a plasmid or vector) comprising a nucleic acid sequence encoding a bicistronic or multicistronic LAMP Construct comprising specific fragments of the LAMP luminal domain and an antigenic domain heterologous to the LAMP protein to provide at least one antigen for priming an immune response, wherein the antigen expressed by the Construct is optionally processed and presented to MHC class II molecules, and also a nucleic acid sequence encoding an immune response enhancing polypeptide that is optionally secreted from a host cell. The nucleic acid molecules can be used, for example, for the treatment of disease and in particular, allergies, infectious disease, diabetes, hyperproliferative disorders and/or cancer.

### CROSS-REFERENCE TO RELATED APPLICATIONS

This application is a national stage entry pursuant to 35 U.S.C. § 371 of International Application No. PCT/US2023/065588, filed Apr. 10, 2023, which claims priority to U.S. Provisional Patent Application No. 63/329,463, filed Apr. 10, 2022, each of which are incorporated by reference in their entirety herein.

### SEQUENCE LISTING

The present application contains a Sequence Listing which has been submitted electronically in XML format. Said XML copy, created on Apr. 22, 2025, is named “2025-04-22_01305-0023-00PCTST26.xml” and is 1,419,384 bytes in size. The information in the electronic format of the sequence listing is incorporated herein by reference in its entirety.

### FIELD

The disclosure relates to isolated nucleic acid molecules (e.g., a plasmid or vector) encoding a bicistronic or multicistronic LAMP (Lysosomal-Associated Membrane Protein) Construct comprising a LAMP fusion protein and a second, optionally secreted protein such as from an immune response enhancing gene (IREG), and their use in treating subjects suffering from infectious disease, diabetes, allergies, hyperproliferative disorders and/or cancer, and in particular COVID-19. Additionally, the bicistronic LAMP construct described herein can be used to generate antibodies in non-human vertebrates, preferably where the genome of the non-human vertebrates comprises at least partially human immunoglobulin regions and/or humanized immunoglobulin regions.

### BACKGROUND

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Vaccines are new and promising candidates for the development of both prophylactic and therapeutic vaccines. They are proven to be safe and the lack of immune responses to a vector backbone may be a definitive advantage if repetitive cycles of vaccination are required to achieve clinical benefits. However, one perceived disadvantage of conventional vaccines is their low immunogenicity in humans. A key limiting step in the immunogenicity of epitope-based vaccines may be the access of epitopes to the major histocompatibility (MHC) class II presentation pathway to T cells, which is likely a stochastic process in the case of a vaccine without targeting technology.

LAMP-antigen constructs of various designs have previously been described, for example, in U.S. Pat. No. 11,203,629 (see FIG. 1 therein). One type of construct, described in U.S. Pat. No. 11,203,629 named ILC-4 (depicted in FIG. 1 herein), comprises at least one antigen of interest fused in between a first homology domain of a LAMP protein and a second homology domain of a LAMP protein (or at least between two Cysteine Conserved Fragments), for example the at least one antigen of interest may be placed in the LAMP hinge region. In some embodiments, this construct also comprises a transmembrane domain of a LAMP protein, and/or the cytosolic tail of a LAMP protein. The two homology domains may be derived from, for example, LAMP-1, LAMP-2, LAMP-3, or an Endolyn protein. Alternatively, two homology domains from two different LAMP proteins may be used. The inventors unexpectedly found that improved LAMP Constructs such as ILC-4 can, for example, elicit strong T-cell and antibody responses against the antigen(s) of interest, making them viable candidates for use as vaccines.

Notwithstanding the above, there is a further needed to design new and further improved LAMP constructs, and the nucleic acid molecules encoding them, that can be used as vaccines to effectively treat, for example, allergies, infectious disease, diabetes, hyperproliferative disorders and/or cancer, and/or be used in the generation of useful antibodies.

### SUMMARY

As described further herein, the inventors have now found that isolated nucleic acid molecules can be designed that not only express a LAMP construct, such as that described, for example, in FIG. 1 herein, but that also express a particular type of second polypeptide, often a secreted polypeptide, encoding a gene such as CD40L, CD80, OX40, IL-12, IL-21, IL-15, or Flt3L or the like that have been found to enhance immune responses against tumors or infectious diseases in vivo, and that expressing these two polypeptides from the isolated nucleic acid molecule unexpectedly enhances the immune response compared to not only earlier LAMP constructs but also compared to bicistronic LAMP constructs comprising certain secreted antigens such as a second disease antigen.

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

One object of this disclosure to provide novel nucleic acid molecules encoding constructs (“bicistronic LAMP constructs”) comprising specific fragments and/or variants of LAMP domains that effectively present an antigen(s) of interest to the immune system to generate an enhanced immune response. These bicistronic LAMP constructs effectively direct the antigens to the lysosomal/endosomal compartment where they are processed and presented to major histocompatibility complex (MHC) class II molecules so that helper T cells are preferentially stimulated and/or antibodies are generated along with the ability to enhance the immune response.

The nucleic acid molecules encoding the bicistronic LAMP constructs and methods described herein may elicit an immune response in a subject. The immune response may be an immune response to an epitope of an antigen encoded in the bicistronic LAMP construct (e.g., vaccine). Vaccines arm the immune system of the subject such that the immune system may detect and destroy that which contains the antigen(s) of a vaccine in the subject. The nucleic acid molecules encoding the bicistronic LAMP constructs and methods described herein may elicit a Th1 immune response in the subject. Th1 immune responses may include secretion of inflammatory cytokines (e.g., IFNγ, TNFa) by a subset of immune cells (e.g., antigen specific T-cells). In some cases, the inflammatory cytokines activate another subtype of immune cells (e.g., cytotoxic T-cells) which may destroy that which contains the antigen in the subject.

In some cases, an antigen used in the bicistronic LAMP constructs and methods described herein may be recognized by the immune system of a subject to elicit a Th1 immune response and release Type I cytokines. The Th1 response may be initiated by the interaction between the epitope and the T-cell, more specifically, the major histocompatibility complex (MHC) expressed by the T-cell. For example, high affinity binding of an epitope to an MHC receptor may stimulate a Th1 response. MHC receptors may be at least one of a plurality of types of MHC receptors. The MHC receptors engaged on a T-cell may vary across individuals in a population.

In some cases, the immune response is a Type 1 immune response. In some cases, the immune response is characterized by a ratio of Type I cytokine production to Type II cytokine production that is greater than 1. In some cases, the immune response is characterized by a ratio of Type I cytokine production to Type II cytokine production that is less than 1. In some cases, the immune response is characterized by a ratio of IFNγ production to IL-10 production that is greater than 1. In some cases, the immune response is characterized by a ratio of IFNγ production to IL-10 production that is less than 1.

The nucleic acid molecules encoding the bicistronic LAMP constructs described herein can also be used in a manner to provide an expression of immunoregulatory elements (IREs) or immune response enhancing-genes (IREGs) elicit an enhanced immune response in a subject (e.g., an immune response comprising a significantly higher antibody titer). For example, a nucleic acid molecule (e.g., a plasmid or vector) may provide for the expression of a bicistronic LAMP construct comprising a LAMP-antigen polypeptide that is processed and presented to MHC class II molecules so that helper T cells are preferentially stimulated, memory cells are initiated and/or antibodies are generated), as well as providing for the expression of a further IREG or IRE polypeptide that may be secreted into the circulation of the subject, and that may, for example, enhance further both the humoral and cellular immune response to the LAMP antigen.

In one aspect, the nucleic acid molecule encoding the bicistronic LAMP construct is a vaccine vector, suitable for vaccinating a subject. In another aspect, the disclosure provides a delivery vehicle for facilitating the introduction of the nucleic acid molecule encoding the bicistronic LAMP construct comprising polynucleotides encoding epitopes and/or antigens into a cell. The delivery vehicle may be lipid-based (e.g., a liposome formulation), viral-based (e.g., comprising viral proteins encapsulating the nucleic acid molecule), or cell-based.

In some embodiments, the disclosure provides an injectable composition comprising a nucleic acid molecule as described herein encoding a bicistronic LAMP construct for eliciting an immune response (e.g., generation of antibodies) in a subject to an antigen. In some embodiments, this vaccine generates a preferential Th1 response to a Th2 response.

The disclosure also provides a cell comprising a nucleic acid molecule as described herein encoding a bicistronic LAMP construct which can be used to generate an immune response. In one aspect, the cell is an antigen presenting cell. The antigen presenting cell may be a professional antigen presenting cell (e.g., a dendritic cell, macrophage, B cell, and the like) or an engineered antigen presenting cell (e.g., a non-professional antigen presenting cell engineered to express molecules required for antigen presentation, such as MHC class II molecules). The molecules required for antigen presentation may be derived from other cells, e.g., naturally occurring, or may themselves be engineered (e.g., mutated or modified to express desired properties, such as higher or lower affinity for an antigenic epitope).

The disclosure additionally provides a kit comprising a plurality of cells comprising a nucleic acid molecule as described herein encoding a bicistronic LAMP construct. At least two of the cells may express different MHC class II molecules, and each cell may comprise the same LAMP Construct. In one aspect, a kit is provided comprising a viral vector encoding a bicistronic LAMP construct.

The disclosure also provides a transgenic animal comprising at least one of the cells and/or at least one of the nucleic acid molecules encoding a bicistronic LAMP construct as described herein. The disclosure also provides a transgenic animal comprising at least one of the cells described herein.

The disclosure further provides a method for generating an enhanced immune response in a subject (e.g., a human or a non-human vertebrate) to an antigen, comprising administering to the subject a cell as described above, wherein the cell expresses, or can be induced to express, the bicistronic LAMP construct in the subject. In one aspect, the cell comprises an MHC class II molecule compatible with MHC proteins of the subject, such that the subject does not generate an immune response against the MHC class II molecule.

In one further aspect, the disclosure provides a method for eliciting an enhanced immune response to an antigen, comprising administering to a subject, such as a human or a non-human vertebrate, a nucleic acid molecule encoding a bicistronic LAMP construct as described herein. Preferably, the nucleic acid molecule is infectious for a cell of the subject. For example, the nucleic acid molecule encoding the bicistronic LAMP construct may be a viral vector, such as a vaccinia vector.

The present disclosure also comprises methods of generating antibodies in a non-human vertebrate wherein the non-human vertebrate is injected with a nucleic acid molecule encoding a bicistronic LAMP construct as described herein. Generated antibodies can be isolated from the blood of the vertebrate (as polyclonals) and then further isolated to generate monoclonal antibodies using standard techniques.

The methods described herein can be used in the production and/or optimization of antibodies, including fully human antibodies, humanized antibodies, chimeric antibodies, for diagnostic and therapeutic uses. Hybridomas producing such antibodies are also a further object of the disclosure.

Specific embodiments of the disclosure include the following:

1. An isolated nucleic acid molecule comprising
a. a first polynucleotide sequence encoding a polypeptide comprising two homology domains of a luminal domain of a LAMP protein, and an antigenic domain heterologous to the LAMP protein (collectively a “LAMP-antigen Construct”), wherein the antigenic domain is placed between the two homology domains; and
b. a second polypeptide sequence encoding at least one second polypeptide comprising an immune response enhancing gene polypeptide (IREG) or an extracellular domain of an IREG comprising a secretion signal sequence.

2. The isolated nucleic acid molecule of embodiment 1, wherein the LAMP protein is selected from LAMP-1, LAMP2, LAMP-3, lysosomal integral membrane protein-2 (“LIMP 2”), Macrosailin, Endolyn, LAMP5 or limbic system-associated membrane protein (“LIMBIC”).

3. The isolated nucleic acid molecule of embodiment 2, wherein the LAMP protein is selected from any one of SEQ ID NO:1-113.

4. The isolated nucleic acid molecule of embodiment 1 or 2, wherein the LAMP protein is at least 70%, at least 75%, at least 80%, at least 85%, at least 90%, at least 95%, at least 96%, at least 97%, at least 98%, or at least 99% identical to SEQ ID NO:1-113.

5. The isolated nucleic acid molecule of embodiment 2, wherein the LAMP protein is LAMP-1 and wherein the two homology domains of the LAMP-antigen Construct comprise LAMP-1 Homology Domain 1 and LAMP-1 Homology Domain 2.

6. The isolated nucleic acid molecule of embodiment 5, wherein the human LAMP-1 Homology Domain 1 comprises the amino acid sequence of residues 29-194 of SEQ ID NO: 1, or comprises the amino acid sequence of residues 29-195 of SEQ ID NO: 198, or comprises an amino acid sequence at least 90%, at least 95%, at least 96%, at least 97%, at least 98%, or at least 99% identical to the amino acid sequence of residues 29-194 of SEQ ID NO: 1 or to the amino acid sequence of residues 29-195 of SEQ ID NO: 198, or wherein the nucleic acid molecule comprises the nucleotide sequence of SEQ ID NO: 199 or a nucleotide sequence at least 90%, at least 95%, at least 96%, at least 97%, at least 98%, or at least 99% identical to that of SEQ ID NO: 199 (wherein if the nucleotide sequence is RNA, T is replaced with U).

7. The isolated nucleic acid molecule of embodiment 5 or 6, wherein the human LAMP-1 Homology Domain 2 comprises the amino acid sequence of residues 228-381 of SEQ ID NO: 1, or of residues 228-382 of SEQ ID NO: 1, or comprises the amino acid sequence of SEQ ID NO: 202, or comprises an amino acid sequence at least 90%, at least 95%, at least 96%, at least 97%, at least 98%, or at least 99% identical to the amino acid sequence of residues 228-381 of SEQ ID NO: 1 or to the amino acid sequence of SEQ ID NO: 202, or wherein the nucleic acid molecule comprises the nucleotide sequence of SEQ ID NO: 203 or a nucleotide sequence at least 90%, at least 95%, at least 96%, at least 97%, at least 98%, or at least 99% identical to that of SEQ ID NO: 203 (wherein if the nucleotide sequence is RNA, T is replaced with U).

8. The isolated nucleic acid molecule of any one of embodiments 1-7, wherein the LAMP-antigen construct comprises a linker between at least one of the two homology domains and the antigenic domain.

9. The isolated nucleic acid molecule of embodiment 8, wherein the linker comprises an amino acid sequence of GPGPG or PMGLP.

10. The isolated nucleic acid molecule of any one of embodiments 1-9, wherein the LAMP-antigen construct further comprises a transmembrane domain of a LAMP Protein.

11. The isolated nucleic acid molecule of embodiment 10, wherein the transmembrane domain comprises residues 383 to 405 of SEQ ID NO: 1.

12. The isolated nucleic acid molecule of any one of embodiments 1-11, wherein the LAMP-antigen construct further comprises a signal sequence.

13. The isolated nucleic acid molecule of embodiment 12, wherein the signal sequence is derived from a LAMP Protein

14. The isolated nucleic acid molecule of any one of embodiments 1-13, wherein the LAMP-antigen construct further comprises cytoplasmic domain of a LAMP Protein.

15. The isolated nucleic acid molecule of embodiment 14, wherein the cytoplasmic domain comprises residues 406-417 of SEQ ID NO: 1.

16. The isolated nucleic acid molecule of any one of embodiments 1-15, wherein the IREG comprises one or more of CD40L, CD80, OX40, Flt3L, GM-CSF, IL-12, IL-21, IL-23, IL-15, CD70, CD86, IL-7, IL-18, or IL-33 (or comprises an amino acid sequence at least 90%, at least 95%, at least 96%, at least 97%, at least 98%, or at least 99% identical, or 100% identical to the amino acid sequence of any one of SEQ ID NOs: 133, 145, 147, 149, 151, 155, 159, 165, 169, 173, 177, 181, 189, 191, 204, 238, 242, 243, 252, or 253), or an extracellular domain thereof, optionally wherein the CD40L, CD80, OX40, Flt3L, GM-CSF, IL-12, IL-21, IL-23, or IL-15 is fused to an Fc domain of an immunoglobulin, or wherein the isolated nucleic acid molecule comprises a nucleotide sequence encoding CD40L, CD80, OX40, Flt3L, GM-CSF, IL-12, IL-21, IL-23, IL-15, CD70, CD86, IL-7, IL-18, or IL-33, optionally fused to an Fc domain, wherein the nucleotide sequence at least 90%, at least 95%, at least 96%, at least 97%, at least 98%, or at least 99% identical, or 100% identical to that of any one of SEQ ID NOs: 134, 146, 148, 150, 152, 160, 166, 170, 174, 178, 182, 190, 192, 205, 239, 244, or 881.

17. The isolated nucleic acid molecule of any one of embodiments 1-16, wherein the secretion signal sequence is heterologous to the IREG.

18. The isolated nucleic acid molecule of embodiment 17, wherein the secretion signal sequence is derived from IgKVIII (e.g., SEQ ID NO: 122), Ig-kappa (e.g., SEQ ID NO: 120), tetranectin, or IL-2, and/or wherein the second polypeptide further comprises pulmonary surfactant associated protein D (SPD) (e.g., SEQ ID NO: 131).

19. The isolated nucleic acid molecule of embodiment 18, wherein the second polypeptide is expressed under the control of an EF-1alpha core promoter, such as that of SEQ ID NO: 124.

20. A composition comprising the isolated nucleic acid molecule any one of embodiments 1-18.

21. A host cell comprising the isolated nucleic acid of any one of embodiments 1-18.

22. A composition comprising the host cell of embodiment 20.

23. A method of treating a subject having a disease or a disorder or of inducing an immune response in a subject with a disease or disorder or at risk of developing a disease or disorder, wherein the method comprises administering to the subject the isolated nucleic acid molecule of any one of embodiments 1-18, the composition of embodiment 19, or the host cell of embodiment 20, in an amount sufficient to treat the disease or disorder or to induce an immune response in the subject.

24. The method of embodiment 23, wherein the method further comprises administering at least one second therapeutic to the subject.

25. An isolated nucleic acid molecule comprising
a. a first polynucleotide sequence encoding a polypeptide comprising two homology domains of a luminal domain of a LAMP protein, and an antigenic domain comprising HER2 extracellular domain (collectively “HER2-LAMP”), wherein the antigenic domain is placed between the two LAMP homology domains; and
b. a second polypeptide sequence encoding at least one second polypeptide comprising an immune response enhancing gene polypeptide (IREG) or an extracellular domain of an IREG comprising a secretion signal sequence.

26. The isolated nucleic acid molecule of embodiment 25, wherein the LAMP protein is selected from LAMP-1, LAMP2, LAMP-3, lysosomal integral membrane protein-2 (“LIMP 2”), Macrosailin, Endolyn, LAMP5 or limbic system-associated membrane protein (“LIMBIC”).

27. The isolated nucleic acid molecule of embodiment 26, wherein the LAMP protein is selected from any one of SEQ ID NO:1-113.

28. The isolated nucleic acid molecule of embodiment 25 or 26, wherein the LAMP protein is at least 70%, at least 75%, at least 80%, at least 85%, at least 90%, at least 95%, at least 96%, at least 97%, at least 98%, or at least 99% identical to SEQ ID NO:1-113.

29. The isolated nucleic acid molecule of embodiment 26, wherein the LAMP protein is LAMP-1 and wherein the two homology domains of the HER2-LAMP comprise LAMP-1 Homology Domain 1 and LAMP-1 Homology Domain 2.

30. The isolated nucleic acid molecule of embodiment 29, wherein the human LAMP-1 Homology Domain 1 comprises the amino acid sequence of residues 29-194 of SEQ ID NO: 1, or comprises the amino acid sequence of residues 29-195 of SEQ ID NO: 198, or comprises an amino acid sequence at least 90%, at least 95%, at least 96%, at least 97%, at least 98%, or at least 99% identical to the amino acid sequence of residues 29-194 of SEQ ID NO: 1 or to the amino acid sequence of residues 29-195 of SEQ ID NO: 198, or wherein the nucleic acid molecule comprises the nucleotide sequence of SEQ ID NO: 199 or a nucleotide sequence at least 90%, at least 95%, at least 96%, at least 97%, at least 98%, or at least 99% identical to that of SEQ ID NO: 199 (wherein if the nucleotide sequence is RNA, T is replaced with U).

31. The isolated nucleic acid molecule of embodiment 29 or 30, wherein the human LAMP-1 Homology Domain 2 comprises the amino acid sequence of residues 228-381 or 228-382 of SEQ ID NO: 1, or of residues 228-382 of SEQ ID NO: 1, or comprises the amino acid sequence of SEQ ID NO: 202, or comprises an amino acid sequence at least 90%, at least 95%, at least 96%, at least 97%, at least 98%, or at least 99% identical to the amino acid sequence of residues 228-381 of SEQ ID NO: 1 or to the amino acid sequence of SEQ ID NO: 202, or wherein the nucleic acid molecule comprises the nucleotide sequence of SEQ ID NO: 203 or a nucleotide sequence at least 90%, at least 95%, at least 96%, at least 97%, at least 98%, or at least 99% identical to that of SEQ ID NO: 203 (wherein if the nucleotide sequence is RNA, T is replaced with U).

32. The isolated nucleic acid molecule of any one of embodiments 25-31, wherein the HER2-LAMP comprises a linker between at least one of the two homology domains and the antigenic domain.

33. The isolated nucleic acid molecule of embodiment 32, wherein the linker comprises an amino acid sequence of GPGPG or PMGLP.

34. The isolated nucleic acid molecule of any one of embodiments 25-33, wherein the HER2-LAMP further comprises a transmembrane domain of a LAMP Protein.

35. The isolated nucleic acid molecule of embodiment 34, wherein the transmembrane domain comprises residues 383 to 405 of SEQ ID NO: 1.

36. The isolated nucleic acid molecule of any one of embodiments 25-35, wherein the HER2-LAMP further comprises a signal sequence.

37. The isolated nucleic acid molecule of embodiment 36, wherein the signal sequence is derived from a LAMP Protein.

38. The isolated nucleic acid molecule of any one of embodiments 25-37, wherein the HER2-LAMP further comprises cytoplasmic domain of a LAMP Protein.

39. The isolated nucleic acid molecule of embodiment 38, wherein the cytoplasmic domain comprises residues 406-417 of SEQ ID NO: 1.

40. The isolated nucleic acid molecule of any one of embodiments 25-39, wherein the antigenic domain comprises or consists of the amino acid sequence of SEQ ID NO: 200.

41. The isolated nucleic acid molecule of any one of embodiments 25-40, wherein the HER2-LAMP comprises or consists of the amino acid sequence of residues 1-194 of SEQ ID NO: 1 or of SEQ ID NO: 198 followed by the amino acid sequence of SEQ ID NO: 200 followed by the amino acid sequence of residues 228-381 of SEQ ID NO: 1 or of SEQ ID NO: 202.

42. The isolated nucleic acid molecule of any one of embodiments 25-41, wherein the IREG comprises one or more of CD40L, CD80, OX40, Flt3L, GM-CSF, IL-12, IL-21, IL-23, IL-15, CD70, CD86, IL-7, IL-18, or IL-33 (or comprises an amino acid sequence at least 90%, at least 95%, at least 96%, at least 97%, at least 98%, or at least 99% identical, or 100% identical to the amino acid sequence of any one of SEQ ID NOs: 133, 145, 147, 149, 151, 155, 159, 165, 169, 173, 177, 181, 189, 191, 193, 204, 238, 242, 243, 252, or 253), or an extracellular domain thereof, optionally wherein the CD40L, CD80, OX40, Flt3L, GM-CSF, IL-12, IL-21, IL-23, or IL-15 is fused to an Fc domain of an immunoglobulin, or wherein the isolated nucleic acid molecule comprises a nucleotide sequence encoding CD40L, CD80, OX40, Flt3L, GM-CSF, IL-12, IL-21, IL-23, IL-15, CD70, CD86, IL-7, IL-18, or IL-33, optionally fused to an Fc domain, wherein the nucleotide sequence at least 90%, at least 95%, at least 96%, at least 97%, at least 98%, or at least 99% identical, or 100% identical to that of any one of SEQ ID NOs: 134, 146, 148, 150, 152, 160, 166, 170, 174, 178, 182, 190, 192, 194, 205, 239, 244, or 881.

43. The isolated nucleic acid molecule of any one of embodiments 25-42, wherein the secretion signal sequence is heterologous to the IREG.

44. The isolated nucleic acid molecule of embodiment 43, wherein the secretion signal sequence is derived from IgKVIII (e.g., SEQ ID NO: 122), Ig-kappa (e.g., SEQ ID NO: 120), tetranectin, or IL-2.

45. The isolated nucleic acid molecule of any one of embodiments 25-44, wherein the second polypeptide comprises a fusion of SPD and soluble CD40L (sCD40L), a fusion of SPD and Flt3L, IL-12, IL-21, OX40L fused to an Fc domain, CD80 fused to an Fc domain, or IL-15 (such as comprising an amino acid sequence at least 90%, at least 95%, at least 96%, at least 97%, at least 98%, or at least 99% identical to, or 100% identical to SEQ ID NO: 233, 238, 242, or 252, or comprising an amino acid sequence at least 90%, at least 95%, at least 96%, at least 97%, at least 98%, or at least 99% identical to, or 100% identical to: the amino acid sequence of SEQ ID NO: 131 followed by the amino acid sequence of one of SEQ ID NOs: 204, 151, 145, 147, 149, 193, 181, 155, 159, 169, 252, or 253), or wherein the nucleic acid comprises a nucleotide sequence encoding a fusion of SPD and soluble CD40L (sCD40L), a fusion of SPD and Flt3L, IL-12, IL-21, OX40L fused to an Fc domain, CD80 fused to an Fc domain, or IL-15 (such as comprising a nucleotide sequence at least 90%, at least 95%, at least 96%, at least 97%, at least 98%, or at least 99% identical to, or 100% identical to the sequence of: SEQ ID NO: 132 followed by one of SEQ ID NOs: 205, 152, 146, 148, 150, 194, 182, 156, 170 or 881).

46. The isolated nucleic acid molecule of embodiment 45, wherein the second polypeptide is expressed under the control of an EF-1alpha core promoter, such as that of SEQ ID NO: 124.

47. A composition comprising the isolated nucleic acid molecule of any one of embodiments 25-46.

48. A host cell comprising the isolated nucleic acid of any one of embodiments 25-46.

49. A composition comprising the host cell of embodiment 48.

50. A method of treating a subject having cancer, wherein the method comprises administering to the subject the isolated nucleic acid molecule of any one of embodiments 25-46, the composition of embodiment 47, or the host cell of embodiment 48, in an amount sufficient to treat the cancer or to induce an immune response in the subject against the cancer.

51. The method of embodiment 50, wherein the method further comprises administering at least one second therapeutic to the subject.

52. An isolated nucleic acid molecule comprising
a. a first polynucleotide sequence encoding a polypeptide comprising two homology domains of a luminal domain of a LAMP protein, and an antigenic domain comprising a coronavirus Spike protein antigen (collectively “Spike-LAMP”), wherein the antigenic domain is placed between the two LAMP homology domains; and
b. a second polypeptide sequence encoding at least one second polypeptide comprising an immune response enhancing gene polypeptide (IREG) or an extracellular domain of an IREG comprising a secretion signal sequence.

53. The isolated nucleic acid molecule of embodiment 52, wherein the LAMP protein is selected from LAMP-1, LAMP2, LAMP-3, lysosomal integral membrane protein-2 (“LIMP 2”), Macrosailin, Endolyn, LAMP5 or limbic system-associated membrane protein (“LIMBIC”).

54. The isolated nucleic acid molecule of embodiment 53, wherein the LAMP protein is selected from any one of SEQ ID NO:1-113.

55. The isolated nucleic acid molecule of embodiment 52 or 53, wherein the LAMP protein is at least 70%, at least 75%, at least 80%, at least 85%, at least 90%, at least 95%, at least 96%, at least 97%, at least 98%, or at least 99% identical to SEQ ID NO:1-113.

56. The isolated nucleic acid molecule of embodiment 53, wherein the LAMP protein is LAMP-1 and wherein the two homology domains of the Spike-LAMP comprise LAMP-1 Homology Domain 1 and LAMP-1 Homology Domain 2.

57. The isolated nucleic acid molecule of embodiment 56, wherein the human LAMP-1 Homology Domain 1 comprises the amino acid sequence of residues 29-194 of SEQ ID NO: 1, or comprises the amino acid sequence of residues 29-195 of SEQ ID NO: 198, or comprises an amino acid sequence at least 90%, at least 95%, at least 96%, at least 97%, at least 98%, or at least 99% identical to the amino acid sequence of residues 29-194 of SEQ ID NO: 1 or to the amino acid sequence of residues 29-195 of SEQ ID NO: 198, or wherein the nucleic acid molecule comprises the nucleotide sequence of SEQ ID NO: 199 or a nucleotide sequence at least 90%, at least 95%, at least 96%, at least 97%, at least 98%, or at least 99% identical to that of SEQ ID NO: 199 (wherein if the nucleotide sequence is RNA, T is replaced with U).

58. The isolated nucleic acid molecule of embodiment 56 or 57, wherein the human LAMP-1 Homology Domain 2 comprises the amino acid sequence of residues 228-381 or 228-382 of SEQ ID NO: 1, or of residues 228-382 of SEQ ID NO: 1, or comprises the amino acid sequence of SEQ ID NO: 202, or comprises an amino acid sequence at least 90%, at least 95%, at least 96%, at least 97%, at least 98%, or at least 99% identical to the amino acid sequence of residues 228-381 of SEQ ID NO: 1 or to the amino acid sequence of SEQ ID NO: 202, or wherein the nucleic acid molecule comprises the nucleotide sequence of SEQ ID NO: 203 or a nucleotide sequence at least 90%, at least 95%, at least 96%, at least 97%, at least 98%, or at least 99% identical to that of SEQ ID NO: 203 (wherein if the nucleotide sequence is RNA, T is replaced with U).

59. The isolated nucleic acid molecule of any one of embodiments 52-58, wherein the Spike-LAMP comprises a linker between at least one of the two homology domains and the antigenic domain.

60. The isolated nucleic acid molecule of embodiment 59, wherein the linker comprises an amino acid sequence of GPGPG or PMGLP.

61. The isolated nucleic acid molecule of any one of embodiments 52-60, wherein the Spike-LAMP further comprises a transmembrane domain of a LAMP Protein.

62. The isolated nucleic acid molecule of embodiment 61, wherein the transmembrane domain comprises residues 383 to 405 of SEQ ID NO: 1.

63. The isolated nucleic acid molecule of any one of embodiments 52-62, wherein the Spike-LAMP further comprises a signal sequence.

64. The isolated nucleic acid molecule of embodiment 63, wherein the signal sequence is derived from a LAMP Protein 65. The isolated nucleic acid molecule of any one of embodiments 52-64, wherein the Spike-LAMP further comprises cytoplasmic domain of a LAMP Protein.

66. The isolated nucleic acid molecule of embodiment 65, wherein the cytoplasmic domain comprises residues 406-417 of SEQ ID NO: 1.

67. The isolated nucleic acid molecule of any one of embodiments 52-66, wherein the antigenic domain comprises Spike S1 and/or S2, or an amino acid sequence comprising the sequence of SEQ ID NO: 118 or 119.

68. The isolated nucleic acid molecule of any one of embodiments 52-67, wherein the Spike-LAMP comprises or consists of the amino acid sequence of residues 1-194 of SEQ ID NO: 1 or of SEQ ID NO: 198 followed by the amino acid sequence of SEQ ID NO: 231 followed by the amino acid sequence of residues 228-381 of SEQ ID NO: 1 or of SEQ ID NO: 202.

69. The isolated nucleic acid molecule of any one of embodiments 52-68, wherein the IREG comprises one or more of CD40L, CD80, OX40, Flt3L, GM-CSF, IL-12, IL-21, IL-23, IL-15, CD70, CD86, IL-7, IL-18, or IL-33 (or comprises an amino acid sequence at least 90%, at least 95%, at least 96%, at least 97%, at least 98%, or at least 99% identical, or 100% identical to the amino acid sequence of any one of SEQ ID NOs: 133, 145, 147, 149, 151, 155, 159, 165, 169, 173, 177, 181, 189, 191, 204, 238, 242, 243, 252, or 253), or an extracellular domain thereof, optionally wherein the CD40L, CD80, OX40, Flt3L, GM-CSF, IL-12, IL-21, IL-23, or IL-15 is fused to an Fc domain of an immunoglobulin, or wherein the isolated nucleic acid molecule comprises a nucleotide sequence encoding CD40L, CD80, OX40, Flt3L, GM-CSF, IL-12, IL-21, IL-23, IL-15, CD70, CD86, IL-7, IL-18, or IL-33, optionally fused to an Fc domain, wherein the nucleotide sequence at least 90%, at least 95%, at least 96%, at least 97%, at least 98%, or at least 99% identical, or 100% identical to that of any one of SEQ ID NOs: 134, 146, 148, 150, 152, 160, 166, 170, 174, 178, 182, 190, 192, 205, 239, 244, or 881.

70. The isolated nucleic acid molecule of any one of embodiments 52-68, wherein the second polypeptide comprises an SPD-sCD40L fusion polypeptide.

71. The isolated nucleic acid molecule of any one of embodiments 52-70, wherein the secretion signal sequence is heterologous to the IREG.

72. The isolated nucleic acid molecule of embodiment 71, wherein the secretion signal sequence is derived from SPD.

73. The isolated nucleic acid molecule of any one of embodiments 52-72, wherein the second polypeptide is expressed under the control of an EF-1alpha core promoter, such as that of SEQ ID NO: 124.

74. A composition comprising the isolated nucleic acid molecule of any one of embodiments 52-73.

75. A host cell comprising the isolated nucleic acid of any one of embodiments 52-73.

76. A composition comprising the host cell of embodiment 75.

77. A method of treating a subject having or at risk of developing a coronavirus infection such as from COVID-19, wherein the method comprises administering to the subject the isolated nucleic acid molecule of any one of embodiments 52-73, the composition of embodiment 74, or the host cell of embodiment 75, in an amount sufficient to treat or prevent onset of or reduce the severity of symptoms of the coronavirus infection such as COVID-19.

78. A method of inducing an immune response against a coronavirus such as SARS Co-V2 in a subject, wherein the method comprises administering to the subject the isolated nucleic acid molecule of any one of embodiments 52-73, the composition of embodiment 74, or the host cell of embodiment 75, in an amount sufficient to induce an immune response against the coronavirus such as SARS Co-V2 in the subject.

79. An isolated nucleic acid molecule comprising
a. a first polynucleotide sequence encoding a polypeptide comprising two homology domains of a luminal domain of a LAMP protein, and an antigenic domain comprising a NY-ESO1 or CD161 protein antigen (a “NY-ESO1-LAMP” or “CD161-LAMP” LAMP-antigen Construct), wherein the antigenic domain is placed between the two LAMP homology domains; and
b. a second polypeptide sequence encoding at least one second polypeptide comprising an immune response enhancing gene polypeptide (IREG) or an extracellular domain of an IREG comprising a secretion signal sequence.

80. The isolated nucleic acid molecule of embodiment 79, wherein the LAMP protein is selected from LAMP-1, LAMP2, LAMP-3, lysosomal integral membrane protein-2 (“LIMP 2”), Macrosailin, Endolyn, LAMP5 or limbic system-associated membrane protein (“LIMBIC”).

81. The isolated nucleic acid molecule of embodiment 80, wherein the LAMP protein is selected from any one of SEQ ID NO:1-113.

82. The isolated nucleic acid molecule of embodiment 79 or 80, wherein the LAMP protein is at least 70%, at least 75%, at least 80%, at least 85%, at least 90%, at least 95%, at least 96%, at least 97%, at least 98%, or at least 99% identical to SEQ ID NO:1-113.

83. The isolated nucleic acid molecule of embodiment 79, wherein the LAMP protein is LAMP-1 and wherein the two homology domains of the NY-ESO1-LAMP or CD161-LAMP comprise LAMP-1 Homology Domain 1 and LAMP-1 Homology Domain 2.

84. The isolated nucleic acid molecule of embodiment 83, wherein the human LAMP-1 Homology Domain 1 comprises the amino acid sequence of residues 29-194 of SEQ ID NO: 1, or comprises the amino acid sequence of residues 29-195 of SEQ ID NO: 198, or comprises an amino acid sequence at least 90%, at least 95%, at least 96%, at least 97%, at least 98%, or at least 99% identical to the amino acid sequence of residues 29-194 of SEQ ID NO: 1 or to the amino acid sequence of residues 29-195 of SEQ ID NO: 198, or wherein the nucleic acid molecule comprises the nucleotide sequence of SEQ ID NO: 199 or a nucleotide sequence at least 90%, at least 95%, at least 96%, at least 97%, at least 98%, or at least 99% identical to that of SEQ ID NO: 199 (wherein if the nucleotide sequence is RNA, T is replaced with U).

85. The isolated nucleic acid molecule of embodiment 83 or 84, wherein the human LAMP-1 Homology Domain 2 comprises the amino acid sequence of residues 228-381 or 228-382 of SEQ ID NO: 1, or of residues 228-382 of SEQ ID NO: 1, or comprises the amino acid sequence of SEQ ID NO: 202, or comprises an amino acid sequence at least 90%, at least 95%, at least 96%, at least 97%, at least 98%, or at least 99% identical to the amino acid sequence of residues 228-381 of SEQ ID NO: 1 or to the amino acid sequence of SEQ ID NO: 202, or wherein the nucleic acid molecule comprises the nucleotide sequence of SEQ ID NO: 203 or a nucleotide sequence at least 90%, at least 95%, at least 96%, at least 97%, at least 98%, or at least 99% identical to that of SEQ ID NO: 203 (wherein if the nucleotide sequence is RNA, T is replaced with U).

86. The isolated nucleic acid molecule of any one of embodiments 79-85, wherein the LAMP-antigen Construct comprises a linker between at least one of the two homology domains and the antigenic domain.

87. The isolated nucleic acid molecule of embodiment 86, wherein the linker comprises an amino acid sequence of GPGPG or PMGLP.

88. The isolated nucleic acid molecule of any one of embodiments 79-87, wherein the LAMP-antigen Construct further comprises a transmembrane domain of a LAMP Protein.

89. The isolated nucleic acid molecule of embodiment 88, wherein the transmembrane domain comprises residues 383 to 405 of SEQ ID NO: 1.

90. The isolated nucleic acid molecule of any one of embodiments 79-89, wherein the LAMP-antigen Construct further comprises a signal sequence.

91. The isolated nucleic acid molecule of embodiment 90, wherein the signal sequence is derived from a LAMP Protein

92. The isolated nucleic acid molecule of any one of embodiments 79-91, wherein the LAMP-antigen Construct further comprises cytoplasmic domain of a LAMP Protein.

93. The isolated nucleic acid molecule of embodiment 92, wherein the cytoplasmic domain comprises residues 406-417 of SEQ ID NO: 1.

94. The isolated nucleic acid molecule of any one of embodiments 79-93, wherein the LAMP-antigen Construct comprises or consists of the amino acid sequence of residues 1-194 of SEQ ID NO: 1 or of SEQ ID NO: 198 followed by the amino acid sequence of SEQ ID NO: 223 followed by the amino acid sequence of residues 228-381 of SEQ ID NO: 1 or of SEQ ID NO: 202 or residues 1-194 of SEQ ID NO: 1 or of SEQ ID NO: 198 followed by the amino acid sequence of SEQ ID NO: 236 followed by the amino acid sequence of residues 228-381 of SEQ ID NO: 1 or of SEQ ID NO: 202.

95. The isolated nucleic acid molecule of any one of embodiments 79-94, wherein the IREG comprises one or more of CD40L, CD80, OX40, Flt3L, GM-CSF, IL-12, IL-21, IL-23, IL-15, CD70, CD86, IL-7, IL-18, or IL-33 (or comprises an amino acid sequence at least 90%, at least 95%, at least 96%, at least 97%, at least 98%, or at least 99% identical, or 100% identical to the amino acid sequence of any one of SEQ ID NOs: 133, 145, 147, 149, 151, 155, 159, 165, 169, 173, 177, 181, 189, 191, 204, 238, 242, 243, 252, or 253), or an extracellular domain thereof, optionally wherein the CD40L, CD80, OX40, Flt3L, GM-CSF, IL-12, IL-21, IL-23, or IL-15 is fused to an Fc domain of an immunoglobulin, or wherein the isolated nucleic acid molecule comprises a nucleotide sequence encoding CD40L, CD80, OX40, Flt3L, GM-CSF, IL-12, IL-21, IL-23, IL-15, CD70, CD86, IL-7, IL-18, or IL-33, optionally fused to an Fc domain, wherein the nucleotide sequence at least 90%, at least 95%, at least 96%, at least 97%, at least 98%, or at least 99% identical, or 100% identical to that of any one of SEQ ID NOs: 134, 146, 148, 150, 152, 160, 166, 170, 174, 178, 182, 190, 192, 205, 239, 244, or 881.

96. The isolated nucleic acid molecule of any one of embodiments 79-95, wherein the second polypeptide comprises an SPD-sCD40L fusion polypeptide or an IL-15 polypeptide (e.g., SEQ ID NO: 233 or 169 or 225).

97. The isolated nucleic acid molecule of any one of embodiments 79-96, wherein the secretion signal sequence is heterologous to the IREG.

98. The isolated nucleic acid molecule of embodiment 97, wherein the secretion signal sequence is derived from SPD or comprises SEQ ID NO: 120 or 122.

99. The isolated nucleic acid molecule of any one of embodiments 79-98, wherein the second polypeptide is expressed under the control of an EF-1alpha core promoter, such as that of SEQ ID NO: 124.

100. A composition comprising the isolated nucleic acid molecule of any one of embodiments 79-99.

101. A host cell comprising the isolated nucleic acid of any one of embodiments 79-99.

102. A composition comprising the host cell of embodiment 101.

103. A method of inducing an immune response in a subject in need thereof, comprising administering to the subject an amount of the isolated nucleic acid molecule of any one of embodiments 79-99, the composition of embodiment 100, or the host cell of embodiment 101 in an amount sufficient to induce an immune response in the subject.

104. An isolated nucleic acid molecule comprising
a. a first polynucleotide sequence encoding a polypeptide comprising two homology domains of a luminal domain of a LAMP protein, and an antigenic domain comprising a pp65 antigen such as comprising SEQ ID NO: 291, 292, or 293 and optionally further comprising one or both of a gB antigen such as comprising SEQ ID NO: 294, 295, 296, or 297 and a 1E1 antigen such as comprising SEQ ID NO: 298, 299, or 300, optionally comprising linker peptides between the pp65 and the gB and/or 1E1 antigen sequences (collectively “pp65-LAMP”), wherein the antigenic domain is placed between the two LAMP homology domains; and
b. a second polypeptide sequence encoding at least one second polypeptide comprising an immune response enhancing gene polypeptide (IREG) or an extracellular domain of an IREG comprising a secretion signal sequence.

105. The isolated nucleic acid molecule of embodiment 104, wherein the LAMP protein is selected from LAMP-1, LAMP2, LAMP-3, lysosomal integral membrane protein-2 (“LIMP 2”), Macrosailin, Endolyn, LAMP5 or limbic system-associated membrane protein (“LIMBIC”).

106. The isolated nucleic acid molecule of embodiment 105, wherein the LAMP protein is selected from any one of SEQ ID NO:1-113.

107. The isolated nucleic acid molecule of embodiment 104 or 105, wherein the LAMP protein is at least 70%, at least 75%, at least 80%, at least 85%, at least 90%, at least 95%, at least 96%, at least 97%, at least 98%, or at least 99% identical to SEQ ID NO:1-113.

108. The isolated nucleic acid molecule of embodiment 105, wherein the LAMP protein is LAMP-1 and wherein the two homology domains of the HER2-LAMP comprise LAMP-1 Homology Domain 1 and LAMP-1 Homology Domain 2.

109. The isolated nucleic acid molecule of embodiment 108, wherein the human LAMP-1 Homology Domain 1 comprises the amino acid sequence of residues 29-194 of SEQ ID NO: 1, or comprises the amino acid sequence of residues 29-195 of SEQ ID NO: 198, or comprises an amino acid sequence at least 90%, at least 95%, at least 96%, at least 97%, at least 98%, or at least 99% identical to the amino acid sequence of residues 29-194 of SEQ ID NO: 1 or to the amino acid sequence of residues 29-195 of SEQ ID NO: 198, or wherein the nucleic acid molecule comprises the nucleotide sequence of SEQ ID NO: 199 or a nucleotide sequence at least 90%, at least 95%, at least 96%, at least 97%, at least 98%, or at least 99% identical to that of SEQ ID NO: 199 (wherein if the nucleotide sequence is RNA, T is replaced with U).

110. The isolated nucleic acid molecule of embodiment 108 or 109, wherein the human LAMP-1 Homology Domain 2 comprises the amino acid sequence of residues 228-381 or 228-382 of SEQ ID NO: 1, or of residues 228-382 of SEQ ID NO: 1, or comprises the amino acid sequence of SEQ ID NO: 202, or comprises an amino acid sequence at least 90%, at least 95%, at least 96%, at least 97%, at least 98%, or at least 99% identical to the amino acid sequence of residues 228-381 of SEQ ID NO: 1 or to the amino acid sequence of SEQ ID NO: 202, or wherein the nucleic acid molecule comprises the nucleotide sequence of SEQ ID NO: 203 or a nucleotide sequence at least 90%, at least 95%, at least 96%, at least 97%, at least 98%, or at least 99% identical to that of SEQ ID NO: 203 (wherein if the nucleotide sequence is RNA, T is replaced with U).

111. The isolated nucleic acid molecule of any one of embodiments 104-110, wherein the pp65-LAMP comprises a linker between at least one of the two homology domains and the antigenic domain.

112. The isolated nucleic acid molecule of embodiment 111, wherein the linker comprises an amino acid sequence of GPGPG or PMGLP.

113. The isolated nucleic acid molecule of any one of embodiments 104-112, wherein the pp65-LAMP further comprises a transmembrane domain of a LAMP Protein.

114. The isolated nucleic acid molecule of embodiment 113, wherein the transmembrane domain comprises residues 383 to 405 of SEQ ID NO: 1.

115. The isolated nucleic acid molecule of any one of embodiments 104-114, wherein the pp65-LAMP further comprises a signal sequence.

116. The isolated nucleic acid molecule of embodiment 115, wherein the signal sequence is derived from a LAMP Protein

117. The isolated nucleic acid molecule of any one of embodiments 104-116, wherein the pp65-LAMP further comprises cytoplasmic domain of a LAMP Protein.

118. The isolated nucleic acid molecule of embodiment 117, wherein the cytoplasmic domain comprises residues 406-417 of SEQ ID NO: 1.

119. The isolated nucleic acid molecule of any one of embodiments 104-118, wherein the IREG comprises one or more of CD40L, CD80, OX40, Flt3L, GM-CSF, IL-12, IL-21, IL-23, IL-15, CD70, CD86, IL-7, IL-18, or IL-33 (or comprises an amino acid sequence at least 90%, at least 95%, at least 96%, at least 97%, at least 98%, or at least 99% identical, or 100% identical to the amino acid sequence of any one of SEQ ID NOs: 133, 145, 147, 149, 151, 155, 159, 165, 169, 173, 177, 181, 189, 191, 204, 238, 242, 243, 252, or 253), or an extracellular domain thereof, optionally wherein the CD40L, CD80, OX40, Flt3L, GM-CSF, IL-12, IL-21, IL-23, or IL-15 is fused to an Fc domain of an immunoglobulin, or wherein the isolated nucleic acid molecule comprises a nucleotide sequence encoding CD40L, CD80, OX40, Flt3L, GM-CSF, IL-12, IL-21, IL-23, IL-15, CD70, CD86, IL-7, IL-18, or IL-33, optionally fused to an Fc domain, wherein the nucleotide sequence at least 90%, at least 95%, at least 96%, at least 97%, at least 98%, or at least 99% identical, or 100% identical to that of any one of SEQ ID NOs: 134, 146, 148, 150, 152, 160, 166, 170, 174, 178, 182, 190, 192, 205, 239, 244, or 881.

120. The isolated nucleic acid molecule of any one of embodiments 104-119, wherein the secretion signal sequence is heterologous to the IREG.

121. The isolated nucleic acid molecule of embodiment 120, wherein the secretion signal sequence is derived from IgKVIII (e.g., SEQ ID NO: 122), Ig-kappa (e.g., SEQ ID NO: 120), tetranectin, or IL-2.

122. The isolated nucleic acid molecule of any one of embodiments 104-121, wherein the second polypeptide comprises a fusion of SPD and soluble CD40L (sCD40L), a fusion of SPD and Flt3L, IL-12, IL-21, OX40L fused to an Fc domain, CD80 fused to an Fc domain, or IL-15 (such as comprising an amino acid sequence at least 90%, at least 95%, at least 96%, at least 97%, at least 98%, or at least 99% identical to, or 100% identical to SEQ ID NO: 233, 238, 242, or 252, or comprising an amino acid sequence at least 90%, at least 95%, at least 96%, at least 97%, at least 98%, or at least 99% identical to, or 100% identical to: the amino acid sequence of SEQ ID NO: 131 followed by the amino acid sequence of one of SEQ ID NOs: 204, 151, 145, 147, 149, 193, 181, 155, 159, 169, 252, or 253), or wherein the nucleic acid comprises a nucleotide sequence encoding a fusion of SPD and soluble CD40L (sCD40L), a fusion of SPD and Flt3L, IL-12, IL-21, OX40L fused to an Fc domain, CD80 fused to an Fc domain, or IL-15 (such as comprising a nucleotide sequence at least 90%, at least 95%, at least 96%, at least 97%, at least 98%, or at least 99% identical to, or 100% identical to the sequence of: SEQ ID NO: 132 followed by one of SEQ ID NOs: 205, 152, 146, 148, 150, 194, 182, 156, 170 or 881).

123. The isolated nucleic acid molecule of embodiment 122, wherein the second polypeptide is expressed under the control of an EF-1alpha core promoter, such as that of SEQ ID NO: 124.

124. A composition comprising the isolated nucleic acid molecule of any one of embodiments 104-123.

125. A host cell comprising the isolated nucleic acid of any one of embodiments 104-123.

126. A composition comprising the host cell of embodiment 125.

127. A method of treating a subject having cancer, wherein the method comprises administering to the subject the isolated nucleic acid molecule of any one of embodiments 104-123, the composition of embodiment 124, or the host cell of embodiment 125, in an amount sufficient to treat the cancer or to induce an immune response in the subject against the cancer.

128. The method of embodiment 127, wherein the method further comprises administering at least one second therapeutic to the subject.

129. The method of embodiment 127 or 128, wherein the cancer is selected from glioblastoma, breast cancer, prostate cancer, colorectal cancer, and head and neck cancer.

130. An isolated nucleic acid molecule comprising
a. a first polynucleotide sequence encoding a polypeptide comprising two homology domains of a luminal domain of a LAMP protein, and an antigenic domain comprising a Large T antigen such as comprising the amino acid sequence of SEQ ID NO: 254, 255, or 256 (“LargeT-LAMP”), wherein the antigenic domain is placed between the two LAMP homology domains; and
b. a second polypeptide sequence encoding at least one second polypeptide comprising an immune response enhancing gene polypeptide (IREG) or an extracellular domain of an IREG comprising a secretion signal sequence.

131. The isolated nucleic acid molecule of embodiment 130, wherein the LAMP protein is selected from LAMP-1, LAMP2, LAMP-3, lysosomal integral membrane protein-2 (“LIMP 2”), Macrosailin, Endolyn, LAMP5 or limbic system-associated membrane protein (“LIMBIC”).

132. The isolated nucleic acid molecule of embodiment 131, wherein the LAMP protein is selected from any one of SEQ ID NO:1-113.

133. The isolated nucleic acid molecule of embodiment 130 or 131, wherein the LAMP protein is at least 70%, at least 75%, at least 80%, at least 85%, at least 90%, at least 95%, at least 96%, at least 97%, at least 98%, or at least 99% identical to SEQ ID NO:1-113.

134. The isolated nucleic acid molecule of embodiment 131, wherein the LAMP protein is LAMP-1 and wherein the two homology domains of the HER2-LAMP comprise LAMP-1 Homology Domain 1 and LAMP-1 Homology Domain 2.

135. The isolated nucleic acid molecule of embodiment 134, wherein the human LAMP-1 Homology Domain 1 comprises the amino acid sequence of residues 29-194 of SEQ ID NO: 1, or comprises the amino acid sequence of residues 29-195 of SEQ ID NO: 198, or comprises an amino acid sequence at least 90%, at least 95%, at least 96%, at least 97%, at least 98%, or at least 99% identical to the amino acid sequence of residues 29-194 of SEQ ID NO: 1 or to the amino acid sequence of residues 29-195 of SEQ ID NO: 198, or wherein the nucleic acid molecule comprises the nucleotide sequence of SEQ ID NO: 199 or a nucleotide sequence at least 90%, at least 95%, at least 96%, at least 97%, at least 98%, or at least 99% identical to that of SEQ ID NO: 199 (wherein if the nucleotide sequence is RNA, T is replaced with U).

136. The isolated nucleic acid molecule of embodiment 134 or 135, wherein the human LAMP-1 Homology Domain 2 comprises the amino acid sequence of residues 228-381 or 228-382 of SEQ ID NO: 1, or of residues 228-382 of SEQ ID NO: 1, or comprises the amino acid sequence of SEQ ID NO: 202, or comprises an amino acid sequence at least 90%, at least 95%, at least 96%, at least 97%, at least 98%, or at least 99% identical to the amino acid sequence of residues 228-381 of SEQ ID NO: 1 or to the amino acid sequence of SEQ ID NO: 202, or wherein the nucleic acid molecule comprises the nucleotide sequence of SEQ ID NO: 203 or a nucleotide sequence at least 90%, at least 95%, at least 96%, at least 97%, at least 98%, or at least 99% identical to that of SEQ ID NO: 203 (wherein if the nucleotide sequence is RNA, T is replaced with U).

137. The isolated nucleic acid molecule of any one of embodiments 130-136, wherein the LargeT-LAMP comprises a linker between at least one of the two homology domains and the antigenic domain.

138. The isolated nucleic acid molecule of embodiment 137, wherein the linker comprises an amino acid sequence of GPGPG or PMGLP.

139. The isolated nucleic acid molecule of any one of embodiments 130-138, wherein the LargeT-LAMP further comprises a transmembrane domain of a LAMP Protein.

140. The isolated nucleic acid molecule of embodiment 140, wherein the transmembrane domain comprises residues 383 to 405 of SEQ ID NO: 1.

141. The isolated nucleic acid molecule of any one of embodiments 130-140, wherein the pp65-LAMP further comprises a signal sequence.

142. The isolated nucleic acid molecule of embodiment 141, wherein the signal sequence is derived from a LAMP Protein

143. The isolated nucleic acid molecule of any one of embodiments 130-142, wherein the LargeT-LAMP further comprises cytoplasmic domain of a LAMP Protein.

144. The isolated nucleic acid molecule of embodiment 143, wherein the cytoplasmic domain comprises residues 406-417 of SEQ ID NO: 1.

145. The isolated nucleic acid molecule of any one of embodiments 130-144, wherein the IREG comprises one or more of CD40L, CD80, OX40, Flt3L, GM-CSF, IL-12, IL-21, IL-23, IL-15, CD70, CD86, IL-7, IL-18, or IL-33 (or comprises an amino acid sequence at least 90%, at least 95%, at least 96%, at least 97%, at least 98%, or at least 99% identical, or 100% identical to the amino acid sequence of any one of SEQ ID NOs: 133, 145, 147, 149, 151, 155, 159, 165, 169, 173, 177, 181, 189, 191, 204, 238, 242, 243, 252, or 253), or an extracellular domain thereof, optionally wherein the CD40L, CD80, OX40, Flt3L, GM-CSF, IL-12, IL-21, IL-23, or IL-15 is fused to an Fc domain of an immunoglobulin, or wherein the isolated nucleic acid molecule comprises a nucleotide sequence encoding CD40L, CD80, OX40, Flt3L, GM-CSF, IL-12, IL-21, IL-23, IL-15, CD70, CD86, IL-7, IL-18, or IL-33, optionally fused to an Fc domain, wherein the nucleotide sequence at least 90%, at least 95%, at least 96%, at least 97%, at least 98%, or at least 99% identical, or 100% identical to that of any one of SEQ ID NOs: 134, 146, 148, 150, 152, 160, 166, 170, 174, 178, 182, 190, 192, 205, 239, 244, or 881.

146. The isolated nucleic acid molecule of any one of embodiments 130-145, wherein the secretion signal sequence is heterologous to the IREG.

147. The isolated nucleic acid molecule of embodiment 146, wherein the secretion signal sequence is derived from IgKVIII (e.g., SEQ ID NO: 122), Ig-kappa (e.g., SEQ ID NO: 120), tetranectin, or IL-2.

148. The isolated nucleic acid molecule of any one of embodiments 130-147, wherein the second polypeptide comprises a fusion of SPD and soluble CD40L (sCD40L), a fusion of SPD and Flt3L, IL-12, IL-21, OX40L fused to an Fc domain, CD80 fused to an Fc domain, or IL-15 (such as comprising an amino acid sequence at least 90%, at least 95%, at least 96%, at least 97%, at least 98%, or at least 99% identical to, or 100% identical to SEQ ID NO: 233, 238, 242, or 252, or comprising an amino acid sequence at least 90%, at least 95%, at least 96%, at least 97%, at least 98%, or at least 99% identical to, or 100% identical to: the amino acid sequence of SEQ ID NO: 131 followed by the amino acid sequence of one of SEQ ID NOs: 204, 151, 145, 147, 149, 193, 181, 155, 159, 169, 252, or 253), or wherein the nucleic acid comprises a nucleotide sequence encoding a fusion of SPD and soluble CD40L (sCD40L), a fusion of SPD and Flt3L, IL-12, IL-21, OX40L fused to an Fc domain, CD80 fused to an Fc domain, or IL-15 (such as comprising a nucleotide sequence at least 90%, at least 95%, at least 96%, at least 97%, at least 98%, or at least 99% identical to, or 100% identical to the sequence of: SEQ ID NO: 132 followed by one of SEQ ID NOs: 205, 152, 146, 148, 150, 194, 182, 156, 170 or 881).

149. The isolated nucleic acid molecule of embodiment 148, wherein the second polypeptide is expressed under the control of an EF-1alpha core promoter, such as that of SEQ ID NO: 124.

150. A composition comprising the isolated nucleic acid molecule of any one of embodiments 130-149.

151. A host cell comprising the isolated nucleic acid of any one of embodiments 130-149.

152. A composition comprising the host cell of embodiment 151.

153. A method of treating a subject having cancer, wherein the method comprises administering to the subject the isolated nucleic acid molecule of any one of embodiments 130-149, the composition of embodiment 150, or the host cell of embodiment 151, in an amount sufficient to treat the cancer or to induce an immune response in the subject against the cancer.

154. The method of embodiment 153, wherein the method further comprises administering at least one second therapeutic to the subject.

155. The method of embodiment 153 or 154, wherein the cancer is skin cancer, such as Merkel cell carcinoma.

156. The isolated nucleic acid of any one of embodiments 1-19, 25-46, 52-73, 79-99, 104-123, or 130-149, wherein the isolated nucleic acid comprises DNA, mRNA, or self-amplifying RNA.

157. A set of polypeptides encoded by the isolated nucleic acid of any one of embodiments 1-19, 25-46, 52-73, or 79-99, 104-123, or 130-149.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

The objects and features of the disclosure can be better understood with reference to the following detailed description and accompanying drawings.

FIG. 1 illustrates the general scheme of different types of improved LAMP-antigen Constructs (identified as ILC-1, ILC-2, ILC-3, ILC-4, ILC-5 and ILC-6) that can be used as described herein. Certain backbone constructs are further described in U.S. Pat. No. 11,203,629, which disclosure is incorporated by reference in its entirety.

FIG. 2B illustrates the domains of the LAMP proteins defined herein while FIG. 2A defines the specific amino acid boundaries of these domains for human LAMP-1 (SEQ ID NO: 1), human LAMP-2 (SEQ ID NO: 2), human LAMP-3 (SEQ ID NO: 3), human LIMP-2 (SEQ ID NO: 4), human Endolyn (SEQ ID NO: 5), human Macrosialin (SEQ ID NO: 80), human LAMP-5 (SEQ ID NO: 93) and human LIMBIC (SEQ ID NO: 67). As described herein the LAMP luminal domains, Homology Domains, transmembrane domains, the cytoplasmic tail and the signal sequences can be used to generate the bicistronic LAMP constructs as described herein.

FIG. 3 provides alignment of LAMP-1 proteins found in other species as compared to human LAMP-1 (SEQ ID NO: 1). The equivalent domains of these other species can be used to generate the bicistronic LAMP constructs described herein and are readily identifiable by comparing the domains identified for human LAMP-1 in FIG. 2 and FIG. 3 to the alignments shown in FIG. 3.

FIG. 4 provides alignment of LAMP-2 proteins found in other species as compared to human LAMP-2 (SEQ ID NO: 2). The equivalent domains of these other species can be used to generate the bicistronic LAMP constructs described herein and are readily identifiable by comparing the domains identified for human LAMP-2 in FIG. 2 and FIG. 4 to the alignments shown in FIG. 4.

FIG. 5 provides alignment of LAMP-3 proteins found in other species as compared to human LAMP-3 (SEQ ID NO: 3). The equivalent domains of these other species can be used to generate the bicistronic LAMP constructs described herein and are readily identifiable by comparing the domains identified for human LAMP-3 in FIG. 2 and FIG. 5 to the alignments shown in FIG. 5.

FIG. 6 provides alignment of LIMP-2 proteins found in other species as compared to human LIMP-2 (SEQ ID NO: 4). The equivalent domains of these other species can be used to generate the bicistronic LAMP constructs described herein and are readily identifiable by comparing the domains identified for human LIMP-2 in FIG. 2 and FIG. 6 to the alignments shown in FIG. 6.

FIG. 7 provides alignment of LIMBIC proteins found in other species as compared to human LIMBIC (SEQ ID NO: 67). The equivalent domains of these other species can be used to generate the bicistronic LAMP constructs described herein and are readily identifiable by comparing the domains identified for human LIMBIC in FIG. 2 and FIG. 7 to the alignments shown in FIG. 7.

FIG. 8 provides alignment of Endolyn proteins found in other species as compared to human Endolyn (SEQ ID NO: 5). The equivalent domains of these other species can be used to generate the bicistronic LAMP constructs described herein and are readily identifiable by comparing the domains identified for human Endolyn in FIG. 2 and FIG. 8 to the alignments shown in FIG. 8.

FIG. 9 provides alignment of Macrosialin proteins found in other species as compared to human Macrosialin (SEQ ID NO:80). The equivalent domains of these other species can be used to generate the bicistronic LAMP constructs described herein and are readily identifiable by comparing the domains identified for human Macrosialin in FIG. 2 and FIG. 9 to the alignments shown in FIG. 9.

FIG. 10 provides alignment of LAMP-5 proteins found in other species as compared to human LAMP-5 (SEQ ID NO: 93). The equivalent domains of these other species can be used to generate the bicistronic LAMP constructs described herein and are readily identifiable by comparing the domains identified for human LAMP-5 in FIG. 2 and FIG. 10 to the alignments shown in FIG. 10.

FIG. 11 shows design of the exemplary bicistronic construct HER2-LAMP-sCD40L.

FIG. 12 shows detection of sCD40L in the supernatant of 293T cells transfected with bicistronic HER2-LAMP-sCD40L vaccine. The asterisk marks the protein band that indicates the presence of sCD40L. Immunoprecipitation experiment with a biotinylated anti-CD40L antibody was used to detect sCD40L in control 293T cells, 293T cells transfected with bicistronic HER2-LAMP-sCD40L, or 293T cells transfected with a control vector expressing GFP. Cell culture supernatant was used as Input (I). Flowthrough (FT) marks proteins that didn't bind to the biotinylated anti-CD40L whereas Bound (B) marks proteins that bind to the biotinylated anti-CD40L antibody.

FIGS. 13A-D show detection of Spike-specific T cell response in mice after one immunization (FIGS. 13A-B) or two immunizations (FIGS. 13C-D) of ITI-bicistronic vaccine or 2-V COVID vaccine. FIGS. 13A and 13C: ELISPOT. FIGS. 13B and 13D: spot counts. CV=control vector (plasmid alone). SFC=spot forming cells. Student T test was used to determine significance. * p<0.05, ** p<0.01, *** p<0.001.

FIG. 14 shows detection of spike specific CD4 and CD8 T cells by flow cytometry. Both CD4+ and CD8+ T cell responses were enhanced after vaccination with 2-V vaccine.

FIG. 15 shows intracellular cytokine staining (ICS) of IFNγ, TNFα, and IL-2 in CD4+ and CD8+ T cells.

FIGS. 16A-F show measurements of S1-specific antibodies after one immunization (FIGS. 16A, 16C, and 16E) vs 2 immunizations (FIGS. 16B, 16D, and 16F) with either ITI-bicistronic vaccine or 2-V vaccine. Student T test was used to determine significance.

FIGS. 17A-B detection of HER2-LAMP-sCD40L in the supernatant of transfected 293T cells. FIG. 17A shows a sCD40L standard curve for ELISA. FIG. 17B shows detection of sCD40L in the supernatant. Control cells=cells without transfection with a bicistronic construct.

FIGS. 18A-F show bicistronic vaccines are capable of eliciting robust T-cell and antibody response. FIG. 18A show an exemplary vaccination schedule where mice were immunized by intradermal (ID) injection with 20 μg of control vector, HER2-LAMP, or bicistronic HER2-LAMP-sCD40L. FIG. 18B shows IFNγ spot forming cells (ELISPOT). FIG. 18C shows mean IFNγ spot-forming cells±SEM. FIG. 18D-F show HER2-specific total IgG (FIG. 18D), IgG1 (FIG. 18E), and IgG2a (FIG. 18F) by ELISA; N=7. One-way ANOVA was used for statistical analysis. * p<0.05, ** p<0.01, *** p<0.001, **** p<0.0001.

FIGS. 19A-B show intracellular staining for cytokines IFNγ (IFNg), TNFα (TNFa), and IL-2 in CD4 and CD8 T cells. FIG. 19A show % of stained cells as mean±SEM. FIG. 19B show representative FACS plots. * p<0.05, ** p<0.01, *** p<0.001.

FIG. 20 shows HER2-specific antibody responses as determined by ELISA.

FIGS. 21A-B show HER2-LAMP-sCD40L protects mice form HER2-expressing breast tumor. FIG. 21A shows results by ELISPOT. FIG. 21B show tumor size as measured with a caliper.

FIGS. 22A-B show HER2-LAMP-sCD40L enhances survival in mice. FIG. 22A is a schematic of the experimental design. FIG. 22B shows mouse survival after tumor challenge in a single experiment. N=10.

FIG. 23 shows the effect of HER2-LAMP vaccines with immune response enhancing-genes (IREG; HER2-LAMP-IREG) on tumor volume. IREG=CD40L, Flt3L, IL-21, IL-12, and OX40L. ** p<0.01; **** p<0.0001.

FIG. 24 shows the effect of HER2-LAMP-IREG on mouse survival.

FIG. 25 shows HER2-LAMP-IL-15 induces strong antigen specific antibody response as determined by ELISA. N=5 per group. T-test was used for statistical analysis. * p<0.05, ** p<0.01.

FIG. 26 shows HER2-LAMP-IL-15 elicits strong T cell response as determined by ELISPOT. Data represent mean IFNγ spot forming cells±SEM. N=5. T-test was used for statistical analysis. * p<0.05, ** p<0.01.

FIGS. 27A-B show two doses of HER2-LAMP-IL-15 elicit strong T cell response as determined by ELISPOT. Data represent original spots (FIG. 27A) and mean IFNγ spot forming cells±SEM (FIG. 27B). N=5. T-test was used for statistical analysis. * p<0.05, ** p<0.01, *** p<0.001, *** p<0.0001.

FIGS. 28A-C relate to the first generation bicistronic LAMP construct comprising SARS CoV-2 S1 spike protein. FIG. 28A illustrates the general scheme of a vector (bicistronic-S1-LAMP-EF1 IgK-S 2P; 7427 bp) encoding a first generation bicistronic LAMP construct (based on ILC-4) comprising a fragment of the SARS COV-2 S1 spike protein as a target antigen, and including an expression cassette for expressing the SARS COV-2 S2 spike protein operably linked to an Ig-kappa leader (secretion signal), as a second antigen for secretion. FIG. 28B is another representation (as a circular plasmid) of the first-generation ITI-COVID bicistronic construct, showing a representative, yet preferred, example of the arrangement of polynucleotides encoding sequences. FIG. 28C shows the complete polynucleotide and encoded polypeptide domains of the first-generation ITI-COVID bicistronic construct.

FIGS. 29A-D show suppression of tumor growth by a bicistronic HER2-LAMP-sCD40L (soluble CD40 ligand) administered as a DNA vector in a murine TSA breast cancer model compared to control (CV) and non-bicistronic HER2-LAMP vectors. FIG. 29A shows the experimental protocol. FIG. 29B shows changes in tumor growth for each mouse, while FIG. 29C shows changes in tumor growth for the seven mice in each group (control, HER2-LAMP and HER2-LAMP-sCD40L). Control (CV; top-most curves) is depicted by closed circles; HER2-LAMP (middle curve in FIG. 29C) by closed squares; HER2-LAMP-sCD40L (bottom curve in FIG. 29C) by closed triangles. FIG. 29D shows tumor weight measured at the termination of the experiment (* indicates p<0.05 while ** indicates p<0.01).

FIG. 30 shows that HER2-LAMP-sCD40L induces production of CD3+ memory T cells compared to the control vector.

FIGS. 31A-C show that HER2-LAMP-sCD40L promotes infiltration of T cells into the tumor microenvironment compared with HER2-LAMP and control vectors. FIG. 31A shows the number of CD3+ T cells in tumors, after tumors were obtained, cleaned, and dissociated.

FIG. 31B shows the number of CD4+ T cells. FIG. 31C shows the number of CD8+ T cells. * indicates p<0.05 while ** indicates p<0.01.

FIG. 32A-C show that soluble CD40 ligand (sCD40L) produced by the HER2-LAMP-sCD40L vector enhances activation of type 1 dendritic cells (DC1) producing IL-12 in draining lymph nodes. FIG. 32A shows the gating protocol used in the experiment, while FIG. 32B shows CD8-expressing DC1 cells and FIG. 32C shows IL-12 producing DC1 cells. * indicates p<0.05.

FIG. 33A-B show that bicistronic HER2-LAMP-sCD40L activates inflammatory signals in the tumor microenvironment. Specifically, FIG. 33A shows the number of CD4+ CD69+ cells in tumors and FIG. 33B shows the number of CD8+ CD69+ cells in tumors, after tumors were obtained, cleaned, and dissociated. * indicates p<0.05.

FIG. 34A-B show that bicistronic HER2-LAMP-sCD40L promotes T cells producing PD-1 in the tumor microenvironment. Specifically, FIG. 33A shows the number of CD4+ PD-1+ cells in tumors and FIG. 33B shows the number of CD8+ PD-1+ cells in tumors, after tumors were obtained, cleaned, and dissociated. * indicates p<0.05 while ** indicates p<0.01.

FIG. 35 shows results of ELISPOT analysis of splenocytes from mice following the protocol described in FIG. 29A after incubation of splenocytes with several different pooled peptides of HER2 extracellular domain. The results show that the bicistronic HER2-LAMP-sCD40L induced a stronger response against certain pooled HER2 peptides than control or HER2-LAMP constructs.

FIG. 36 shows results from a parallel experiment to those described in FIGS. 29A-C, with 5 mice per control, HER2-LAMP and HER2-LAMP-sCD40L group, confirming that the bicistronic construct suppresses tumor growth more than the other two groups with p<0.05.

FIG. 37A-D show results of FACS analysis of splenocytes from the experiment shown in FIG. 36 (following the protocol of FIG. 29A), and shows that HER2-LAMP-sCD40L induces polyfunctional CD4 effector memory T cells (“TEM” cells) in the spleen. FIG. 37A shows percentage of CD4 TEM cells; FIG. 37B shows percentage of CD8 TEM cells. The amount of the CD4 or CD8 TEM cells expressing IFNg, TNFa, or both IFNg and TNFa are denoted in the bar graphs. The remaining figure panels show response to pooled HER2 peptides by CD4 TEM cells (FIG. 37C) or CD8 TEM cells (FIG. 37D) expressing both IFNg and TNFa. * P<0.05, ** p<0.01, **** p<0.0001.

FIG. 38A-B show that soluble CD40L expressed from the HER2-LAMP-sCD40L construct in the murine TSA model of FIG. 29A enhances activation of DC1 dendritic cells in the spleen. The FACS gating strategy is shown in FIG. 38A while the percentage of DC1 dendritic cells is shown in FIG. 38B. * indicates p<0.05 while ** indicates p<0.01.

FIG. 39A-B show results from cell staining experiments indicating that soluble CD40L expressed from the HER2-LAMP-sCD40L construct in the murine TSA model of FIG. 29A increases the presence of CD4+ (FIG. 39A) and CD8+ T cells (FIG. 39B) in the tumors.

FIGS. 40A-B show results from experiments in which mice were injected with a control vector (CV), Her2-LAMP (i.e., HER2-Hinge-LAMP), Her2-LAMP-sCD40L, Her2-LAMP-mFlt3L, or a combination of both Her2-LAMP-sCD40L and Her2-LAMP-mFlt3L (7 mice per group), followed by ELISPOT analysis of splenocytes. FIG. 40A shows mean IFNg forming cells +/−SEM for each group, while FIG. 40B shows the number of cells recognizing various HER2 peptide pools from each of the 5 groups.

FIG. 41 shows antibody titer determined by ELISA for each of the groups of FIG. 40A.

FIGS. 42A-D show the percentage of various CD4 and CD8 TEM cells recognizing various HER2 extracellular domain peptide pools following the experiment of FIG. 40A, specifically CD8 IFNg TNFa cells (FIG. 42A), CD8 IFNg cells (FIG. 42B), CD4 IFNg TNFa cells (FIG. 42C), and CD4 IFNg cells (FIG. 42D).

FIG. 43 shows the effect on serum antibody titer of combined administration of two bicistronic constructs HER2-LAMP-IL-12 and HER2-LAMP-mFlt3L, as measured by ELISA.

### DETAILED DESCRIPTION

The disclosure encompasses, for example, nucleic acid molecules encoding bicistronic or multicistronic LAMP constructs which can be used to generate vaccines and/or used to raise antibodies and/or a humoral immune response. The nucleic acid molecules can be used to induce an immune response. In one aspect, the disclosure provides methods for treating a subject with an allergy, infectious disease such as a coronavirus or Covid-19, diabetes, cancer or a hyperproliferative disorder by providing a nucleic acid molecule (e.g., a plasmid or vector) encoding a bicistronic LAMP construct described herein. The nucleic acid molecules encoding bicistronic LAMP constructs can also be used to raise antibodies in non-human vertebrates, and in preferably, non-human mammals.

### A. Definitions

The following definitions are provided for specific terms which are used in the following written description.

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

As used herein, the term “comprising” is intended to mean that the nucleic acid molecules or the bicistronic LAMP constructs and methods include the recited elements, but do not exclude other elements. In the case of an amino acid or nucleotide sequence, it is intended to mean that other sequence elements may be added to either end of the sequence. “Consisting essentially of”, when used to define nucleic acid molecules, bicistronic LAMP constructs and methods, shall mean excluding other elements of any essential significance to the combination or its function. Thus, a bicistronic LAMP construct consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering nucleic acid molecules encoding the bicistronic LAMP constructs described herein. In the case of an amino acid or nucleotide sequence, “consisting of” indicates that no further sequence elements are added to either end of the sequence, but the recited sequence would be allowed to incorporate modifications to the amino acids or nucleotides that occur physiologically such as DNA methylations or glycosylations or the like. Embodiments defined by each of these transition terms are within the scope of this disclosure.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2 fold, of a value. Unless otherwise stated, the term “about” means within an acceptable error range for the particular value, such as ±1-20%, preferably ±1-10% and more preferably ±1-5%.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding both of those included limits are also included in the disclosure.

As used herein, “the lysosomal/endosomal compartment” refers to membrane-bound acidic vacuoles containing LAMP molecules in the membrane, hydrolytic enzymes that function in antigen processing, and MHC class II molecules for antigen recognition and presentation. This compartment functions as a site for degradation of foreign materials internalized from the cell surface by any of a variety of mechanisms including endocytosis, phagocytosis and pinocytosis, and of intracellular material delivered to this compartment by specialized autolytic phenomena (de Duve, Eur. J. Biochem. 137:391, 1983). The term “endosome” as used herein encompasses a lysosome.

As used herein, a “lysosome-related organelle” refers to any organelle which comprises lysosymes and includes, but is not limited to, MIIC, CIIV, melanosomes, secretory granules, lytic granules, platelet-dense granules, basophil granules, Birbeck granules, phagolysosomes, secretory lysosomes, and the like. Preferably, such an organelle lacks mannose 6-phosphate receptors and comprises LAMP, but may or may not comprise an MHC class II molecule. For reviews, see, e.g., Blott and Griffiths, Nature Reviews, Molecular Cell Biology, 2002; Dell'Angelica, et al., The FASEB Journal 14:1265-1278, 2000.

As used herein, the terms “polynucleotide” and “nucleic acid molecule” are used interchangeably to refer to polymeric forms of nucleotides of any length. The polynucleotides may contain deoxyribonucleotides, ribonucleotides, and/or their analogs. Thus, in some cases, bicistronic LAMP construct nucleic acid molecule herein can be a DNA molecule, such as a DNA vector, e.g., a DNA virus vector, and in other cases it can be an RNA molecule, including a self-amplifying RNA vector (also known as a self-replicating RNA vector).

Nucleotides may have any three-dimensional structure, and may perform any function, known or unknown. Depending on context, the term “polynucleotide” also includes, for example, single-, double-stranded and triple helical molecules, a gene or gene fragment, exons, introns, mRNA, tRNA, rRNA, ribozymes, antisense molecules, cDNA, recombinant polynucleotides, branched polynucleotides, aptamers, plasmids, vectors, isolated DNA of any sequence, isolated RNA of any sequence, nucleic acid probes, and primers. A nucleic acid molecule may also comprise modified nucleic acid molecules (e.g., comprising modified bases, sugars, and/or internucleotide linkers).

As used herein, the term “peptide” refers to a compound of two or more subunit amino acids, amino acid analogs, or peptidomimetics. The subunits may be linked by peptide bonds or by other bonds (e.g., as esters, ethers, and the like).

As used herein, the term “amino acid” refers to either natural and/or unnatural or synthetic amino acids, including glycine and both D or L optical isomers, and amino acid analogs and peptidomimetics. A peptide of three or more amino acids is commonly called an oligopeptide if the peptide chain is short. If the peptide chain is long (e.g., greater than about 10 amino acids), the peptide is commonly called a polypeptide or a protein. While the term “protein” encompasses the term “polypeptide”, a “polypeptide” may be a less than full-length protein.

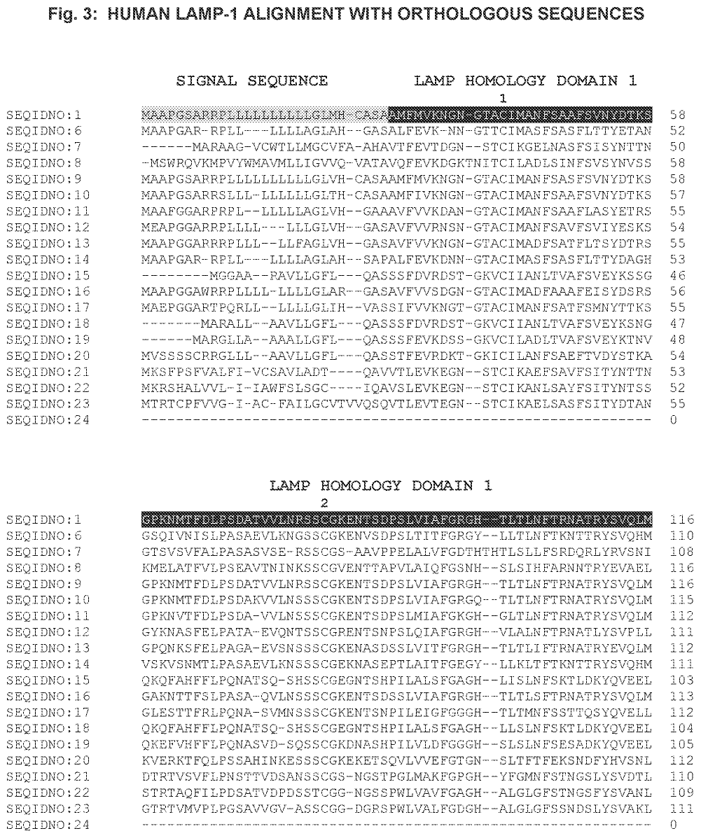

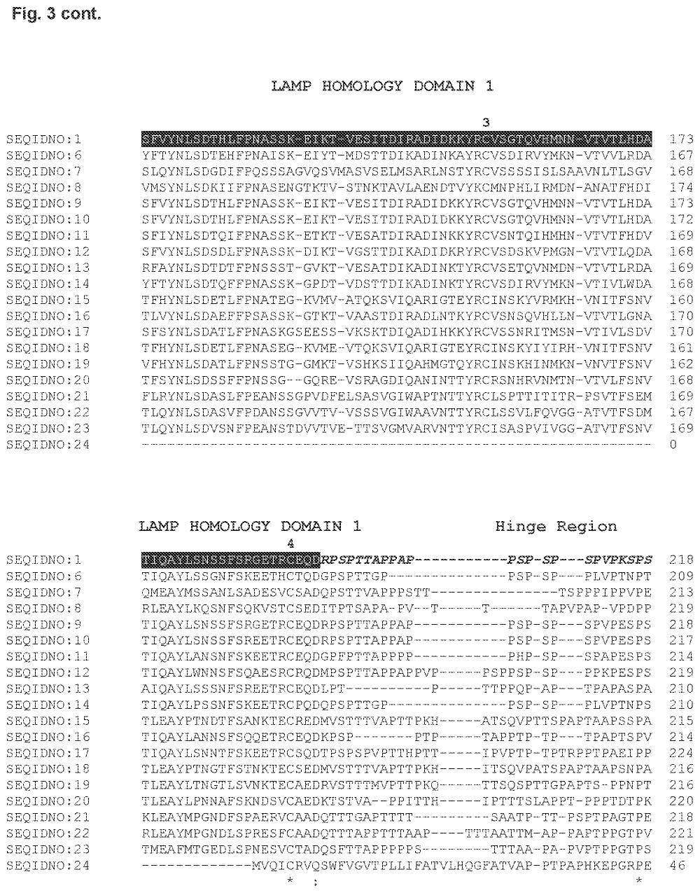

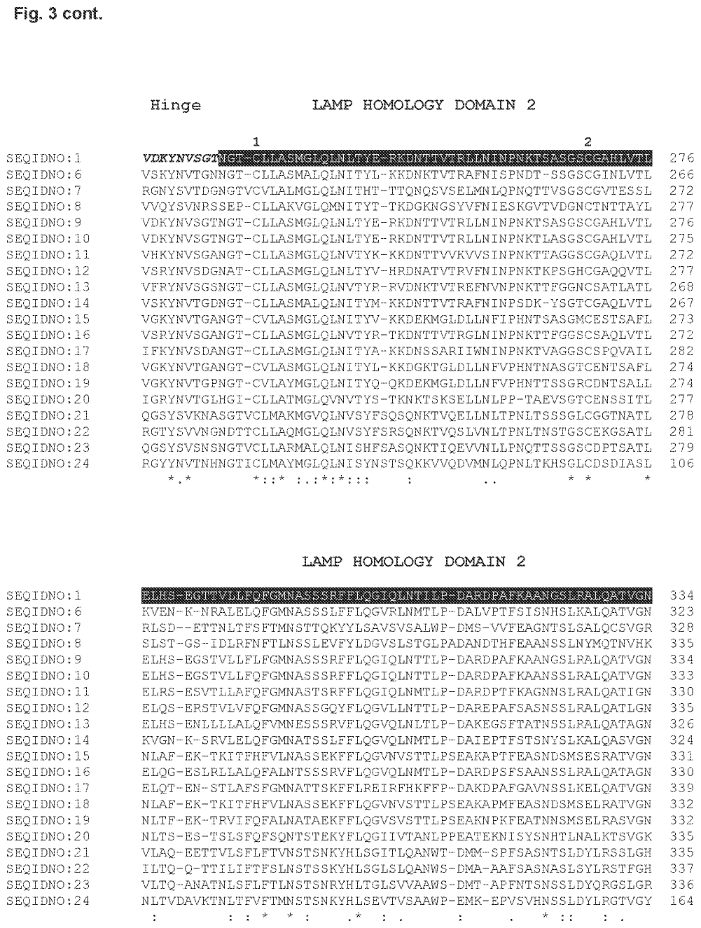

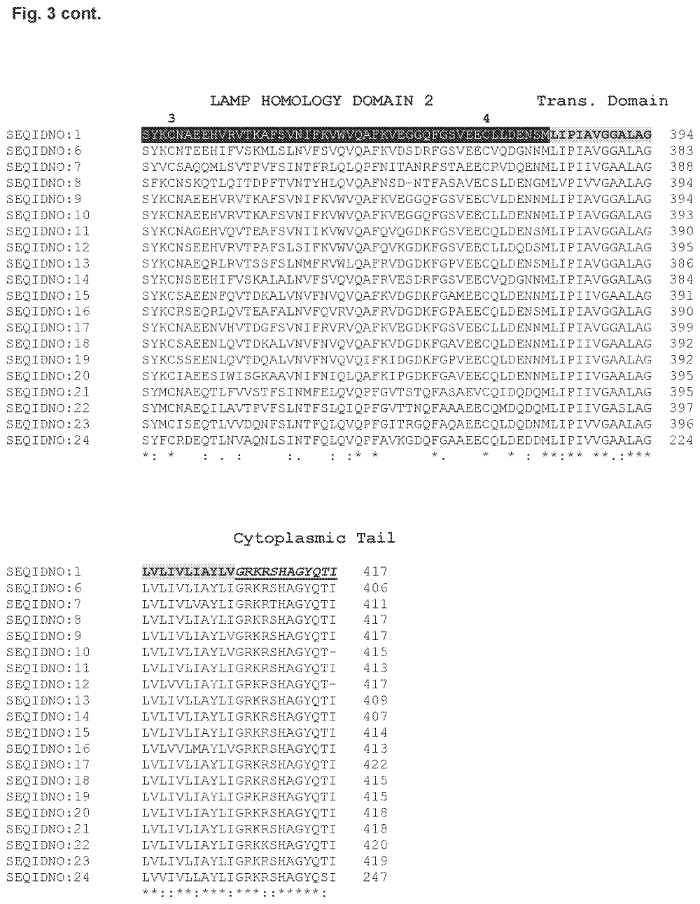

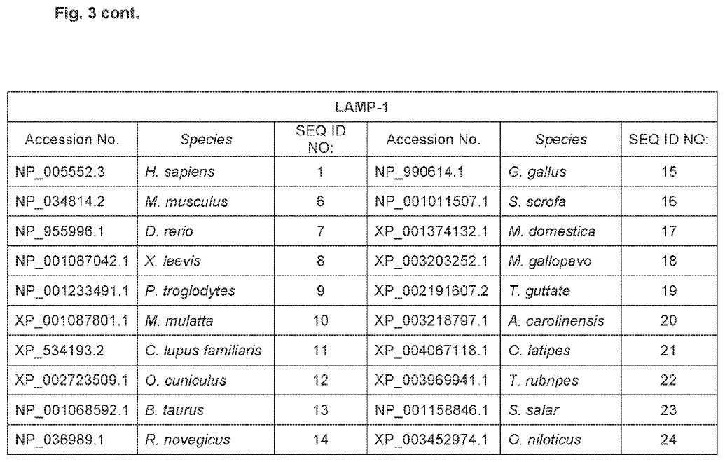

As used herein a “LAMP protein” or “LAMP polypeptide” refers to any of the mammalian lysosomal associated membrane proteins human LAMP-1, human LAMP-2, human LAMP-3, human LIMP-2, human Endolyn, human LIMBIC, human LAMP-5, or human Macrosialin as described herein, as well as orthologs (such as, for example, the LAMP proteins shown in FIGS. 3-10), and their allelic variants. As used herein, LAMP-1, LAMP2, LAMP-3, LIMP 2, Macrosialin, Endolyn, LAMP5 or LIMBIC refer to the human proteins and their allelic variants as noted in FIGS. 3-10 unless explicitly noted otherwise.

As used herein, a LAMP “homology domain” comprises at least the 4 uniformly spaced cysteine residues shown in FIGS. 3-10. These cysteine resides are labeled 1, 2, 3, and 4 (and in LIMP-2 and Macrosialin-five cysteines are identified, LIMBIC-six cysteines are identified and Endolyn-eight cysteines are identified) in each Homology Domain as shown in FIGS. 3-10 and are defined herein as the “Cysteine Conserved Fragment.” Additional amino acids can be included to either the N-terminus end and/or the C-terminus end of the Cysteine Conserved Fragment to generate, up to and including a full Homology Domain of a LAMP protein. These additional added amino acids can be derived from the Homology Domain from which the Cysteine Conserved Fragment is derived or from other LAMP Protein Homology Domains. Thus, as used herein, a LAMP Homology Domain comprises and/or consists of one Cysteine Conserved Fragment. At least two LAMP Homology Domains make up the Luminal Domain of LAMP-1, LAMP-2, LAMP-3, or Endolyn.

As used herein, “expression” refers to the process by which polynucleotides are transcribed into mRNA and/or translated into peptides, polypeptides, or proteins. If the polynucleotide is derived from genomic DNA, expression may include splicing of the mRNA transcribed from the genomic DNA.

As used herein, “under transcriptional control” or “operably linked” refers to expression (e.g., transcription or translation) of a polynucleotide sequence which is controlled by an appropriate juxtaposition of an expression control element and a coding sequence. In one aspect, a nucleic acid sequence is “operably linked” to an expression control sequence when the expression control sequence controls and regulates the transcription of that sequence. In another context, the term “operably linked” refers to the linkage of a peptide, polypeptide or proteins such as an epitope or antigen with a signal sequence, such as a secretion signal sequence to bring about the secretion of the peptide, polypeptide or protein from a host cell.

As used herein, “signal sequence” or “signal peptide” denotes an endoplasmic reticulum translocation sequence. This sequence encodes a signal peptide that communicates to a cell to direct a polypeptide to which it is linked (e.g., via a chemical bond) to an endoplasmic reticulum vesicular compartment, to enter an exocytic/endocytic organelle, to be delivered either to a cellular vesicular compartment, the cell surface or to secrete the polypeptide. This signal sequence is sometimes clipped off by the cell in the maturation of a polypeptide. Signal sequences can be found associated with a variety of proteins native to prokaryotes and eukaryotes. A “secretion signal sequence” refers to a signal sequence that results in the polypeptide to which it is attached being secreted by a cell.

As used herein, “coding sequence” is a sequence which is transcribed and translated into a peptide, polypeptide or protein when placed under the control of appropriate expression control sequences. The boundaries of a coding sequence are determined by a start codon at the 5′ (amino) terminus and a translation stop codon at the 3′ (carboxyl) terminus. A coding sequence can include, but is not limited to, a prokaryotic sequence, cDNA from eukaryotic mRNA, a genomic DNA sequence from eukaryotic (e.g., mammalian) DNA, and even synthetic DNA sequences. A polyadenylation signal and transcription termination sequence will usually be located 3′ to the coding sequence.

As used herein, two coding sequences “correspond” to each other if the sequences or their complementary sequences encode the same amino acid sequences.

As used herein, “trafficking” denotes movement or progression of the polypeptide encoded by the bicistronic LAMP construct through cellular organelles or compartments in the pathway from the rough endoplasmic reticulum to the endosomal/lysosomal compartment or related organelles where antigen processing and binding to MHC II occurs.

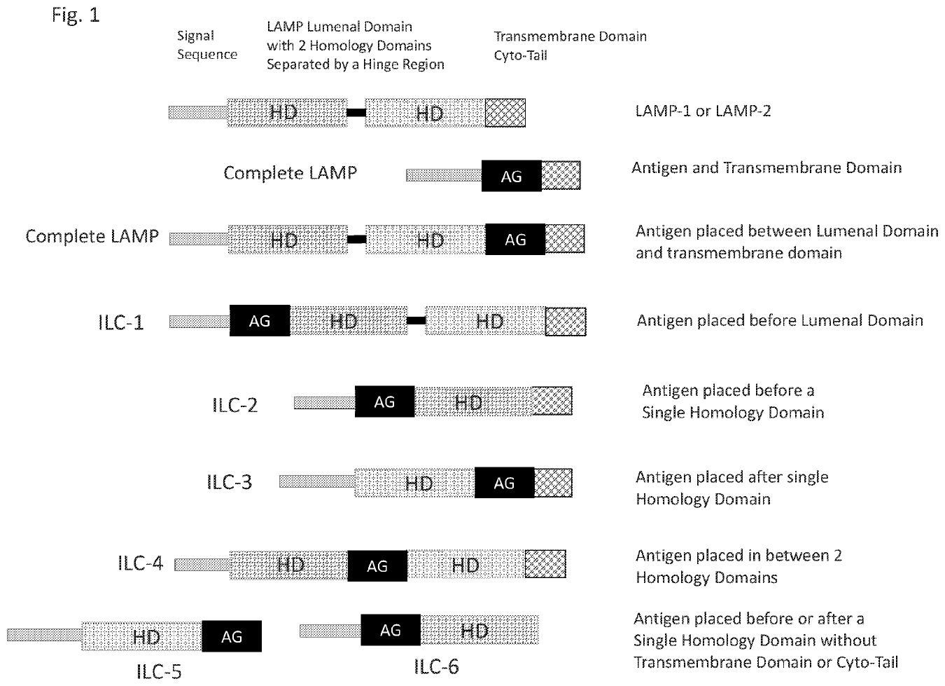

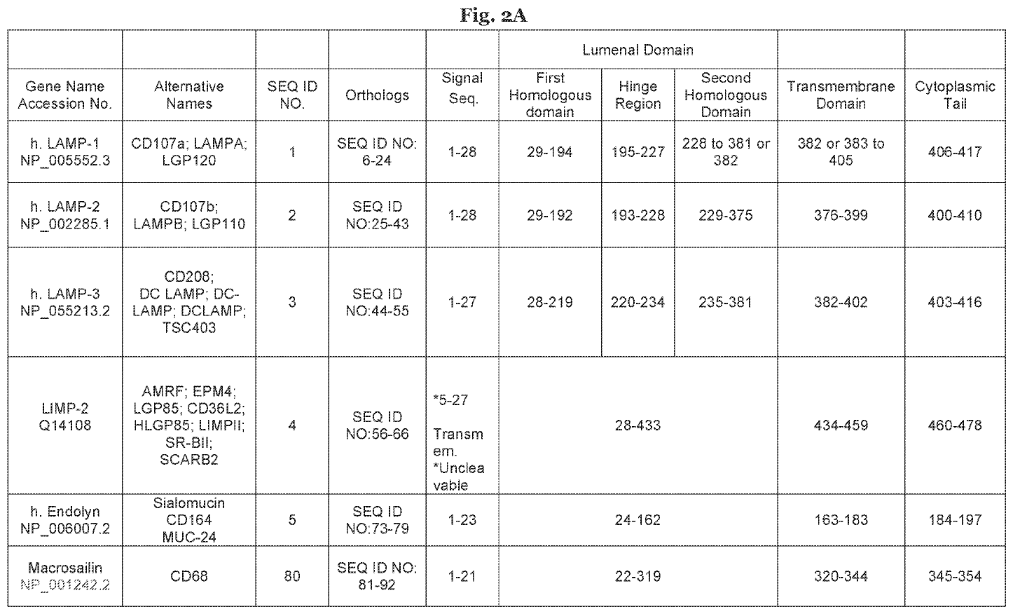

The term “antigen” or “antigen of interest” as used herein covers any polypeptide sequence encoded by a polynucleotide sequence cloned into the nucleic acid molecules encoding a bicistronic LAMP construct which is used to elicit an innate or adaptive immune response. An “antigen” encompasses both a single antigen as well as multiple antigenic sequences (derived from the same or different proteins). In some cases, the “antigen” provides particular “epitopes” or antibody recognition sites. In some cases, the “antigen” is a “target antigen,” meaning that it represents a specific protein expressed by diseased cells, such as a tumor antigen expressed by tumor cells, or a particular foreign antigen from an infectious disease such as a Spike protein from a coronavirus or other type of virus. In some cases, the antigen is expressed within a LAMP fusion protein, creating a “LAMP-antigen Construct.” The different arrangements of LAMP-antigen Constructs that can be used herein are illustrated in FIG. 1 as ILC-1-ILC-6.

As used herein, a “bicistronic LAMP construct” and a “bicistronic LAMP construct comprising an antigen” and a “bicistronic LAMP construct comprising a target antigen” and a “bicistronic LAMP construct comprising an antigen of interest” and a “bicistronic LAMP-antigen construct” are used interchangeably, and refer to a nucleic acid construct that encodes or expresses two polypeptides, i.e. a first polypeptide comprising a LAMP-antigen construct and a second polypeptide, which in some embodiments may encode an IREG polypeptide or a second antigen. In some cases, a bicistronic LAMP construct nucleic acid molecule herein can be a DNA molecule, such as a DNA vector, e.g., a DNA virus vector, and in other cases it can be an RNA molecule, including a self-amplifying RNA vector (also known as a self-replicating RNA vector). In other cases, based on the context, a “bicistronic LAMP construct” refers to the polypeptides that are collectively expressed from the nucleic acid construct.

As used herein, an “immune response element,” or “immune response enhancing gene” or “IREG” broadly refers to a gene encoding a protein that may enhance an immune response in a subject, such as a humoral and/or cellular immune response. In some cases, the abbreviation IREG may also be used to refer to the encoded polypeptide of such a gene, or an extracellular domain of such a protein, or a fusion molecule comprising such a protein or extracellular domain. Examples of IREGs include certain cytokines or immune proteins, such as CD40L, CD80, OX40, Flt3L, GM-CSF, IL-12, IL-21, IL-23, IL-15, CD70, CD86, IL-7, IL-18, or IL-33.

As used herein, an “bicistronic LAMP construct delivery vehicle” is defined as any molecule or group of molecules or macromolecules that can carry a nucleic acid molecule encoding a bicistronic LAMP construct into a host cell.

As used herein, “bicistronic LAMP construct delivery,” or “bicistronic LAMP construct transfer,” refers to the introduction of a nucleic acid molecule encoding a bicistronic LAMP construct into a host cell, irrespective of the method used for the introduction. The introduced nucleic acid molecule may be stably or transiently maintained in the host cell. Stable maintenance typically requires that the introduced nucleic acid molecule encoding the bicistronic LAMP construct either contains an origin of replication compatible with the host cell or integrates into a replicon of the host cell such as an extrachromosomal replicon (e.g., a plasmid) or a nuclear or mitochondrial chromosome.

As used herein, a “viral bicistronic LAMP construct” refers to a virus or viral particle that comprises a nucleic acid molecule comprising the bicistronic LAMP construct to be delivered into a host cell, either in vivo, ex vivo or in vitro. Examples of viral bicistronic LAMP constructs include, but are not limited to, adenovirus vectors, adeno-associated virus vectors, retroviral vectors, and the like. In aspects where gene transfer is mediated by an adenoviral vector, a viral bicistronic LAMP construct includes the adenovirus genome or part thereof, and a selected, non-adenoviral gene, in association with adenoviral capsid proteins.

As used herein, “adenoviral-mediated gene transfer” or “adenoviral transduction” refers to the process by which a nucleic acid molecule encoding a bicistronic LAMP construct is transferred into a host cell by virtue of the adenovirus entering the cell. Preferably, the nucleic acid molecule is able to replicate and/or integrate and be transcribed within the cell.

As used herein, “adenovirus particles” are individual adenovirus virions comprised of an external capsid and a nucleic acid molecule encoding a bicistronic LAMP construct, where the capsid is further comprised of adenovirus envelope proteins. The adenovirus envelope proteins may be modified to comprise a fusion polypeptide which contains a polypeptide ligand covalently attached to the viral protein, e.g., for targeting the adenoviral particle to a particular cell and/or tissue type.

As used herein, the term “administering” or “immunizing” or “injecting” a nucleic acid molecule encoding a bicistronic LAMP construct refers to transducing, transfecting, microinjecting, electroporating, or shooting the cell with the nucleic acid molecule. In some aspects, nucleic acid molecules encoding a bicistronic LAMP construct are introduced into a target cell by contacting the target cell with a delivery cell (e.g., by cell fusion or by lysing the delivery cell when it is in proximity to the target cell).

The term “treat” or “treatment” other like, as used herein, refers broadly to an improvement or amelioration of a disease or disorder in a subject, such as the improvement or amelioration of at least one symptom or marker associated with the disease or disorder, such as, in the case of a tumor, for example, reduction in the size of the tumor, or a change in biochemical markers associated with the tumor, or reduction in disease symptoms. In the case of a disease, such as a cancer or infectious disease, treat or treatment also refers to the reduction in at least one symptom of the disease or disorder. Treat or treatment also refers to prevention of the onset or slowing of the onset of a disease or disorder, or prevention or reduction of one or more symptoms upon onset (e.g, including development an asymptomatic disease vs. a symptomatic one), for example. Treat or treatment also refers to use in immunization or vaccination, for example, to induce an immune response in a subject that may, for example, prevent onset of symptoms, reduce severity of symptoms, or improve at least one existing symptom of a disease or disorder in a subject.

As used herein, the phrase “target enhancement” or simply “enhancement” of an immune response describes the use of a nucleic acid molecule encoding a “LAMP-antigen Construct” comprising a target antigen related to the disease or disorder to be treated in a LAMP fusion polypeptide, and (2) a further secreted polypeptide encoding a protein intended to enhance an immune response (i.e. an IREG protein). In general, such target enhancement allows for delivery of target antigens and secreted IREGs simultaneously. In some embodiments, this approach may improve both humoral and cellular immune responses.

The term “secreted” as used herein refers to processes and pathways within cells which result in a peptide, polypeptide or protein being transported through the cell wall such that the peptide, polypeptide or protein is released into the extracellular environment and may, for example, enter the circulation of a subject. A peptide, polypeptide or protein destined for the extracellular environment (i.e., to be secreted) will typically be provided with a secretion signal sequence, generally located at the N-terminus, which directs the ribosomes translating the peptide, polypeptide or protein to the rough endoplasmic reticulum (rough ER), from where newly made peptide, polypeptide or protein may be incorporated into small transport or secretory vesicles which transport the peptide, polypeptide or protein to the cell surface for release.

As used herein, “hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

As used herein, a polynucleotide or polynucleotide region (or a polypeptide or polypeptide region) which has a certain percentage (for example, at least 50%, at least 60%, at least 70%, at least 80%, at least 85%, at least 90%, at least 95%, at least 99%) of “sequence identity” to another sequence means that, when maximally aligned, using software programs routine in the art, that percentage of bases (or amino acids) are the same in comparing the two sequences.

Two sequences are “substantially homologous” or “substantially similar” when at least 50%, at least 60%, at least 70%, at least 75%, at least 80%, at least 90 or at least 95% of the nucleotides match over the defined length of the DNA sequences. Similarly, two polypeptide sequences are “substantially homologous” or “substantially similar” when at least 50%, at least 60%, at least 66%, at least 70%, at least 75%, at least 80%, at least 90 or at least 95% of the amino acid residues of the polypeptide match over a defined length of the polypeptide sequence. Sequences that are substantially homologous can be identified by comparing the sequences using standard software available in sequence data banks. Substantially homologous nucleic acid sequences also can be identified in a Southern hybridization experiment under, for example, stringent conditions as defined for that particular system. Defining appropriate hybridization conditions is within the skill of the art. For example, stringent conditions can be: hybridization at 5×SSC and 50% formamide at 42° C., and washing at 0.1×SSC and 0.1% sodium dodecyl sulfate at 60° C. Further examples of stringent hybridization conditions include: incubation temperatures of about 25 degrees C. to about 37 degrees C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40 degrees C. to about 50 degrees C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55 degrees C. to about 68 degrees C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. Similarity can be verified by sequencing, but preferably, is also or alternatively, verified by function (e.g., ability to traffic to an endosomal compartment, and the like), using assays suitable for the particular domain in question.

The terms “percent (%) sequence similarity”, “percent (%) sequence identity”, and the like, generally refer to the degree of identity or correspondence between different nucleotide sequences of nucleic acid molecules or amino acid sequences of polypeptides that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wisconsin), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990, 87:2264, modified as in Karlin and Altschul, Proc. Natl. Acad. Sci. USA 1993, 90:5873-5877. Such an algorithm is incorporated into the NBLAST and XBLAST programs of Altschul et al, J. Mol. Biol. 1990; 215:403. BLAST nucleotide searches can be performed with the NBLAST program, score=100, wordlength=12, to obtain nucleotide sequences homologous to sequences of the disclosure. BLAST protein searches can be performed with the NBLAST program, score=50, wordlength=3, to obtain amino acid sequences homologous to protein sequences of the disclosure. To obtain gapped alignments for comparison purposes, Gapped BLAST can be utilized as described in Altschul et al, Nucleic Acids Res. 1997, 25:3389. Alternatively, PSI-Blast can be used to perform an iterated search that detects distant relationship between molecules. See Altschul et al. (1997) supra. When utilizing BLAST, Gapped BLAST, and PSI-Blast programs, the default parameters of the respective programs (e.g., XBLAST and NBLAST) can be used. See ncbi.nlm.nih.gov/BLAST/on the WorldWideWeb.

Another non-limiting example of a mathematical algorithm utilized for the comparison of sequences is the algorithm of Myers and Miller, CABIOS 1988; 4: 11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In one embodiment, the percent identity between two amino acid sequences is determined using the algorithm of Needleman and Wunsch (J. Mol. Biol. 1970, 48:444-453), which has been incorporated into the GAP program in the GCG software package (Accelrys, Burlington, MA; available at accelrys.com on the WorldWideWeb), using either a Blossum 62 matrix or a PAM250 matrix, a gap weight of 16, 14, 12, 10, 8, 6, or 4, and a length weight of 1, 2, 3, 4, 5, or 6. In yet another embodiment, the percent identity between two nucleotide sequences is determined using the GAP program in the GCG software package using a NWSgapdna.CMP matrix, a gap weight of 40, 50, 60, 70, or 80, and a length weight of 1, 2, 3, 4, 5, or 6. A particularly set of parameters (and the one that can be used if the practitioner is uncertain about what parameters should be applied to determine if a molecule is a sequence identity or homology limitation of the disclosure) is using a Blossum 62 scoring matrix with a gap open penalty of 12, a gap extend penalty of 4, and a frameshift gap penalty of 5.

Another non-limiting example of how percent identity can be determined is by using software programs such as those described in Current Protocols In Molecular Biology (F. M. Ausubel et al., eds., 1987) Supplement 30, section 7.7.18, Table 7.7.1. Preferably, default parameters are used for alignment. An alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

Statistical analysis of the properties described herein may be carried out by standard tests, for example, t-tests, ANOVA, or Chi squared tests. Typically, statistical significance will be measured to a level of p=0.05 (5%), more preferably p=0.01, p=0.001, p=0.0001, p=0.000001

“Conservatively modified variants” of domain sequences also can be provided. With respect to particular nucleic acid sequences, conservatively modified variants refer to those nucleic acids which encode identical or essentially identical amino acid sequences, or where the nucleic acid does not encode an amino acid sequence, to essentially identical sequences.

Specifically, degenerate codon substitutions can be achieved by generating sequences in which the third position of one or more selected (or all) codons is substituted with mixed-base and/or deoxyinosine residues (Batzer, et al., 1991, Nucleic Acid Res. 19:5081; Ohtsuka, et al., 1985, J. Biol. Chem. 260:2605-2608; Rossolini et al., 1994, Mol. Cell. Probes 8:91-98). The term “biologically active fragment”, “biologically active form”, “biologically active equivalent” of and “functional derivative” of a wild-type protein, possesses a biological activity that is at least substantially equal (e.g., not significantly different from) the biological activity of the wild type protein as measured using an assay suitable for detecting the activity.

As used herein, “in vivo nucleic acid delivery, nucleic acid transfer, nucleic acid therapy” and the like, refer to the introduction of a nucleic acid molecule encoding a bicistronic LAMP construct as described herein directly into the body of a subject, such as a human or non-human mammal, whereby the nucleic acid molecule is introduced to a cell of such organism in vivo.

As used herein, the term “in situ” refers to a type of in vivo nucleic acid delivery in which the nucleic acid molecule encoding a bicistronic LAMP construct is brought into proximity with a target cell (e.g., the nucleic acid is not administered systemically). For example, in situ delivery methods include, but are not limited to, injecting a nucleic acid molecule encoding a bicistronic LAMP construct directly at a site (e.g., into a tissue, such as a tumor or heart muscle), contacting the nucleic acid molecule with cell(s) or tissue through an open surgical field, or delivering the nucleic acid molecule to a site using a medical access device such as a catheter.

As used herein, the term “isolated” or “purified” means separated (or substantially free) from constituents, cellular and otherwise, in which the polynucleotide, peptide, polypeptide, protein, antibody, or fragments thereof, are normally associated with in nature. For example, with respect to an isolated nucleic acid molecule encoding a bicistronic LAMP construct as described herein, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. By substantially free or substantially purified, it is meant at least 50% of the population, preferably at least 70%, more preferably at least 80%, and even more preferably at least 90%, are free of the components with which they are associated in nature.

As used herein, a “target cell” or “recipient cell” refers to an individual cell or cell which is desired to be, or has been, a recipient of a nucleic acid molecule encoding a bicistronic LAMP construct described herein. The term is also intended to include progeny of a single cell, and the progeny may not necessarily be completely identical (in morphology or in genomic or total DNA complement) to the original parent cell due to natural, accidental, or deliberate mutation. A target cell may be in contact with other cells (e.g., as in a tissue) or may be found circulating within the body of an organism.

As used herein, a “subject” is a human unless specifically noted otherwise. In such other cases, a subject may be a mammal. Mammals include, but are not limited to, murines, simians, humans, farm animals, sport animals, and pets. In some cases, the “subject” is a rodent (e.g., a rat, a mouse or rabbit), a llama, camel, a cow, a guinea pig, a hamster, a dog, a cat, a horse, a non-human primate, a simian (e.g., a monkey or ape), a monkey (e.g., marmoset, baboon, rhesus macaque), or an ape (e.g., gorilla, chimpanzee, orangutan, gibbon). In some embodiments, non-human mammals, especially mammals that are conventionally used as models for demonstrating therapeutic efficacy in humans (e.g., murine, primate, porcine, canine, or rabbit animals) may be employed.

The terms “cancer,” “neoplasm,” and “tumor,” are used interchangeably and in either the singular or plural form, refer to cells that have undergone a malignant transformation that makes them pathological to the host organism. Primary cancer cells transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass, e.g., by procedures such as CAT scan, MR imaging, X-ray, ultrasound or palpation, and/or which is detectable because of the expression of one or more cancer-specific antigens in a sample obtainable from a patient.

In some embodiments, the cancer (including all stages of progression, including hyperplasia) is an adenocarcinoma, sarcoma, skin cancer, melanoma, bladder cancer, brain cancer, breast cancer, uterine cancer, ovarian cancer, prostate cancer, lung cancer (including, but not limited to NSCLC, SCLC, squamous cell cancer), colorectal cancer, anal cancer, rectal cancer, cervical cancer, liver cancer, head and neck cancer, oral cancer, salivary gland cancer, esophageal cancer, pancreas cancer, pancreatic ductal adenocarcinoma (PDA), renal cancer, stomach cancer, kidney cancer, multiple myeloma or cerebral cancer.

The nucleic acid molecules encoding bicistronic LAMP constructs as described herein can also be used to treat allergies, such as for example, food allergies (e.g., peanut allergens, such as Ara H1, H2 and/or H3), or environmental allergens, such as for example pollen (tree pollen, such as for example CRY J1 or CRY J2), dog dander, cat saliva, or dust mites.

In some cases, bicistronic LAMP constructs may include antigens useful in treatment of infectious diseases such as viral or bacterial diseases. In one example, bicistronic LAMP constructs may be used to treat coronavirus infections, such as from Covid-19.

Other diseases and/or disorders that can be treated with the bicistronic LAMP construct described herein include, for example, infectious disease and diabetes.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. Compositions comprising nucleic acid molecules encoding the bicistronic LAMP constructs also can include stabilizers and preservatives. For examples of carriers, stabilizers and adjuvants, see Martin Remington's Pharm. Sci., 15th Ed. (Mack Publ. Co., Easton (1975).

A cell has been “transformed”, “transduced”, or “transfected” by a nucleic acid molecule encoding a bicistronic LAMP construct when such a nucleic acid molecule has been introduced inside the cell. Transforming DNA may or may not be integrated (covalently linked) with chromosomal DNA making up the genome of the cell. In prokaryotes, yeast, and mammalian cells for example, the nucleic acid molecule encoding a bicistronic LAMP construct may be maintained on an episomal element, such as a plasmid. In a eukaryotic cell, a stably transformed cell is one in which the nucleic acid molecule encoding a bicistronic LAMP construct has become integrated into a chromosome so that it is inherited by daughter cells through chromosome replication. This stability is demonstrated by the ability of the eukaryotic cell to establish cell lines or clones comprised of a population of daughter cells containing the nucleic acid molecule. A “clone” is a population of cells derived from a single cell or common ancestor by mitosis. A “cell line” is a clone of a primary cell that is capable of stable growth in vitro for many generations (e.g., at least 10).

As used herein, an “effective amount” or “therapeutically effective amount” is an amount sufficient to affect beneficial or desired results, e.g., to treat a subject or induce an immune response in a subject.

An “antibody” is any immunoglobulin, including antibodies and fragments thereof, that binds a specific antigen. The term encompasses polyclonal, monoclonal, and chimeric antibodies (e.g., bispecific antibodies). An “antibody combining site” is that structural portion of an antibody molecule comprised of heavy and light chain variable and hypervariable regions that specifically binds antigen. Exemplary antibody molecules are intact immunoglobulin molecules, substantially intact immunoglobulin molecules, and those portions of an immunoglobulin molecule that contains the paratope, including Fab, Fab′, F(ab′)₂ and F (v) portions. Thus, the term antibody encompasses not only whole antibody molecules, but also antibody fragments as well as variants (including derivatives such as fusion proteins) of antibodies and antibody fragments. Examples of molecules which are described by the term “antibody” in this application include, but are not limited to: single chain Fvs (scFvs), Fab fragments, Fab′ fragments, F(ab′)₂, disulfide linked Fvs (sdFvs), Fvs, and fragments comprising or alternatively consisting of, either a VL or a VH domain. The term “single chain Fv” or “scFv” as used herein refers to a polypeptide comprising a VL domain of an antibody linked to a VH domain of an antibody. See Carter (2006) Nature Rev. Immunol. 6:243.

Additionally, antibodies that may be generated using the nucleic acid molecule encoding a bicistronic LAMP construct described herein include, but are not limited to, monoclonal, multi-specific, bi-specific, human, humanized, mouse, or chimeric antibodies, single chain antibodies, camelid antibodies, Fab fragments, F(ab′) fragments, anti-idiotypic (anti-Id) antibodies (including, e.g., anti-Id antibodies to antibodies of the disclosure), domain antibodies and epitope-binding fragments of any of the above. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule.

As used herein, “human” antibodies include antibodies having the amino acid sequence of a human immunoglobulin and include antibodies isolated from human immunoglobulin libraries and xenomice or other organisms that have been genetically engineered to produce human antibodies. The nucleic acid molecules encoding a bicistronic LAMP construct described herein can be used in combination with known techniques for generating human antibodies and human monoclonal antibodies as described in the exemplified protocols, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598; and Lonberg and Huszar, Int. Rev. Immunol. 13:65-93 (1995).

Human antibodies or “humanized” chimeric monoclonal antibodies can be produced using the nucleic acid molecules encoding bicistronic LAMP constructs in combination with techniques described herein or otherwise known in the art. For example, standard methods for producing chimeric antibodies are known in the art. See, for review the following references: Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

Antibodies that may be generated using a nucleic acid molecule encoding a bicistronic LAMP construct described herein may be monovalent, bivalent, trivalent or multivalent. For example, monovalent scFvs can be multimerized either chemically or by association with another protein or substance. A scFv that is fused to a hexahistidine tag or a Flag tag can be multimerized using Ni-NTA agarose (Qiagen) or using anti-Flag antibodies (Stratagene, Inc.). Additionally, the nucleic acid molecule encoding a bicistronic LAMP construct can be used to generate monospecific, bispecific, trispecific or of greater multispecificity for the encoded antigen(s) contained in the bicistronic LAMP construct. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et.al., J. Immunol. 148:1547-1553 (1992).

An “epitope” is a structure, usually made up of a short peptide sequence or oligosaccharide, that is specifically recognized or specifically bound by a component of the immune system. T-cell epitopes have generally been shown to be linear oligopeptides. Two epitopes correspond to each other if they can be specifically bound by the same antibody. Two epitopes correspond to each other if both are capable of binding to the same B cell receptor or to the same T cell receptor, and binding of one antibody to its epitope substantially prevents binding by the other epitope (e.g., less than about 30%, preferably, less than about 20%, and more preferably, less than about 10%, 5%, 1%, or about 0.1% of the other epitope binds). It will be understood by the one of ordinary skill in the art that multiple epitopes can make up an antigen.

The term “antigen presenting cell” as used herein includes any cell which presents on its surface an antigen in association with a major histocompatibility complex molecule, or portion thereof, or, alternatively, one or more non-classical MHC molecules, or a portion thereof. Examples of suitable APCs are discussed in detail below and include, but are not limited to, whole cells such as macrophages, dendritic cells, B cells, hybrid APCs, and foster antigen presenting cells.

As used herein an “engineered antigen-presenting cell” refers to an antigen-presenting cell that has a non-natural molecular moiety on its surface. For example, such a cell may not naturally have a costimulator on its surface or may have an additional artificial costimulator in addition to a natural costimulator on its surface, or may express a non-natural class II molecule on its surface. In some embodiments, the engineered antigen-presenting cell has the antigen expressed from the bicistronic LAMP construct on its surface.

As used herein, “immune effector cells” refers to cells capable of binding an antigen and which mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates.

As used herein, “partially human” refers to a nucleic acid having sequences from both a human and a non-human vertebrate. In the context of partially human sequences, the partially human nucleic acids have sequences of human immunoglobulin coding regions and sequences based on the non-coding sequences of the endogenous immunoglobulin region of the non-human vertebrate. The term “based on” when used with reference to endogenous non-coding sequences from a non-human vertebrate refers to sequences that correspond to the non-coding sequence and share a relatively high degree of homology with the non-coding sequences of the endogenous loci of the host vertebrate, e.g., the non-human vertebrate from which the ES cell is derived. Preferably, the non-coding sequences share at least an 80%, more preferably 90% homology with the corresponding non-coding sequences found in the endogenous loci of the non-human vertebrate host cell into which a partially human molecule comprising the non-coding sequences has been introduced.

The term “immunoglobulin variable region” as used herein refers to a nucleotide sequence that encodes all or a portion of a variable region of an antibody molecule or all or a portion of a regulatory nucleotide sequence that controls expression of an antibody molecule. Immunoglobulin regions for heavy chains may include but are not limited to all or a portion of the V, D, J, and switch regions, including introns. Immunoglobulin region for light chains may include but are not limited to the V and J regions, their upstream flanking sequences, introns, associated with or adjacent to the light chain constant region gene.

By “transgenic animal” is meant a non-human animal, usually a mammal, having an exogenous nucleic acid sequence present as an extrachromosomal element in a portion of its cells or stably integrated into its germ line DNA (i.e., in the genomic sequence of most or all of its cells). In generating a transgenic animal comprising human sequences, a partially human nucleic acid is introduced into the germ line of such transgenic animals by genetic manipulation of, for example, embryos or embryonic stem cells of the host animal according to methods well known in the art.

A “vector” includes plasmids and viruses and any DNA or RNA molecule, whether self-replicating or not, which can be used to transform or transfect a cell.

As used herein, a “genetic modification” refers to any addition, deletion or disruption to a cell's normal nucleotides. Art recognized methods include viral mediated gene transfer, liposome mediated transfer, transformation, transfection and transduction, e.g., viral-mediated gene transfer such as the use of the nucleic acid molecules encoding bicistronic LAMP constructs based on DNA viruses such as adenovirus, adeno-associated virus and herpes virus, as well as retroviral based vectors.

The practice of the present disclosure employs in certain aspects, unless otherwise indicated, conventional molecular biology, microbiology, and recombinant DNA techniques within the skill of the art. Such techniques are explained fully in the literature. See, e.g., Maniatis, Fritsch & Sambrook, In Molecular Cloning: A Laboratory Manual (1982); DNA Cloning: A Practical Approach, Volumes I and II (D. N. Glover, ed., 1985); Oligonucleotide Synthesis (M. J. Gait, ed., 1984); Nucleic Acid Hybridization (B. D. Hames & S. J. Higgins, eds., 1985); Transcription and Translation (B. D. Hames & S. I. Higgins, eds., 1984); Animal Cell Culture (R. I. Freshney, ed., 1986); Immobilized Cells and Enzymes (IRL Press, 1986); B. Perbal, A Practical Guide to Molecular Cloning (1984).

Unless defined otherwise, all technical and scientific terms used herein have the same meaning as commonly understood by one of ordinary skill in the art to which this disclosure belongs. All publications mentioned herein are incorporated by reference for the purpose of describing and disclosing devices, formulations and methodologies that may be used in connection with the presently described disclosure.

### B. Exemplary LAMP-Antigen Fusion Polypeptides

The following are representative embodiments:

An isolated nucleic acid encoding a bicistronic LAMP construct, i.e. encoding a LAMP-antigen fusion protein (a LAMP Construct) and an immune enhancing protein such that both are expressed in a host cell or subject. In some cases, a nucleic acid herein may encode a multicistronic construct if a third polypeptide is also expressed, for example.

In some cases, the antigen is a target antigen for a particular disease or disorder. In some cases, the immune enhancing protein is a polypeptide or polypeptide domain, e.g., an extracellular domain, from an IREG, optionally fused to a further molecule such as an Fc domain of an immunoglobulin. In some cases, the immune enhancing protein may be secreted, and thus is operably linked to a secretory signal sequence. In some cases, the LAMP-antigen fusion protein may have the backbone structure of any one of ILC-1, ILC-2, ILC-3, ILC-4, ILC-5 or ILC-6 of FIG. 1 herein. In some cases, it has the backbone structure of ILC-4. In some cases, the LAMP-antigen construct comprises the antigen placed between two homology domains of a luminal domain of a LAMP protein. In other cases, the antigen is placed at the before the N-terminus of a LAMP homology domain or after the C-terminus of a LAMP homology domain but prior to a LAMP transmembrane domain. As used herein, the LAMP protein can be selected from LAMP-1, LAMP2, LAMP-3, LIMP 2, Macrosialin, Endolyn, LAMP5 or LIMBIC. In additional embodiments, the LAMP protein is selected from an amino acid sequence having at least 60%, at least 70%, at least 75%, at least 80%, at least 85%, at least 90%, at least 95%, at least 96%, at least 97%, at least 98%, at least 99%, or 100% sequence identity to any one of the amino acid SEQ ID NOS: 1-113. In some cases, the LAMP protein is LAMP-1.

In some cases, the LAMP components include, in addition to at least two homology domains of LAMP, a transmembrane domain of LAMP or of a heterologous protein, as well as optionally a signal sequence of LAMP or a heterologous protein, and in some cases a cytoplasmic domain of LAMP. In some such cases, the LAMP protein is LAMP-1.

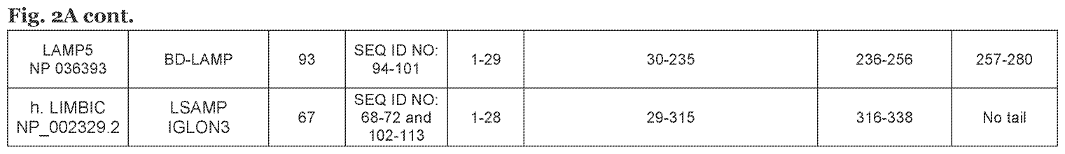

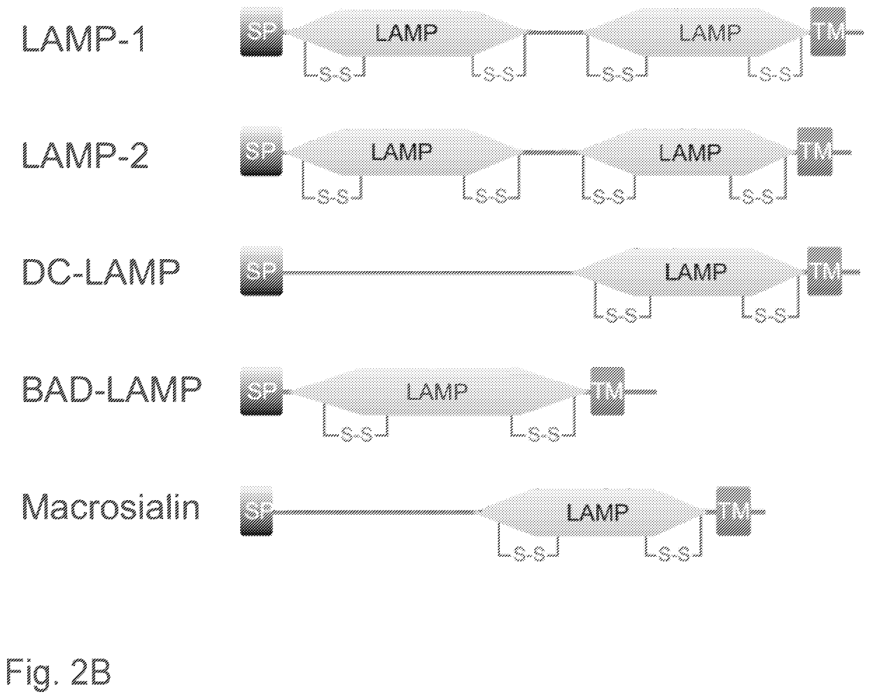

In particular, LAMP-1, as deduced from a cDNA clone (Chen, et al., J. Biol. Chem. 263:8754, 1988) consists of a polypeptide core of about 382 amino acids with a large (346-residue) luminal amino-terminal domain followed by a 24-residue hydrophobic transmembrane region and short (12-residue) carboxyl-terminal cytoplasmic tail. See, FIG. 2A and FIG. 2B. The luminal domain is highly glycosylated, being substituted with about 20 asparagine linked complex-type oligosaccharides and consists of two approximately 160-residue “homology domains” that are separated by a proline/serine-rich 22-residue “hinge” region. Each of these “homology domains” contains 4 uniformly spaced cysteine residues, disulfide bonded to form four 36-38-residue loops symmetrically placed within the two halves of the luminal domain (Arterburn, et al., J. Biol. Chem. 265:7419, 1990; see, also Chen, et al., J. Biol. Chem. 25: 263 (18): 8754-8, 1988). FIG. 2A schematically shows the conserved domains between LAMP-1, LAMP-2, LAMP-3, Endolyn, LIMBIC, LAMP5, or Macrosialin.

Previously reported LAMP constructs comprised the following elements in this specific arrangement: (a) a full luminal domain of LAMP-1 protein, the antigen and then the full transmembrane/cytoplasmic tail of LAMP-1 protein; or (b) the antigen and the full transmembrane/cytoplasmic tail of a LAMP-1 protein.

In example (a), the antigenic sequence is inserted in between the full luminal domain of a LAMP-1 protein and the LAMP-1 full transmembrane domain/cytoplasmic tail. Both constructs have been shown to successfully target an antigenic sequence to the lysosome/endosome and will be referred to as “complete LAMP Constructs” as shown in FIG. 1 as compared to the improved LAMP Constructs ILC-1-ILC-6 described herein. The bicistronic LAMP constructs described herein do not include the complete LAMP Constructs. Instead, the bicistronic LAMP constructs described herein may comprise at least one antigen fused to the N-terminus of the luminal domain of a LAMP protein, the N- or C-terminus of at least one homology domain of a LAMP protein, or the N- or C-terminus of at least one Cysteine Conserved Fragment of a LAMP protein (see, for example ILC-1-ILC-6 of FIG. 1). However, some bicistronic LAMP constructs comprise at least one antigen fused between a first homology domain of a LAMP protein and a second homology domain of a LAMP protein (or at least between two Cysteine Conserved Fragments) (see, for example, ILC-4 of FIG. 1). For example, the at least one antigen may be placed in, or may replace, the LAMP hinge region. In some embodiments, this construct also comprises a transmembrane domain of a LAMP protein, and/or the cytosolic tail of a LAMP protein.

Specifically, in some embodiments, the bicistronic LAMP constructs comprise two homology domains (e.g., ILC-4 of FIG. 1). In some embodiments, these constructs also comprise a transmembrane domain of a LAMP protein, and/or the cytosolic tail of a LAMP protein. In other embodiments, when an antigen contains a transmembrane domain, the transmembrane domain of a LAMP protein and/or the cytosolic tail of a LAMP protein is unnecessary. In further other embodiments, the two homology domains are derived from a LAMP-1, LAMP-2, LAMP-3, or an Endolyn protein. Alternatively, the two homology domains are derived from different LAMP proteins. In these constructs comprising two homology domains, a LAMP hinge domain may also be included.

In cases in which the LAMP-antigen portion of the bicistronic LAMP construct comprises an ILC-4 structure (as shown in FIG. 1), such as wherein the first polynucleotide sequence of the LAMP construct comprises or encodes a polypeptide comprising two homology domains of a luminal domain of a LAMP protein, and an antigenic domain heterologous to the LAMP protein, wherein the antigenic domain is placed between the two homology domains, the two LAMP protein homology domains may be chosen from the homology domain 1 and homology domain 2 amino acid sequences as shown in FIG. 3, derived from SEQ ID NOs: 1-113, or amino acid sequences that are at least 90%, at least 95%, at least 96%, at least 97%, at least 98% or at least 99% identical to those sequences, for example. Thus, for example, where the LAMP in such a construct is LAMP-1, a native human LAMP-1 homology domain 1 and homology domain 2 may surround the antigenic domain of the LAMP-antigen construct. In some cases, the homology domains may comprise amino acid residues 29-194 and 228-381 of SEQ ID NO: 1, or amino acid sequences that are at least 90%, at least 95%, at least 96%, at least 97%, at least 98% or at least 99% identical to those sequences, or one of the sequences of such domains depicted in FIGS. 2A and 3, or residues 29-195 of SEQ ID NOs: 198 and 202, for example. Where the construct is a polynucleotide, the construct may further encode a signal sequence prior to the start of the LAMP homology domain 1 coding sequence, for example residues 1-28 of SEQ ID NO: 1 or the signal sequences depicted otherwise in FIG. 2A and FIG. 3, or for example residues 1-28 of SEQ ID NO: 198. In some cases, a heterologous (i.e. non-LAMP) signal sequence may also be used. Therefore, in some embodiments, the first homology domain of LAMP comprises a polypeptide sequence with at least 90%, at least 95%, at least 96%, at least 97%, at least 98%, at least 99%, or with 100% sequence identity of residues 29 to the C-terminal of SEQ ID NO: 198 or residues 29-194 of SEQ ID NO: 1. In some cases, a LAMP signal sequence such as residues 1-28 of SEQ NO: 198 or SEQ ID NO: 1 may also be included. In some cases, the second homology domain of LAMP comprises a polypeptide sequence with at least 90%, at least 95%, at least 96%, at least 97%, at least 98%, at least 99%, or with 100% sequence identity of SEQ ID NO: 202 or residues 228-381 of SEQ ID NO: 1. In some cases, a LAMP transmembrane domain may be included, such as depicted in FIGS. 2A and 3, i.e., comprising residues 383-405 of SEQ ID NO:1, or comprising another native LAMP transmembrane domain sequence. In some cases, a cytoplasmic tail of LAMP may also be included, such as comprising residues 406-417 of SEQ ID NO: 1, or as depicted in FIGS. 2A and 3, or otherwise comprising a native LAMP cytoplasmic tail sequence.

In some other embodiments, the bicistronic LAMP constructs comprise at least one antigen fused to the C-terminus of a single homology domain of a LAMP protein or a single Cysteine Conserved Fragment of a LAMP protein. See, for example, ILC-3 and ILC-5 of FIG. 1. In some embodiments, these constructs also comprise a transmembrane domain of a LAMP protein, and/or the cytosolic tail of a LAMP protein. In other embodiments, when an antigen contains a transmembrane domain, the transmembrane domain of a LAMP protein and/or the cytosolic tail of a LAMP protein is unnecessary.

The bicistronic LAMP constructs described above can be generated using the domains defined in the Figures. For example, it is specifically contemplated that the domains included in the bicistronic LAMP constructs illustrated in FIG. 1, for example, can originate from sequences derived from orthologous sequences. See, FIGS. 3-10 for example. It is expressly contemplated that the equivalent domains defined in FIG. 2A and FIG. 2B be used to generate the bicistronic LAMP constructs using the vector backbones illustrated in FIG. 1 for orthologous sequences.

Moreover, the orthologous sequences shown in FIGS. 3-10 are representative of the sequences that can be used to generate the domains. It is well within the skill in the art to identify other orthologous sequences and/or isotypes and comparing them to the alignments shown in FIGS. 3-10. Thus, by identifying the equivalent boundaries defined in FIG. 2A and FIG. 2B for a human LAMP protein with the alignments shown in FIGS. 3-10, one can generate the bicistronic LAMP constructs described herein.

As would be well understood by the skilled artisan, the boundaries of each domain are an approximation and may be adjusted at least 1, 2, 3, 4, 5, 6, 7, 8, 9, or 10 amino acids based on cloning considerations and restriction enzyme placement. Therefore, when a particular domain (e.g., a LAMP Homology Domain) is included in the bicistronic LAMP construct, the amino acids beginning and ending of the domain may be adjust by at least 1, 2, 3, 4, 5, 6, 7, 8, 9, or 10 amino acids as those boundaries defined in FIG. 2A.

Each of the bicistronic LAMP constructs described herein can additionally comprise a signal sequence and/or additional amino acids in between each domain for cloning purposes as is well known in the art. Additionally, the LAMP homologous domains, the LAMP luminal domain, the LAMP transmembrane domain, and/or the LAMP cytosolic tail domain can originate from the same LAMP protein (e.g., human LAMP-1) or different LAMP proteins (e.g., luminal domain from human LAMP-1 and transmembrane domain from human LAMP-2, and/or mixing of orthologous domains in the same gene family (e.g., LAMP-1) or different gene family (LAMP-1 and LAMP-2).

Polypeptide variants of the described LAMP Constructs are contemplated. For example, polypeptides at least 60%, at least 70%, at least 75%, at least 80%, at least 85%, at least 90%, at least 95%, at least 96%, at least 97%, at least 98% or at least 99% identity to any of the bicistronic LAMP constructs described herein as well as polynucleotides encoding these variants. Variants of the bicistronic LAMP constructs retain the ability to function by targeting the antigenic sequence to the lysosome. For example, a modified luminal sequence must retain the ability to traffic both membrane and non-membrane antigenic materials to an endosomal compartment with at least 50%, at least 60%, at least 70%, at least 80%, at least 90%, or 100% efficacy compared to the original domain sequence, i.e., an efficacy that results in sufficient antigen presentation by a cell comprising the chimeric sequence for it to mount an immune response. In one aspect, sequences containing a suitable trafficking signal may be identified by constructing a bicistronic LAMP construct containing the well-characterized antigenic domain of ovalbumin, a transmembrane domain, and the cytoplasmic domain of a protein containing a putative lysosomal/endosomal targeting signal. Efficiency of targeting can be measured by determining the ability of antigen presenting cells, expressing the bicistronic LAMP construct, to stimulate HA epitope-specific, MHC class II restricted T-cells (see, e.g., Example 5 of U.S. Pat. No. 5,633,234).

Polynucleotides encoding any of the described bicistronic LAMP constructs are some embodiments of the disclosure, along with polynucleotides at least 60%, at least 70%, at least 75%, at least 80%, at least 85%, at least 90%, at least 95%, 96%, 97%, 98% or 99% identity to any of the bicistronic LAMP construct polynucleotides described herein. Variants of the bicistronic LAMP constructs retain the ability to function by targeting the antigenic sequence to the lysosome. For example, a modified luminal sequence must retain the ability to traffic both membrane and non-membrane antigenic materials to an endosomal compartment with at least 50%, at least 60%, at least 70%, at least 80%, at least 90%, or at least 100% efficacy compared to the original domain sequence, i.e., an efficacy that results in sufficient antigen presentation by a cell comprising the chimeric sequence for it to mount an immune response. In one aspect, sequences containing a suitable trafficking signal may be identified by constructing a bicistronic LAMP construct containing the well-characterized antigenic domain of ovalbumin, a transmembrane domain, and the cytoplasmic domain of a protein containing a putative lysosomal/endosomal targeting signal. Efficiency of targeting can be measured by determining the ability of antigen presenting cells, expressing the bicistronic LAMP construct, to stimulate HA epitope-specific, MHC class II restricted T-cells (see, e.g., Example 5 of U.S. Pat. No. 5,633,234).

### C. Construction of Exemplary Bicistronic LAMP Constructs

Bicistronic LAMP constructs herein may be constructed, for example, from an isolated nucleotide sequence in which the promoter/enhancer and coding sequences for the LAMP-antigen construct are followed or preceded in the nucleic acid by a second promoter/enhancer and coding sequences for a second polypeptide. In some cases, the second polypeptide may be a polypeptide intended to enhance an immune response, i.e., a protein or extracellular domain of a protein expressed from an immune response enhancing gene (IREG). In some cases, the second polypeptide may be secreted, and thus the nucleotide may include an appropriate secretory signal sequence for the second polypeptide, which may be already included in the polypeptide coding sequence, or which may be from a different protein. In some cases the second polypeptide is a fusion protein, such as an extracellular domain or complete IREG polypeptide fused to another polypeptide sequence such as an Fc domain of an immunoglobulin, a further antigen, or the like.

As described below, the antigen in the LAMP-antigen construct used in the bicistronic LAMP constructs may be a target antigen for an infectious disease such as a viral spike protein, or it could alternatively be a cancer antigen such as a polypeptide overexpressed in certain tumors or tumor cells.

For example, as described in the Examples below, certain bicistronic LAMP proteins were made using a spike protein or spike protein subunit from a SARS Co-V2 virus as well as using a cancer antigen such as a HER2 extracellular domain, or using NY-ESO1 or CD161. Thus, in some embodiments, the LAMP-antigen construct comprises LAMP fused to a cancer antigen or viral spike protein antigen. Coding regions for such antigens may, in some cases, be combined with an IREG coding region. Examples of IREGs include certain cytokines or immune proteins, such as CD40L, CD80, OX40, Flt3L, GM-CSF, IL-12, IL-21, IL-23, IL-15, CD70, CD86, IL-7, IL-18, or IL-33. In some embodiments, the IREG comprises a homologous or heterologous secretory signal sequence coding region so that the second polypeptide is secreted when expressed.

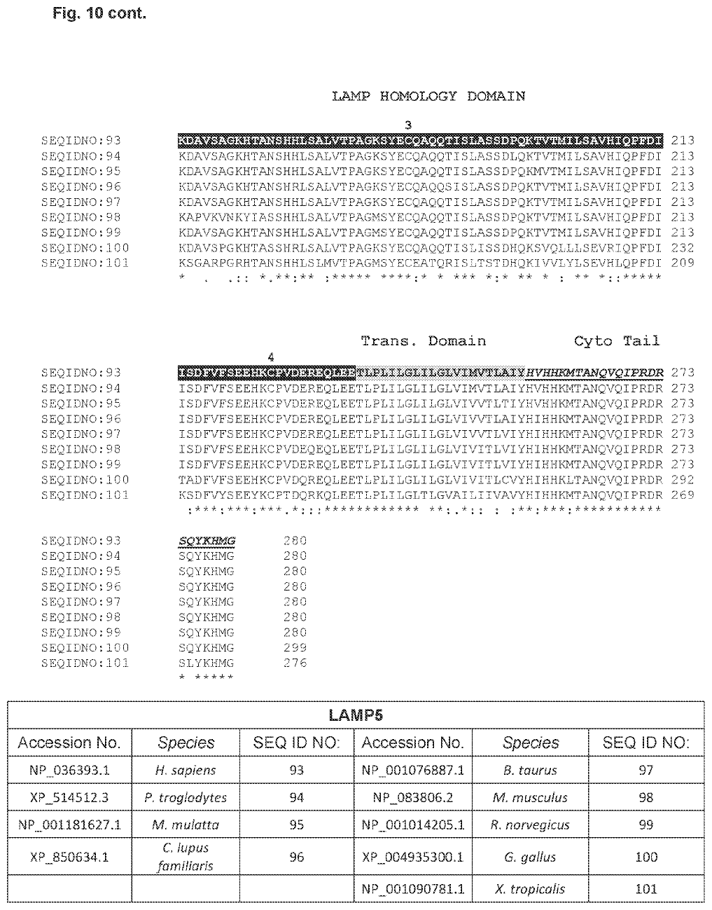

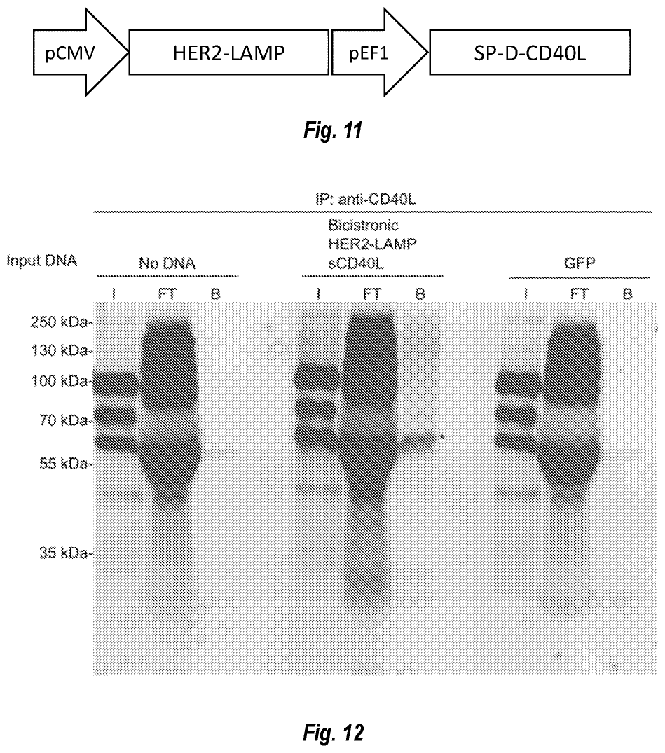

As described in the Examples that follow, the following bicistronic LAMP constructs were constructed: (1) HER2-LAMP-sCD40L (FIG. 11; SEQ ID NO: 197), (2) HER2-LAMP-mFLT3L (SEQ ID NO: 208), (3) HER2-LAMP-IL-12 (SEQ ID NO: 212), (4) HER2-LAMP-IL-21 (SEQ ID NO: 216), (5) HER2-LAMP-OX40L (SEQ ID NO: 241), (6) HER2-LAMP-CD80 (SEQ ID NO: 251), (7) NY-ESO1-LAMP-IL-15 (SEQ ID NO: 222), (8) CD161-LAMP-sCD40L (SEQ ID NO: 235), (9) Spike-LAMP-sCD40L (2-V Covid vaccine; SEQ ID NO: 230), and (10) ITI-COVID-19 bicistronic vaccine (ITI-Bicistronic-S1-LAMP-RBG pA-EF2-S2P BHG pA; first generation COVID-19 vaccine; SEQ ID NO: 228). Sequences for these constructs and their components are provided in the table below.

For example, a viral vector can be constructed comprising a first polynucleotide sequence to generate the different structures ILC-1 to ILC-6 shown in FIG. 1, including the structure of ILC-4 that was present in some embodiments or similar structures comprising a first antigen of interest (a priming antigen) in, or replacing, the LAMP hinge region between first and second homology domains of a LAMP protein (or at least between two Cysteine Conserved Fragments). The LAMP domains illustrated in FIG. 1 were derived from the amino acid sequences shown in FIGS. 3-10. Corresponding domains can also be cloned from the orthologous sequences by identifying the equivalent domains when compared to the human sequence. An antigen of interest (including one or more antigens of interest) can be cloned into the described LAMP construct either individually or in combination. The viral vector can also be constructed to encode an expression cassette comprising the second polynucleotide sequence encoding an IREG or a second antigen, which may be operably linked to a secretion signal sequence.

The relatively “compact” size of the ILC-4 LAMP construct is advantageous in some embodiments inasmuch as it may reduce size constraints associated with including a second polynucleotide sequence. Moreover, as described in the abovementioned U.S. Pat. No. 11,203,629, ILC-4 LAMP constructs have been found to provide stronger immune responses (e.g., stronger T-cell and/or antibody responses) than other LAMP constructs tested. Thus, in some embodiments, the bicistronic LAMP construct is uses an ILC-4 design, i.g., comprises an ILC-4 LAMP-antigen construct general structure.

In some embodiments, an isolated nucleic acid, e.g. a vector or vaccine, comprising expression cassettes, that encodes the bicistronic LAMP construct is a DNA vector, while in other cases it is an RNA vector, including a self-amplifying RNA vector.

### D. Antigens for Use in Bicistronic LAMP Constructs

### 1. SARS-COV-2 Viral Antigens and Bicistronic Constructs

In some embodiments, the antigen in a LAMP-antigen construct may comprise an antigen from a SARS COV2 or other viral infectious agent, such as a viral spike protein. In some cases, a bicistronic construct may also express a second antigen from the same infectious agent for secretion. In other cases, the bicistronic construct may express an IREG polypeptide as a second polypeptide.

In one particular example, a first generation vector was constructed for use as a vaccine against the severe acute respiratory syndrome coronavirus (SARS-COV-2 virus, otherwise known as COVID-19). Except for generation neutralizing antibodies by an effective vaccine, T cells are an important component of naturally acquired protective immunity to many infectious diseases, many vaccines and vaccines in-development against viral infections are often to elicit virus-specific T cell responses that have the potential to activate innate immunity, have direct effector functions, as well as help the antibody responses, which can be used as preventive and therapeutic propose.

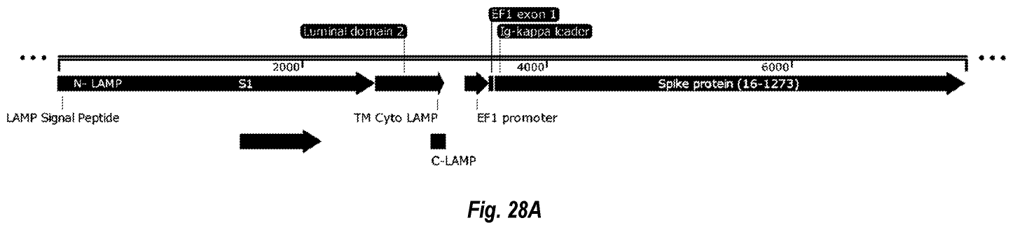

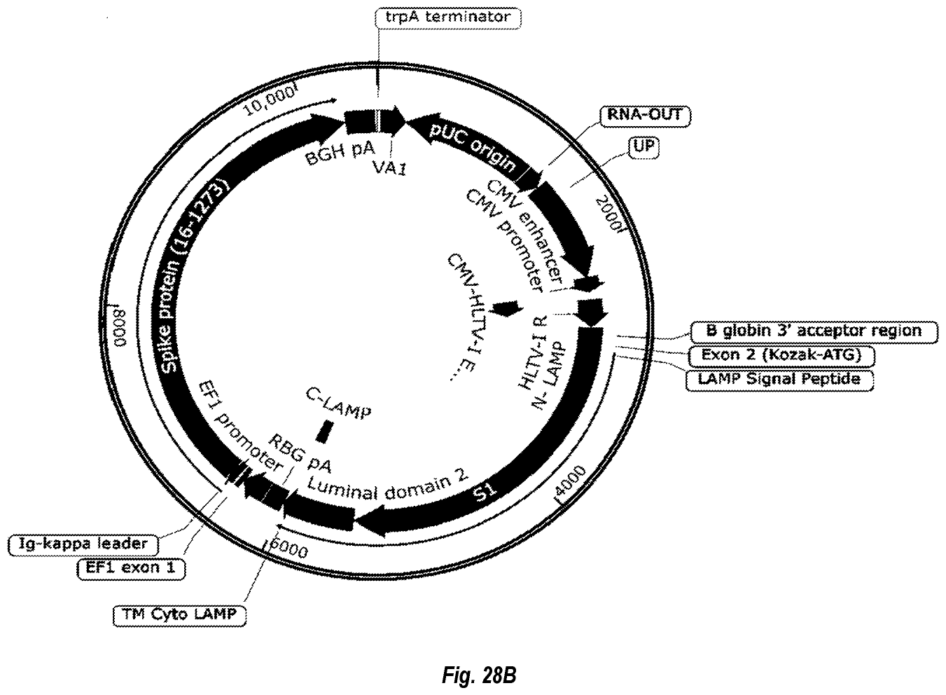

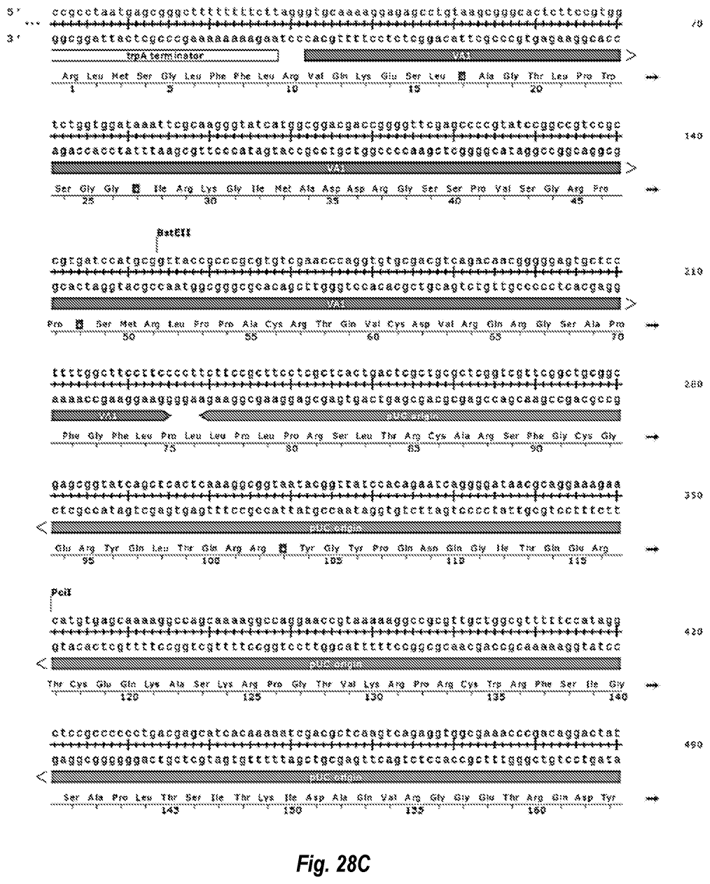

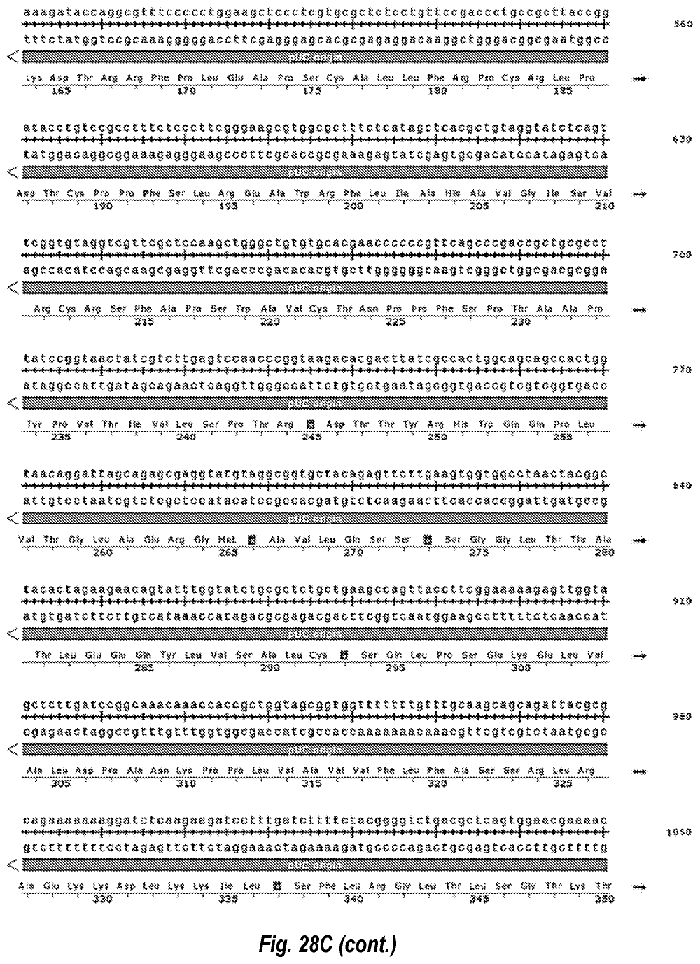

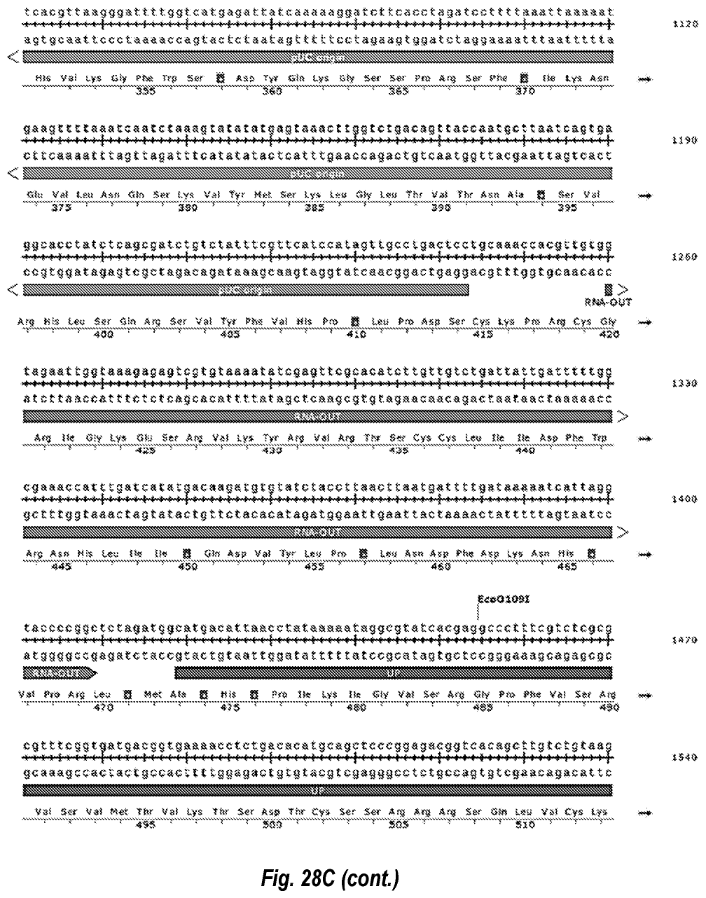

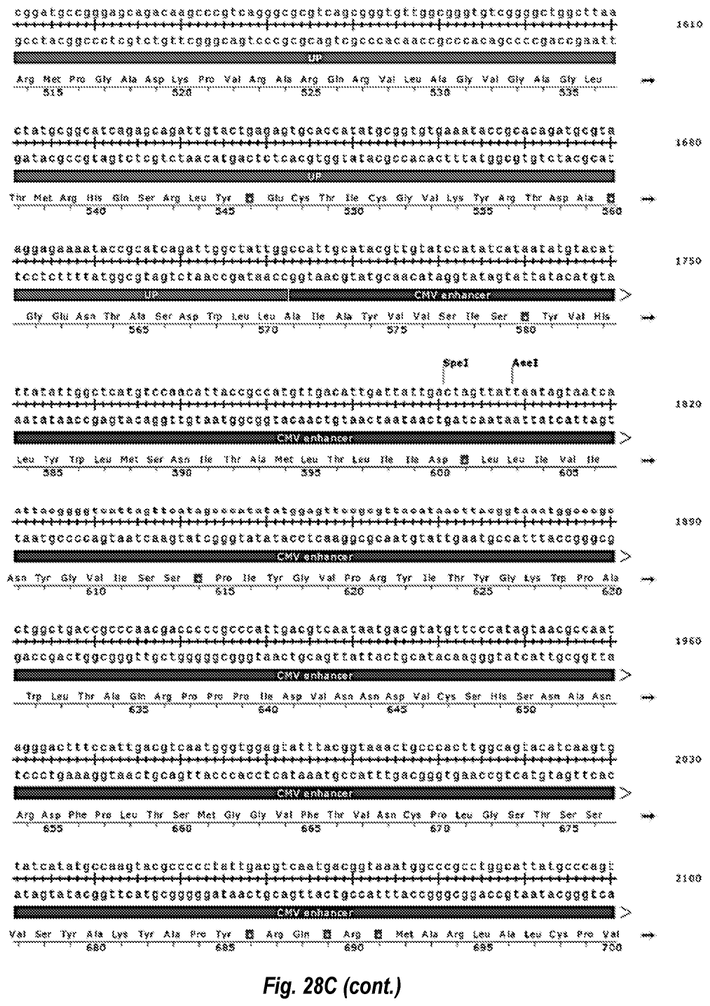

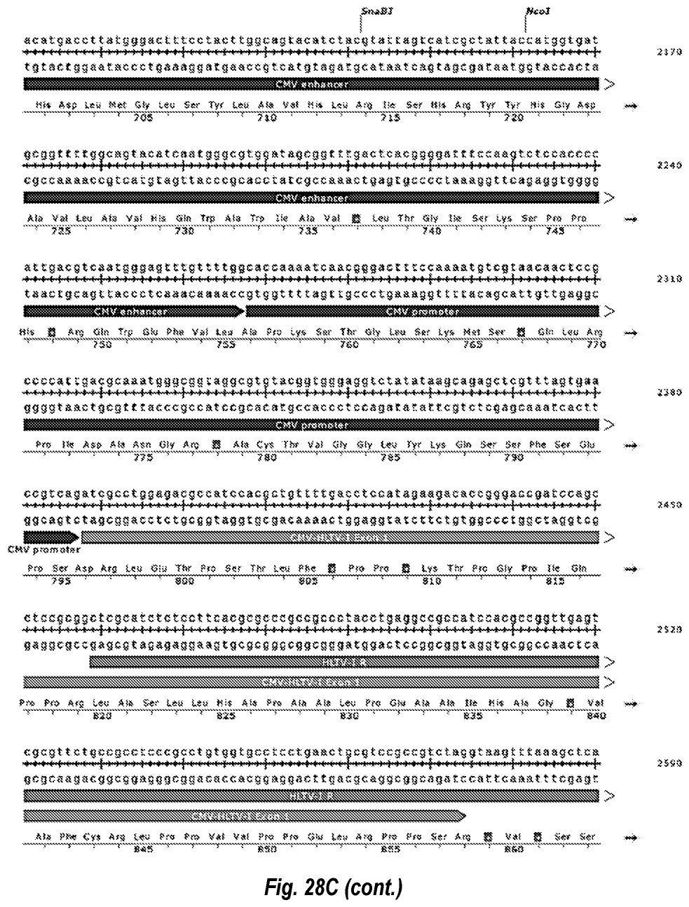

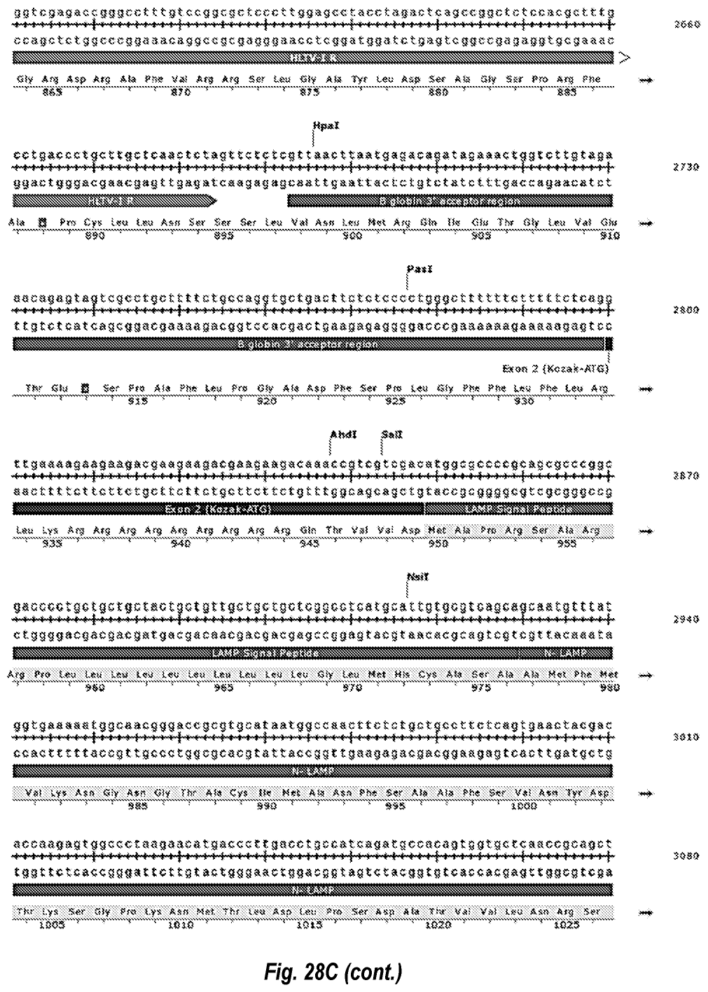

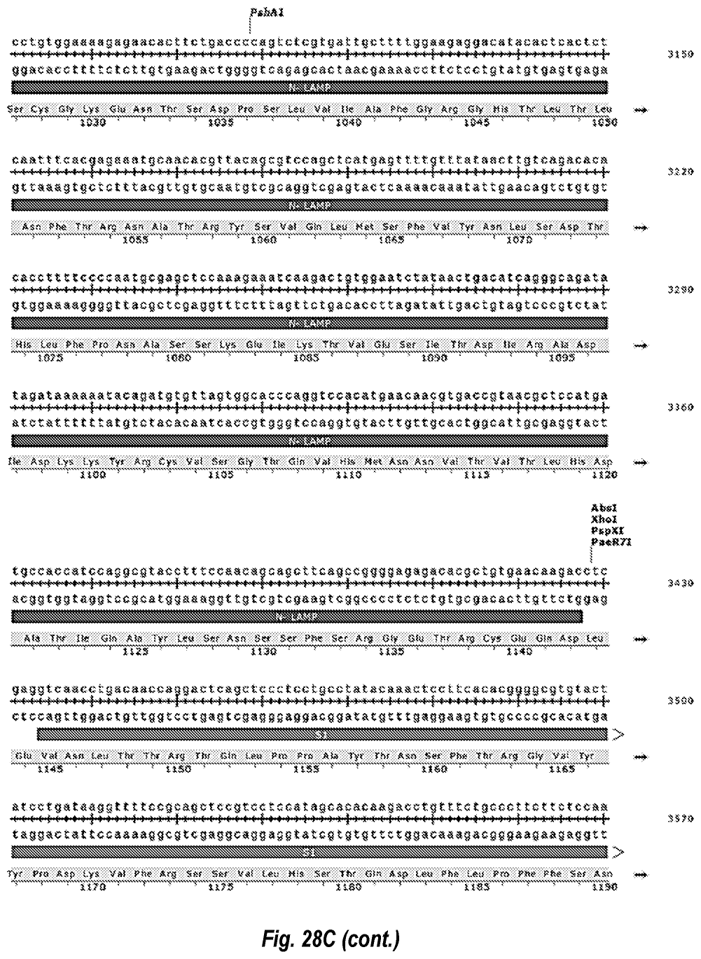

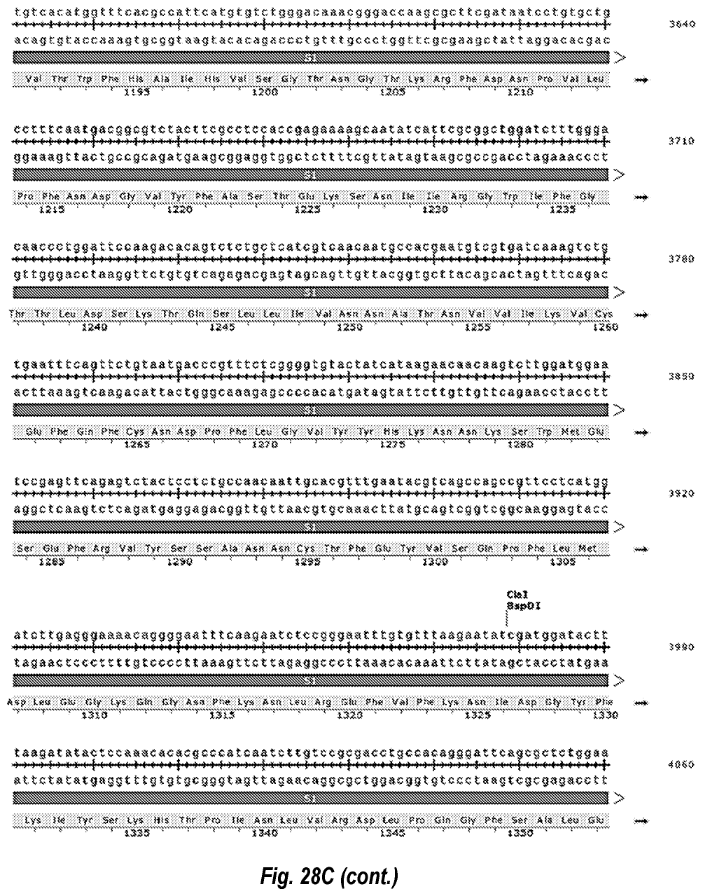

In order to induce both T cell and antibody responses to prevent the infection of SARS-Cov-2 or reduce symptoms associated with infection, a vector was designed, in which two viral proteins were expressed separately. The first fusion protein composed of LAMP and viral spike S1 subunit protein aimed to elicit rapid and robust S1-specific CD4+ T cell responses. The second protein was full-length spike protein with two proline substitution, which was driven by an independent promoter Human elongation factor-1 alpha (EF1) and also had a sequence peptide (IgK SP) on its N-terminus.54nub design enabled the generation of prefusion-stabilized spike protein and its secretion to present the antigens to B cells. The robust S1-specific CD4 T cells elicited by first promoter helped not only the function of CD8 T cell but also potentiated the neutralizing antibodies to SARS-Cov-2. (See FIGS. 28A-C for description of the vector.)

The first generation ITI-COVID-19 bicistronic vaccine encodes for the expression of the S1 and S2 subunits of the virus surface-anchored spike glycoprotein. The S1 and S2 subunits of spike mediate entry of the SARS-COV-2 virus into a host cell. Using a nucleic acid molecule for an ILC-4 LAMP construct, the S1 coding sequence (GenBank MN908974) was located between the polynucleotide sequences encoding two LAMP homology domains (N-Lamp and Luminal domain 2). The S1 coding sequence was operably linked to a CMV promoter under the influence of a CMV enhancer sequence, so that expression in a host cell resulted in an ILC-4 LAMP construct comprising the S1 antigen for processing and presentation to MHC class II molecules (i.e., to provide the “priming antigen”). The S2 coding sequence was provided elsewhere on the vector and was operably linked to a polynucleotide sequence encoding an Ig-kappa secretion signal (leader sequence) and an EF1 promoter sequence, so that expression in a host cell resulted in an S2 antigen for secretion (i.e., to provide the “boosting antigen”). The vector thereby provided a single nucleic acid molecule for introduction into a suitable host or target cell capable of providing both priming and boosting antigens to elicit an enhanced immune response. This may therefore confer a significant advantage over the use of a vector which only encodes a bicistronic LAMP construct inasmuch as any desire or requirement to enhance the immune response elicited by the LAMP construct will require the administration of a separately administered booster vaccine (e.g., comprising the antigen) at one or more time intervals.

A second generation COVID-19 vector, Spike-LAMP-sCD40L, was later designed and tested, as described in Examples 1 and 2 below, and was found to have unexpectedly even higher immune response to the first generation vector. This second generation vector encodes a LAMP-antigen sequence as provided in SEQ ID NO: 229 in an ILC-4 format, in which the particular Spike was derived from the B1.351 variant. It is to be understood that, as COVID-19 evolves further, other variant Spike antigens could be used to replace the one used for this vector. And, instead of a second COVID-19 antigen as a second polypeptide, this second generation vector encodes a fusion protein of lung surfactant binding protein D (SPD) and CD40L extracellular domain, as provided in SEQ ID NO: 233.

It is to be understood that variants of such isolated nucleic acids are encompassed herein, in which the LAMP-antigen construct comprises a COVID-19 Spike protein or similar viral antigen protein and an IREG polypeptide, such as an SPD-CD40L or an extracellular domain or complete protein sequence of another IREG such as CD80, OX40, Flt3L, GM-CSF, IL-12, IL-21, IL-23, IL-15, CD70, CD86, IL-7, IL-18, or IL-33, which can in some cases optionally be further fused to a domain such as an immunoglobulin Fc domain. Linkers may also be present between the domains of the first or second polypeptide in some embodiments. And in some embodiments, the second polypeptide may be secreted, and thus operably linked to a secretory signal sequence.

### a) Exemplary Spike Protein Sequences

In some embodiments, a LAMP-antigen construct may comprise an infectious disease antigen, such as a bacterial or viral antigen. In some embodiments, the viral antigen is a spike protein or domain of a spike protein. In some cases, the viral antigen is derived from a coronavirus, such as a SARS virus, such as SARS-COV-2 (COVID-19) virus. In some embodiments, an antigen used in a bicistronic construct herein is selected from antigens encoded by the SARS-COV-2 virus, such as the S1 spike subunit or S2 spike subunit. Examples of such S1 spike subunit include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 118 and/or an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 119 or SEQ ID NO: 231. In some embodiments, the bicistronic construct comprises a polynucleotide encoding an amino acid sequence at least 60%, at least 70%, at least 75%, at least 80%, at least 85%, at least 90%, at least 95%, at least 96%, at least 97%, at least 98%, or at least 99% sequence identity of SEQ ID NO: 229. In some embodiments, the bicistronic construct comprises a polynucleotide comprising at least 60%, at least 70%, at least 75%, at least 80%, at least 85%, at least 90%, at least 95%, at least 96%, at least 97%, at least 98%, or at least 99% sequence identity of SEQ ID NO: 232.

In some cases, the corresponding second polypeptide expressed by the bicistronic construct is an IREG polypeptide, such as an SPD-CD40L or an extracellular domain or complete protein sequence of another IREG such as CD80, OX40, Flt3L, GM-CSF, IL-12, IL-21, IL-23, IL-15, CD70, CD86, IL-7, IL-18, or IL-33, which can in some cases optionally be further fused to a domain such as an immunoglobulin Fc domain.

### 2. Cancer-Related Antigens

In other embodiments, the LAMP-antigen construct may comprise a cancer antigen. Candidates for cancer immunotherapy, using the vaccines comprising the bicistronic LAMP construct described herein, would be any patient with a cancer such as, for example, patients with documented Epstein-Barr virus associated lymphomas, patients with HPV associated cervical carcinomas, patients with chronic HCV, or patients with a defined re-arrangement or mutation in an oncogene or tumor suppressor gene.

In some embodiments, cancers that can be treated using the vaccines comprising the bicistronic LAMP construct described herein include, but are not limited to all stages of progression, including hyperplasia of an adenocarcinoma, sarcoma, skin cancer, melanoma, Merkel cell carcinoma, bladder cancer, brain cancer, breast cancer, uterine cancer, ovarian cancer, prostate cancer, lung cancer (including, but not limited to NSCLC, SCLC, squamous cell cancer), colorectal cancer, anal cancer, rectal cancer, cervical cancer, liver cancer, head and neck cancer, oral cancer, salivary gland cancer, esophageal cancer, pancreatic (pancreas) cancer, pancreatic ductal adenocarcinoma (PDA), renal cancer, stomach cancer, kidney cancer, multiple myeloma or cerebral cancer.

It is envisioned that therapy with a vaccine composition comprising a nucleic acid molecule as described herein could be utilized at any period during the course of the individual's cancer, once it is identified. It is also possible that in high-risk patients, vaccination in order to prevent the subsequent emergence of a cancer.

Examples of such cancer antigens include HER2, CD161, and NY-ESO1, or their extracellular domains.

### a) HER2

In some embodiments, a LAMP-antigen construct comprises a HER2 antigen. In some cases, the HER2 antigen comprises an extracellular domain (ECD) portion of HER2. In some cases, the antigen is selected from an amino acid sequence at least 60%, at least 70%, at least 75%, at least 80%, at least 85%, at least 90%, at least 95%, at least 96%, at least 97%, at least 98%, at least 99%, or with 100% sequence identity of SEQ ID NO: 200. In some embodiments, the HER2 antigen coding nucleotide sequence has at least 60%, at least 70%, at least 75%, at least 80%, at least 85%, at least 90%, at least 95%, at least 96%, at least 97%, at least 98%, or at least 99% sequence identity of SEQ ID NO: 201.

In some embodiments, a HER2-LAMP-antigen construct encodes an amino acid sequence at least 60%, at least 70%, at least 75%, at least 80%, at least 85%, at least 90%, at least 95%, at least 96%, at least 97%, at least 98%, at least 99%, or with 100% sequence identity of SEQ ID NO: 195. In some embodiments, a LAMP-antigen construct encodes a HER2-LAMP and comprises a nucleotide sequence at least 60%, at least 70%, at least 75%, at least 80%, at least 85%, at least 90%, at least 95%, at least 96%, at least 97%, at least 98%, at least 99%, or with 100% sequence identity of SEQ ID NO: 196. In some embodiments, the HER2-LAMP antigen construct has a nucleotide coding sequence at least 60%, at least 70%, at least 75%, at least 80%, at least 85%, at least 90%, at least 95%, at least 96%, at least 97%, at least 98%, at least 99%, or with 100% sequence identity of SEQ ID NO: 199 followed by SEQ ID NO: 201 followed by SEQ ID NO: 203. In some embodiments, the HER2-LAMP antigen construct has a polypeptide sequence at least 60%, at least 70%, at least 75%, at least 80%, at least 85%, at least 90%, at least 95%, at least 96%, at least 97%, at least 98%, at least 99%, or with 100% sequence identity of SEQ ID NO: 198 followed by SEQ ID NO: 200 followed by SEQ ID NO: 202, optionally with one or two linker sequences between these segments.

In some cases, the corresponding second polypeptide expressed by the bicistronic construct is an IREG polypeptide, such as an SPD-CD40L or an extracellular domain or complete protein sequence of another IREG such as CD80, OX40, Flt3L, GM-CSF, IL-12, IL-21, IL-23, IL-15, CD70, CD86, IL-7, IL-18, or IL-33, which can in some cases optionally be further fused to a domain such as an immunoglobulin Fc domain.

### b) NY-ESO1

In some embodiments, the LAMP-antigen construct comprises an antigen of NY-ESO1. Examples of such NY-ESO1 antigen sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 223. In some embodiments, the NY-ESO1 nucleotide coding sequence is at least 60%, at least 70%, at least 75%, at least 80%, at least 85%, at least 90%, at least 95%, at least 96%, at least 97%, at least 98%, at least 99%, or 100% sequence identity of SEQ ID NO: 224.

In some embodiments, the LAMP-antigen construct comprises: (a) a polynucleotide encoding an amino acid sequence at least 60%, at least 70%, at least 75%, at least 80%, at least 85%, at least 90%, at least 95%, at least 96%, at least 97%, at least 98%, or at least 99% sequence identity of SEQ ID NO: 221. In some embodiments, the LAMP-antigen construct coding sequence is at least 60%, at least 70%, at least 75%, at least 80%, at least 85%, at least 90%, at least 95%, at least 96%, at least 97%, at least 98%, at least 99%, or with 100% sequence identity of SEQ ID NO: 199 followed by SEQ ID NO: 224 followed by SEQ ID NO: 203.

In some cases, the corresponding second polypeptide expressed by the bicistronic construct is an IREG polypeptide, such as an IL-15 or an extracellular domain or complete protein sequence of another IREG such as CD40, CD80, OX40, Flt3L, GM-CSF, IL-12, IL-21, IL-23, CD70, CD86, IL-7, IL-18, or IL-33, which can in some cases optionally be further fused to a domain such as an immunoglobulin Fc domain.

### c) CD161

In some embodiments, the LAMP-antigen construct includes an antigen of CD161, such as an ECD of CD161. Examples of such CD161 sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 236. In some embodiments, a CD161 ECD nucleotide coding sequence is at least 60%, at least 70%, at least 75%, at least 80%, at least 85%, at least 90%, at least 95%, at least 96%, at least 97%, at least 98%, at least 99%, or 100% sequence identity of SEQ ID NO: 237.

In some embodiments, the LAMP-antigen construct comprises: (a) a polynucleotide encoding an amino acid sequence at least 60%, at least 70%, at least 75%, at least 80%, at least 85%, at least 90%, at least 95%, at least 96%, at least 97%, at least 98%, or at least 99% sequence identity of SEQ ID NO: 234. In some embodiments, the LAMP-antigen construct coding sequence is at least 60%, at least 70%, at least 75%, at least 80%, at least 85%, at least 90%, at least 95%, at least 96%, at least 97%, at least 98%, at least 99%, or with 100% sequence identity of SEQ ID NO: 199 followed by SEQ ID NO: 237 followed by SEQ ID NO: 203.

In some cases, the corresponding second polypeptide expressed by the bicistronic construct is an IREG polypeptide, such as an SPD-CD40L or an extracellular domain or complete protein sequence of another IREG such as CD80, OX40, Flt3L, GM-CSF, IL-12, IL-21, IL-23, IL-15, CD70, CD86, IL-7, IL-18, or IL-33, which can in some cases optionally be further fused to a domain such as an immunoglobulin Fc domain.

### Additional Cancer Antigens

The following antigens shown in Table A can also be cloned into each of the bicistronic LAMP-antigen constructs described herein using techniques known to the skilled artisan. The sequences/fragments/epitopes described in the fourth column, for example, can be also cloned into the LAMP-antigen constructs as described herein. Moreover, any one of the cancer antigens listed in Table A can be combined with any other antigen listed in Table A including the sequences/fragments/epitopes described in the fourth column) and inserted into the LAMP-antigen constructs as described herein. Or any one of the cancer antigens of Table A can be combined with any other cancer antigen described in the instant disclosure and inserted into the LAMP-antigen constructs herein.

In some cases, the corresponding second polypeptide expressed by the bicistronic construct that includes a cancer antigen from Table A is an IREG polypeptide, such as an SPD-CD40L or an extracellular domain or complete protein sequence of another IREG such as CD80, OX40, Flt3L, GM-CSF, IL-12, IL-21, IL-23, IL-15, CD70, CD86, IL-7, IL-18, or IL-33, which can in some cases optionally be further fused to a domain such as an immunoglobulin Fc domain.

| SEQ | | | |
|---|---|---|---|
| ID | Protein Name | Accession/ | Exemplary Epitopes/Fragments that can be cloned into the LAMP |
| NO: | of Antigen | Derived From | Constructs described herein, comprise at least: . . |
| 254 | Large T antigen | YP_009111421 | Amino acids 1-327 of SEQ ID NO: 254, with or without mutations at amino acid |
| | | | 220, wherein the wild type serine is preferably replaced with an alanine (or other |
| | | | inactivating amino acid) (e.g., SEQ ID NO: 255-256) so that phosphorylation of the |
| | | | Rb binding motif is inhibited as described in Sharma et al. 38, 1153-1162 (IJC |
| | | | 2016). Fragments of amino acids 1-327 of SEQ ID NO: 254 are also contemplated, |
| | | | such as those exemplified as SEQ ID NO: 255-256, so long as these fragments |
| | | | retain the ability to raise an antibody response to the Large T Antigen of Merkel cell |
| | | | polyomavirus (MCPγV). |
| | | | One example of a Large T antigen LAMP construct that may be used with a |
| | | | bicistronic construct herein is a construct of SEQ ID NO: 879 or 880. |
| 255 | Large T antigen | YP_009111421 | Amino acids 1-327 of SEQ ID NO: 254 wherein S220 is any amino acid (X) |
| 256 | Large T antigen | YP_009111421 | Amino acids 1-327 of SEQ ID NO: 254 wherein S220 is alanine (A) |
| 257 | Small T antigen | YP_009111422 | MCPyVgp4 small T antigen, full length and fragments. |
| 258 | MAGE-A10 | UniProtKB: A0A024RC14 | |
| 259 | MAGE-A12 | UniProtKB: P43365 | |
| 260 | MAGE-A1 | UniProtKB: P43355 | |
| 261 | MAGE-A2 | UniProtKB: P43356 | |
| 262 | MAGE-A3 | UniProtKB: P43357 | |
| 263 | MAGE-A4 | UniProtKB: A0A024RC12 | |
| 264 | MAGE-A4 | UniProtKB: P43358 | |
| 265 | MAGE-A4 | UniProtKB: Q1RN33 | |
| 266 | MAGE-A6 | UniProtKB: A8K072 | |
| 267 | MAGE-A6 | UniProtKB: P43360 | |
| 268 | MAGE-A6 | UniProtKB: Q6FHI5 | |
| 269 | MAGE-A9 | UniProtKB: P43362 | |
| 270 | MAGE-B10 | UniProtKB: Q96LZ2 | |
| 271 | MAGE-B16 | UniProtKB: A2A368 | |
| 272 | MAGE-B17 | UniProtKB: A8MXT2 | |
| 273 | MAGE-_B1 | UniProtKB: Q96TG1 | |
| 274 | MAGE-B2 | UniProtKB: O15479 | |
| 275 | MAGE-B3 | UniProtKB: O15480 | |
| 276 | MAGE-B4 | UniProtKB: O15481 | |
| 277 | MAGE-B5 | UniProtKB: Q9BZ81 | |
| 278 | MAGE-B6 | UniProtKB: Q8N7X4 | |
| 279 | MAGE-C1 | UniProtKB: O60732 | |
| 280 | MAGE-C2 | UniProtKB: Q9UBF1 | |
| 281 | MAGE-C3 | UniProtKB: Q8TD91 | |
| 282 | MAGE-D1 | UniProtKB: Q9Y5V3 | |
| 283 | MAGE-D2 | UniProtKB: Q9UNF1 | |
| 284 | MAGE-D4 | UniProtKB: Q96JG8 | |
| 285 | MAGE-_E1 | UniProtKB: Q6IAI7 | |
| 286 | MAGE- | UniProtKB: Q9HCI5 | |
| | E1_(MAGE1) | | |
| 287 | MAGE-E2 | UniProtKB: Q8TD90 | |
| 288 | MAGE-F1 | UniProtKB: Q9HAY2 | |
| 289 | MAGE-H1 | UniProtKB: Q9H213 | |
| 290 | MAGEL2 | UniProtKB: Q9UJ55 | |
| 291-293 | pp65 | ABQ23593 | Exemplary pp65 antigen sequences useful in pp65-LAMP constructs herein also |
| | | | include those comprising: LLQTGIHVRVSQPSL (SEQ ID NO: 291, pos 41-55) |
| | | | and/or ALPLKMLNIPSINVH (SEQ ID NO: 291, pos 115-129) and/or |
| | | | DQYVKVYLESFCEDV (SEQ ID NO: 291, pos 221-235) and/or |
| | | | IIKPGKISHIMLDVAFTSH (SEQ ID NO: 291, pos 281-299) and/or |
| | | | PQYSEHPTFTSQYRIQGKL (SEQ ID NO: 291, pos 361-379) and/or |
| | | | PPWQAGILARNLVPMV (SEQ ID NO: 291, pos 485-500) and/or |
| | | | KYQEFFWDANDIYRIFA (SEQ ID NO: 291, pos 509-525), wherein each |
| | | | epitope can be combined in any order and/or in any combination and cloned into a |
| | | | LAMP Construct described herein, wherein each epitope is optionally separated by |
| | | | a linker, such as, for example GPGPG (SEQ ID NO: 292, pos 36-40) or PMGLP |
| | | | (SEQ ID NO: 293, pos 36-40). Representative construct inserts are shown as SEQ |
| | | | ID NO: 292 and SEQ ID NO: 293 |
| 294-297 | gB | P06473 | Exemplary gB antigen sequences herein also include those comprising: |
| | | | TTSAQTRSVYSQHVT (SEQ ID NO: 294, pos 44-58) and/or |
| | | | QLIPDDYSNTHSTRYV (SEQ ID NO: 294, pos 212-227) and/or |
| | | | VSVFETSGGLVVFWQ (SEQ ID NO: 294, pos 418-432) and/or |
| | | | NSAYEYVDYLFKRMIDLS (SEQ ID NO: 294, pos 622-639), wherein each |
| | | | epitope can be combined in any order and/or in any combination and cloned into a |
| | | | LAMP Construct described herein, wherein each epitope is preferably separated by |
| | | | a linker, such as, for example GPGPG (SEQ ID NO: 296, pos 16-20) or PMGLP |
| | | | (SEQ ID NO: 297, pos 16-20). Representative construct inserts are shown as SEQ |
| | | | ID NO: 296 and SEQ ID NO: 297. |
| 298-300 | IE1 | P13202 | Exemplary 1E1 antigen sequences also include those comprising: |
| | | | VLAELVKQIKVRVDMVRHRIKEHMLKKYTQ (SEQ ID NO: 298, pos 81- |
| | | | 110) and/or IVPEDKREMWMACIKELH (SEQ ID NO: 298, pos 158-175) |
| | | | and/or KDELRRKMMYMCYRNIEFFTKNSAFPKTT (SEQ ID NO: 298, pos |
| | | | 197-225) and/or SVMKRRIEEICMKVFAQYI (SEQ ID NO: 298, pos 337-355) |
| | | | and/or AIAEESDEEEAIVAY (SEQ ID NO: 298, pos 373-387) and/or |
| | | | VKSEPVSEIEEVAPEEEEDG (SEQ ID NO: 298, pos 449-468) wherein each |
| | | | epitope can be combined in any order and/or in any combination and cloned into a |
| | | | LAMP Construct described herein, wherein each epitope is preferably separated by |
| | | | a linker, such as, for example GPGPG (SEQ ID NO: 299, pos 31-35) or PMGLP |
| | | | (SEQ ID NO: 300, pos 31-35). Representative construct inserts are shown as SEQ |
| | | | ID NO: 299 and SEQ ID NO: 300. |
| 301 | Morphological | AAA66543 | |
| | transforming | | |
| | region II | | |
| | (“MTRII”), | | |
| | Human | | |
| | betaher- | | |
| | pesvirus ”) | | |
| 302 | Representative | | |
| | MTRII LAMP | | |
| | Construct Insert | | |
| 303 | Human | AMJ53524 | |
| | betaherpesvirus | | |
| | 5 | | |
| 304 | US28 | | |
| 305 | IGFBP2 | NP_000588 | Exemplary IGFBP2 antigen sequences also comprise amino acids 39-328 of SEQ |
| | | | ID NO: 128; (b) amino acids 40-328 of SEQ ID NO: 128; (c) amino acids 43-135 of |
| | | | SEQ ID NO: 128; (d) amino acids 1-163 of SEQ ID NO: 128; (e) amino acids 2-163 |
| | | | of SEQ ID NO: 128; (f) amino acids 39-163 of SEQ ID NO: 128; (g) amino acids |
| | | | 40-163 of SEQ ID NO: 128; (h) amino acids 229-309 of SEQ ID NO: 128; (i) |
| | | | amino acids 2-328 of SEQ ID NO: 128; and/or (j) amino acids 1-328 of SEQ ID |
| | | | NO: 128 |
| 306 | HCMVA Viral | P17150 | |
| | interleukin-10 | | |
| | (“IL10”) | | |
| 307 | Representative | | |
| | HCMVA Viral | | |
| | IL-10 LAMP | | |
| | Construct Insert | | |
| 308 | HCMVM Membrane | F5HAM0 | |
| | glycoprotein | | |
| | UL144 (“UL144”) | | |
| 309 | Representative | | |
| | HCMV Membrane | | |
| | glycoprotein | | |
| | UL144 LAMP | | |
| | Construct Insert | | |
| 310 | HCMVM Protein | Q6RJQ3 | |
| | UL141 | | |
| | (“UL141”) | | |
| 311 | Representative | | |
| | HCMV Protein | | |
| | UL141 LAMP | | |
| | Construct Insert | | |
| 312 | HCMVA Unique | P09727 | |
| | short US11 | | |
| | glycoprotein | | |
| | (“US11”) | | |
| 313 | Representative | | |
| | HCMVA Unique | | |
| | Short US11 | | |
| | Glycoprotein | | |
| | LAMP Construct | | |
| | Insert | | |
| 314-317 | HCMV Envelope | A0A0G2TM81 | Exemplary HCMV envelope glycoprotein HOS antigen sequences also include |
| | glycoprotein | | those comprising: TYNSSLRNSTVVRENAISFNFFQSYNQYYVFHMPR (SEQ |
| | H OS | | ID NO: 314, pos 60-94) and/or DLTETLERYQQRLNTYALVSKDLASYRSFS |
| | (“HOS”) | | (SEQ ID NO: 314, pos 110-139) and/or SHTTSGLHRPHFNQTCILFD (SEQ ID |
| | | | NO: 314, pos 180-199) and/or QLNRHSYLKDPDFLDAALDF (SEQ ID NO: |
| | | | 314, pos 285-304), wherein each epitope can be combined in any order and/or in |
| | | | any combination and cloned into a LAMP Construct described herein, wherein |
| | | | each epitope is preferably separated by a linker, such as, for example GPGPG (SEQ |
| | | | ID NO: 316, pos 36-40) or PMGLP (SEQ ID NO: 317, pos 36-40). See, for |
| | | | instance SEQ ID NOs: 315-317. |
| 318-320 | HCMVT Viral | Q6SWP7 | Exemplary IE2 antigen sequences also include those |
| | transcription | | comprising: RRGRVKIDEVSRMFR (SEQ ID NO: 318, pos 356-370) and/or |
| | factor IE2 | | GIQIIYTRNHEVKSE (SEQ ID NO: 318, pos 408-422) and/or |
| | (“IE2”) | | LSTPFLMEHTMPVTHPPEVA (SEQ ID NO: 318, pos 438-457) wherein each |
| | | | epitope can be combined in any order and/or in any combination and cloned into a |
| | | | LAMP Construct described herein, wherein each epitope is preferably separated by |
| | | | a linker, such as, for example GPGPG (SEQ ID NO: 319, pos 16-20) or PMGLP |
| | | | (SEQ ID NO: 320, pos 16-20). See, for instance SEQ ID NOs: 319-320. |
| 321-323 | TERT Isoform 1 | NP_937983.2 | |
| | (“TERT”) | | |
| 324 | Survivin | NP_001125727 | |
| 325-327 | Tetanus | 1AF9_A | Exemplary tetanus antigen sequences also include those |
| | | | comprising:PGINGKAIHLVNNESSE (SEQ ID NO: 325, pos 53-69) and/or |
| | | | FNNFTVSFWLRVPKVSASHLEQYGT (SEQ ID NO: 325, pos 84-108) and/or |
| | | | YVSIDKFRIFCKALNPKEIEKLYTSYLS (SEQ ID NO: 325, pos 220-247) |
| | | | and/or ILRVGYNAPGIPLYKKMEAVKLRDLK (SEQ ID NO: 325, pos 361- |
| | | | 386) wherein each epitope can be combined in any order and/or in any |
| | | | combination and cloned into a LAMP Construct described herein, wherein each |
| | | | epitope is preferably separated by a linker, such as, for example GPGPG (SEQ ID |
| | | | NO: 326, pos 18-22) or PMGLP (SEQ ID NO: 327, pos 18-22). Representative |
| | | | construct inserts are shown as SEQ ID NO: 326 and SEQ ID NO: 327. |
| 328 | Cancer Testis | NP-001318.1 | |
| | Antigen | | |
| | NY-ESO-1 (“NY- | | |
| | ESO-1”) | | |
| 329-331 | HER2 | AAA75493.1 | A further example HER2 antigen sequence comprises SEQ ID NO: 198, and is |
| | | | encoded by SEQ ID NO: 199. |
| 332-334 | HER3 | NP_001973.2 | Construct comprising at least one of: amino acids 20-643, 665-1201, and/or 1209- |
| | | | 1342 of SEQ ID NO: 332, wherein each epitope can be combined in any order |
| | | | and/or in any combination and cloned into a LAMP Construct described herein, |
| | | | wherein each epitope is preferably separated by a linker, such as, for example |
| | | | GPGPG or PMGLP. Representative construct inserts are shown as SEQ ID |
| | | | NO: 333 and SEQ ID NO: 334. |
| 335 | HVEM | NP_003811.2 | An example is a sequence comprising amino acids 39-202 of SEQ ID NO: 335 |
| 336 | | | |
| 337-340 | HPV Constructs | | Examples include sequences used to create the constructs: (a) HPV 16 E6 (SEQ |
| | | | ID NO: 337); (b) HPV 18 E6 (SEQ ID NO: 338); (c) HPV 16 E7 (SEQ ID |
| | | | NO: 339); (d) HPV 18 E7 (SEQ ID NO: 340) |
| | | | Representative Constructs: |
| | | | SEQ ID NO: 341 (Representative HPV 16 E6-E7 Construct) |
| | | | SEQ ID NO: 342 (Representative HPV 18 E6-E7 Construct) |
| | | | SEQ ID NO: 343 (HPV 16 E6-linker-HPV 18 E6-linker-HPV 16 E7-linker- |
| | | | HPV 18 E7) |
| 346-349 | EBV Constructs | | Any of the following sequences, in any combination, could be used to create the |
| | | | constructs (a) EBV EBNA1 (SEQ ID NO: 346); (b) EBV Truncated EBNA-1 |
| | | | (SEQ ID NO: 347); (c) EBV gp350 (SEQ ID NO: 348); (d) EBV LMP2 (SEQ ID |
| | | | NO: 349). For example, a representative construct could comprise truncated |
| | | | EBNA-1 and LMP2. |
| 351-352 | HBV Constructs | | Any of the following sequences could be used (in either orientation) to create the |
| | | | constructs: |
| | | | HBV Middle S Protein (SEQ ID NO: 351) and HBV X Protein (SEQ ID NO: |
| | | | 352). For example, a representative construct could comprise Middle S Protein-X |
| | | | protein. |
| 353 | TIGIT | | |
| 354 | TEM8 | | |
| 355 | TEM1 | | |
| 356 | HER2 ECD + TM | | |
| 357 | CEA | | |
| 358 | TARP | | |
| 359 | PROSTEIN | | |
| 360 | PSMA | | |
| 361 | BIRC4 | | |
| 362 | Mucin-1 | | |
| 363 | Mucin-1 Isoform | | |
| 364 | CD40 Ligand | | |
| 352 | WT-1 | | |
| 366 | WT-1 Truncated | | |
| 367 | PRAME | | |
| 368 | LAGE-1 | | |
| 369 | MAGE A3 | | |
| 370-374 | Survivin | NP_001125727 | Representative constructs include those comprising one of SEQ ID NO: 370-374 |
| 375 | 1A01_HLA-A/m | UniProtKB: P30443 | |
| 376 | 1A02 | UniProtKB: P01892 | |
| 377 | 5T4 | UniProtKB: Q13641 | |
| 378 | ACRBP | UniProtKB: Q8NEB7 | |
| 379 | AFP | UniProtKB: P02771 | |
| 380 | AKAP4 | UniProtKB: Q5JQC9 | |
| 381 | alpha-actinin-_ | UniProtKB: B4DSX0 | |
| | 4/m | | |
| 382 | alpha-actinin-_ | UniProtKB: B4E337 | |
| | 4/m | | |
| 383 | alpha-actinin-_ | UniProtKB: O43707 | |
| | 4/m | | |
| 384 | alpha- | UniProtKB: A0A024RE16 | |
| | methylacyl- | | |
| | coenzyme_A_ | | |
| | racemase | | |
| 385 | alpha- | UniProtKB: A8KAC3 | |
| | methylacyl- | | |
| | coenzyme_A_ | | |
| | racemase | | |
| 386 | ANDR | UniProtKB: P10275 | |
| 387 | ART-4 | UniProtKB: Q9ULX3 | |
| 388 | ARTC1/m | UniProtKB: P52961 | |
| 389 | AURKB | UniProtKB: Q96GD4 | |
| 390 | B2MG | UniProtKB: P61769 | |
| 391 | B3GN5 | UniProtKB: Q9BYG0 | |
| 392 | B4GN1 | UniProtKB: Q00973 | |
| 393 | B7H4 | UniProtKB: Q7Z7D3 | |
| 394 | BAGE-1 | UniProtKB: Q13072 | |
| 395 | BASI | UniProtKB: P35613 | |
| 396 | BCL-2 | UniProtKB: A9QXG9 | |
| 397 | bcr/abl | UniProtKB: A9UEZ4 | |
| 398 | bcr/abl | UniProtKB: A9UEZ7 | |
| 399 | bcr/abl | UniProtKB: A9UEZ8 | |
| 400 | bcr/abl | UniProtKB: A9UEZ9 | |
| 401 | bcr/abl | UniProtKB: A9UF00 | |
| 402 | bcr/abl | UniProtKB: A9UF01 | |
| 403 | bcr/abl | UniProtKB: A9UF03 | |
| 404 | bcr/abl | UniProtKB: A9UF04 | |
| 405 | bcr/abl | UniProtKB: A9UF05 | |
| 406 | bcr/abl | UniProtKB: A9UF06 | |
| 407 | bcr/abl | UniProtKB: A9UF08 | |
| 408 | beta-catenin/m | UniProtKB: P35222 | |
| 409 | beta-catenin/m | UniProtKB: Q8WYA6 | |
| 410 | BING-4 | UniProtKB: O15213 | |
| 411 | BIRC7 | UniProtKB: Q96CA5 | |
| 412 | BRCA1/m | UniProtKB: A0A024R1V0 | |
| 413 | BRCA1/m | UniProtKB: A0A024R1V7 | |
| 414 | BRCA1/m | UniProtKB: A0A024R1Z8 | |
| 415 | BRCA1/m | UniProtKB: A0A068BFX7 | |
| 416 | BRCA1/m | UniProtKB: C6YB45 | |
| 417 | BRCA1/m | UniProtKB: C6YB47 | |
| 418 | BRCA1/m | UniProtKB: G3XAC3 | |
| 419 | BY55 | UniProtKB: O95971 | |
| 420 | CAMEL | UniProtKB: O95987 | |
| 421 | CASPA | UniProtKB: Q92851-4 | |
| 422 | cathepsin_B | UniProtKB: A0A024R374 | |
| 423 | cathepsin_B | UniProtKB: P07858 | |
| 424 | cathepsin_L | UniProtKB: A0A024R276 | |
| 425 | cathepsin_L | UniProtKB: P07711 | |
| 426 | cathepsin_L | UniProtKB: Q9HBQ7 | |
| 427 | CD1A | UniProtKB: P06126 | |
| 428 | CD1B | UniProtKB: P29016 | |
| 429 | CD1C | UniProtKB: P29017 | |
| 430 | CD1D | UniProtKB: P15813 | |
| 431 | CD1E | UniProtKB: P15812 | |
| 432 | CD20 | UniProtKB: P11836 | |
| 433 | CD22 | UniProtKB: O60926 | |
| 434 | CD22 | UniProtKB: P20273 | |
| 435 | CD22 | UniProtKB: QUEAF5 | |
| 436 | CD276 | UniProtKB: Q5ZPR3 | |
| 437 | CD33 | UniProtKB: B4DF51 | |
| 438 | CD33 | UniProtKB: P20138 | |
| 439 | CD33 | UniProtKB: Q546G0 | |
| 440 | CD3E | UniProtKB: P07766 | |
| 441 | CD3Z | UniProtKB: P20963 | |
| 442 | CD44_Isoform_1 | UniProtKB: P16070 | |
| 443 | CD44_Isoform_6 | UniProtKB: P16070-6 | |
| 444 | CD4 | UniProtKB: P01730 | |
| 445 | CD52 | UniProtKB: P31358 | |
| 446 | CD52 | UniProtKB: Q6IBD0 | |
| 447 | CD52 | UniProtKB: V9HWN9 | |
| 448 | CD55 | UniProtKB: B1AP15 | |
| 449 | CD55 | UniProtKB: D3DT85 | |
| 450 | CD55 | UniProtKB: D3DT86 | |
| 451 | CD55 | UniProtKB: P08174 | |
| 452 | CD56 | UniProtKB: P13591 | |
| 453 | CD80 | UniProtKB: A0N0P2 | |
| 454 | CD80 | UniProtKB: P33681 | |
| 455 | CD86 | UniProtKB: P42081 | |
| 456 | CD8A | UniProtKB: P01732 | |
| 457 | CDC27/m | UniProtKB: G5EA36 | |
| 458 | CDC27/m | UniProtKB: P30260 | |
| 459 | CDE30 | UniProtKB: P28908 | |
| 460 | CDK4/m | UniProtKB: A0A024RBB6 | |
| 461 | CDK4/m | UniProtKB: P11802 | |
| 462 | CDK4/m | UniProtKB: Q6LC83 | |
| 463 | CDK4/m | UniProtKB: Q96BE9 | |
| 464 | CDKN2A/m | UniProtKB: D1LYX3 | |
| 465 | CDKN2A/m | UniProtKB: G3XAG3 | |
| 466 | CDKN2A/m | UniProtKB: K7PML8 | |
| 467 | CDKN2A/m | UniProtKB: L8E941 | |
| 468 | CDKN2A/m | UniProtKB: Q8N726 | |
| 469 | CEA | RefSeq: NP_004354 | |
| 470 | CEAM6 | UniProtKB: P40199 | |
| 471 | CH3L2 | UniProtKB: Q15782 | |
| 472 | CLCA2 | UniProtKB: Q9UQC9 | |
| 473 | CML28 | UniProtKB: Q9NQT4 | |
| 474 | CML66 | UniProtKB: Q96RS6 | |
| 475 | COA-1/m | UniProtKB: Q5T124 | |
| 476 | coactosin- | UniProtKB: Q14019 | |
| | like_protein | | |
| 477 | collagen_XXIII | UniProtKB: L8EAS4 | |
| 478 | collagen_XXIII | UniProtKB: Q86Y22 | |
| 479 | COX-2 | UniProtKB: Q6ZYK7 | |
| 480 | CP1B1 | UniProtKB: Q16678 | |
| 481 | CSAG2 | UniProtKB: Q9Y5P2-2 | |
| 482 | CSAG2 | UniProtKB: Q9Y5P2 | |
| 483 | CT45A1 | UniProtKB: Q5HYN5 | |
| 484 | CT55 | UniProtKB: Q8WUE5 | |
| 485 | CT-_9/BRD6 | UniProtKB: Q58F21 | |
| 48 | CTAG2_Isoform_ | UniProtKB: O75638-2 | |
| | LAGE-1A | | |
| 487 | CTAG2_Isoform_ | UniProtKB: O75638 | |
| | LAGE-1B | | |
| 488 | CTCFL | UniProtKB: Q8NI51 | |
| 489 | Cten | UniProtKB: Q8IZW8 | |
| 490 | cyclin_B1 | UniProtKB: P14635 | |
| 491 | cyclin_D1 | UniProtKB: P24385 | |
| 492 | cyp-B | UniProtKB: P23284 | |
| 493 | DAM-10 | UniProtKB: P43366 | |
| 494 | DEP1A | UniProtKB: Q5TB30 | |
| 495 | E7 | UniProtKB: P03129 | |
| 496 | E7 | UniProtKB: P06788 | |
| 497 | E7 | UniProtKB: P17387 | |
| 498 | E7 | UniProtKB: P06429 | |
| 499 | E7 | UniProtKB: P27230 | |
| 500 | E7 | UniProtKB: P24837 | |
| 501 | E7 | UniProtKB: P21736 | |
| 502 | E7 | UniProtKB: P26558 | |
| 503 | E7 | UniProtKB: P36831 | |
| 504 | E7 | UniProtKB: P36833 | |
| 505 | E7 | UniProtKB: Q9QCZ1 | |
| 506 | E7 | UniProtKB: Q81965 | |
| 507 | E7 | UniProtKB: Q80956 | |
| 508 | EF1A2 | UniProtKB: Q05639 | |
| 509 | EFTUD2/m | UniProtKB: Q15029 | |
| 510 | EGFR | UniProtKB: A0A0B4J1Y5 | |
| 511 | EGFR | UniProtKB: E7BSV0 | |
| 512 | EGFR | UniProtKB: L0R6G1 | |
| 513 | EGFR | UniProtKB: P00533-2 | |
| 514 | EGFR | UniProtKB: P00533 | |
| 515 | EGFR | UniProtKB: Q147T7 | |
| 516 | EGFR | UniProtKB: Q504U8 | |
| 517 | EGFR | UniProtKB: Q8NDU8 | |
| 518 | EGLN3 | UniProtKB: Q9H6Z9 | |
| 519 | ELF2/m | UniProtKB: B7Z720 | |
| 520 | EMMPRIN | UniProtKB: Q54A51 | |
| 521 | EpCam | UniProtKB: P16422 | |
| 522 | EphA2 | UniProtKB: P29317 | |
| 523 | EphA3 | UniProtKB: P29320 | |
| 524 | EphA3 | UniProtKB: Q6P4R6 | |
| 525 | ErbB3 | UniProtKB: B3KWG5 | |
| 526 | ErbB3 | UniProtKB: B4DGQ7 | |
| 527 | ERBB4 | UniProtKB: Q15303 | |
| 528 | ERG | UniProtKB: P11308 | |
| 529 | ETV6 | UniProtKB: P41212 | |
| 530 | EWS | UniProtKB: Q01844 | |
| 531 | EZH2 | UniProtKB: F2YMM1 | |
| 532 | EZH2 | UniProtKB: G3XAL2 | |
| 533 | EZH2 | UniProtKB: L0R855 | |
| 534 | EZH2 | UniProtKB: Q15910 | |
| 535 | EZH2 | UniProtKB: S4S3R8 | |
| 536 | FABP7 | UniProtKB: O15540 | |
| 537 | FCGR3A_Version_1 | UniProtKB: P08637 | |
| 538 | FCGR3A_Version_2 | CCDS: CCDS1232.1 | |
| 539 | FGF5 | UniProtKB: P12034 | |
| 540 | FGF5 | UniProtKB: Q60518 | |
| 541 | FGFR2 | UniProtKB: P21802 | |
| 542 | fibronectin | UniProtKB: A0A024R516 | |
| 543 | fibronectin | UniProtKB: A0A024RB01 | |
| 544 | fibronectin | UniProtKB: A0A024RDT9 | |
| 545 | fibronectin | UniProtKB: A0A024RDV5 | |
| 546 | fibronectin | UniProtKB: A6NH44 | |
| 547 | fibronectin | UniProtKB: A8K6A5 | |
| 548 | fibronectin | UniProtKB: B2R627 | |
| 549 | fibronectin | UniProtKB: B3KXM5 | |
| 550 | fibronectin | UniProtKB: B4DIC5 | |
| 551 | fibronectin | UniProtKB: B4DN21 | |
| 552 | fibronectin | UniProtKB: B4DS98 | |
| 553 | fibronectin | UniProtKB: B4DTH2 | |
| 554 | fibronectin | UniProtKB: B4DTK1 | |
| 555 | fibronectin | UniProtKB: B4DU16 | |
| 556 | fibronectin | UniProtKB: B7Z3W5 | |
| 557 | fibronectin | UniProtKB: B7Z939 | |
| 558 | fibronectin | UniProtKB: G5E9X3 | |
| 559 | fibronectin | UniProtKB: Q9H382 | |
| 560 | FOS | UniProtKB: P01100 | |
| 561 | FOXP3 | UniProtKB: Q9BZS1 | |
| 562 | FUT1 | UniProtKB: P19526 | |
| 563 | G250 | UniProtKB: Q16790 | |
| 564 | GAGE-1 | Genbank: AAA82744 | |
| 565 | GAGE-2 | UniProtKB: Q6NT46 | |
| 566 | GAGE-3 | UniProtKB: Q13067 | |
| 567 | GAGE-4 | UniProtKB: Q13068 | |
| 568 | GAGE-5 | UniProtKB: Q13069 | |
| 569 | GAGE-6 | UniProtKB: Q13070 | |
| 570 | GAGE7b | UniProtKB: O76087 | |
| 571 | GAGE-8_ | UniProtKB: Q9UEU5 | |
| | (GAGE-2D) | | |
| 572 | GASR | UniProtKB: P32239 | |
| 573 | GnT-V | UniProtKB: Q09328 | |
| 574 | GPC3 | UniProtKB: I6QJG3 | |
| 575 | GPC3 | UniProtKB: P51654 | |
| 576 | GPC3 | UniProtKB: Q8IYG2 | |
| 577 | GPNMB/m | UniProtKB: A0A024RA55 | |
| 578 | GPNMB/m | UniProtKB: Q14956 | |
| 579 | GPNMB/m | UniProtKB: Q8IXJ5 | |
| 580 | GPNMB/m | UniProtKB: Q96F58 | |
| 581 | GRM3 | UniProtKB: Q14832 | |
| 582 | HAGE | UniProtKB: Q9NXZ2 | |
| 583 | hepsin | UniProtKB: B2ZDQ2 | |
| 584 | hepsin | UniProtKB: P05981 | |
| 585 | Her2/neu | UniProtKB: B4DTR1 | |
| 586 | Her2/neu | UniProtKB: L8E8G2 | |
| 587 | Her2/neu | UniProtKB: P04626 | |
| 588 | Her2/neu | UniProtKB: Q9UK79 | |
| 589 | HLA-A2/m | UniProtKB: Q95387 | |
| 590 | HLA-A2/m | UniProtKB: Q9MYF8 | |
| 591 | homeobox_NKX3.1 | UniProtKB: Q99801 | |
| 592 | HOM-TES-85 | UniProtKB: B2RBQ6 | |
| 593 | HOM-TES-85 | UniProtKB: Q9P127 | |
| 594 | HPG1 | Pubmed: 12543784 | |
| 595 | HS71A | UniProtKB: P0DMV8 | |
| 596 | HS71B | UniProtKB: P0DMV9 | |
| 597 | HST-2 | UniProtKB: P10767 | |
| 598 | hTERT | UniProtKB: O94807 | |
| 599 | iCE | UniProtKB: O00748 | |
| 600 | IF2B3 | UniProtKB: O00425 | |
| 601 | IL-13Ra2 | UniProtKB: Q14627 | |
| 602 | IL2-RA | UniProtKB: P01589 | |
| 603 | IL2-RB | UniProtKB: P14784 | |
| 604 | IL2-RG | UniProtKB: P31785 | |
| 605 | IMP3 | UniProtKB: Q9NV31 | |
| 606 | ITA5 | UniProtKB: P08648 | |
| 607 | ITB1 | UniProtKB: P05556 | |
| 608 | ITB6 | UniProtKB: P18564 | |
| 609 | kallikrein-2 | UniProtKB: A0A024R4J4 | |
| 610 | kallikrein-2 | UniProtKB: A0A024R4N3 | |
| 611 | kallikrein-2 | UniProtKB: B0AZU9 | |
| 612 | kallikrein-2 | UniProtKB: B4DU77 | |
| 613 | kallikrein-2 | UniProtKB: P20151 | |
| 614 | kallikrein-2 | UniProtKB: Q6T774 | |
| 615 | kallikrein-2 | UniProtKB: Q6T775 | |
| 616 | kallikrein-4 | UniProtKB: A0A0C4DFQ5 | |
| 617 | kallikrein-4 | UniProtKB: Q5BQA0 | |
| 618 | kallikrein-4 | UniProtKB: Q96PT0 | |
| 619 | kallikrein-4 | UniProtKB: Q96PT1 | |
| 620 | kallikrein-4 | UniProtKB: Q9Y5K2 | |
| 621 | KI20A | UniProtKB: O95235 | |
| 622 | KIAA0205 | UniProtKB: Q92604 | |
| 623 | KIF2C | UniProtKB: Q99661 | |
| 624 | KK-LC-1 | UniProtKB: Q5H943 | |
| 625 | LDLR | UniProtKB: P01130 | |
| 626 | LGMN | UniProtKB: Q99538 | |
| 627 | LIRB2 | UniProtKB: Q8N423 | |
| 628 | LY6K | UniProtKB: Q17RY6 | |
| 629 | MAGA5 | UniProtKB: P43359 | |
| 630 | MAGA8 | UniProtKB: P43361 | |
| 631 | MAGAB | UniProtKB: P43364 | |
| 632 | MAGE-A10 | UniProtKB: A0A024RC14 | |
| 633 | MAGE-A12 | UniProtKB: P43365 | |
| 634 | MAGE-A1 | UniProtKB: P43355 | |
| 635 | MAGE-A2 | UniProtKB: P43356 | |
| 636 | MAGE-A3 | UniProtKB: P43357 | |
| 637 | MAGE-A4 | UniProtKB: A0A024RC12 | |
| 638 | MAGE-A4 | UniProtKB: P43358 | |
| 639 | MAGE-A4 | UniProtKB: Q1RN33 | |
| 640 | MAGE-A6 | UniProtKB: A8K072 | |
| 641 | MAGE-A6 | UniProtKB: P43360 | |
| 642 | MAGE-A6 | UniProtKB: Q6FHI5 | |
| 643 | MAGE-A9 | UniProtKB: P43362 | |
| 644 | MAGE-B10 | UniProtKB: Q96LZ2 | |
| 645 | MAGE-B16 | UniProtKB: A2A368 | |
| 646 | MAGE-B17 | UniProtKB: A8MXT2 | |
| 647 | MAGE-_B1 | UniProtKB: Q96TG1 | |
| 648 | MAGE-B2 | UniProtKB: O15479 | |
| 649 | MAGE-B3 | UniProtKB: O15480 | |
| 650 | MAGE-B4 | UniProtKB: O15481 | |
| 651 | MAGE-B5 | UniProtKB: Q9BZ81 | |
| 652 | MAGE-B6 | UniProtKB: Q8N7X4 | |
| 653 | MAGE-C1 | UniProtKB: O60732 | |
| 654 | MAGE-C2 | UniProtKB: Q9UBF1 | |
| 655 | MAGE-C3 | UniProtKB: Q8TD91 | |
| 656 | MAGE-D1 | UniProtKB: Q9Y5V3 | |
| 657 | MAGE-D2 | UniProtKB: Q9UNF1 | |
| 658 | MAGE-D4 | UniProtKB: Q96JG8 | |
| 659 | MAGE-_E1 | UniProtKB: Q6IAI7 | |
| 660 | MAGE- | UniProtKB: Q9HCI5 | |
| | E1_(MAGE1) | | |
| 661 | MAGE-E2 | UniProtKB: Q8TD90 | |
| 662 | MAGE-F1 | UniProtKB: Q9HAY2 | |
| 663 | MAGE-H1 | UniProtKB: Q9H213 | |
| 664 | MAGEL2 | UniProtKB: Q9UJ55 | |
| 665 | mammaglobin_A | UniProtKB: Q13296 | |
| 666 | mammaglobin_A | UniProtKB: Q6NX70 | |
| 667 | MART-1/melan-A | UniProtKB: Q16655 | |
| 668 | MART-2 | UniProtKB: Q5VTY9 | |
| 669 | MC1_R | UniProtKB: Q01726 | |
| 670 | MC1_R | UniProtKB: Q1JUL4 | |
| 671 | MC1_R | UniProtKB: Q1JUL6 | |
| 672 | MC1_R | UniProtKB: Q1JUL8 | |
| 673 | MC1_R | UniProtKB: Q1JUL9 | |
| 674 | MC1 R | UniProtKB: Q1JUM0 | |
| 675 | MC1_R | UniProtKB: Q1JUM2 | |
| 676 | MC1_R | UniProtKB: Q1JUM3 | |
| 677 | MC1_R | UniProtKB: Q1JUM4 | |
| 678 | MC1_R | UniProtKB: Q1JUM5 | |
| 679 | MC1_R | UniProtKB: Q6UR92 | |
| 680 | MC1_R | UniProtKB: Q6UR94 | |
| 681 | MC1_R | UniProtKB: Q6UR95 | |
| 682 | MC1_R | UniProtKB: Q6UR96 | |
| 683 | MC1_R | UniProtKB: Q6UR97 | |
| 684 | MC1_R | UniProtKB: Q6UR98 | |
| 685 | MC1_R | UniProtKB: Q6UR99 | |
| 686 | MC1_R | UniProtKB: Q6URA0 | |
| 687 | MC1_R | UniProtKB: Q86YW1 | |
| 688 | MC1 R | UniProtKB: V9Q5S2 | |
| 689 | MC1_R | UniProtKB: V9Q671 | |
| 690 | MC1_R | UniProtKB: V9Q783 | |
| 691 | MC1_R | UniProtKB: V9Q7F1 | |
| 692 | MC1_R | UniProtKB: V9Q8N1 | |
| 693 | MC1_R | UniProtKB: V9Q977 | |
| 694 | MC1_R | UniProtKB: V9Q9P5 | |
| 695 | MC1_R | UniProtKB: V9Q9R8 | |
| 696 | MC1_R | UniProtKB: V9QAE0 | |
| 697 | MC1_R | UniProtKB: V9QAR2 | |
| 698 | MC1_R | UniProtKB: V9QAW3 | |
| 699 | MC1_R | UniProtKB: V9QB02 | |
| 700 | MC1_R | UniProtKB: V9QB58 | |
| 701 | MC1_R | UniProtKB: V9QBY6 | |
| 702 | MC1_R | UniProtKB: V9QC17 | |
| 703 | MC1_R | UniProtKB: V9QC66 | |
| 704 | MC1_R | UniProtKB: V9QCQ4 | |
| 705 | MC1_R | UniProtKB: V9QDF4 | |
| 706 | MC1_R | UniProtKB: V9QDN7 | |
| 707 | MC1_R | UniProtKB: V9QDQ6 | |
| 708 | mesothelin | UniProtKB: Q13421 | |
| 709 | MITF | UniProtKB: O75030-8 | |
| 710 | MITF | UniProtKB: O75030-9 | |
| 711 | MITF | UniProtKB: O75030 | |
| 712 | MMP1_1 | UniProtKB: B3KQS8 | |
| 713 | MMP7 | UniProtKB: P09237 | |
| 714 | MUC-1 | Genbank: AAA60019 | |
| 715 | MUM-1/m | RefSeq: NP_116242 | |
| 716 | MUM-2/m | UniProtKB: Q9Y5R8 | |
| 717 | MYO1A | UniProtKB: Q9UBC5 | |
| 718 | MYO1B | UniProtKB: O43795 | |
| 719 | MYO1C | UniProtKB: O00159 | |
| 720 | MYO1D | UniProtKB: O94832 | |
| 721 | MYO1E | UniProtKB: Q12965 | |
| 722 | MYO1F | UniProtKB: O00160 | |
| 723 | MYO1G | UniProtKB: B0I1T2 | |
| 724 | MYO1H | RefSeq: NP_001094891 | |
| 725 | NA17 | UniProtKB: Q3V5L5 | |
| 726 | NA88-A | Pubmed: 10790436 | |
| 727 | Neo-PAP | UniProtKB: Q9BWT3 | |
| 728 | NFYC/m | UniProtKB: Q13952 | |
| 729 | NGEP | UniProtKB: Q6IWH7 | |
| 730 | NPM | UniProtKB: P06748 | |
| 731 | NRCAM | UniProtKB: Q92823 | |
| 732 | NSE | UniProtKB: P09104 | |
| 733 | NUF2 | UniProtKB: Q9BZD4 | |
| 734 | NY-ESO-1 | UniProtKB: P78358 | |
| 735 | OA1 | UniProtKB: P51810 | |
| 736 | OGT | UniProtKB: O15294 | |
| 737 | OS-9 | UniProtKB: B4DH11 | |
| 738 | OS-9 | UniProtKB: B4E321 | |
| 739 | OS-9 | UniProtKB: B7Z8E7 | |
| 740 | OS-9 | UniProtKB: Q13438 | |
| 741 | osteocalcin | UniProtKB: P02818 | |
| 742 | osteopontin | UniProtKB: A0A024RDE2 | |
| 743 | osteopontin | UniProtKB: A0A024RDE6 | |
| 744 | osteopontin | UniProtKB: A0A024RDJ0 | |
| 745 | osteopontin | UniProtKB: B7Z351 | |
| 746 | osteopontin | UniProtKB: F2YQ21 | |
| 747 | osteopontin | UniProtKB: P10451 | |
| 748 | p53 | UniProtKB: P04637 | |
| 749 | PAGE-4 | UniProtKB: O60829 | |
| 750 | PAI-1 | UniProtKB: P05121 | |
| 751 | PAI-2 | UniProtKB: P05120 | |
| 752 | PAP | UniProtKB: Q06141 | |
| 753 | PAP | UniProtKB: Q53S56 | |
| 754 | PATE | UniProtKB: Q8WXA2 | |
| 755 | PAX3 | UniProtKB: P23760 | |
| 756 | PAX5 | UniProtKB: Q02548 | |
| 757 | PD1L1 | UniProtKB: Q9NZQ7 | |
| 758 | PDCD1 | UniProtKB: Q15116 | |
| 759 | PDEF | UniProtKB: O95238 | |
| 760 | PECA1 | UniProtKB: P16284 | |
| 761 | PGCB | UniProtKB: Q96GW7 | |
| 762 | PGFRB | UniProtKB: P09619 | |
| 763 | Pim-1 -Kinase | UniProtKB: A0A024RD25 | |
| 764 | Pin-1 | UniProtKB: O15428 | |
| 765 | Pin-1 | UniProtKB: Q13526 | |
| 766 | Pin-1 | UniProtKB: Q49AR7 | |
| 767 | PLAC1 | UniProtKB: Q9HBJ0 | |
| 768 | PMEL | UniProtKB: P40967 | |
| 769 | PML | UniProtKB: P29590 | |
| 770 | POTEF | UniProtKB: A5A3E0 | |
| 771 | POTE | UniProtKB: Q86YR6 | |
| 772 | PRAME | UniProtKB: A0A024R1E6 | |
| 773 | PRAME | UniProtKB: P78395 | |
| 774 | PRDX5/m | UniProtKB: P30044 | |
| 775 | PRM2 | UniProtKB: P04554 | |
| 776 | prostein | UniProtKB: Q96JT2 | |
| 777 | proteinase-3 | UniProtKB: D6CHE9 | |
| 778 | proteinase-3 | UniProtKB: P24158 | |
| 779 | PSA | UniProtKB: P55786 | |
| 780 | PSB9 | UniProtKB: P28065 | |
| 781 | PSCA | UniProtKB: D3DWI6 | |
| 782 | PSCA | UniProtKB: O43653 | |
| 783 | PSGR | UniProtKB: Q9H255 | |
| 784 | PSM | UniProtKB: Q04609 | |
| 785 | PTPRC | RefSeq: NP_002829 | |
| 786 | RAB8A | UniProtKB: P61006 | |
| 787 | RAGE-1 | UniProtKB: Q9UQ07 | |
| 788 | RARA | UniProtKB: P10276 | |
| 789 | RASH | UniProtKB: P01112 | |
| 790 | RASK | UniProtKB: P01116 | |
| 791 | RASN | UniProtKB: P01111 | |
| 792 | RGS5 | UniProtKB: O15539 | |
| 793 | RHAMM/CD168 | UniProtKB: O75330 | |
| 794 | RHOC | UniProtKB: P08134 | |
| 795 | RSSA | UniProtKB: P08865 | |
| 796 | RU1 | UniProtKB: Q9UHJ3 | |
| 797 | RU2 | UniProtKB: Q9UHG0 | |
| 798 | RUNX1 | UniProtKB: Q01196 | |
| 799 | S-100 | UniProtKB: V9HW39 | |
| 800 | SAGE | UniProtKB: Q9NXZ1 | |
| 801 | SART-_1 | UniProtKB: O43290 | |
| 802 | SART-2 | UniProtKB: Q9UL01 | |
| 803 | SART-3 | UniProtKB: Q15020 | |
| 804 | SEPR | UniProtKB: Q12884 | |
| 805 | SIA7F | UniProtKB: Q969X2 | |
| 806 | SIA8A | UniProtKB: Q92185 | |
| 807 | SIAT9 | UniProtKB: Q9UNP4 | |
| 808 | SIRT2/m | UniProtKB: A0A024R0G8 | |
| 809 | SIRT2/m | UniProtKB: Q8IXJ6 | |
| 810 | SOX10 | UniProtKB: P56693 | |
| 811 | SP17 | UniProtKB: Q15506 | |
| 812 | SPNXA | UniProtKB: Q9NS26 | |
| 813 | SPXN3 | UniProtKB: Q5MJ09 | |
| 814 | SSX-1 | UniProtKB: Q16384 | |
| 815 | SSX-2 | UniProtKB: Q16385 | |
| 816 | SSX3 | UniProtKB: Q99909 | |
| 817 | SSX-4 | UniProtKB: O60224 | |
| 818 | ST1A1 | UniProtKB: P50225 | |
| 819 | STAG2 | UniProtKB: Q8N3U4-2 | |
| 820 | STAMP-1 | UniProtKB: Q8NFT2 | |
| 821 | STEAP-1 | UniProtKB: A0A024RA63 | |
| 822 | STEAP-1 | UniProtKB: Q9UHE8 | |
| 823 | Survivin-2B | UniProtKB: O15392-2 | |
| 824 | survivin | UniProtKB: O15392 | |
| 825 | SYCP1 | UniProtKB: A0A024R0I2 | |
| 826 | SYCP1 | UniProtKB: B7ZLS9 | |
| 827 | SYCP1 | UniProtKB: Q15431 | |
| 828 | SYCP1 | UniProtKB: Q3MHC4 | |
| 829 | SYT-SSX-1 | UniProtKB: A4PIV7 | |
| 830 | SYT-SSX-1 | UniProtKB: A4PIV8 | |
| 831 | SYT-SSX-2 | UniProtKB: A4PIV9 | |
| 832 | SYT-SSX-2 | UniProtKB: A4PIW0 | |
| 833 | TARP | UniProtKB: Q0VGM3 | |
| 834 | TCRg | UniProtKB: A2JGV3 | |
| 835 | TF2AA | UniProtKB: P52655 | |
| 836 | TGFR2 | UniProtKB: P37173 | |
| 837 | TGM-4 | UniProtKB: B2R7D1 | |
| 838 | TIE2 | UniProtKB: Q02763 | |
| 839 | TKTL1 | UniProtKB: P51854 | |
| 840 | TPI/m | UniProtKB: P60174 | |
| 841 | TRGV11 | UniProtKB: Q99601 | |
| 842 | TRGV9 | UniProtKB: A4D1X2 | |
| 843 | TRGV9 | UniProtKB: Q99603 | |
| 844 | TRGV9 | UniProtKB: Q99604 | |
| 845 | TRPC1 | UniProtKB: P48995 | |
| 846 | TRP-p8 | UniProtKB: Q7Z2W7 | |
| 847 | TSG10 | UniProtKB: Q9BZW7 | |
| 848 | TSPY1 | UniProtKB: Q01534 | |
| 849 | TVC_(TRGV3) | Genbank: M13231.1 | |
| 850 | TX101 | UniProtKB: Q9BY14-2 | |
| 851 | tyrosinase | UniProtKB: A0A024DBG7 | |
| 853 | tyrosinase | UniProtKB: L8B082 | |
| 853 | tyrosinase | UniProtKB: L8B086 | |
| 854 | tyrosinase | UniProtKB: L8B0B9 | |
| 855 | tyrosinase | UniProtKB: O75767 | |
| 856 | tyrosinase | UniProtKB: P14679 | |
| 857 | tyrosinase | UniProtKB: U3M8N0 | |
| 858 | tyrosinase | UniProtKB: U3M9D5 | |
| 859 | tyrosinase | UniProtKB: U3M9J2 | |
| 860 | TYRP1 | UniProtKB: P17643 | |
| 861 | TYRP2 | UniProtKB: P40126 | |
| 862 | UPA | UniProtKB: Q96NZ9 | |
| 863 | VEGFR1 | UniProtKB: B5A924 | |
| 864 | WT1 | UniProtKB: A0A0H5AUY0 | |
| 865 | WT1 | UniProtKB: P19544 | |
| 866 | WT1 | UniProtKB: Q06250 | |
| 867 | XAGE1 | UniProtKB: Q9HD64 | |
| 868 | IL-10 | UniProtKB: P22301 | |
| 869 | IL-5 | UniProtKB: P05113 | |
| 870 | M-CSF | UniProtKB: P09603 | |
| 871 | TGFbeta1 | UniProtKB: P01137 | |
| 872 | Caspase_8 | UniProtKB: Q14790 | |
| 873 | SERPINB5 | UniProtKB: P36952 | |
| 874 | calreticulin | UniProtKB: B4DHR1 | |
| 875 | calreticulin | UniProtKB: B4E2Y9 | |
| 876 | calreticulin | UniProtKB: P27797 | |
| 877 | calreticulin | UniProtKB: Q96L12 | |
| 878 | N-myc | UniProtKB: P04198 | |

Additionally, the antigens (including the sequences/fragments/epitopes shown in column 4) described in Table A can be cloned into the LAMP-antigen constructs described herein either individually, or in combination with one another. Thus, each one of the sequences shown in column 1 of Table A, including the epitopes/fragments described in column 4 of Table 1 can be used to generate a LAMP-antigen construct in combination with another sequence also selected from column 1 or column 4 of Table A. In the case of a nucleic acid construct or a cell harboring such a nucleic acid construct, the construct may encode one or more of the antigens listed in Table A or otherwise herein, for example. Furthermore, an IREG sequence may also be included as a second polypeptide (or in the case of a nucleic acid vector or cell harboring such a vector, a coding sequence for an IREG), in order to generate a bicistronic LAMP construct.

The order of the combination of antigens in a particular LAMP-antigen construct can also vary. For example, the names pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3 refer to the proteins described in Table 1 and are explicitly intended to refer generically not only to the full-length sequences shown in Column 1 of Table A, but also, to the sequences/fragments/epitopes as described in the fourth Column of Table A. Thus, each one of the sequences shown in Column 1 of Table A, including the epitopes/fragments described in Column 4 of Table 1 can be used to generate a LAMP construct, which can then be incorporated with, for example, an IREG sequence to create a bicistronic LAMP construct.

To illustrate different, additional possible antigen combinations, but in no way limiting the disclosure, the combinations of antigens (including the sequences shown in Column 1 of Table A and/or the sequences/fragments/epitopes described in Column 4 of Table A) can be cloned into the LAMP Constructs as follows: (a) pp65 and at least one of gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (b) gB and at least one of pp65, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (c) IE1 and at least one of pp65, gB, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (d) MTRII, and at least one of pp65, gB, IE1, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (e) US28 and at least one of pp65, gB, IE1, MTRII, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (f) IGFBP2 and at least one of pp65, gB, IE1, MTRII, US28, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (g) IL10 and at least one of pp65, gB, IE1, MTRII, US28, IGFBP2, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (h) UL144 and at least one of pp65, gB, IE1, MTRII, US28, IGFBP2, IL10, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (i) UL141 and at least one of pp65, gB, IE1, MTRII, US28, IGFBP2, IL10, UL144, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (j) US11 and at least one of pp65, gB, IE1, MTRII, US28, IGFBP2, IL10, UL144, UL141, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (k) IE2 and at least one of pp65, gB, IE1, MTRII, US28, IGFBP2, IL10, UL144, UL141, US11, HOS, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (l) TERT and at least one of pp65, gB, IE1, MTRII, US28, IGFBP2, IL10, UL144, UL141, US11, HOS, IE2, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (m) Survivin and at least one of pp65, gB, IE1, MTRII, US28, IGFBP2, IL10, UL144, UL141, US11, HOS, IE2, TERT, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (n) Tetanus and at least one of pp65, gB, IE1, MTRII, US28, IGFBP2, IL10, UL144, UL141, US11, HOS, IE2, TERT, Survivin, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (o) NY-ESO-1 and at least one of pp65, gB, IE1, MTRII, US28, IGFBP2, IL10, UL144, UL141, US11, HOS, IE2, TERT, Survivin, Tetanus, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (p) HER2 and at least one of pp65, gB, IE1, MTRII, US28, IGFBP2, IL10, UL144, UL141, US11, HOS, IE2, TERT, Survivin, Tetanus, NY-ESO-1, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (q) HER3 and at least one of pp65, gB, IE1, MTRII, US28, IGFBP2, IL10, UL144, UL141, US11, HOS, IE2, TERT, Survivin, Tetanus, NY-ESO-1, HER2, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (r) HVEM and at least one of pp65, gB, IE1, MTRII, US28, IGFBP2, IL10, UL144, UL141, US11, HOS, IE2, TERT, Survivin, Tetanus, NY-ESO-1, HER2, HER3, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (s) HOS and at least one of pp65, gB, IE1, MTRII, US28, IGFBP2, IL10, UL144, UL141, US11, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (t) HPV16E6 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (u) HPV18E6 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (v) HPV16E7 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (w) HPV18E7 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (x) EBNA1 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (y) EBNA1 trunc and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (z) gp350 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (aa) LMP2 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (ab) GCP3 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (ac) Middle S and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (ad) X Protein and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (ae) TIGIT and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (af) TEM8 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM1, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (ag) TEM1 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, HER2 ECD+TM, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (ah) HER2 ECD+TM and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, CEA, TARP, PROSTEIN, PSMA, BIRC4, MUCIN-1, MUCIN-1 iso, CD40-L, WT-1, WT-1 trunc., PRAME, LAGE-1, and/or MAGE A3; (ai) CEA and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, TARP, PROSTEIN, PSMA, BIRC4, MUCIN-1, MUCIN-1 iso, CD40-L, WT-1, WT-1 trunc., PRAME, LAGE-1, and/or MAGE A3; (aj) TARP and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, PROSTEIN, PSMA, BIRC4, MUCIN-1, MUCIN-1 iso, CD40-L, WT-1, WT-1 trunc., PRAME, LAGE-1, and/or MAGE A3; (ak) PROSTEIN and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PSMA, BIRC4, MUCIN-1, MUCIN-1 iso, CD40-L, WT-1, WT-1 trunc., PRAME, LAGE-1, and/or MAGE A3; (al) PSMA and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, BIRC4, MUCIN-1, MUCIN-1 iso, CD40-L, WT-1, WT-1 trunc., PRAME, LAGE-1, and/or MAGE A3; (am) BIRC4 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, MUCIN-1, MUCIN-1 iso, CD40-L, WT-1, WT-1 trunc., PRAME, LAGE-1, and/or MAGE A3; (an) MUCIN-1 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1 iso, CD40-L, WT-1, WT-1 trunc., PRAME, LAGE-1, and/or MAGE A3; (ao) MUCIN-1 iso and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, CD40-L, WT-1, WT-1 trunc., PRAME, LAGE-1, and/or MAGE A3; (ap) CD40-L and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, WT-1, WT-1 trunc., PRAME, LAGE-1, and/or MAGE A3; (aq) WT-1 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1 trunc., PRAME, LAGE-1, and/or MAGE A3; (ar) WT-1 trunc and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, PRAME, LAGE-1, and/or MAGE A3; (as) PRAME and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., LAGE-1, and/or MAGE A3; (at) LAGE-1 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, and/or MAGE A3; and/or (au) MAGE A3 and at least one of pp65, gB, IE1, MTRII, US28, IGFBP2, IL10, UL144, UL141, US11, HOS, IE2, TERT, Survivin, Tetanus, NY-ESO-1, HER2, HER3, HVEM, HPV16E6, HPV18E6, HPV16E7, HPV18E7, EBNA1, EBNA trunc, gp350, LMP2, GCP3, Middle S, X Protein, TIGIT, TEM8, TEM1, HER2 ECD+TM, CEA, TARP, PROSTEIN, PSMA, BIRC4, MUCIN-1, MUCIN-1 iso, CD40-L, WT-1, WT-1 trunc., PRAME, and/or LAGE-1. The order of the combination of antigens as described above in a particular LAMP construct can vary as this list describes what a LAMP construct comprises and not necessarily to describe the arrangement of the antigens within a particular construct. Moreover, it is specifically envisioned that these antigens can be combined within a single LAMP construct, or can be delivered in a composition comprising multiple LAMP constructs.

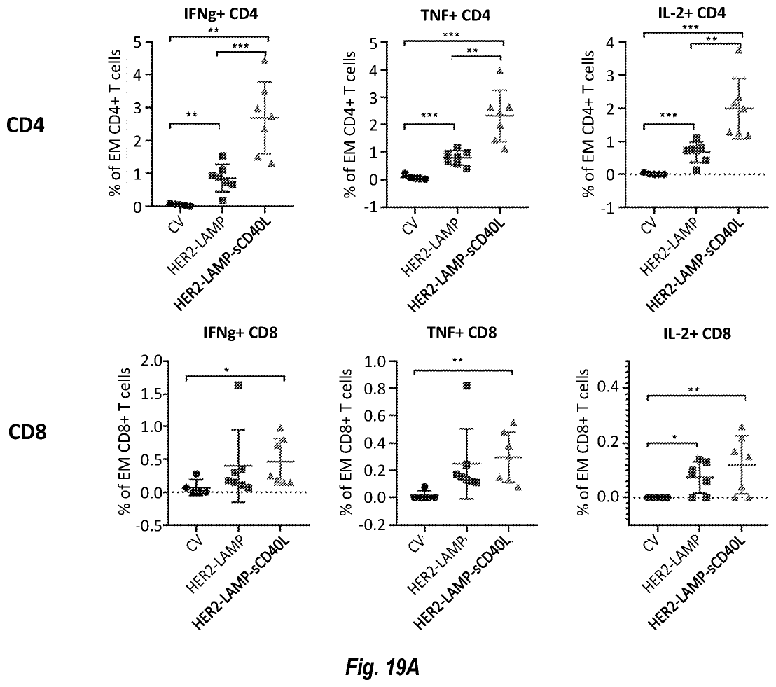

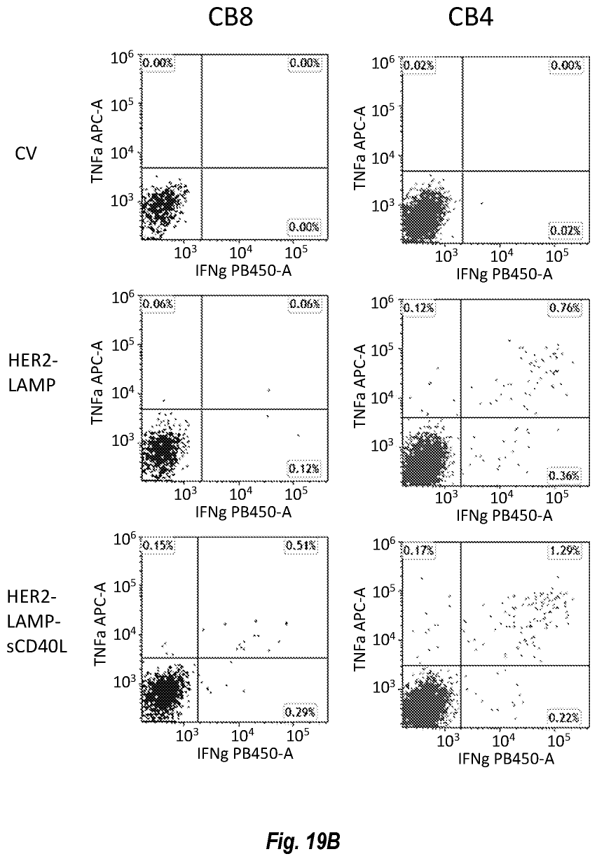

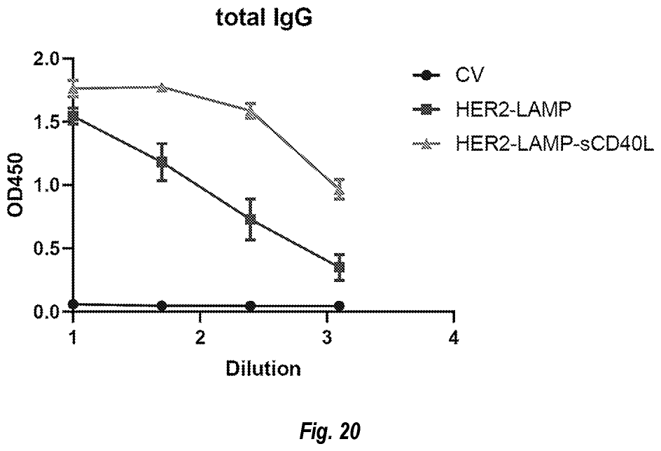

Additional examples of antigens that may be used in the bicistronic LAMP constructs herein include those disclosed, for example, in international publication WO 2018/204534, such as in Table 1 and FIGS. 19-20 of that publication, or in international publication WO 2021/077051, such as in Table 1 and FIG. 11A of that publication. Both of these publications are incorporated herein by reference in their entireties.

In some cases the antigen used in the bicistronic LAMP constructs comprises a pp65 antigen, such as comprising SEQ ID NO: 291, 292, or 293, or one or more of the portions of SEQ ID NO: 291 shown in column 4 of Table A. In come cases, the antigen comprises SEQ ID NO: 292 or 293. In some cases the antigen used in the bicistronic LAMP constructs comprises a gB antigen, such as comprising SEQ ID NO: 294, 295, 296, or 297, or one or more of the antigen fragments from SEQ ID NO: 294 shown in column 4 of Table A. In come cases, the antigen comprises SEQ ID NO: 296 or 297. In some cases the antigen used in the bicistronic LAMP constructs comprises a 1E1 antigen, such as comprising SEQ ID NO: 298, 299, or 300, or one of more of the 1E1 polypeptide sequences shown in column 4 of Table A. In some cases, the antigen comprises SEQ ID NO: 299 or 300. In some cases, the antigen comprises more than one of a pp65, gB, and 1E1 antigen sequence, for example, joined by one or more linker peptide sequences, such as those shown in column 4 of Table A. In some cases, the antigen comprises each of a pp65, gB, and 1E1 antigen sequence, such as sequences selected from a set of antigen sequences (a) comprising SEQ ID NOs: 292 or 293, (b) comprising SEQ ID NOs: 296 or 297, and (c) comprising SEQ ID NOs: 299 or 300. Polynucleotide coding sequences for such pp65, gB, and 1E1 antigens, for example, may be used in a bicistronic LAMP construct along with an IREG protein coding sequence. For example, such constructs, in polypeptide, polynucleotide (i.e. DNA vector or self-replicating RNA vector), or cellular form may be used for treatment of a variety of cancers, such as those listed above, including, for example, cases in which the cancer (including all stages of progression, including hyperplasia) is an adenocarcinoma, sarcoma, skin cancer, melanoma, bladder cancer, brain cancer, breast cancer, uterine cancer, ovarian cancer, prostate cancer, lung cancer (including, but not limited to NSCLC, SCLC, squamous cell cancer), colorectal cancer, anal cancer, rectal cancer, cervical cancer, liver cancer, head and neck cancer, oral cancer, salivary gland cancer, esophageal cancer, pancreas cancer, pancreatic ductal adenocarcinoma (PDA), renal cancer, stomach cancer, kidney cancer, multiple myeloma or cerebral cancer. In some cases, the cancer is glioblastoma multiforme. In some cases, the cancer is breast cancer. In some cases, the cancer is prostate cancer. In some cases, the cancer is head and neck cancer. In some cases, the cancer is colorectal cancer.

In other cases, the antigen comprises a Large T antigen, such as comprising the amino acid sequence of SEQ ID NO: 254, 255, or 256. In some cases, the antigen comprises the amino acid sequence of SEQ ID NO: 255 or SEQ ID NO: 256. In some cases, the LAMP-antigen construct within the bicistronic LAMP construct comprises the amino acid sequence of SEQ ID NO: 879 or SEQ ID NO: 880, both of which comprise the amino acid sequence of SEQ ID NO: 256 flanked by the homology domains of LAMP1, and including a signal sequence. SEQ ID NO: 879 further comprises a LAMP transmembrane domain and cytoplasmic region. In bicistronic LAMP constructs herein, the constructs may further comprise or encode an IREG protein. Constructs in which the antigen is a Large T antigen, for example, may be used in treatment of cancer, such as skin cancer, such as Merkel cell carcinoma. Such antigens may be combined with an IREG as a second polypeptide, examples of which are provided below and elsewhere in the disclosure.

### E. Exemplary Immune Response Enhancing-Genes (IREGs) for Use in Bicistronic LAMP Constructs

In some embodiments, the second polypeptide may include a domain or antigen encoded by an immune response enhancing-gene (IREG), which may increase T cell response and/or antibody response to the bicistronic LAMP-antigen construct. Examples of IREG polypeptides include, for example, CD40L, CD80, OX40, Flt3L, GM-CSF, IL-12, IL-21, IL-23, IL-15, CD70, CD86, IL-7, IL-18, IL-33, GM-CSF, 4-1BB, 4-1BBL, IL-27, or CCL20.

### 1. CD40 Ligand (CD40L)

In some embodiments, the IREG is CD40L. CD40L is a transmembrane protein expressed on the surface of activated T cells, particularly CD4 T cells. CD40L stimulates CD40-dependent activation of antigen-presenting cells (APCs), such as dendritic cells (DCs) and macrophages, as well as B cells for enhancing T cell and antibody responses. In some embodiments, the CD40L is a soluble version of CD40L (sCD40L). In some embodiments, the sCD40L is a 4-trimer, i.e., a protein complex comprising a tetramer of trimers of CD40L. In some embodiments, the sCD40L is more soluble and/or has better secretion than native CD40L. Examples of such CD40L sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 204.

In some cases, the sCD40L is fused to another polypeptide, such as SPD. In some embodiments, the bicistronic construct comprises a second polynucleotide encoding an amino acid sequence at least 60%, at least 70%, at least 75%, at least 80%, at least 85%, at least 90%, at least 95%, at least 96%, at least 97%, at least 98%, or at least 99% or 100% sequence identity of SEQ ID NO: 196, 233, or 238, or a combination of SEQ ID NO: 131 or 133 followed by SEQ ID NO: 204. In some embodiments, the bicistronic construct comprises a second polynucleotide encoding an amino acid sequence at least 60%, at least 70%, at least 75%, at least 80%, at least 85%, at least 90%, at least 95%, at least 96%, at least 97%, at least 98%, or at least 99% or with 100% sequence identity of SEQ ID NO: 196, 233, or 238, or a combination of SEQ ID NO: 131 or 133 followed by SEQ ID NO: 204. In some cases, the coding sequence for the second polypeptide comprises a nucleotide sequence at least 60%, at least 70%, at least 75%, at least 80%, at least 85%, at least 90%, at least 95%, at least 96%, at least 97%, at least 98%, or at least 99% or with 100% sequence identity of SEQ ID NO: 239 or 237 or 205.

### 2. FLT3L

In some embodiments, the IREG is FLT3L. Examples of such FLT3L sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 151 or 209.

In some embodiments, the bicistronic construct encodes a second polypeptide that includes a human Flt3L polypeptide preceded by an SPD polypeptide, thus creating a fusion protein. In some such cases, the bicistronic construct encodes a second polypeptide with an amino acid sequence at least 60%, at least 70%, at least 75%, at least 80%, at least 85%, at least 90%, at least 95%, at least 96%, at least 97%, at least 98%, or at least 99% or with 100% sequence identity of SEQ ID NO: 207.

### 3. IL-12

In some embodiments, the IREG is IL-12. Examples of such IL-12 sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 137, 139, 143, 145, 147, 149, 187, 189, 193, or 213.

In some embodiments, the nucleotide coding sequence for the IL-12 comprises a polynucleotide sequence with at least 60%, at least 70%, at least 75%, at least 80%, at least 85%, at least 90%, at least 95%, at least 96%, at least 97%, at least 98%, or at least 99% or 100% sequence identity of SEQ ID NO: 138, 140, 144, 146, 148, 150, 188, 190, 194, or 214.

### 4. IL-21

In some embodiments, the IREG is IL-21. Examples of such IL-21 sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 179, 181, or 217.

In some embodiments, the nucleotide coding sequence for the IL-21 comprises a polynucleotide sequence with at least 60%, at least 70%, at least 75%, at least 80%, at least 85%, at least 90%, at least 95%, at least 96%, at least 97%, at least 98%, or at least 99% or 100% sequence identity of SEQ ID NO: 180, 182, or 218.

### 5. OX40 Ligand (OX40L)

In some embodiments, the IREG is OX40L. Examples of such OX40L sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 153, 155, or 243. In some cases, the OX40L is fused to a heterologous signal peptide, such as that from IL-2. In some cases, the OX40L sequence is an extracellular domain sequence. In some cases, the OX40L extracellular domain is also fused to an Fc domain of an immunoglobulin. In some cases, the bicistronic construct comprises a second polynucleotide encoding an amino acid sequence at least 60%, at least 70%, at least 75%, at least 80%, at least 85%, at least 90%, at least 95%, at least 96%, at least 97%, at least 98%, or at least 99% or with 100% sequence identity of SEQ ID NO: 242 or a combination of SEQ ID NO: 246 or 248 followed by SEQ ID NO: 243.

### 6. IL-15

In some embodiments, the IREG is IL-15. Examples of such IL-15 sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 167 or 169. In some embodiments, the nucleotide coding sequence for the IL-15 comprises a polynucleotide sequence with at least 60%, at least 70%, at least 75%, at least 80%, at least 85%, at least 90%, at least 95%, at least 96%, at least 97%, at least 98%, or at least 99% or 100% sequence identity of SEQ ID NO: 168 or 170.

In some cases, the IL-15 is expressed behind a heterologous signal sequence, such as IgKVIII or Ig-kappa. Examples of such IL-15 sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 225. In some embodiments, the nucleotide coding sequence for the IL-15 comprises a polynucleotide sequence with at least 60%, at least 70%, at least 75%, at least 80%, at least 85%, at least 90%, at least 95%, at least 96%, at least 97%, at least 98%, or at least 99% or 100% sequence identity of SEQ ID NO: 226.

### 7. CD80

In some embodiments, the IREG is CD80. Examples of such CD80 sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 157, 159, or 253. In some embodiments, the nucleotide coding sequence for the CD80 comprises a polynucleotide sequence with at least 60%, at least 70%, at least 75%, at least 80%, at least 85%, at least 90%, at least 95%, at least 96%, at least 97%, at least 98%, or at least 99% or 100% sequence identity of SEQ ID NO: 158 or 160 or 254.

In some cases, the CD80 is expressed behind a heterologous signal sequence, such as IL-2 signal sequence. In some cases, the CD80 is an extracellular domain of CD80. In some cases, the extracellular domain of CD80 is further fused to the Fc domain of an immunoglobulin. Examples of such sequences include, but are not limited to an amino acid sequence at least 60%, at least 70%, at least 75%, at least 80%, at least 85%, at least 90%, at least 95%, at least 96%, at least 97%, at least 98%, at least 99%, or 100% sequence identity of SEQ ID NO: 252 or 253.

### F. Exemplary Bicistronic LAMP Construct Sequences

In some embodiments, the bicistronic construct comprises a polynucleotide sequence that encodes a LAMP-antigen polypeptide comprising two homology domains of a luminal domain of a LAMP protein, and an antigenic domain heterologous to the LAMP protein, wherein the antigenic domain is placed between the two homology domains. In some cases, the first homology domain of LAMP comprises a polypeptide sequence with at least 90%, at least 95%, at least 96%, at least 97%, at least 98%, at least 99%, or with 100% sequence identity of residues 29 to the C-terminal of SEQ ID NO: 198 or residues 29-194 of SEQ ID NO: 1. In some cases, the second homology domain of LAMP comprises a polypeptide sequence with at least 90%, at least 95%, at least 96%, at least 97%, at least 98%, at least 99%, or with 100% sequence identity of SEQ ID NO: 202 or residues 228-381 of SEQ ID NO: 1.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 230.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 228.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 197.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 208.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 212.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 216.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 222.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 241.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 251.

In some embodiments, the bicistronic construct comprises a polynucleotide sequence with at least 60%, at least 70%, at least 75%, at least 80%, at least 85%, at least 90%, at least 95%, at least 96%, at least 97%, at least 98%, at least 99%, or with 100% sequence identity of SEQ ID NO: 235.

### G. Assembly of Sequences Encoding Bicistronic LAMP Constructs

Procedures for constructing bicistronic LAMP constructs comprising the antigen of interest are well known in the art (see e.g., Williams, et al., J. Cell Biol. 111:955, 1990). DNA sequences encoding the desired segments can be obtained from readily available recombinant DNA materials such as those available from the American Type Culture Collection, 12301 Parklawn Drive, Rockville, Md. 20852, U.S.A., or from DNA libraries that contain the desired DNA.

For example, the DNA segments corresponding to the desired domain sequences can be assembled with appropriate control and signal sequences using routine procedures of recombinant DNA methodology. See, e.g., as described in U.S. Pat. No. 4,593,002, and Langford, et al., Molec. Cell. Biol. 6:3191, 1986.

A DNA sequence encoding a protein or polypeptide can be synthesized chemically or isolated by one of several approaches. The DNA sequence to be synthesized can be designed with the appropriate codons for the desired amino acid sequence. In general, one will select preferred codons for the intended host in which the sequence will be used for expression. The complete sequence may be assembled from overlapping oligonucleotides prepared by standard methods and assembled into a complete coding sequence. See, e.g., Edge, Nature 292:756, 1981; Nambair, et al. Science 223:1299, 1984; Jay, et al., J. Biol. Chem. 259:6311, 1984.

In one aspect, one or more of the polynucleotides encoding the domain sequences of a bicistronic LAMP construct are isolated individually using the polymerase chain reaction (M. A. Innis, et al., In PCR Protocols: A Guide to Methods and Applications, Academic Press, 1990). The domains are preferably isolated from publicly available clones known to contain them, but they may also be isolated from genomic DNA or cDNA libraries. Preferably, isolated fragments are bordered by compatible restriction endonuclease sites which allow a bicistronic LAMP construct encoding the antigen sequence to be constructed. This technique is well known to those of skill in the art. Domain sequences may be fused directly to each other (e.g., with no intervening sequences), or inserted into one another (e.g., where domain sequences are discontinuous), or may be separated by intervening sequences (e.g., such as linker sequences).

The basic strategies for preparing oligonucleotide primers, probes and DNA libraries, as well as their screening by nucleic acid hybridization, are well known to those of ordinary skill in the art. See, e.g., Sambrook, et al., 1989, supra; Perbal, 1984, supra. The construction of an appropriate genomic DNA or cDNA library is within the skill of the art. See, e.g., Perbal, 1984, supra. Alternatively, suitable DNA libraries or publicly available clones are available from suppliers of biological research materials, such as Clonetech and Stratagene, as well as from public depositories such as the American Type Culture Collection.

Selection may be accomplished by expressing sequences from an expression library of DNA and detecting the expressed peptides immunologically. Clones which express peptides that bind to MHC II molecules and to the desired antibodies/T cell receptors are selected. These selection procedures are well known to those of ordinary skill in the art (see, e.g., Sambrook, et al., 1989, supra).

Once a clone containing the coding sequence for the desired polypeptide sequence has been prepared or isolated, the sequence can be cloned into any suitable vector, preferably comprising an origin of replication for maintaining the sequence in a host cell.

### H. Nucleic Acid Delivery Vehicles

In one aspect, the disclosure provides a nucleic acid molecule (e.g. a plasmid or vector) comprising (i) a first polynucleotide sequence encoding an antigen as described herein fused in between a first homology domain of a LAMP protein and a second homology domain of a LAMP protein (or at least between two Cysteine Conserved Fragments), for example the at least one antigen of interest may be placed in, or may replace, the LAMP hinge region); and (ii) a second polynucleotide sequence encoding at least one IREG or further antigen operably linked to a secretion signal sequence, wherein the IREG or further antigen is secreted into the circulation of the subject. Further exemplary nucleic acid embodiments are described in the Summary and claims sections and throughout the disclosure herein.

The nucleic acid molecule can be provided as a vaccine composition and introduced into a cell. The cell may be a host cell for replicating the nucleic acid molecule or for expressing the bicistronic LAMP construct (providing the LAMP-antigen Construct) and the IREG or second antigen operably linked to a secretion signal sequence (such that a second polypeptide comprising the IREG or second antigen is secreted from the cell). Preferably, the host cell is an antigen presenting cell (described further below). In some embodiments, the vaccine comprises DNA, mRNA, or self-amplifying RNA.

In some embodiments, the first polynucleotide sequence encoding the LAMP-antigen Construct further comprises a polynucleotide sequence for insertion into a target cell and an expression control sequence operably linked thereto to control expression of the first polynucleotide sequence (e.g., transcription and/or translation) in the cell. Similarly, in some embodiments, the second polynucleotide sequence encoding the second polypeptide comprising the IREG or further antigen further comprises a polynucleotide sequence for insertion into a target cell and an expression control sequence operably linked thereto to control expression of the second polynucleotide sequence (e.g., transcription and/or translation) in the cell. The nucleic acid molecule comprising the first and second polynucleotide sequences may be provided as, for example, a plasmid, phage, autonomously replicating sequence (ARS), centromeres, and other sequences which are able to replicate or be replicated in vitro or in a host cell (e.g., such as a bacterial, yeast, or insect cell) and/or target cell (e.g., such as a mammalian cell, preferably an antigen presenting cell) and/or to convey the sequences expressed to a desired location within the target cell.

Recombinant expression vectors may be derived from micro-organisms which readily infect animals, including man, horses, cows, pigs, llamas, giraffes, dogs, cats or chickens. Certain vectors herein include those which have already been used as live vaccines, such as vaccinia. These recombinants can be directly inoculated into a host, conferring immunity not only to the microbial vector, but also to express foreign antigens. Some vectors contemplated herein as live recombinant vaccines include RNA viruses, adenovirus, herpesviruses, poliovirus, and vaccinia and other pox viruses, as taught in Flexner, Adv. Pharmacol. 21:51, 1990, for example.

Expression control sequences include, but are not limited to, promoter sequences to bind RNA polymerase, enhancer sequences or negative regulatory elements to bind to transcriptional activators and repressors, respectively, and/or translation initiation sequences for ribosome binding. For example, a bacterial expression vector can include a promoter such as the lac promoter and for transcription initiation, the Shine-Dalgarno sequence and the start codon AUG (Sambrook, et al., 1989, supra). Similarly, a eukaryotic expression vector preferably includes a heterologous, homologous, or chimeric promoter for RNA polymerase II, a downstream polyadenylation signal, the start codon AUG, and a termination codon for detachment of a ribosome.

Expression control sequences may be obtained from naturally occurring genes or may be designed. Designed expression control sequences include, but are not limited to, mutated and/or chimeric expression control sequences or synthetic or cloned consensus sequences. Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.).

In order to optimize expression and/or transcription, it may be necessary to remove, add or alter 5′ and/or 3′ untranslated portions of the vectors to eliminate extra, or alternative translation initiation codons or other sequences that may interfere with, or reduce, expression, either at the level of transcription or translation. Alternatively, consensus ribosome binding sites can be inserted immediately 5′ of the start codon to enhance expression. A wide variety of expression control sequences—sequences that control the expression of a polynucleotide sequence operatively linked to it—may be used in these vectors to express the polynucleotide sequences of this disclosure. Such useful expression control sequences include, for example, the early or late promoters of SV40, CMV, vaccinia, polyoma, adenovirus, herpes virus and other sequences known to control the expression of genes of mammalian cells, and various combinations thereof.

The first and second polynucleotide sequences (encoding the LAMP-antigen Construct and the second polypeptide) may be expressed from the same or different expression control sequences. For example, a single promoter may be used for transcription of a bicistronic mRNA molecule encoding both polypeptides, or different promoters may be used to control expression of the two different polypeptides. Those skilled in the art will be aware that translation of the “second” of the encoded proteins may be achieved by the inclusion of a translation-enhancing element such as an internal ribosome entry site (IRES) (Plank et al., Wiley Interdiscip. Rev. RNA 3:195-212, 2012) or an unstructured junction sequence to achieve post-termination re-initiation of translation (Onishi et al., G3 (Bethesda) 6 (12): 4115-4125, 2016). However, in some embodiments, the polynucleotide sequences encoding the two polypeptides are expressed from different expression control sequences (e.g., different promoters).

In order to achieve secretion of the second polynucleotide, its coding sequence may include a polynucleotide sequence encoding a secretion signal sequence (also known as a leader sequence) typically 16-30 amino acids in length, so that expression of the second polynucleotide sequence being operably linked to the secretion signal sequence. Those skilled in the art are well aware of suitable secretion signal sequences and include, for example, the signal sequence of interleukin-2, CD5, the Immunoglobulin Kappa light chain (hereinafter referred to as the Ig-kappa leader), trypsinogen, serum albumin, and prolactin (Stern et al., Trends Cell Mol. Biol. 2:1-17, 2007; Kober et al., Biotechnol. Bioengin. 110:1164-1173, 2013). The secretion signal sequence may, in some cases, be a secretion signal sequence that is “native” to the IREG or second polypeptide antigen.

In one aspect, the nucleic acid molecule comprises an origin of replication for replication. Preferably, the origin functions in at least one type of host cell which can be used to generate sufficient numbers of copies of the sequence for use in delivery to a target cell. Suitable origins therefore include, but are not limited to, those which function in bacterial cells (e.g., such as Escherichia sp., Salmonella sp., Proteus sp., Clostridium sp., Klebsiella sp., Bacillus sp., Streptomyces sp., and Pseudomonas sp.), yeast (e.g., such as Saccharomyces sp. or Pichia sp.), insect cells, and mammalian cells. In one aspect, an origin of replication is provided which functions in the target cell into which the nucleic acid delivery vehicle is introduced (e.g., a mammalian cell, such as a human cell). In another aspect, at least two origins of replication are provided, one that functions in a host cell and one that functions in a target cell.

The nucleic acid molecule may alternatively, or additionally, comprise a polynucleotide sequence(s) to facilitate integration of at least a portion of the nucleic acid molecule (e.g., delivery vector) into a target cell chromosome. For example, the nucleic acid molecule may comprise regions of homology to target cell chromosomal DNA. In one aspect, the nucleic acid molecule is provided as a delivery vector which comprises two or more recombination sites which flank a nucleic acid sequence encoding the LAMP-antigen construct and the second polypeptide, and/or the bicistronic LAMP construct itself.

The vector may additionally comprise a detectable and/or selectable marker to verify that the vector has been successfully introduced in a target cell and/or can be expressed by the target cell. These markers can encode an activity, such as, but not limited to, production of RNA, peptide, or protein, or can provide a binding site for RNA, peptides, proteins, inorganic and organic compounds or compositions and the like.

Examples of detectable/selectable markers genes include, but are not limited to: DNA segments that encode products which provide resistance against otherwise toxic compounds (e.g., antibiotics); DNA segments that encode products which are otherwise lacking in the recipient cell (e.g., tRNA genes, auxotrophic markers); DNA segments that encode products which suppress the activity of a gene product; DNA segments that encode products which can be readily identified (e.g., phenotypic markers such as beta-galactosidase, a fluorescent protein (GFP, CFP, YFG, BFP, RFP, EGFP, EYFP, EBFP, dsRed, mutated, modified, or enhanced forms thereof, and the like), and cell surface proteins); DNA segments that bind products which are otherwise detrimental to cell survival and/or function; DNA segments that otherwise inhibit the activity of other nucleic acid segments (e.g., antisense oligonucleotides); DNA segments that bind products that modify a substrate (e.g., restriction endonucleases); DNA segments that can be used to isolate or identify a desired molecule (e.g., segments encoding specific protein binding sites); primer sequences; DNA segments, which when absent, directly or indirectly confer resistance or sensitivity to particular compounds; and/or DNA segments that encode products which are toxic in recipient cells.

The marker gene can be used as a marker for conformation of successful gene transfer and/or to isolate cells expressing transferred genes and/or to recover transferred genes from a cell. For example, in one aspect, the marker gene is used to isolate and purify antigen presenting cells expressing a bicistronic LAMP construct described herein.

Substantially similar genes may be provided, e.g., genes with greater than about 50%, greater than about 70%, greater than 80%, greater than about 90%, and preferably, greater than about 95% identity to a known gene. Substantially similar domain sequences may initially be identified by selecting a sequence which specifically hybridizes to a domain sequence of interest under stringent hybridization conditions. Performing assays to determine the suitability of homologous, variant, or modified domain sequences is merely a matter of screening for sequences which express the appropriate activity. Such screening is routine in the art.

The bicistronic LAMP construct encoding the LAMP-antigen construct and the second polypeptide may be provided as a naked nucleic acid molecule or in a delivery vehicle associated with one or more molecules for facilitating entry of a nucleic acid into a cell. Suitable delivery vehicles include, but are not limited to: liposomal formulations, polypeptides, polysaccharides, lipopolysaccharides, viral formulations (e.g., including viruses, viral particles, artificial viral envelopes and the like), cell delivery vehicles, and the like.

### I. Lipid-Based Formulations

Delivery vehicles designed to facilitate intracellular delivery of a nucleic acid molecule encoding a bicistronic LAMP construct must interact with both non-polar and polar environments (in or on, for example, the plasma membrane, tissue fluids, compartments within the cell, and the like). Therefore, preferably, delivery vehicles are designed to contain both polar and non-polar domains or a translocating sequence for translocating a nucleic acid molecule encoding a bicistronic LAMP construct into a cell.

Compounds having polar and non-polar domains are termed amphiphiles. Cationic amphiphiles have polar groups that are capable of being positively charged at, or around, physiological pH for interacting with negatively charged polynucleotides such as DNA.

The nucleic acid molecules comprising the bicistronic LAMP constructs can be provided in formulations comprising lipid monolayers or bilayers to facilitate transfer of the vectors across a cell membrane. Liposomes or any form of lipid membrane, such as planar lipid membranes or the cell membrane of an intact cell, e.g., a red blood cell, can be used. Liposomal formulations can be administered by any means, including administration intravenously or orally.

Liposomes and liposomal formulations can be prepared according to standard methods and are well known in the art, see, e.g., Remington's; Akimaru, 1995, Cytokines Mol. Ther. 1:197-210; Alving, 1995, Immunol. Rev. 145:5-31; Szoka, 1980, Ann. Rev. Biophys. Bioeng. 9:467; U.S. Pat. Nos. 4,235,871; 4,501,728; and 4,837,028. In one aspect, the liposome comprises a targeting molecule for targeting a liposome:nucleic acid molecule (bicistronic LAMP construct herein) complex to a particular cell type. In a particular aspect, a targeting molecule comprises a binding partner (e.g., a ligand or receptor) for a biomolecule (e.g., a receptor or ligand) on the surface of a blood vessel or a cell found in a target tissue.

Liposome charge is an important determinant in liposome clearance from the blood, with negatively charged liposomes being taken up more rapidly by the reticuloendothelial system (Juliano, 1975, Biochem. Biophys. Res. Commun. 63:651) and thus having shorter half-lives in the bloodstream. Incorporating phosphatidylethanolamine derivatives enhances the circulation time by preventing liposomal aggregation. For example, incorporation of N-(omega-carboxy) acylamidophosphatidylethanolamines into large unilamellar vesicles of L-alpha-distearoylphosphatidylcholine dramatically increases the in vivo liposomal circulation lifetime (see, e.g., Ahl, 1997, Biochim. Biophys. Acta 1329:370-382). Liposomes with prolonged circulation half-lives are typically desirable for therapeutic and diagnostic uses. For a general discussion of pharmacokinetics, see, e.g., Remington's, Chapters 37-39, Lee, et al., In Pharmacokinetic Analysis: A Practical Approach (Technomic Publishing AG, Basel, Switzerland 1996).

Typically, liposomes are prepared with about 5 to 15 mole percent negatively charged phospholipids, such as phosphatidylglycerol, phosphatidylserine or phosphatidyl-inositol. Added negatively charged phospholipids, such as phosphatidylglycerol, also serve to prevent spontaneous liposome aggregation, and thus minimize the risk of undersized liposomal aggregate formation. Membrane-rigidifying agents, such as sphingomyelin or a saturated neutral phospholipid, at a concentration of at least 50 mole percent, and 5 to 15 mole percent of monosialylganglioside can also impart desirably liposome properties, such as rigidity (see, e.g., U.S. Pat. No. 4,837,028).

Additionally, the liposome suspension can include lipid-protective agents which protect lipids against free-radical and lipid-peroxidative damages on storage. Lipophilic free-radical quenchers, such as alpha-tocopherol and water-soluble iron-specific chelators, such as ferrioxianine, may be used.

The bicistronic LAMP constructs described herein can also be incorporated into multilamellar vesicles of heterogeneous sizes. For example, vesicle-forming lipids can be dissolved in a suitable organic solvent or solvent system and dried under vacuum or an inert gas to form a thin lipid film. If desired, the film can be redissolved in a suitable solvent, such as tertiary butanol, and then lyophilized to form a more homogeneous lipid mixture which is in a more easily hydrated powder like form. This film is covered with an aqueous solution of the peptide or polypeptide complex and allowed to hydrate, typically over a 15 to 60 minute period with agitation. The size distribution of the resulting multilamellar vesicles can be shifted toward smaller sizes by hydrating the lipids under more vigorous agitation conditions or by adding solubilizing detergents such as deoxycholate. The hydration medium preferably comprises the nucleic acid at a concentration which is desired in the interior volume of the liposomes in the final liposome suspension.

Following liposome preparation, the liposomes can be sized to achieve a desired size range and relatively narrow distribution of liposome sizes. One exemplary size range is about 0.2 to 0.4 microns, which allows the liposome suspension to be sterilized by filtration through a conventional filter, typically a 0.22-micron filter. Filter sterilization can be carried out on a high throughput basis if the liposomes have been sized down to about 0.2 to 0.4 microns. Several techniques are available for sizing liposome to a desired size (see, e.g., U.S. Pat. No. 4,737,323).

Suitable lipids include, but are not limited to, DOTMA (Felgner, et al., 1987, Proc. Natl. Acad. Sci. USA 84:7413-7417), DOGS or Transfectain™ (Behr, et al., 1989, Proc. Natl. Acad. Sci. USA 86:6982-6986), DNERIE or DORIE (Felgner, et al., Methods 5:67-75), DC-CHOL (Gao and Huang, 1991, BBRC 179:280-285), DOTAP™ (McLachlan, et al., 1995, Gene Therapy 2:674-622), Lipofectamine™. and glycerolipid compounds (see, e.g., EP901463 and WO98/37916).

Other molecules suitable for complexing with the bicistronic LAMP constructs may include cationic molecules, such as, polyamidoamine (Haensler and Szoka, 1993, Bioconjugate Chem. 4:372-379), dendritic polylysine (WO 95/24221), polyethylene irinine or polypropylene h-nine (WO 96/02655), polylysine (U.S. Pat. No. 5,595,897; FR 2 719 316), chitosan (U.S. Pat. No. 5,744,166), DNA-gelatin coacervates (see, e.g., U.S. Pat. Nos. 6,207,195; 6,025,337; 5,972,707) or DEAE dextran (Lopata, et al., 1984, Nucleic Acid Res. 12:5707-5717).

### J. Viral-Based Gene Delivery Vehicles

In one aspect, the nucleic acid molecule comprising the bicistronic LAMP construct is provided as a delivery vehicle comprising a virus or viral particle. In this aspect, preferably, the nucleic acid molecule comprises a viral vector. Viral vectors, such as retroviruses, adenoviruses, adeno-associated viruses and herpes viruses, are often made up of two components, a modified viral genome and a coat structure surrounding it (see, e.g., Smith et al., 1995, Ann. Rev. Microbiol. 49:807-838), although sometimes viral vectors are introduced in naked form or coated with proteins other than viral proteins. Most current vectors have coat structures similar to a wild-type virus. This structure packages and protects the viral nucleic acid and provides the means to bind and enter target cells.

Preferably, viral vectors comprising the bicistronic LAMP construct described herein are modified from wild-type viral genomes to disable the growth of the virus in a target cell while enabling the virus to grow in a host cell (e.g., such as a packaging or helper cell) used to prepare infectious particles. Vector nucleic acids generally essential cis-acting viral sequences for replication and packaging in a helper line and expression control sequences for regulating the expression of a polynucleotide being delivered to a target cell. Other viral functions are expressed in trans in specific packaging or helper cell lines as are known in the art.

Viral vectors may be derived from a virus selected from the group consisting of herpes viruses, cytomegaloviruses, foamy viruses, lentiviruses, Semliki forest virus, AAV (adeno-associated virus), poxviruses, adenoviruses and retroviruses. Such viral vectors are well known in the art.

In one aspect, a viral vector used is an adenoviral vector. The adenoviral genome consists of a linear double-stranded DNA molecule of approximately 36 kb carrying more than about thirty genes necessary to complete the viral replication cycle. The early genes are divided into 4 regions (E1 to E4) that are essential for viral replication with the exception of the E3 region, which is believed to modulate the anti-viral host immune response. The E1 region (EIA and EIB) encodes proteins responsible for the regulation of transcription of the viral genome. Expression of the E2 region genes (E2A and E2B) leads to the synthesis of the polypeptides needed for viral replication. The proteins encoded by the E3 region prevent cytolysis by cytotoxic T cells and tumor necrosis factor (Wold and Gooding, 1991, Virology 184:1-8). The proteins encoded by the E4 region are involved in DNA replication, late gene expression and splicing and host cell shut off (Halbert, et al., 1985, J. Virol. 56:250-257). The late genes generally encode structural proteins contributing to the viral capsid. In addition, the adenoviral genome carries at cis-acting 5′ and 3′ ITRs (Inverted Terminal Repeat) and packaging sequences essential for DNA replication. The ITRs harbor origins of DNA replication while the encapsidation region is required for the packaging of adenoviral DNA into infectious particles.

Adenoviral vectors can be engineered to be conditionally replicative (CRAd vectors) in order to replicate selectively in specific cells (e.g., such as proliferative cells) as described in Heise and Kim (2000, J. Clin. Invest. 105:847-85 1). In another aspect, an adenoviral vector is replication-defective for the E1 function (e.g., by total or partial deletion or mutagenesis of E1). The adenoviral backbone of the vector may comprise additional modifications (deletions, insertions or mutations in one or more viral genes). An example of an E2 modification is illustrated by the thermosensitive mutation localized on the DBP (DNA Binding Protein) encoding gene (Ensinger et al., 1972, J. Virol. 10:328-339). The adenoviral sequence may also be deleted of all or part of the E4 region (see, e.g., EP 974 668; Christ, et al., 2000, Human Gene Ther. 11:415-427; Lusky, et al., 1999, J. Virol. 73:8308-8319). Additional deletions within the non-essential E3 region may allow the size of the polynucleotide being delivered to be increased (Yeh, et al., 1997, FASEB Journal 11:615 623). However, it may be advantageous to retain all or part of the E3 sequences coding for polypeptides (e.g., such as gp19k) allowing the virus to escape the immune system (Gooding, et al., 1990, Critical Review of Immunology 10:53-71) or inflammatory reactions (EP 00440267.3).

Second generation vectors retaining the ITRs and packaging sequences and comprising substantial genetic modifications to abolish the residual synthesis of the viral antigens also may be used in order to improve long-term expression of the expressed gene in the transduced cells (see, e.g., WO 94/28152; Lusky, et al., 1998, J. Virol 72:2022-2032).

The nucleic acid molecules of the disclosure being introduced into the cell may be inserted in any location of the viral genome, with the exception of the cis-acting sequences. Preferably, it is inserted in replacement of a deleted region (E1, E3 and/or E4), preferably, within a deleted E1 region.

Adenoviruses can be derived from any human or animal source, in particular canine (e.g. CAV-1 or CAV-2 Genbank ref. CAVIGENOM and CAV77082, respectively), avian (Genbank ref. AAVEDSDNA), bovine (such as BAV3; Reddy, et al., 1998, J. Virol. 72:1394 1402), murine (Genbank ref. ADRMUSMAVI), ovine, feline, porcine or simian sources or alternatively, may be a hybrid virus. Any serotype can be employed. In some cases, the human adenoviruses of the C sub-group are used, especially adenoviruses 2 (Ad2) and 5 (Ad5). Such viruses are available, for example, from the ATCC.

Adenoviral particles or empty adenoviral capsids also can be used to transfer nucleic acid molecules encoding a bicistronic LAMP construct by a virus-mediated cointernalization process as described in U.S. Pat. No. 5,928,944. This process can be accomplished in the presence of cationic agent(s) such as polycarbenes or lipid vesicles comprising one or more lipid layers.

Adenoviral particles may be prepared and propagated according to any conventional technique in the field of the art (e.g., WO 96/17070) using a complementation cell line or a helper virus, which supplies in trans the missing viral genes necessary for viral replication. The cell lines 293 (Graham et al., 1977, J. Gen. Virol. 36:59-72) and PERC6 (Fallaux et al., 1998, Human Gene Therapy 9:1909-1917) are commonly used to complement E1 deletions. Other cell lines have been engineered to complement defective vectors (Yeh, et al., 1996, J. Virol. 70:559-565; Kroughak and Graham, 1995, Human Gene Ther. 6:1575-1586; Wang, et al., 1995, Gene Ther. 2:775-783; Lusky, et al., 1998, J. Virol. 72:2022-203; EP 919627 and WO 97/04119). The adenoviral particles can be recovered from the culture supernatant but also from the cells after lysis and optionally further purified according to standard techniques (e.g., chromatography, ultracentrifugation, as described in WO 96/27677, WO 98/00524 WO 98/26048 and WO 00/50573).

Cell-type specific targeting may be achieved with vectors derived from adenoviruses having a broad host range by the modification of viral surface proteins. For example, the specificity of infection of adenoviruses is determined by the attachment to cellular receptors present at the surface of permissive cells. In this regard, the fiber and penton present at the surface of the adenoviral capsid play a critical role in cellular attachment (Defer, et al., 1990, J. Virol. 64:3661-3673). Thus, cell targeting of adenoviruses can be carried out by genetic modification of the viral gene encoding fiber and/or penton, to generate modified fiber and/or penton capable of specific interaction with unique cell surface receptors. Examples of such modifications are described in Wickarn, et al., 1997, J. Virol. 71:8221-8229; Arriberg, et al., 1997, Virol. Chem 268:6866-6869; Roux, et al., 1989, Proc. Natl. Acad. Sci. USA 86:9079-9083; Miller and Vile, 1995, FASEB J. 9:190-199; WO 93/09221, and in WO 95/28494.

In a particular aspect, adeno-associated viral sequences are used as vectors. Vectors derived from the human parvovirus AAV-2 (adeno-associated virus type 2) are among the most promising gene delivery vehicles currently being developed. Several of the features of this system for packaging a single-stranded DNA suggest it as a possible alternative to naked DNA for delivery. A primary attractive feature, in contrast to other viral vectors such as vaccinia or adenovirus, is that AAV vectors do not express any viral genes. The only viral DNA sequences included in the vaccine construct are the 145 bp inverted terminal repeats (ITR). Thus, as in immunization with naked DNA, the only gene expressed is that of the antigen, or antigen chimera. Additionally, AAV vectors are known to transduce both dividing and non-dividing cells, such as human peripheral blood monocyte-derived dendritic cells, with persistent transgene expression, and with the possibility of oral and intranasal delivery for generation of mucosal immunity. Moreover, the amount of DNA required appears to be much less by several orders of magnitude, with maximum responses at doses of 10¹⁰ to 10¹¹ particles or copies of DNA in contrast to naked DNA doses of 50 μg or about 10¹⁵ copies.

In one aspect, AAV vectors are packaged by co-transfection of a suitable cell line (e.g., human 293 cells) with the DNA contained in the AAV ITR chimeric protein encoding constructs and an AAV helper plasmid ACG2 containing the AAV coding region (AAV rep and cap genes) without the ITRs. The cells are subsequently infected with the adenovirus Ad5. Vectors can be purified from cell lysates using methods known in the art (e.g., such as cesium chloride density gradient ultracentrifugation) and are validated to ensure that they are free of detectable replication-competent AAV or adenovirus (e.g., by a cytopathic effect bioassay). AAV titer may be determined by quantitative PCR with virus DNA samples prepared after digestion with proteinase K. Preferably, vector titers produced by such a method are approximately 5×10¹² to 1×10¹³ DNase resistant particles per ml.

In other aspects, retroviral vectors are used. Retroviruses are a class of integrative viruses which replicate using a virus-encoded reverse transcriptase, to replicate the viral RNA genome into double stranded DNA which is integrated into chromosomal DNA of the infected cells (e.g., target cells). Such vectors include those derived from murine leukemia viruses, especially Moloney (Gilboa, et al., 1988, Adv. Exp. Med. Biol. 241:29) or Friend's FB29 strains (WO 95/01447). Generally, a retroviral vector is deleted of all or part of the viral genes gag, pol and env and retains 5′ and 3′ LTRs and an encapsidation sequence. These elements may be modified to increase expression level or stability of the retroviral vector. Such modifications include the replacement of the retroviral encapsidation sequence by one of a retrotransposon such as VL30 (see, e.g., U.S. Pat. No. 5,747,323). Preferably, the nucleic acid molecule of the disclosure is inserted downstream of the encapsidation sequence, preferably in opposite direction relative to the retroviral genome. Cell specific targeting may be achieved by the conjugation of antibodies or antibody fragments to the retroviral envelope protein as is known in the art.

Retroviral particles are prepared in the presence of a helper virus or in an appropriate complementation (packaging) cell line which contains integrated into its genome the retroviral genes for which the retroviral vector is defective (e.g., gag/pol and env). Such cell lines are described in the prior art (Miller and Rosman, 1989, BioTechniques 7:980; Danos and Mulligan, 1988, Proc. Natl. Acad. Sci. USA 85:6460; Markowitz, et al., 1988, Virol. 167:400). The product of the env gene is responsible for the binding of the viral particle to the viral receptors present on the surface of the target cell and, therefore determines the host range of the retroviral particle. in the context of the disclosure, it is advantageous to use a packaging cell line, such as the PA317 cells (ATCC CRL 9078) or 293E16 (WO97/35996) containing an amphotropic envelope protein, to allow infection of human and other species' target cells. The retroviral particles are preferably recovered from the culture supernatant and may optionally be further purified according to standard techniques (e.g., chromatography, ultracentrifugation).

Other suitable viruses include poxviruses. The genome of several members of poxyviridae has been mapped and sequenced. A poxyviral vector may be obtained from any member of the poxyviridae, in particular canarypox, fowlpox and vaccinia virus. Suitable vaccinia viruses include, but are not limited to, the Copenhagen strain (Goebel, et al., 1990, Virol. 179:247-266; Johnson, et al., 1993, Virol. 196:381-401), the Wyeth strain and the modified Ankara (MVA) strain (Antoine, et al., 1998, Virol. 244:365-396). The general conditions for constructing a vaccinia virus vector are known in the art (see, e.g., EP 83 286 and EP 206 920; Mayr et al., 1975, Infection 3:6-14; Sutter and Moss, 1992, Proc. Natl. Acad. Sci. USA 89:10847-10851). Preferably, the polynucleotide of interest is inserted within a non-essential locus such as the noncoding intergenic regions or any gene for which inactivation or deletion does not significantly impair viral growth and replication.

Poxyviral particles are prepared as described in the art (Piccini, et al., 1987, Methods of Enzymology 153:545-563; U.S. Pat. Nos. 4,769,330; 4,772,848; 4,603,112; 5,100,587 and 5,179,993). Generally, a donor plasmid is constructed, amplified by growth in E. coli and isolated by conventional procedures. Then, it is introduced into a suitable cell culture (e.g., chicken embryo fibroblasts) together with a poxvirus genome, to produce, by homologous recombination, poxyviral particles. These can be recovered from the culture supernatant or from the cultured cells after a lysis step (e.g., chemical lysis, freezing/thawing, osmotic shock, sonication and the like). Consecutive rounds of plaque purification can be used to remove contaminating wild type virus. Viral particles can then be purified using the techniques known in the art (e.g., chromatographic methods or ultracentrifugation on cesium chloride or sucrose gradients).

The use of vaccinia as a live virus vaccine in the global campaign to eradicate smallpox made vaccinia an obvious choice for development as a live recombinant vaccine vector. Live recombinant vaccinia viruses expressing close to 100 different foreign proteins have been reported, and a number of these are effective experimental vaccines (reviewed by Moss and Flexner, 1987). Vaccinia is particularly versatile as an expression vector because of its large genomic size, capability of accepting at least 25,000 base pairs of foreign DNA, and its ability to infect most eukaryotic cell types, including insect cells (ibid.). Unlike other DNA viruses, poxviruses replicate exclusively in the cytoplasm of infected cells, reducing the possibility of genetic exchange of recombinant viral DNA with the host chromosome. Recombinant vaccinia vectors have been shown to properly process and express proteins from a variety of sources including man, other mammals, parasites, RNA and DNA viruses, bacteria and bacteriophage.

The expression of DNA encoding a foreign protein is controlled by host virus regulatory elements, including upstream promoter sequences and, where necessary, RNA processing signals. Insertion of foreign DNA into nonessential regions of the vaccinia virus genome has been carried out by homologous recombination (Panicali, et al., Proc. Nat'l. Acad. Sci, USA, 79:4927, 1982; Mackett, et al., Proc. Nat'l. Acad. Sci. USA, 79:7415, 1982).

Expression of polypeptides by the nucleic acid molecule of the disclosure may occur because of transcriptional regulatory elements at or near the site of insertion or by more precise genetic engineering. Plasmid vectors that greatly facilitate insertion and expression of foreign genes have been constructed (Mackett, et al., J. Virol, 49:857, 1982). These vectors contain an expression site, composed of a vaccinia transcriptional promoter and one or more unique restriction endonuclease sites for insertion of the foreign coding sequence flanked by DNA from a nonessential region of the vaccinia genome. The choice of promoter determines both the time (e.g., early or late) and level of expression, whereas the flanking DNA sequence determines the site of homologous recombination.

Only about one in a thousand virus particles produced by this procedure is a recombinant. Although recombinant virus plaques can be identified by DNA hybridization, efficient selection procedures have been developed. By using segments of nonessential vaccinia virus thymidine kinase (TK) gene as flanking sequences, the foreign gene recombines into the TK locus and by insertion inactivates the TK gene. Selection of TK virus is achieved by carrying out the virus plaque assay in TK cells in the presents of 5-bromodeoxyuridine. Phosphorylation of the nucleoside analogue and consequent lethal incorporation into viral DNA occurs only in cells infected with TK+ parental virus. Depending on the efficiency of the transfection and recombination, up to 80 of the plaques are desired recombinants, and the rest are spontaneous TK mutants.

Plasmid vectors that contain the E. coli beta-galactosidase gene, as well as an expression site for a second gene, permit an alternative method of distinguishing recombinant from parental virus (Chakrabarti, et al., Mol. Cell. Biol., 5:3403, 1985). Plaques formed by such recombinants can be positively identified by the blue color that forms upon addition of an appropriate indicator. By combining both TK selection and beta-galactosidase expression, recombinant virus is readily and quickly isolated. The recombinants are then amplified by propagation in suitable cell lines and expression of the inserted gene is checked by appropriate enzymological, immunological or physical procedures.

An upper limit to the amount of genetic information that can be added to the vaccinia virus genome is not yet known. However, the addition of nearly 25,000 base pairs of foreign DNA had no apparent deleterious effect on virus yield (Smith, et al., Gene, 25:21, 1983). Were it necessary, large segments of the vaccinia virus genome could be deleted to provide additional capacity (Moss, et al., J. Virol. 40:387, 1981).

Viral capsid molecules may include targeting moieties to facilitate targeting and/or entry into cells. Suitable targeting molecules, include, but are not limited to: chemical conjugates, lipids, glycolipids, hormones, sugars, polymers (e.g., PEG, polylysine, PEI and the like), peptides, polypeptides (see, e.g., WO 94/40958), vitamins, antigens, lectins, antibodies and fragments thereof. Preferably, such targeting molecules recognize and bind to cell-specific markers, tissue-specific markers, cellular receptors, viral antigens, antigenic epitopes or tumor-associated markers.

Compositions comprising a bicistronic LAMP construct, based on viral particles may be formulated in the form of doses of between 10 and 1014 i.u. (infectious units), and preferably, between 10 and 10¹¹ i.u. The titer may be determined by conventional techniques. The doses of bicistronic LAMP constructs are preferably comprised between 0.01 and 10 mg/kg, more especially between 0.1 and 2 mg/kg.

### K. Self-Replicating RNA

Self-replicating RNA virus vectors (also called self-amplifying RNA virus vectors) can also be constructed using the bicistronic LAMP construct described herein. For example, alphaviruses, flaviviruses, measle virus and rhabdoviruses can be used to generate self-replicating RNA virus vaccines. Exemplary strains of self-replicating RNA viruses include, but are not limited to rabies virus (RABV), vesicular stomatitis virus (VSV), West Nile virus, Kunjin virus, Semliki Forest virus (SFV), Sindbis virus (SIN) and/or Venezuelan equine encephalitis virus (VEE).

Self-replicating RNA viruses express the native antigen upon delivery into tissue, thus mimicking live attenuated vaccines without having the risk of reversion to pathogenicity. They also stimulate the innate immune system, thus potentiating responses. See, e.g., Ljungberg, K. “Self-replicating alphavirus RNA vaccines,” Expert Rev Vaccines (2): 177-94 (2015); Lundstrom, K., “Oncolytic Alphaviruses in Cancer Immunotherapy”, Vaccines 5:9 (2017); Lundstrom, K. “Replicon RNA Viral Vectors as Vaccines,” Vaccines 4:39 (2016) (hereby incorporated by reference in their entirety). Use of self-replicating vaccines comprising the bicistronic LAMP constructs described herein can also be used in prime-boost protocols.

Moreover, self-replicating RNA viruses can also be encapsulated by liposomes, as described herein, to improve delivery and targeting. Immunization with self-replicating RNA viruses comprising a nucleic acid molecule described herein may provide higher transient expression levels of antigens resulting in generation of neutralizing antibody responses and protection against lethal challenges under safe conditions.

### L. Cell-Based Delivery Vehicles

The nucleic acid molecules according to the disclosure can be delivered to target cells by means of other cells (“delivery cells”) which comprise the constructs. Methods for introducing nucleic acid molecules into cells are known in the art and include microinjection of DNA into the nucleus of a cell (Capechi, et al., 1980, Cell 22:479-488); transfection with CaP0₄ (Chen and Okayama, 1987, Mol. Cell Biol. 7:2745 2752), electroporation (Chu, et al., 1987, Nucleic Acid Res. 15:1311-1326); lipofection/liposome fusion (Feigner, et al., 1987, Proc. Natl. Acad. Sci. USA 84:7413-7417) and particle bombardment (Yang, et al., 1990, Proc. Natl. Acad. Sci. USA 87:9568-9572). Suitable cells include autologous and non-autologous cells, and may include xenogenic cells. Delivery cells may be induced to deliver their contents to the target cells by inducing their death (e.g., by providing inducible suicide genes to these cells).

### M. Accessory Molecules

The compositions comprising the nucleic acid molecules according to the disclosure may comprise one or more accessory molecules for facilitating the introduction of the nucleic acid molecule into a cell and/or for enhancing a particular therapeutic effect and/or enhancing antibody production.

In addition, the composition may include one or more stabilizing substance(s), such as lipids, nuclease inhibitors, hydrogels, hyaluronidase (WO 98/53853), collagenase, polymers, chelating agents (EP 890362), in order to inhibit degradation within the animal/human body and/or improve transfection/infection of the vector into a target cell. Such substances may be used alone or in combination (e.g., cationic and neutral lipids).

It has also been shown that adenovirus proteins are capable of destabilizing endosomes and enhancing the uptake of DNA into cells. The mixture of adenoviruses to solutions containing a lipid-complexed DNA vector or the binding of DNA to polylysine covalently attached to adenoviruses using protein cross-linking agents may substantially improve the uptake and expression of a bicistronic LAMP construct comprising a nucleic acid molecule (see, e.g., Curiel, et al., 1992, Am. I. Respir. Cell. Mol. Biol. 6:247-252).

### N. Host Cells

Nucleic acid molecules according to the disclosure can be expressed in a variety of host cells, including, but not limited to: prokaryotic cells (e.g., E. coli, Staphylococcus sp., Bacillus sp.); yeast cells (e.g., Saccharomyces sp.); insect cells; nematode cells; plant cells; amphibian cells (e.g., Xenopus); avian cells; and mammalian cells (e.g., human cells, mouse cells, mammalian cell lines, primary cultured mammalian cells, such as from dissected tissues).

The molecules can be expressed in host cells isolated from an organism, host cells which are part of an organism, or host cells which are introduced into an organism. In one aspect, the nucleic acid molecules are expressed in host cells in vitro, e.g., in culture. In another aspect, the nucleic acid molecules are expressed in a transgenic organism (e.g., a transgenic mouse, rat, rabbit, pig, primate, etc.) that comprises somatic and/or germline cells comprising nucleic acids encoding the bicistronic LAMP construct herein. Methods for constructing transgenic animals are well known in the art and are routine.

Nucleic acid molecules as described herein also can be introduced into cells in vitro, and the cells (e.g., such as stem cells, hematopoietic cells, lymphocytes, and the like) can be introduced into the host organism. The cells may be heterologous or autologous with respect to the host organism. For example, cells can be obtained from the host organism, a nucleic acid molecule introduced into the cells in vitro, and then reintroduced into the host organism.

### O. Antigen Presenting Cells

In an aspect of the disclosure, a nucleic acid molecule as described herein is introduced into a natural or engineered antigen presenting cell.

The term “antigen presenting cell” (APC) as used herein intends any cell which presents on its surface an antigen in association with a major histocompatibility complex molecule, preferably a MHC class II molecule, or portion thereof. Examples of suitable APCs are discussed in detail below and include, but are not limited to, whole cells such as macrophages, dendritic cells, B cells, hybrid APCs, and foster antigen presenting cells. Methods of making hybrid APCs are described and known in the art.

Dendritic cells (DCs) are potent antigen-presenting cells. It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC” defined above) class I or II protein on the surface of APCs. This interaction is necessary, but not sufficient, for T cell activation to occur. In fact, without the second type of signals, the first type of signals can result in T cell anergy. The second type of signals, called co-stimulatory signals, is neither antigen-specific nor MHC-restricted, and can lead to a full proliferation response of T cells and induction of T cell effector functions in the presence of the first type of signals.

Several molecules have been shown to enhance co-stimulatory activity. These include, but are not limited to, heat stable antigen (HSA), chondroitin sulfate-modified MHC invariant chain (Ii-CS), intracellular adhesion molecule I (ICAM-1), and B7 co-stimulatory molecule on the surface of APCs and its counter-receptor CD28 or CTLA-4 on T cells.

Other important co-stimulatory molecules are CD40, CD54, CD80, CD86. As used herein, the term “co-stimulatory molecule” encompasses any single molecule or combination of molecules which, when acting together with a peptide/MHC complex bound by a TCR on the surface of a T cell, provides a co-stimulatory effect which achieves activation of the T cell that binds the peptide. The term thus encompasses B7, or other co-stimulatory molecule(s) on an APC, fragments thereof (alone, complexed with another molecule(s), or as part of a fusion protein) which, together with peptide/MHC complex, binds to a cognate ligand and result in activation of the T cell when the TCR on the surface of the T cell specifically binds the peptide. Co-stimulatory molecules are commercially available from a variety of sources, including, for example, Beckman Coulter.

In one aspect of the disclosure, the method described in Romani et al., J. Immunol. Methods 196:135-151, 1996, and Bender et al, J. Immunol. Methods 196:121-135, 1996, are used to generate both immature and mature dendritic cells from the peripheral blood mononuclear cells (PBMCs) of a mammal, such as a murine, simian or human. Briefly, isolated PBMCs are pre-treated to deplete T- and B-cells by means of an immunomagnetic technique. Lymphocyte-depleted PBMC are then cultured for in RPMI medium 9 e.g., about 7 days), supplemented with human plasma (preferably autologous plasma) and GM-CSF/IL-4, to generate dendritic cells. Dendritic cells are nonadherent when compared to their monocyte progenitors. Thus, on approximately day 7, non-adherent cells are harvested for further processing.

The dendritic cells derived from PBMC in the presence of GM-CSF and IL-4 are immature, in that they can lose the nonadherence property and revert back to macrophage cell fate if the cytokine stimuli are removed from the culture. The dendritic cells in an immature state are very effective in processing native protein antigens for the MHC class II restricted pathway (Romani, et al., J. Exp. Med. 169:1169, 1989). Further maturation of cultured dendritic cells is accomplished by culturing for 3 days in a macrophage-conditioned medium (CM), which contains the necessary maturation factors. Mature dendritic cells are less able to capture new proteins for presentation but are much better at stimulating resting T cells (both CD4 and CD8) to grow and differentiate.

Mature dendritic cells can be identified by their change in morphology, such as the formation of more motile cytoplasmic processes; by their nonadherence; by the presence of at least one of the following markers: CD83, CD68, HLA-DR or CD86; or by the loss of Fc receptors such as CD 115 (reviewed in Steinman, Annu. Rev. Immunol. 9:271, 1991). Mature dendritic cells can be collected and analyzed using typical cytofluorography and cell sorting techniques and devices, such as FACScan and FACStar. Primary antibodies used for flow cytometry are those specific to cell surface antigens of mature dendritic cells and are commercially available. Secondary antibodies can be biotinylated Igs followed by FITC- or PE-conjugated streptavidin.

Alternatively, others have reported that a method for upregulating (activating) dendritic cells and converting monocytes to an activated dendritic cell phenotype. This method involves the addition of calcium ionophore to the culture media convert monocytes into activated dendritic cells. Adding the calcium 21 ionophore A23187, for example, at the beginning of a 24-48 hour culture period resulted in uniform activation and dendritic cell phenotypic conversion of the pooled “monocyte plus DC” fractions: characteristically, the activated population becomes uniformly CD 14 (Leu M3) negative, and upregulates HLA-DR, HLA-DQ, ICAM-1,137.1, and 137.2. Furthermore, this activated bulk population functions as well on a small numbers basis as a further purified. Specific combination(s) of cytokines have been used successfully to amplify (or partially substitute) for the activation/conversion achieved with calcium ionophore: these cytokines include but are not limited to G-CSF, GM-CSF, IL-2, and IL-4. Each cytokine when given alone is inadequate for optimal upregulation.

The second approach for isolating APCs is to collect the relatively large numbers of precommitted APCs already circulating in the blood. Previous techniques for isolating committed APCs from human peripheral blood have involved combinations of physical procedures such as metrizamide gradients and adherence/nonadherence steps (Freudenthal et al. PNAS 87:7698-7702, 1990); Percoll gradient separations (Mehta-Damani, et al., J. Immunol. 153:996-1003, 1994); and fluorescence activated cell sorting techniques (Thomas et al., J. Immunol. 151:6840-52, 1993).

There are many other methods routine in the art for isolating professional antigen presenting cells (or their precursors) and that such methods and others which may be developed are not limiting and are encompassed within the scope of the disclosure.

In one embodiment, the APCs and therefore the cells presenting one or more antigens are autologous. In another embodiment, the APCs presenting the antigen are allogeneic, i.e., derived from a different subject.

As discussed herein, a nucleic acid molecule as described herein can be introduced into APCs using the methods described above or others known in the art, including, but not limited to, transfection, electroporation, fusion, microinjection, viral-based delivery, or cell based delivery. Arthur et al., Cancer Gene Therapy 4 (1): 17-25, 1997, reports a comparison of gene transfer methods in human dendritic cells.

Known, partial and putative human leukocyte antigen (HLA), the genetic designation for the human MHC, amino acid and nucleotide sequences, including the consensus sequence, are published (see, e.g., Zemmour and Parham, Immunogenetics 33:310-320, 1991), and cell lines expressing HLA variants are known and generally available as well, many from the American Type Culture Collection (“ATCC”). Therefore, using PCR, MHC class II-encoding nucleotide sequences are readily operatively linked to an expression vector of this disclosure that is then used to transform an appropriate cell for expression therein.

Professional APCs can be used, such as macrophages, B cells, monocytes, dendritic cells, and Langerhans cells. These are collected from the blood or tissue of 1) an autologous donor; 2) a heterologous donor having a different HLA specificity then the host to be treated; or 3) from a xenogeneic donor of a different species using standard procedures (Coligan, et. al., Current Protocols in Immunology, sections 3 and 14, 1994). The cells may be isolated from a normal host or a patient having an infectious disease, cancer, autoimmune disease, or allergy.

Professional APCs may be obtained from the peripheral blood using leukopheresis and “FICOLL/HYPAQUE” density gradient centrifugation (stepwise centrifugation through Ficoll and discontinuous Percoll density gradients). Procedures are utilized which avoid the exposure of the APCs to antigens which could be internalized by the APCs, leading to activation of T cells not specific for the antigens of interest.

Cells which are not naturally antigen presenting can be engineered to be antigen presenting by introducing sequences encoding appropriate molecules. For example, nucleic acid sequences encoding MHC class II molecules, accessory molecules, co-stimulatory molecules and antigen processing assisting molecules can be introduced after direct synthesis, cloning, purification of DNA from cells containing such genes, and the like. One expedient means to obtain genes for encoding the molecules used in the bicistronic LAMP constructs and methods described herein is by polymerase chain reaction (PCR) amplification on selected nucleic acid templates with selected oligonucleotide primer pairs. For example, epithelial cells, endothelial cells, tumor cells, fibroblasts, activated T cells, eosinophils, keratinocytes, astrocytes, microglial cells, thymic cortical epithelial cells, Schwann cells, retinal pigment epithelial cells, myoblasts, vascular smooth muscle cells, chondrocytes, enterocytes, thyrocytes and kidney tubule cells can be used. These may be primary cells recently explanted from a host and not extensively passaged in cell culture to form an established cell line, or established cell lines that are relatively homogeneous and capable of proliferating for many generations or indefinitely.

Cells that are not professional APCs are isolated from any tissue of an autologous donor; a heterologous donor or a xenogeneic donor, where they reside using a variety of known separation methods (Darling, Animal Cells: Culture and Media. J. Wiley, New York, 1994; Freshney, Culture of Animal Cells. Alan R. Liss, Inc., New York, 1987). Non-autologous cells, e.g., heterologous or xenogeneic cells, can be engineered ex vivo to express HLA class I and class II molecules that match known human HLA specificities. These cells can then be introduced into a human subject matching the HLA specificity of the engineered cells. The cells are further engineered ex vivo to express one or more LAMP Constructs according to the disclosure.

The engineered cells are maintained in cell culture by standard cell culture methods (Darling, Animal Cells: Culture and Media, J. Wiley, New York, 1994; Freshney, Culture of Animal Cells, Alan R. Liss, Inc., New York, 1987). Cell lines for use in the present disclosure are obtained from a variety of sources (e.g., ATCC Catalogue of Cell Lines & Hybidomas, American Type Culture Collection, 8th edition, 1995), or are produced using standard methods (Freshney, Culture of Immortalized Cells, Wiley-Liss, New York, 1996). Non-transformed cell lines are preferred for use in human subjects.

In one aspect, CD34+ precursors that are differentiating under the influence of GM-CSF into dendritic cells are obtained from the body of a subject and nucleic acid molecules encoding a bicistronic LAMP construct are introduced into the cells, which are then injected into the subject. Use of the nucleic acid molecules as described herein will enhance the association of peptides derived from a particular antigen with MHC class II molecules on the transduced antigen presenting cells, resulting in significantly more potent systemic T cell dependent immune responses and/or antibody production. While the antigen presenting cells transfected in this strategy are preferably autologous cells, any MHC class II cells that effectively present antigen in the host may be used as described above.

### P. Administration

Vaccine material according to this disclosure may contain the nucleic acid molecules encoding immune stimulatory bicistronic LAMP constructs described herein or may be recombinant microorganisms, or antigen presenting cells which express the immune stimulatory bicistronic LAMP constructs. Preparation and administration of such nucleic acid molecules for immunization of individuals are accomplished according to principles of immunization that are well known to those skilled in the art.

Large quantities of these materials may be obtained by culturing recombinant or transformed cells containing the nucleic acid molecules. Culturing methods are well-known to those skilled in the art and are taught in one or more of the documents cited above. The vaccines comprising nucleic acid molecules as described herein are generally produced by culture of recombinant or transformed cells and formulated in a pharmacologically acceptable solution or suspension, which is usually a physiologically-compatible aqueous solution, or in coated tablets, tablets, capsules, suppositories or ampules, as described in the art, for example in U.S. Pat. No. 4,446,128, incorporated herein by reference. Administration may be any suitable route, including oral, rectal, intranasal or by injection where injection may be, for example, transdermal, subcutaneous, intramuscular or intravenous.

The nucleic acid molecules as described herein may be administered to a mammal in an amount sufficient to induce an immune response in the mammal. In some embodiments, a minimum amount for administration is the amount required to elicit antibody formation to a concentration at least 4 times that which existed prior to administration. A typical initial dose for administration would be 10⁵ to 10¹¹ plaque forming units of a recombinant vector, although this amount may be adjusted by a clinician doing the administration as commonly occurs in the administration of vaccines and other agents which induce immune responses. A single administration may usually be sufficient to induce immunity.

Vaccines comprising nucleic acid molecules as described herein may be tested initially in a non-human mammal (e.g., a mouse or primate). For example, assays of the immune responses of inoculated mice can be used to demonstrate greater antibody, T cell proliferation, and cytotoxic T cell responses to the bicistronic LAMP constructs than to wild-type antigen. The vaccines can be evaluated in Rhesus monkeys to determine whether the vaccine formulation that is highly effective in mice will also elicit an appropriate monkey immune response. In one aspect, each monkey receives a total of 5 mg nucleic acid molecules per immunization, delivered IM and divided between 2 sites, with immunizations at day 0 and at weeks 4, 8, and 20, with an additional doses optional. Antibody responses, ADCC, CD4+ and CD8+ T-cell cytokine production, CD4+ and CD8+ T-cell antigen-specific cytokine staining can be measured to monitor immune responses to the vaccine.

Further description of suitable methods of formulation and administration according to this disclosure may be found in U.S. Pat. No. 4,454,116 (constructs), U.S. Pat. No. 4,681,762 (recombinant bacteria), and U.S. Pat. Nos. 4,592,002 and 4,920,209 (recombinant viruses).

### Q. Procedure for Therapy

In one embodiment, a nucleic acid molecule encoding a bicistronic LAMP construct as described herein could be injected into the patient at any suitable time during the course of their malignancy. For example, a nucleic acid molecule as described herein would be injected at a stage when the tumor burden was low. In an alternative embodiment in which the nucleic acid molecule is introduced into the individual's antigen presenting cells, precursors to the antigen presenting cells or mature antigen presenting cells are drawn either from the individual's bone marrow or peripheral blood by vena puncture. These cells are established in culture followed by transduction with the nucleic acid molecule. Once transduction had occurred, these antigen presenting cells are injected back into the patient.

In a particular embodiment, the disclosure provides a method of treatment for a cancer patient having low tumor burden, such as early in the disease, after resection of a neoplastic tumor, or when the burden of tumor cells is otherwise reduced. In this method, a cell population containing autologous stem cells capable of differentiation into antigen presenting cells which will express MHC class II molecules is obtained from the patient. These cells are cultured and transformed by introducing a bicistronic LAMP construct described herein to deliver the antigen to be associated with an MHC class II molecule either within the compartment/organelle or within another compartment/organelle to which the antigen is delivered, and secrete a second antigen or IREG into the circulation.

The transfected stem cell population is then reintroduced into the patient, where the stem cells differentiate into antigen presenting cells which express MHC class II molecules complexed with Tₕ epitopes from the antigen. The immune response to the antigen will be enhanced by enhanced stimulation of the helper T cell population. The secreted antigen or IREG enhances the immune response by, for example, expanding the memory response.

More generally, in one embodiment, this disclosure provides a vaccine composition comprising a nucleic acid molecule encoding a bicistronic LAMP construct for modulating an immune response in a mammal to an antigen (i.e., stimulating, enhancing, or reducing such a response).

### R. Kits

The disclosure further comprises kits to facilitate performing the methods described herein. In one aspect, a kit comprises a nucleic acid molecule encoding a bicistronic LAMP construct as described herein and a cell for receiving the nucleic acid molecule. In one aspect, the cell is a professional APC. The cell may or may not express co-stimulatory molecules. In an aspect, when the cell does not express co-stimulatory molecules, the antigen encoded by the bicistronic LAMP construct is an auto-antigen. In another aspect, a panel of cells is provided expressing different MHC molecules (e.g., known to be expressed in human beings). In a further aspect, the kit comprises reagents to facilitate entry of the nucleic acid molecule into a cell (e.g., lipid-based formulations, viral packaging materials, cells, and the like). In still a further aspect, one or more T cell lines specific for the antigen encoded by the nucleic acid molecule is provided, to verify the ability of the bicistronic LAMP construct to elicit, modulate, or enhance an immune response.

### S. Additional Embodiments

Additional embodiments herein include the following:

1. A nucleic acid molecule comprising:
a. a first polynucleotide sequence encoding a LAMP-antigen Construct comprising an antigen and a Cysteine Conserved Fragment of a LAMP protein; and
b. a second polynucleotide sequence encoding at least one IREG polypeptide operably linked to a secretion signal sequence;
c, wherein said first and second polynucleotide sequences are operably linked to expression control sequence(s) for expression of the LAMP-antigen Construct and IREG in a host or target cell.

2. The nucleic acid molecule of embodiment 1, wherein:
a. the antigen is placed at the N-terminus of the Cysteine Conserved Fragment;
b. the antigen is placed at the C-terminus of the Cysteine Conserved Fragment; or
c. the antigen is placed in between two Cysteine Conserved Fragments.

3. A nucleic acid molecule comprising:
a. a first polynucleotide sequence encoding a LAMP-antigen Construct comprising an antigen placed between two Cysteine Conserved Fragments; and
b. a second polynucleotide sequence encoding at least one IREG polypeptide operably linked to a secretion signal sequence;
c, wherein said first and second polynucleotide sequences are operably linked to expression control sequence(s) for expression of the LAMP-antigen Construct and the IREG polypeptide in a host or target cell.

4. The nucleic acid molecule of any one of the preceding embodiments, wherein the improved LAMP Construct comprises the structure shown in FIG. 1 of ILC-1, ILC-2, ILC-3, ILC-4, ILC-5, or ILC-6 (i.e., wherein the first polynucleotide sequence encodes a polypeptide comprising the structure shown in FIG. 1 of ILC-1, ILC-2, ILC-3, ILC-4, ILC-5, or ILC-6).

5. The nucleic acid molecule of any one of the preceding embodiments, wherein the Cysteine Conserved Fragment(s) comprise a Homology Domain of a LAMP Protein.

6. The nucleic acid molecule of any one of the preceding embodiments, wherein the improved LAMP Construct further comprises a Transmembrane Domain of a LAMP Protein.

7. The nucleic acid molecule of any one of the preceding embodiments, wherein the improved LAMP Construct further comprises a signal sequence.

8. The nucleic acid molecule of embodiment 7, wherein the signal sequence is derived from a LAMP Protein.

9. The nucleic acid molecule of any one of the preceding embodiments, wherein the antigen comprising the LAMP-antigen Construct is placed in, or replaces, a LAMP hinge region.

10. The nucleic acid molecule of any one of the preceding embodiments, wherein the LAMP protein is selected from LAMP-1, LAMP2, LAMP-3, LIMP 2, Macrosailin, Endolyn, LAMP5, or LIMBIC.

11. The nucleic acid molecule of embodiment 10, wherein the LAMP protein is selected from any one of SEQ ID NOS: 1-113.

12. The nucleic acid molecule of embodiment 10, wherein the LAMP protein is at least 70%, at least 75%, at least 80%, at least 85%, at least 90%, at least 95%, 96%, 97%, 98% or 99% identical to SEQ ID NOS: 1-113.

13. The nucleic acid molecule of any one of the preceding embodiments, wherein expression of the LAMP-antigen Construct in a host or target cell results in processing and presentation of the antigen to the MHC class II pathway to elicit an immune response.

14. The nucleic acid molecule of any one of the preceding embodiments, wherein expression of the IREG in a host or target cell results in the secretion of the IREG.

15. The nucleic acid molecule of embodiment 14, wherein secretion of the IREG may enhance an immune response elicited by the antigen.

16. The nucleic acid molecule of any one of embodiments 1-15, wherein the antigen is an antigen associated with an infectious disease such as Covid-19, an antigen associated with cancer, SARS-COV-2 virus S1 spike subunit, HER2, NY-ESO1, or CD161 or a domain of SARS-COV-2 virus S1 spike subunit, HER2, NY-ESO1, or CD161.

17. The nucleic acid molecule of any one of the preceding embodiments, wherein the nucleic acid molecule is a plasmid or vector.

18. The nucleic acid molecule of embodiment 17, wherein the nucleic acid molecule is a viral vector.

19. A host cell comprising the nucleic acid molecule of any one of embodiments 1 to 18.

20. A composition comprising the nucleic acid molecule of any one of embodiments 1 to 18, or the host cell of embodiment 19.

21. A method of treating a subject having a disease or a disorder, wherein the method comprises administering to a subject in need thereof the nucleic acid molecule of any one of embodiments 1-18 or the host cell of embodiment 19, or the composition of embodiment 20 in an amount sufficient to reduce or treat the disease or disorder.

### EXAMPLES

The disclosure will now be further illustrated with reference to the following examples. It will be appreciated that what follows is by way of example only and that modifications to detail may be made while still falling within the scope of the disclosure.

### Example 1. Construction of Vectors Encoding Bicistronic Lamp Constructs

A first generation COVID-19 vaccine candidate was designed, encoding one COVID-19 antigen as a LAMP fusion protein and a second COVID-19 antigen as a secreted protein. The construct is named the ITI-COVID-19 bicistronic construct (ITI-Bicistronic-S1-LAMP-RBG pA-EF1-S2P BGH pA; SEQ ID NO: 228, FIG. 28C), and was demonstrated to both induce SARS-Cov-2-specific antibodies and T cell responses. To obtain optimal antibody and T cell responses, the dose of the vaccine (20 μg) and the period of time between priming and boost (e.g., 14 days) were studied. After two immunizations with 20 μg ITI-COVID-19 bicistronic vaccine, robust SARS-Cov-2 spike-specific T cells and antibodies were induced. Importantly, the ITI-COVID-19 bicistronic vaccine elicited antibody responses that neutralized SARS-Cov-2.

The S1 and S2 subunits of spike mediate entry of the SARS-COV-2 virus into a host cell. Using a nucleic acid molecule for an ILC-4 LAMP construct, the S1 coding sequence (GenBank MN908974) was located between the polynucleotide sequences encoding two LAMP homology domains (N-Lamp and Luminal domain 2). The S1 coding sequence was operably linked to a CMV promoter under the influence of a CMV enhancer sequence, so that expression in a host cell resulted in an ILC-4 LAMP construct comprising the S1 antigen for processing and presentation to MHC class II molecules (i.e., to provide the “priming antigen”). The S2 coding sequence was provided elsewhere on the vector and was operably linked to a polynucleotide sequence encoding an Ig-kappa secretion signal (leader sequence) and an EF1 promoter sequence, so that expression in a host cell resulted in an S2 antigen for secretion (i.e., to provide the “boosting antigen”). The vector thereby provided a single nucleic acid molecule for introduction into a suitable host or target cell capable of providing both priming and boosting antigens to elicit an enhanced immune response. This may therefore confer a significant advantage over the use of a vector which only encodes a bicistronic LAMP construct inasmuch as any desire or requirement to boost the immune response elicited by the LAMP construct will require the administration of a separately administered booster vaccine (e.g., comprising the antigen) at one or more time intervals.

The first generation ITI-COVID-19 bicistronic vaccine expresses the S1 and S2 subunits of the virus surface-anchored spike glycoprotein. The S1 and S2 subunits of spike mediate entry of the SARS-COV-2 virus into a host cell. Using a nucleic acid molecule for an ILC-4 LAMP construct, the S1 coding sequence (GenBank MN908974) was located between the polynucleotide sequences encoding two LAMP homology domains (N-Lamp and Luminal domain 2). The S1 coding sequence is operably linked to a CMV promoter under the influence of a CMV enhancer sequence, so that expression in a host cell results in an ILC-4 LAMP construct comprising the S1 antigen for processing and presentation to MHC class II molecules (i.e., to provide the “priming antigen”). The S2 coding sequence is provided elsewhere on the vector and is operably linked to a polynucleotide sequence encoding an Ig-kappa secretion signal (leader sequence) and an EF1 promoter sequence, so that expression in a host cell results in an S2 antigen for secretion (i.e., to provide the “boosting antigen”). The vector thereby provides a single nucleic acid molecule for introduction into a suitable host or target cell capable of providing target and enhancement antigens to elicit an enhanced immune response. This may therefore confer a significant advantage over the use of a vector which only encodes a bicistronic LAMP construct inasmuch as any desire or requirement to enhance the immune response elicited by the LAMP construct will require the administration of a separately administered booster vaccine (e.g., comprising the antigen) at one or more time intervals.

The polynucleotide sequence of this first generation COVID-19 bicistronic construct is shown in FIG. 28C. DNA sequences for S1 and S2 were obtained from, for example, Genbank (ncbi.nlm.nih.gov/genbank/sars-cov-2-seqs/#nucleotide-sequences). In the vector shown in FIG. 28A, the S2 polynucleotide sequence encodes an S2 variant including two proline (P) substitutions. Specifically, the ITI-COVID-19 bicistronic vaccine (first generation vaccine) expresses an S1-LAMP sequence (residues 950-2004 of SEQ ID NO: 227 shown in FIG. 28C), as described in more detail below and a Spike protein (residues 16-1273) with an Ig-kappa leader sequence (see FIG. 28C; residues 2145-3423 of SEQ ID NO: 227).

Table 1 below provides exemplary DNA and protein sequences of the above bicistronic LAMP constructs as well as various exemplary promoter/enhancer sequences and polypeptide sequences that may be used in constructing bicistronic LAMP constructs described herein.

The following additional bicistronic LAMP constructs were constructed using standard molecular biology techniques well known to the skilled artisan: (1) HER2-LAMP-sCD40L (FIG. 11; SEQ ID NO: 197), (2) HER2-LAMP-mFLT3L (SEQ ID NO: 208), (3) HER2-LAMP-IL-12 (SEQ ID NO: 212), (4) HER2-LAMP-IL-21 (SEQ ID NO: 216), (5) HER2-LAMP-OX40L (SEQ ID NO: 241), (6) HER2-LAMP-CD80 (SEQ ID NO: 251), (7) NY-ESO1-LAMP-IL-15 (SEQ ID NO: 222), (8) CD161-LAMP-sCD40L (SEQ ID NO: 235), and a second generation Covid-19 construct (9) Spike-LAMP-sCD40L (2-V Covid vaccine; SEQ ID NO: 230). Sequences for these constructs and their components are provided in the table below.

HER2-LAMP-sCD40L expresses a HER2-LAMP polypeptide SEQ ID NO: 195 and a mSPD-sCD40L fusion protein SEQ ID NO: 196. HER2-LAMP-mFLT3L expresses a HER2-LAMP polypeptide (SEQ ID NO: 195) and an SPD-mFLT3L polypeptide SEQ ID NO: 207. HER2-LAMP-IL-12 expresses a HER2-LAMP polypeptide (SEQ ID NO: 195) and a murine IL-12 p36-P2A-IL-12p40 polypeptide SEQ ID NO: 213. HER2-LAMP-IL-21 expresses a HER2-LAMP polypeptide (SEQ ID NO: 195) and a murine IL-21 polypeptide SEQ ID NO: 217. HER2-LAMP-OX40L expresses a HER2-LAMP polypeptide (SEQ ID NO: 195) and an OX40L extracellular domain (ECD) Fc fusion protein with a murine IL-2 signal peptide (SP) SEQ ID NO: 242. HER2-LAMP-CD80 expresses a HER2-LAMP polypeptide (SEQ ID NO: 195) and a CD80 ECD with a murine IL-2 SP SEQ ID NO: 252. The Spike-LAMP-sCD40L expresses a Spike-LAMP polypeptide SEQ ID NO: 229 and an SPD-sCD40L polypeptide SEQ ID NO: 233. A table illustrating these and other sequences now follows. In comparison to the US provisional priority application, SEQ ID NO: 195 has been updated to reflect that the HER2-LAMP polypeptide sequence comprises SEQ ID NO: 198 followed by SEQ ID NO: 200 followed by SEQ ID NO: 202, and duplicate HER2-LAMP sequences that were included in the provisional priority application have been deleted without changing the overall numbering of the surrounding sequences. SEQ ID NOs: 221 and 229 are similarly updated to reflect that the NY-ESO1-LAMP and Spike-LAMP polypeptide sequences comprise SEQ ID NO: 198 followed by the NY-ESO1 antigen or Spike antigen followed by SEQ ID NO: 202.

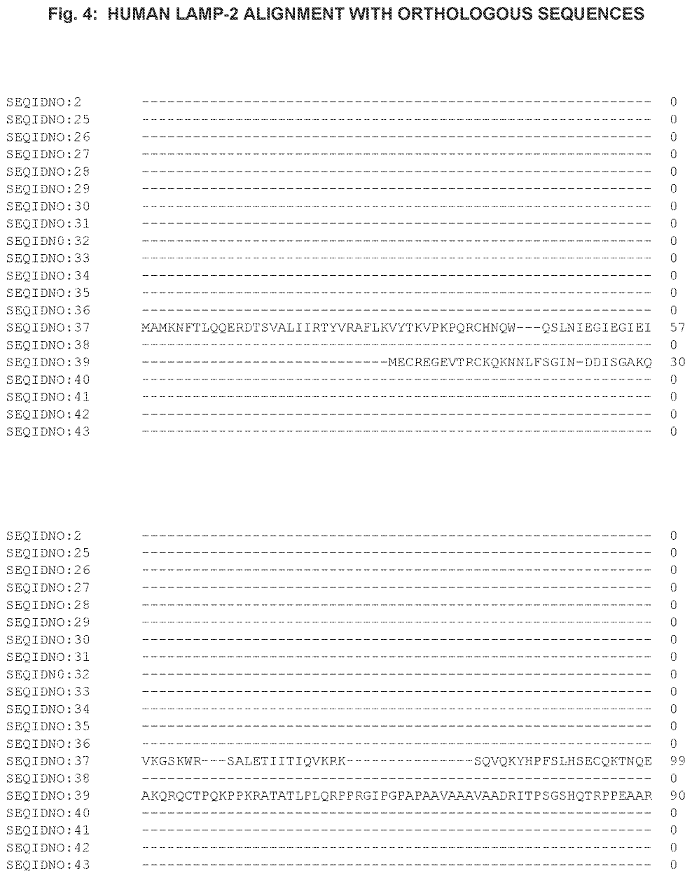

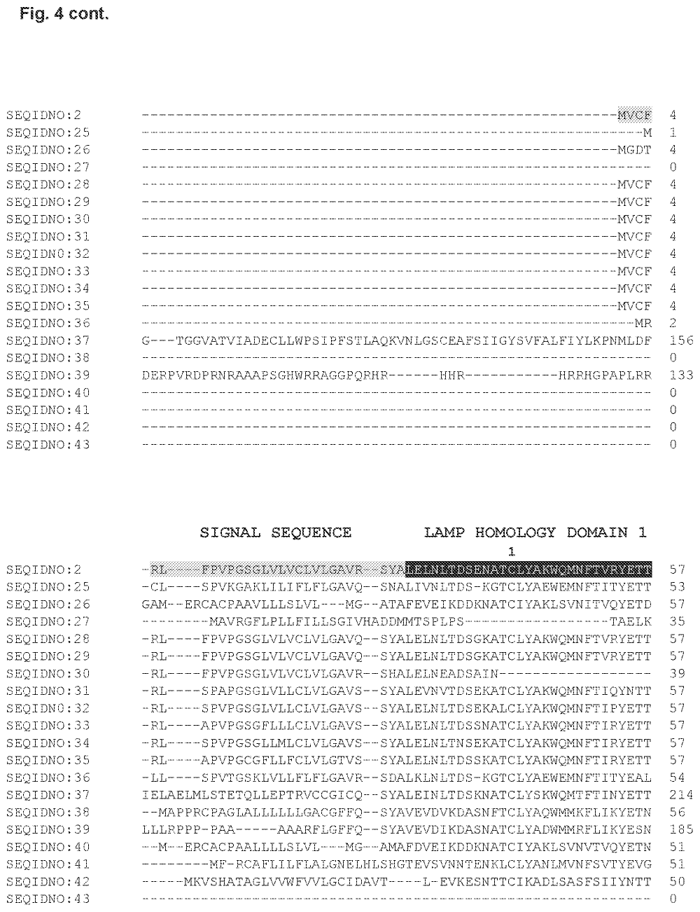

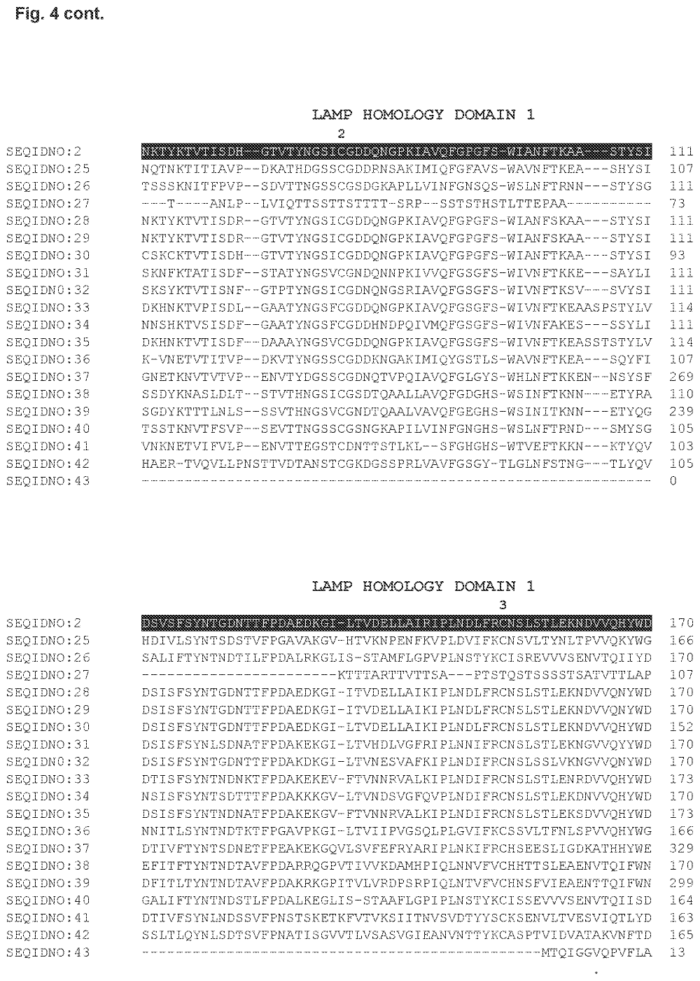

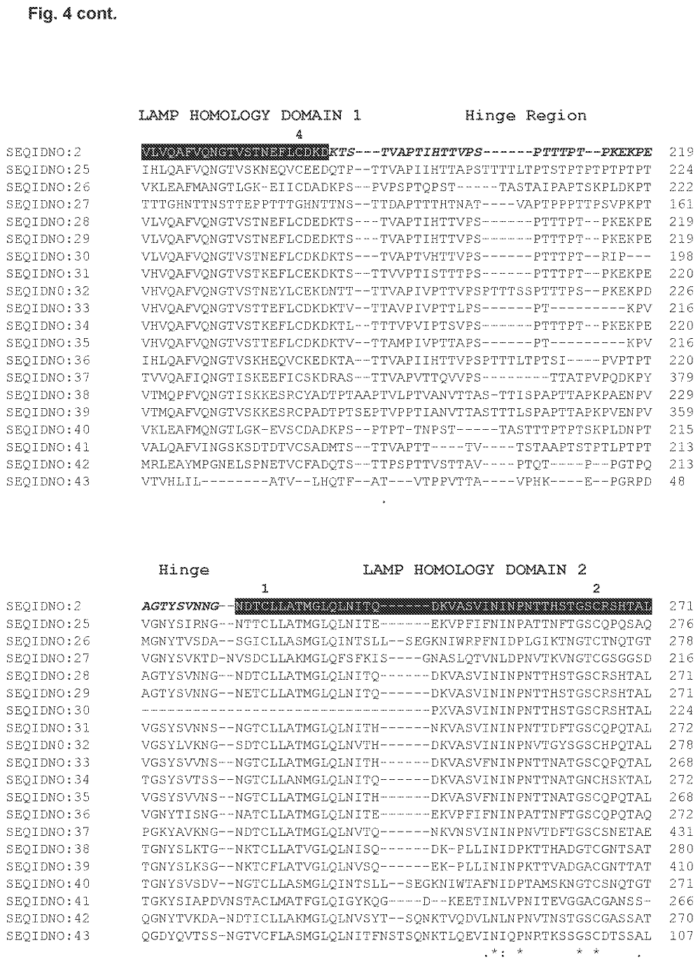

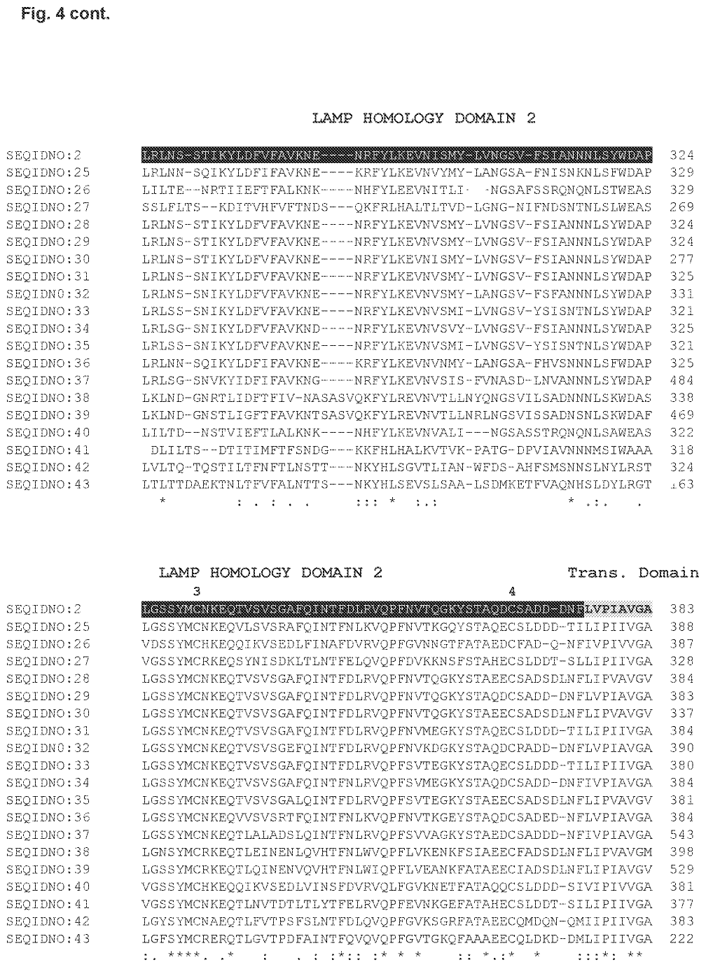

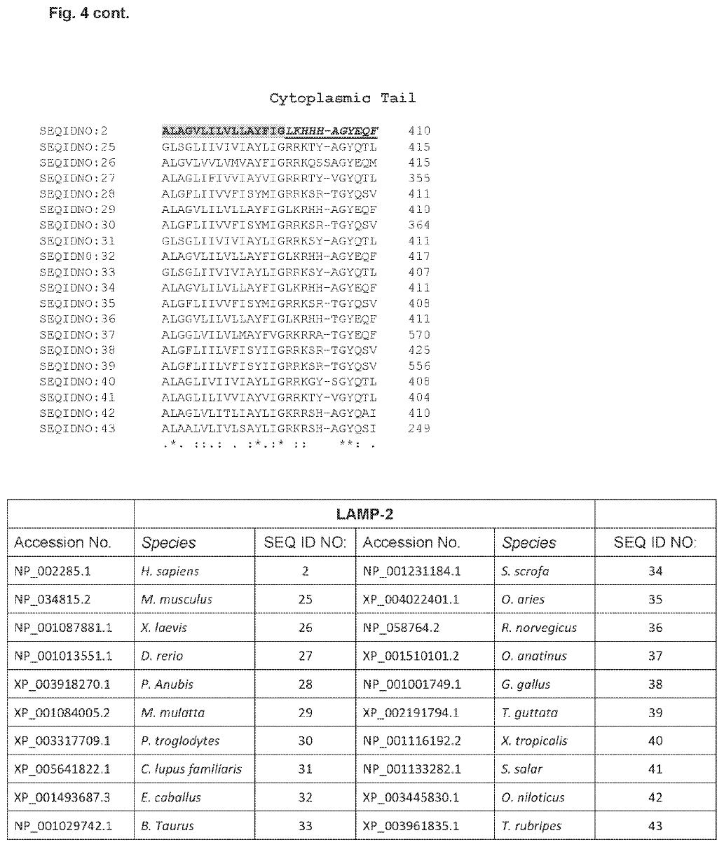

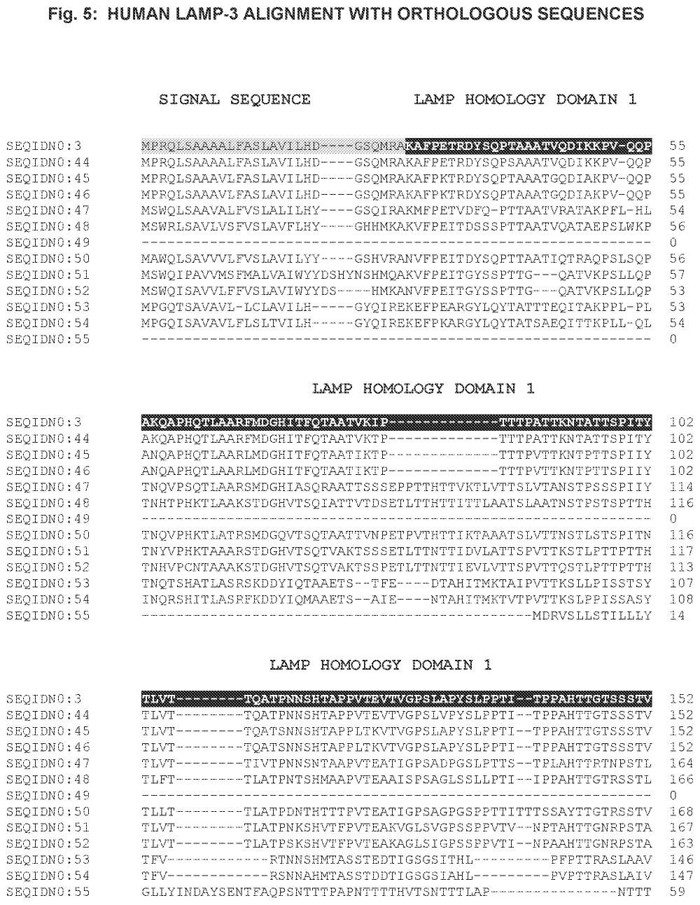

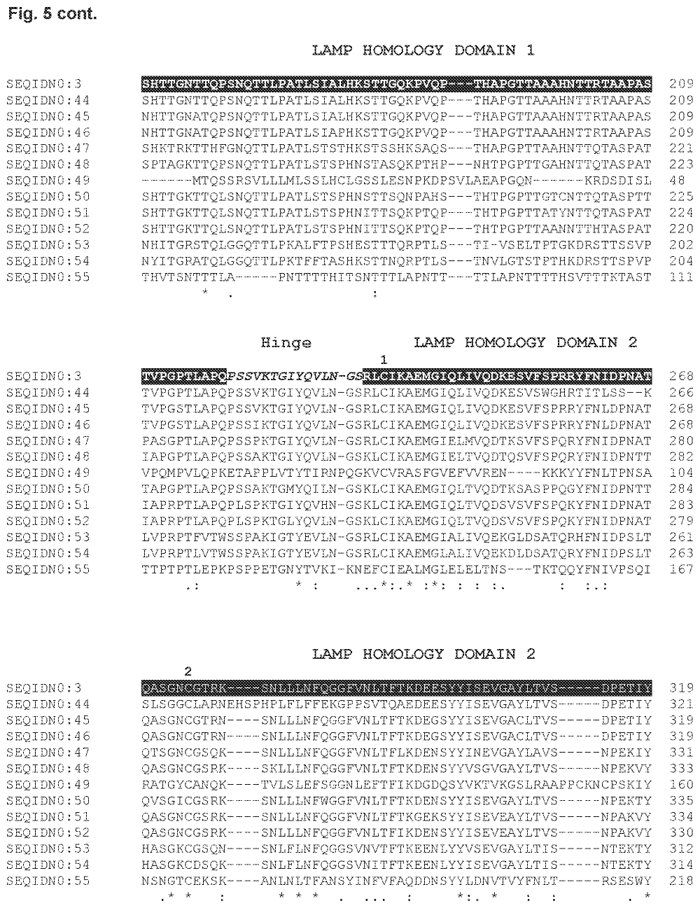

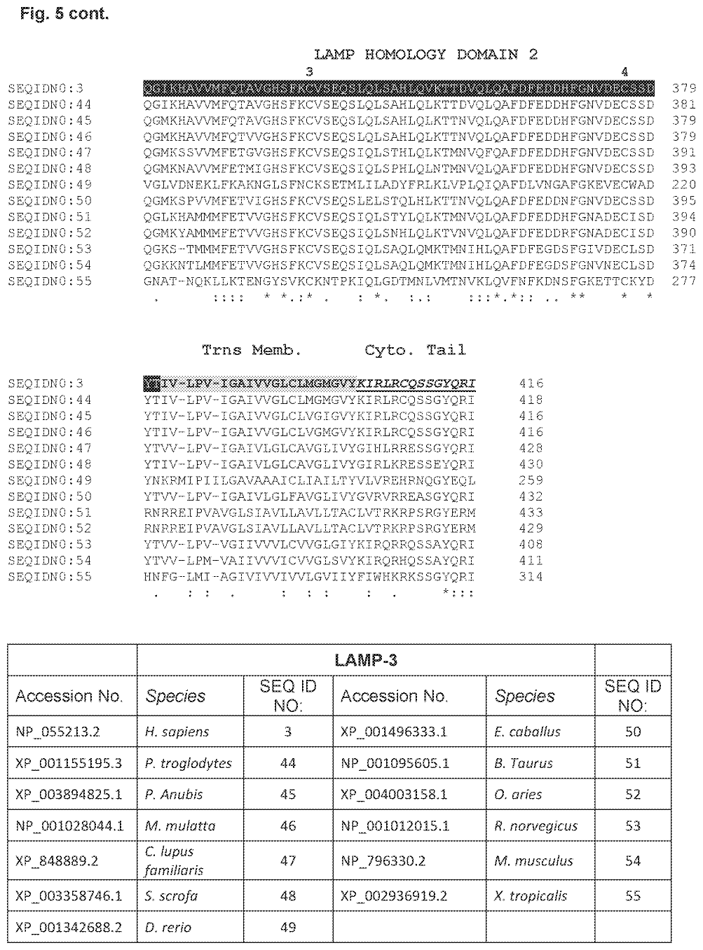

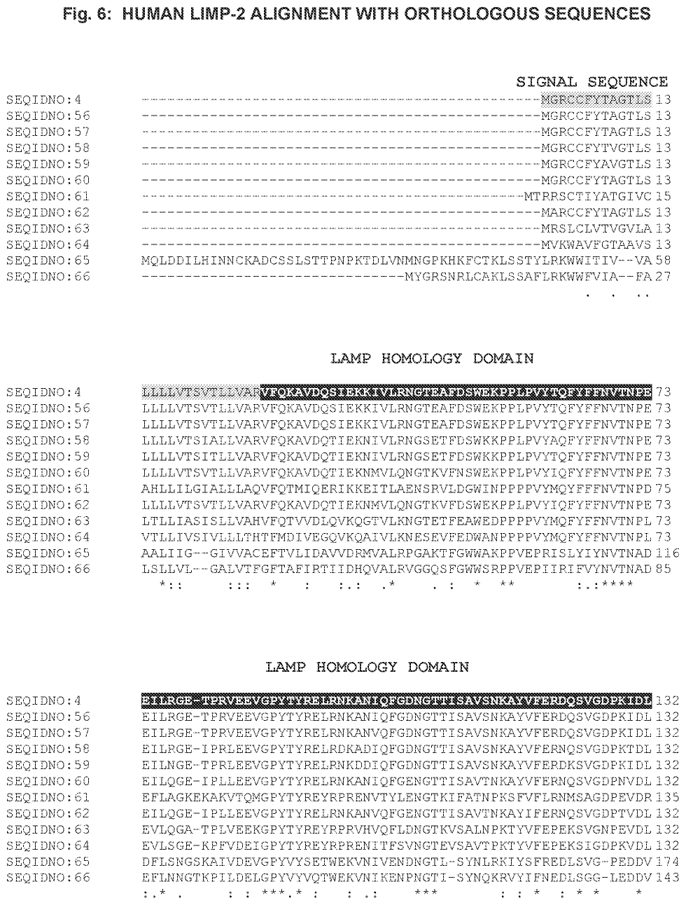

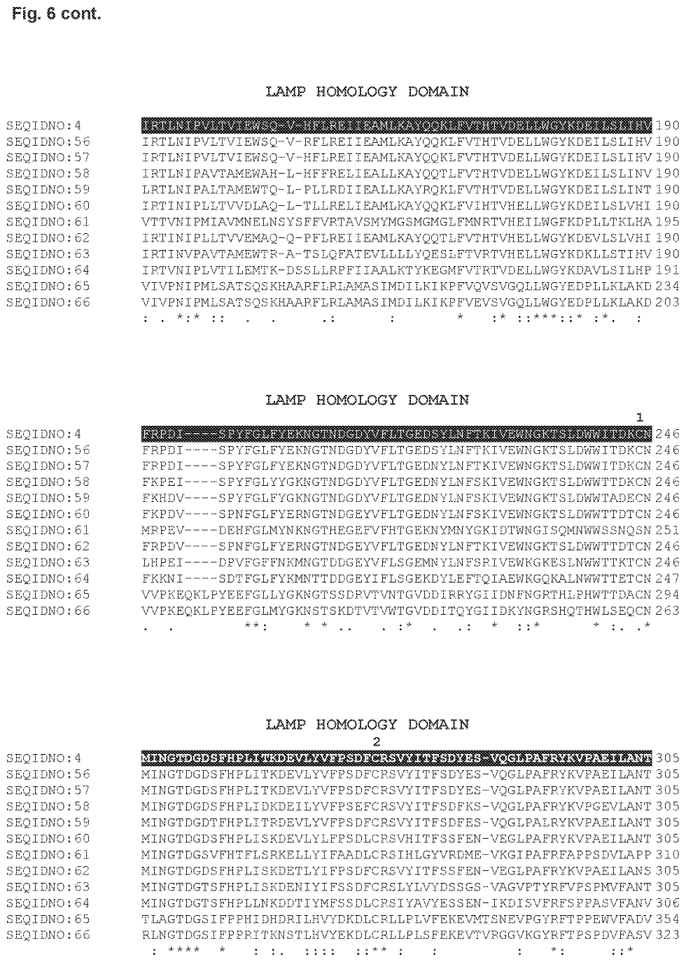

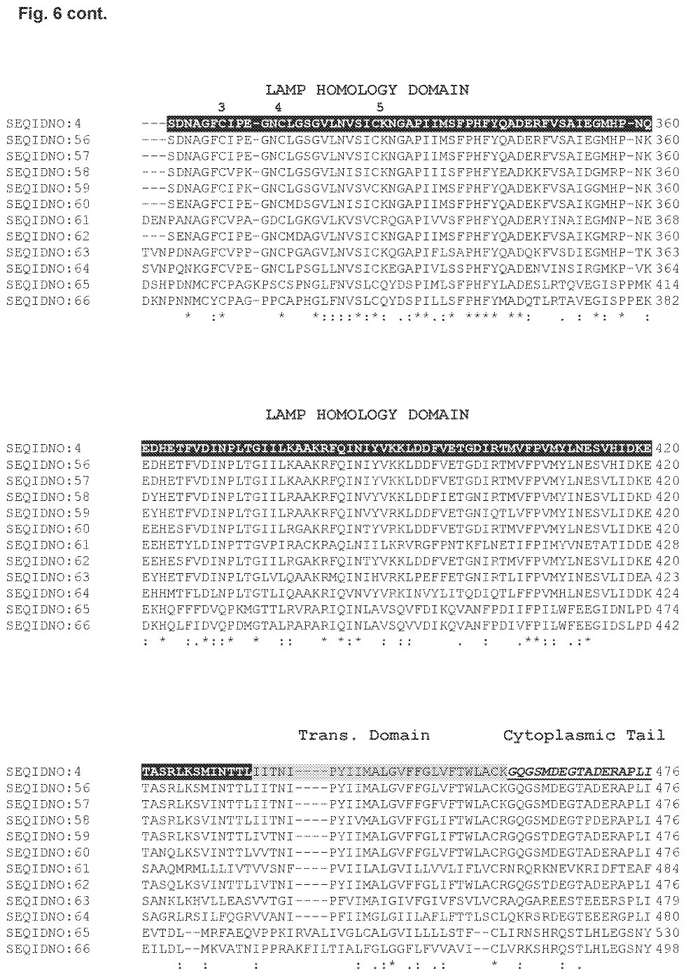

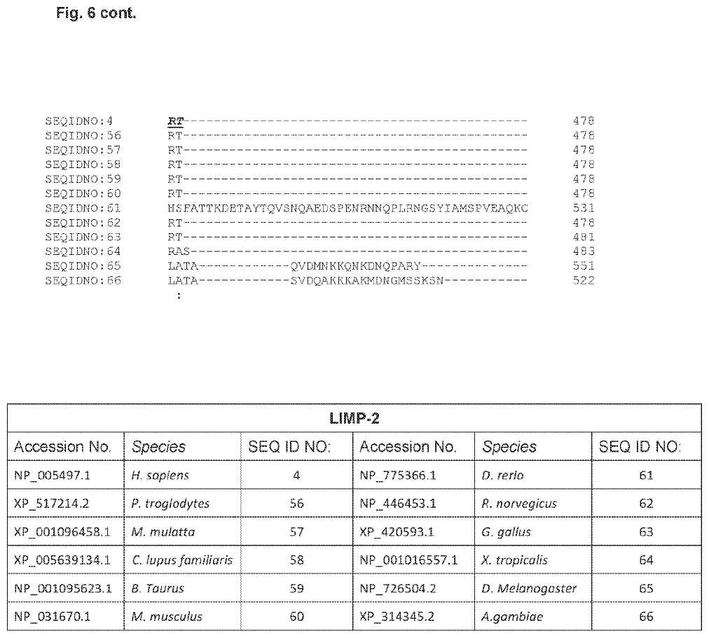

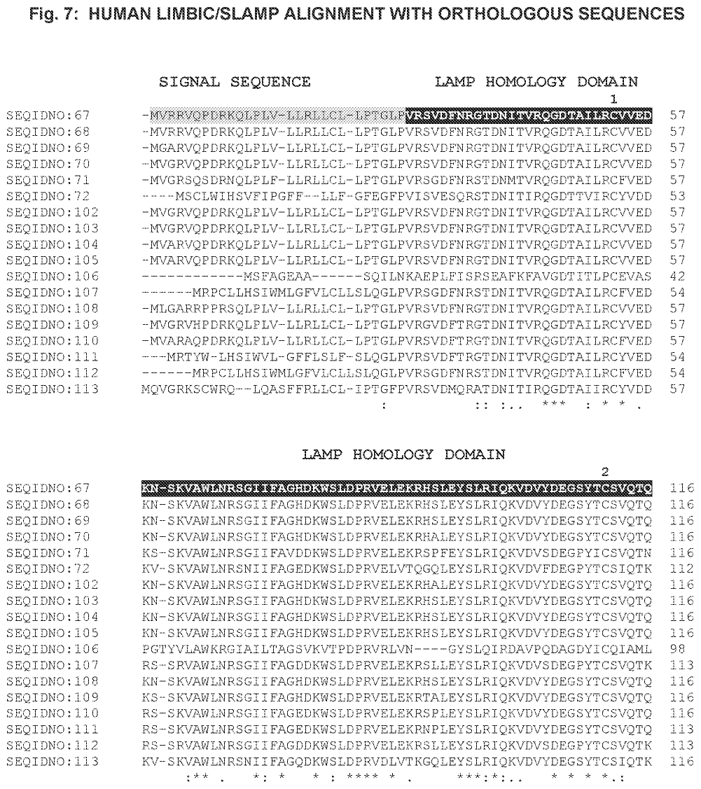

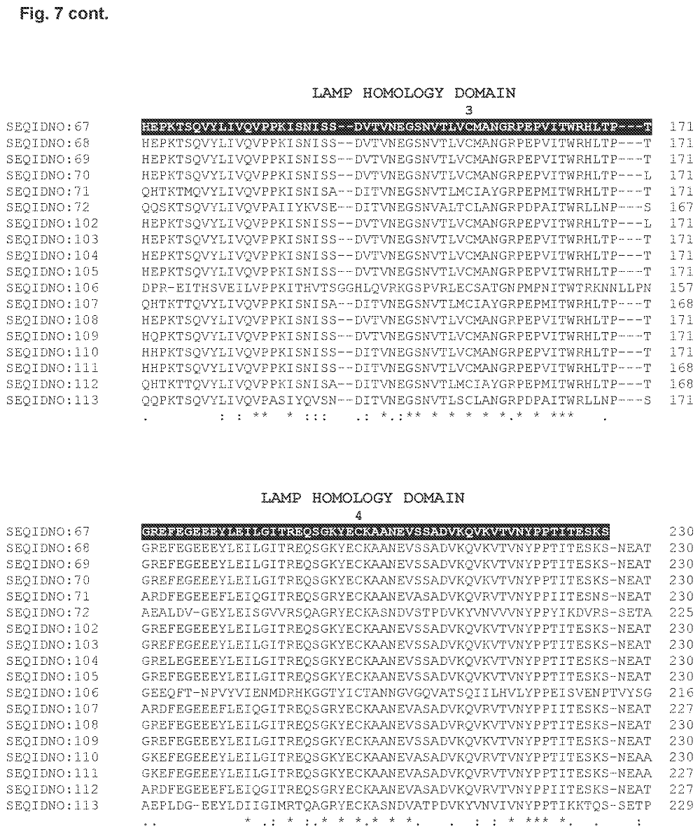

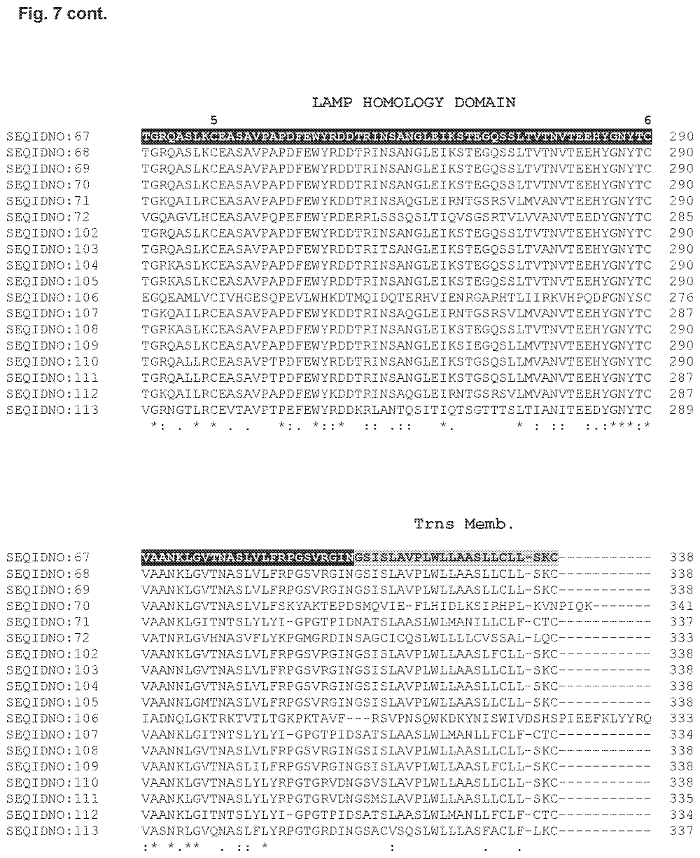

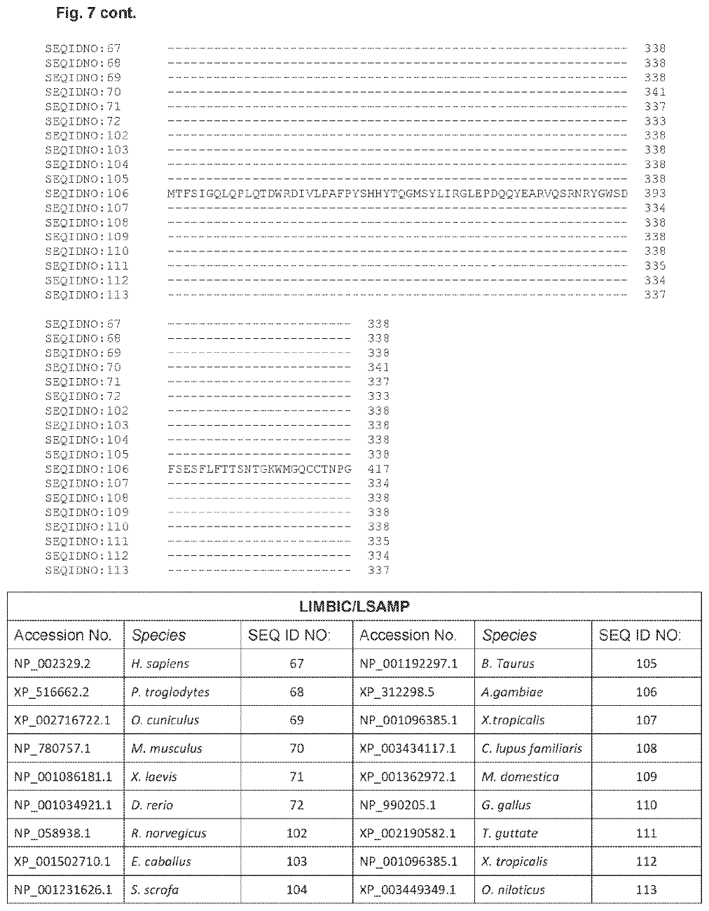

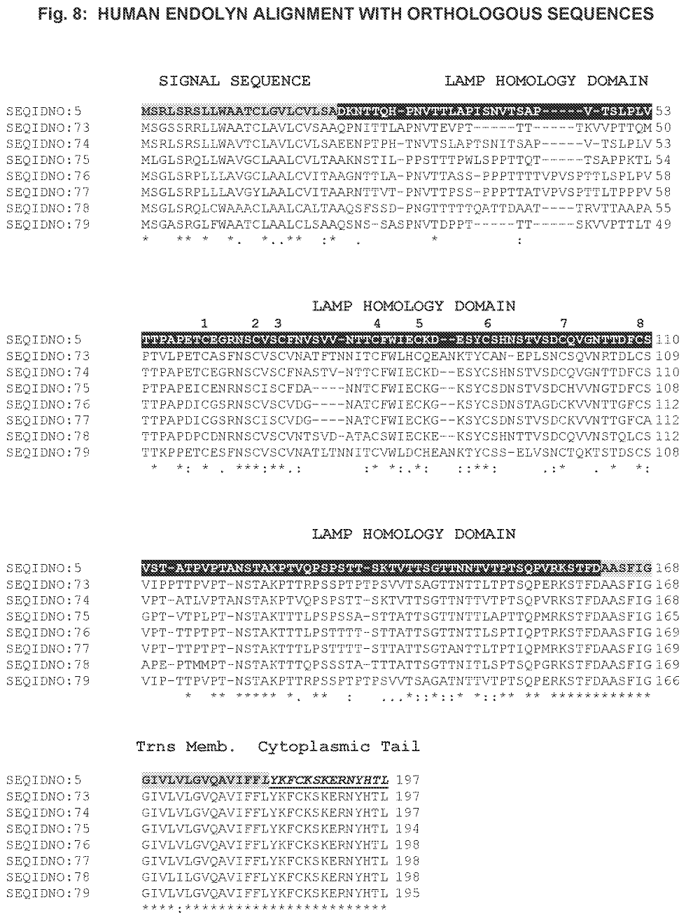

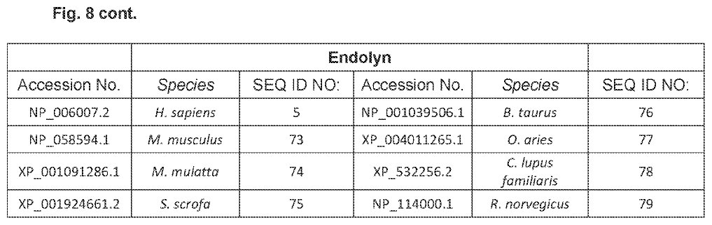

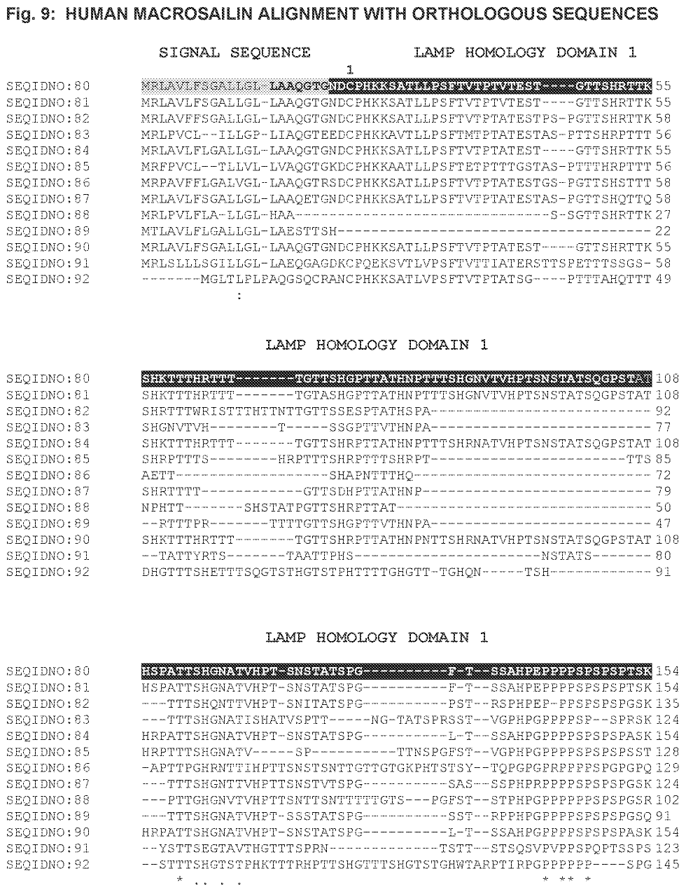

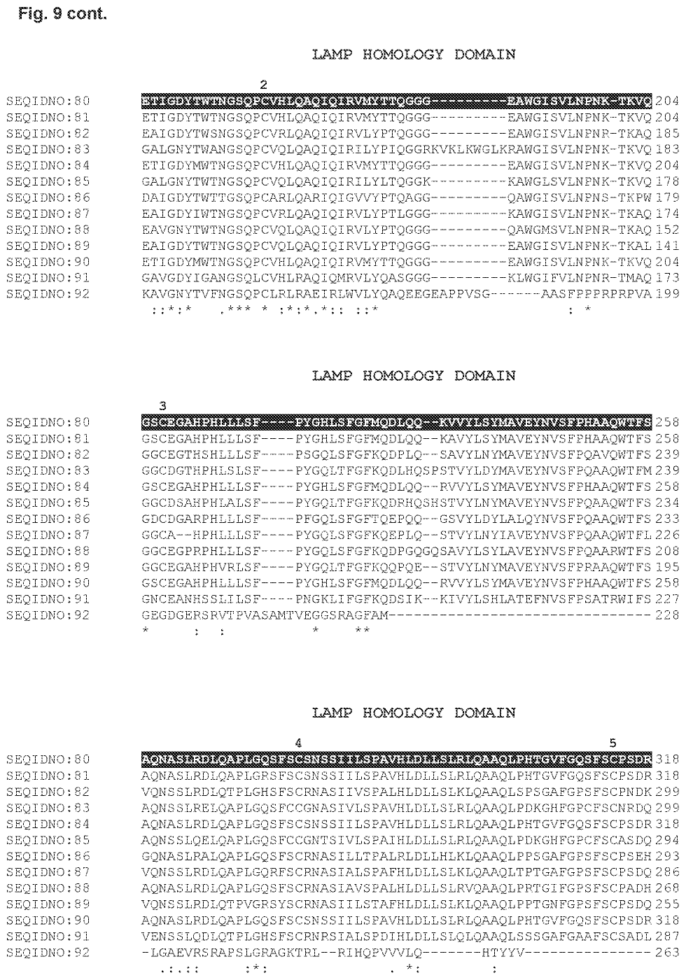

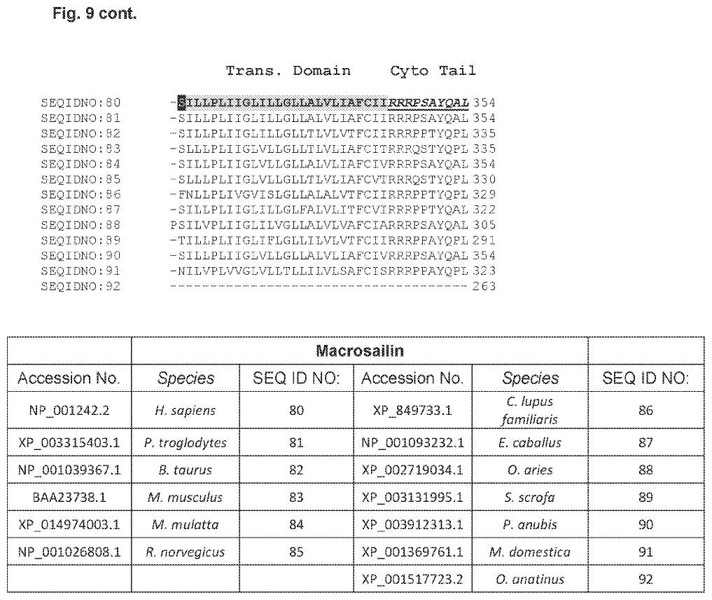

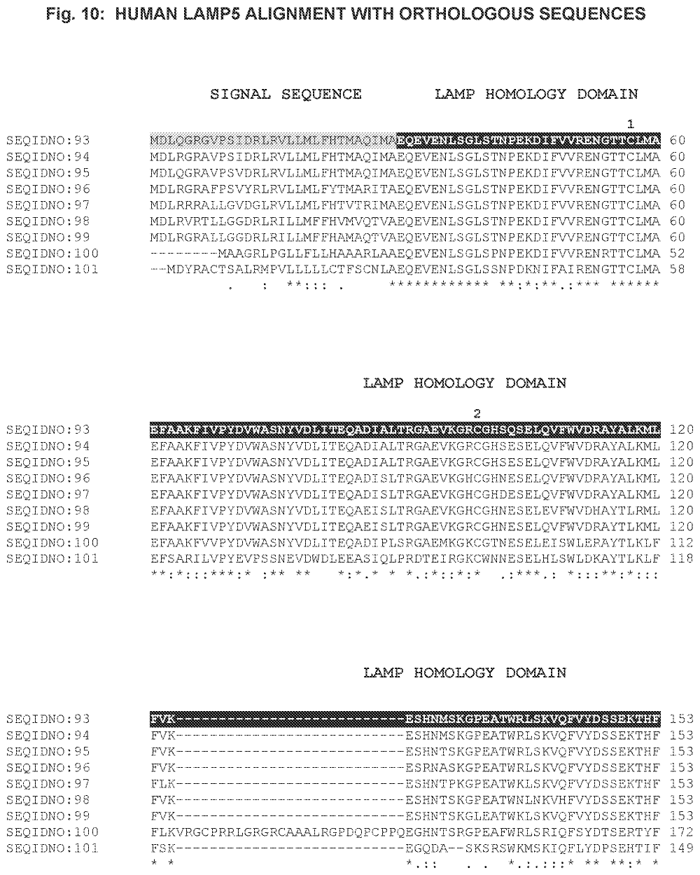

TABLE 1
DNA and Protein Sequences in Bicistronic Constructs

| | SEQ | |
|---|---|---|
| Protein | ID NO: | Sequence |
| H. sapiens LAMP-1 Protein | 1 | See FIG. 3 |
| Sequence; Accession No. | | |
| NP_005552.3 | | |
| H. sapiens LAMP-2 Protein | 2 | See FIG. 4 |
| Sequence; Accession No. | | |
| NP_002285.1 | | |
| H. sapiens LAMP-3 Protein | 3 | See FIG. 5 |
| Sequence; Accession No. | | |
| NP_055213.2 | | |
| H. sapiens LIMP-2 Protein | 4 | See FIG. 6 |
| Sequence; Accession No. | | |
| NP_005497.1 | | |
| H. sapiens Endolyn Protein | 5 | See FIG. 8 |
| Sequence; Accession No. | | |
| NP_006007.2 | | |
| M. musculus LAMP-1 Protein | 6 | See FIG. 3 |
| Sequence; Accession No. | | |
| NP_034814.2 | | |
| D. rerio LAMP-1 Protein | 7 | See FIG. 3 |
| Sequence; Accession No. | | |
| NP_955996.1 | | |
| X. Laevis LAMP-1 Protein | 8 | See FIG. 3 |
| Sequence; Accession No. | | |
| NP_001087042.1 | | |
| P. troglodytes LAMP-1 Protein | 9 | See FIG. 3 |
| Sequence; Accession No. | | |
| NP_001233491.1 | | |
| M. mulatta LAMP-1 Protein | 10 | See FIG. 3 |
| Sequence; Accession No. | | |
| XP_001087801.1 | | |
| C, lupus familiaris LAMP-1 | 11 | See FIG. 3 |
| Protein Sequence; Accession | | |
| No. XP_534193.2 | | |
| O. cuniculus LAMP-1 Protein | 12 | See FIG. 3 |
| Sequence; Accession No. | | |
| XP_002723509.1 | | |
| B. taurus LAMP-1 Protein | 13 | See FIG. 3 |
| Sequence; Accession No. | | |
| NP_001068592.1 | | |
| R. novegicus LAMP-1 Protein | 14 | See FIG. 3 |
| Sequence; Accession No. | | |
| NP_036989.1 | | |
| G. gallus LAMP-1 Protein | 15 | See FIG. 3 |
| Sequence; Accession No. | | |
| NP_990614.1 | | |
| S. scrofa LAMP-1 Protein | 16 | See FIG. 3 |
| Sequence; Accession No. | | |
| NP_001011507.1 | | |
| M. domestica LAMP-1 Protein | 17 | See FIG. 3 |
| Sequence; Accession No. | | |
| XP_001374132.1 | | |
| M. gallopavo LAMP-1 Protein | 18 | See FIG. 3 |
| Sequence; Accession No. | | |
| XP_003203252.1 | | |
| T. guttate LAMP-1 Protein | 19 | See FIG. 3 |
| Sequence; Accession No. | | |
| XP_002191607.2 | | |
| A. carolinensis LAMP-1 | 20 | See FIG. 3 |
| Protein Sequence; Accession | | |
| No. XP_003218797.1 | | |
| O. latipes LAMP-1 Protein | 21 | See FIG. 3 |
| Sequence; Accession No. | | |
| XP_004067118.1 | | |
| T. rubripes LAMP-1 Protein | 22 | See FIG. 3 |
| Sequence; Accession No. | | |
| XP_003969941.1 | | |
| S. salar LAMP-1 Protein | 23 | See FIG. 3 |
| Sequence; Accession No. | | |
| NP_001158846.1 | | |
| O. niloticus LAMP-1 Protein | 24 | See FIG. 3 |
| Sequence; Accession No. | | |
| XP_003452974.1 | | |
| M. musculus LAMP-2 Protein | 25 | See FIG. 4 |
| Sequence; Accession No. | | |
| NP_034815.2 | | |
| X. Laevis LAMP-2 Protein | 26 | See FIG. 4 |
| Sequence; Accession No. | | |
| NP_001087881.1 | | |
| D. rerio LAMP-2 Protein | 27 | See FIG. 4 |
| Sequence; Accession No. | | |
| NP_001013551.1 | | |
| 142nubisbis LAMP-2 Protein | 28 | See FIG. 4 |
| Sequence; Accession No. | | |
| XP_003918270.1 | | |
| M. mulatta LAMP-2 Protein | 29 | See FIG. 4 |
| Sequence; Accession No. | | |
| XP_001084005.2 | | |
| P. troglodytes LAMP-2 Protein | 30 | See FIG. 4 |
| Sequence; Accession No. | | |
| XP_003317709.1 | | |
| C. lupus familiaris LAMP-2 | 31 | See FIG. 4 |
| Protein Sequence; Accession | | |
| No. XP_005641822.1 | | |
| E. caballus LAMP-2 Protein | 32 | See FIG. 4 |
| Sequence; Accession No. | | |
| XP_001493687.3 | | |
| B. taurus LAMP-2 Protein | 33 | See FIG. 4 |
| Sequence; Accession No. | | |
| NP_001029742.1 | | |
| S. scrofa LAMP-2 Protein | 34 | See FIG. 4 |
| Sequence; Accession No. | | |
| NP_001231184.1 | | |
| O. aries LAMP-2 Protein | 35 | See FIG. 4 |
| Sequence; Accession No. | | |
| XP_004022401.1 | | |
| R. norvegicus LAMP-2 Protein | 36 | See FIG. 4 |
| Sequence; Accession No. | | |
| NP_058764.2 | | |
| O. anatinus LAMP-2 Protein | 37 | See FIG. 4 |
| Sequence; Accession No. | | |
| XP_001510101.2 | | |
| G. gallus LAMP-2 Protein | 38 | See FIG. 4 |
| Sequence; Accession No. | | |
| NP_001001749.1 | | |
| T. guttata LAMP-2 Protein | 39 | See FIG. 4 |
| Sequence; Accession No. | | |
| XP_002191794.1 | | |
| X. tropicalis LAMP- Protein | 40 | See FIG. 4 |
| Sequence 2; Accession No. | | |
| NP_001116192.2 | | |
| S. salar LAMP-2 Protein | 41 | See FIG. 4 |
| Sequence; Accession No. | | |
| NP_001133282.1 | | |
| O. niloticus LAMP-2 Protein | 42 | See FIG. 4 |
| Sequence; Accession No. | | |
| XP_003445830.1 | | |
| T. rubripes LAMP-2 Protein | 43 | See FIG. 4 |
| Sequence; Accession No. | | |
| XP_003961835.1 | | |
| P. troglodytes LAMP-3 Protein | 44 | See FIG. 5 |
| Sequence; Accession No. | | |
| XP_001155195.3 | | |
| 143nubisbis LAMP-3 Protein | 45 | See FIG. 5 |
| Sequence; Accession No. | | |
| XP_003894825.1 | | |
| M. mulatta LAMP-3 Protein | 46 | See FIG. 5 |
| Sequence; Accession No. | | |
| NP_001028044.1 | | |
| C. lupus familiaris LAMP-3 | 47 | See FIG. 5 |
| Protein Sequence; Accession | | |
| No. XP_848889.2 | | |
| S. scrofa LAMP-3 Protein | 48 | See FIG. 5 |
| Sequence; Accession No. | | |
| XP_003358746.1 | | |
| D. rerio LAMP-3 Protein | 49 | See FIG. 5 |
| Sequence; Accession No. | | |
| XP_001342688.2 | | |
| E. caballus LAMP-3 Protein | 50 | See FIG. 5 |
| Sequence; Accession No. | | |
| XP_001496333.1 | | |
| B. taurus LAMP-3 Protein | 51 | See FIG. 5 |
| Sequence; Accession No. | | |
| NP_001095605.1 | | |
| O. aries LAMP-3 Protein | 52 | See FIG. 5 |
| Sequence; Accession No. | | |
| XP_004003158.1 | | |
| R. norvegicus LAMP-3 Protein | 53 | See FIG. 5 |
| Sequence; Accession No. | | |
| NP_001012015.1 | | |
| M. musculus LAMP-3 Protein | 54 | See FIG. 5 |
| Sequence; Accession No. | | |
| NP_796330.2 | | |
| X. tropicalis LAMP-3 Protein | 55 | See FIG. 5 |
| Sequence; Accession No. | | |
| XP_002936919.2 | | |
| P. troglodytes LIMP-2 Protein | 56 | See FIG. 6 |
| Sequence; Accession No. | | |
| XP_517214.2 | | |
| M. mulatta LIMP-2 Protein | 57 | See FIG. 6 |
| Sequence; Accession No. | | |
| XP_001096458.1 | | |
| C. lupus familiaris LIMP-2 | 58 | See FIG. 6 |
| Protein Sequence; Accession | | |
| No. XP_005639134.1 | | |
| B. taurus LIMP-2 Protein | 59 | See FIG. 6 |
| Sequence; Accession No. | | |
| NP_001095623.1 | | |
| M. musculus LIMP-2 Protein | 60 | See FIG. 6 |
| Sequence; Accession No. | | |
| NP_031670.1 | | |
| D. rerio LIMP-2 Protein | 61 | See FIG. 6 |
| Sequence; Accession No. | | |
| NP_775366.1 | | |
| R. norvegicus LIMP-2 Protein | 62 | See FIG. 6 |
| Sequence; Accession No. | | |
| NP_446453.1 | | |
| G. gallus LIMP-2 Protein | 63 | See FIG. 6 |
| Sequence; Accession No. | | |
| XP_420593.1 | | |
| X. tropicalis LIMP-2 Protein | 64 | See FIG. 6 |
| Sequence; Accession No. | | |
| NP_001016557.1 | | |
| D. melanogaster LIMP-2 | 65 | See FIG. 6 |
| Protein Sequence; Accession | | |
| No. NP_726504.2 | | |
| A. gambiae LIMP-2 Protein | 66 | See FIG. 6 |
| Sequence; Accession No. | | |
| XP_314345.2 | | |
| H. sapiens LIMBIC/SLAMP | 67 | See FIG. 7 |
| Protein Sequence; Accession | | |
| No. NP_002329.2 | | |
| P. troglodytes | 68 | See FIG. 7 |
| LIMBIC/SLAMP Protein | | |
| Sequence; Accession No. | | |
| XP_516662.2 | | |
| O. cuniculus LIMBIC/SLAMP | 69 | See FIG. 7 |
| Protein Sequence; Accession | | |
| No. XP_002716722.1 | | |
| M. musculus LIMBIC/SLAMP | 70 | See FIG. 7 |
| Protein Sequence; Accession | | |
| No. NP_780757.1 | | |
| X. Laevis LIMBIC/SLAMP | 71 | See FIG. 7 |
| Protein Sequence; Accession | | |
| No. NP_001086181.1 | | |
| D. rerio LIMBIC/SLAMP | 72 | See FIG. 7 |
| Protein Sequence; Accession | | |
| No. NP_0010349421.1 | | |
| M. musculus Endolyn Protein | 73 | See FIG. 8 |
| Sequence; Accession No. | | |
| NP_058594.1 | | |
| M. mulatta Endolyn Protein | 74 | See FIG. 8 |
| Sequence; Accession No. | | |
| XP_001091286.1 | | |
| S. scrofa Endolyn Protein | 75 | See FIG. 8 |
| Sequence; Accession No. | | |
| XP_001924661.2 | | |
| B. taurus Endolyn Protein | 76 | See FIG. 8 |
| Sequence; Accession No. | | |
| NP_001039506.1 | | |
| O. aries Endolyn Protein | 77 | See FIG. 8 |
| Sequence`; Accession No. | | |
| XP_004011265.1 | | |
| C. lupus familiaris Endolyn | 78 | See FIG. 8 |
| Protein Sequence; Accession | | |
| No. XP_532256.2 | | |
| R. norvegicus Endolyn Protein | 79 | See FIG. 8 |
| Sequence; Accession No. | | |
| NP_114000.1 | | |
| H. sapiens Macrosialin Protein | 80 | See FIG. 9 |
| Sequence; Accession No. | | |
| NP_001242.2 | | |
| P. troglodytes Macrosialin | 81 | See FIG. 9 |
| Protein Sequence; Accession | | |
| No. XP_003315403.1 | | |
| B. taurus Macrosialin Protein | 82 | See FIG. 9 |
| Sequence; Accession No. | | |
| NP_001039367.1 | | |
| M. musculus Macrosialin | 83 | See FIG. 9 |
| Protein Sequence; Accession | | |
| No. BAA23738.1 | | |
| M. mulatta Macrosialin | 84 | See FIG. 9 |
| Protein Sequence; Accession | | |
| No. XP_014974003.1 | | |
| R. norvegicus Macrosialin | 85 | See FIG. 9 |
| Protein Sequence; Accession | | |
| No. NP_001026808.1 | | |
| C. lupus familiaris Macrosialin | 86 | See FIG. 9 |
| Protein Sequence; Accession | | |
| No. XP_849733.1 | | |
| E. caballus Macrosialin | 87 | See FIG. 9 |
| Protein Sequence; Accession | | |
| No. NP_001093232.1 | | |
| O. aries Macrosialin Protein | 88 | See FIG. 9 |
| Sequence; Accession No. | | |
| XP_002719034.1 | | |
| S. scrofa Macrosialin Protein | 89 | See FIG. 9 |
| Sequence; Accession No. | | |
| XP_003131995.1 | | |
| 147nubisbis Macrosialin | 90 | See FIG. 9 |
| Protein Sequence; Accession | | |
| No. XP_03912313.1 | | |
| M. domestica Macrosialin | 91 | See FIG. 9 |
| Protein Sequence; Accession | | |
| No. XP_001369761.1 | | |
| O. anatinus Macrosialin | 92 | See FIG. 9 |
| Protein Sequence; Accession | | |
| No. XP_001517723.2 | | |
| H. sapiens LAMP5 Protein | 93 | See FIG. 10 |
| Sequence; Accession No. | | |
| NP_036393.1 | | |
| P. troglodytes LAMP5 Protein | 94 | See FIG. 10 |
| Sequence; Accession No. | | |
| NP_036393.1 | | |
| M. mulatta LAMP5 Protein | 95 | See FIG. 10 |
| Sequence; Accession No. | | |
| NP_001181627.1 | | |
| C. lupus familiaris LAMP5 | 96 | See FIG. 10 |
| Protein Sequence; Accession | | |
| No. XP_850634.1 | | |
| B. taurus LAMP5 Protein | 97 | See FIG. 10 |
| Sequence; Accession No. | | |
| NP_001076887.1 | | |
| M. musculus LAMP5 Protein | 98 | See FIG. 10 |
| Sequence; Accession No. | | |
| NP_083806.2 | | |
| R. norvegicus LAMP5 Protein | 99 | See FIG. 10 |
| Sequence; Accession No. | | |
| NP_001014205.1 | | |
| G. gallus LAMP5 Protein | 100 | See FIG. 10 |
| Sequence; Accession No. | | |
| XP_004935300.1 | | |
| X. tropicalis LAMP5 Protein | 101 | See FIG. 10 |
| Sequence; Accession No. | | |
| NP_001090781.1 | | |
| R. norvegicus LIMBIC/SLAMP | 102 | See FIG. 7 |
| Protein Sequence; Accession | | |
| No. NP_058938.1 | | |
| E. caballus LIMBIC/SLAMP | 103 | See FIG. 7 |
| Protein Sequence; Accession | | |
| No. XP_001502710.1 | | |
| S. scrofa LIMBIC/SLAMP | 104 | See FIG. 7 |
| Protein Sequence; Accession | | |
| No. NP_001231626.1 | | |
| B. taurus LIMBIC/SLAMP | 105 | See FIG. 7 |
| Protein Sequence; Accession | | |
| No. NP_001192297.1 | | |
| A. gambiae LIMBIC/SLAMP | 106 | See FIG. 7 |
| Protein Sequence; Accession | | |
| No. XP_312298.5 | | |
| X. tropicalis LIMBIC/SLAMP | 107 | See FIG. 7 |
| Protein Sequence; Accession | | |
| No. NP_001096385.1 | | |
| C. lupus familiaris | 108 | See FIG. 7 |
| LIMBIC/SLAMP Protein | | |
| Sequence; Accession No. | | |
| XP_003434117.1 | | |
| M. domestica | 109 | See FIG. 7 |
| LIMBIC/SLAMP Protein | | |
| Sequence; Accession No. | | |
| XP_001362972.1 | | |
| G. gallus LIMBIC/SLAMP | 110 | See FIG. 7 |
| Protein Sequence; Accession | | |
| No. NP_990205.1 | | |
| T. guttate LIMBIC/SLAMP | 111 | See FIG. 7 |
| Protein Sequence; Accession | | |
| No. XP_002190582.1 | | |
| X. tropicalis LIMBIC/SLAMP | 112 | See FIG. 7 |
| Protein Sequence; Accession | | |
| No. NP_001096385.1 | | |
| O. niloticus LIMBIC/SLAMP | 113 | See FIG. 7 |
| Protein Sequence; Accession | | |
| No. XP_003449349.1 | | |
| Exemplary LAMP Signal | 114 | MAPRSARRPLLLLLLLLLLGLMHCASA |
| Sequence; Protein Sequence | | |
| N-LAMP Protein Sequence | 115 | AMFMVKNGNGTACIMANFSAAFSVNYDTKSGPKNMTLDLPSDATVVLNRSSCGKENTSDPSLVIAFGRGHTLTLNFTRN |
| | | ATRYSVQLMSFVYNLSDTHLFPNASSKEIKTVESITDIRADIDKKYRCVSGTQVHMNNVTVTLHDATIQAYLSNSSFSR |
| | | GETRCEQD |
| C-LAMP Protein Sequence | 116 | ADPHRCGWCPGGAGPHRPHRLPRRQEEESRRLPDYL |
| TM Cyto LAMP Protein | 117 | LIPIAVGGALAGLVLIVLIAYLVGRKRSHAGYQTI |
| Sequence | | |
| SARS CoV-2 S1 Spike | 118 | VNLTTRTQLPPAYTNSFTRGVYYPDKVFRSSVLHSTQDLFLPFFSNVTWFHAIHVSGTNGTKRFDNPVLPFNDGVYFAS |
| Protein Sequence | | TEKSNIIRGWIFGTTLDSKTQSLLIVNNATNVVIKVCEFQFCNDPFLGVYYHKNNKSWMESEFRVYSSANNCTFEYVSQ |
| | | PFLMDLEGKQGNFKNLREFVFKNIDGYFKIYSKHTPINLVRDLPQGFSALEPLVDLPIGINITRFQTLLALHRSYLTPG |
| | | DSSSGWTAGAAAYYVGYLQPRTFLLKYNENGTITDAVDCALDPLSETKCTLKSFTVEKGIYQTSNFRVQPTESIVRFPN |
| | | ITNLCPFGEVFNATRFASVYAWNRKRISNCVADYSVLYNSASFSTFKCYGVSPTKLNDLCFTNVYADSFVIRGDEVRQI |
| | | APGQTGKIADYNYKLPDDFTGCVIAWNSNNLDSKVGGNYNYLYRLFRKSNLKPFERDISTEIYQAGSTPCNGVEGENCY |
| | | FPLQSYGFQPTNGVGYQPYRVVVLSFELLHAPATVCGPKKSTNLVKNKCVNFNFNGLTGTGVLTESNKKFLPFQQFGRD |
| | | IADTTDAVRDPQTLEILDITPCSFGGVSVITPGTNTSNQVAVLYQDVNCTEVPVAIHADQLTPTWRVYSTGSNVFQTRA |
| | | GCLIGAEHVNNSYECDIPIGAGICASYQTQTNSPRRAR |
| Exemplary SARS CoV-2 | 119 | VNLTTRTQLPPAYTNSFTRGVYYPDKVFRSSVLHSTQDLFLPFFSNVTWFHAIHVSGTNGTKRFDNPVLPFNDGVYFAS |
| Spike Protein Sequence (16- | | TEKSNIIRGWIFGTTLDSKTQSLLIVNNATNVVIKVCEFQFCNDPFLGVYYHKNNKSWMESEFRVYSSANNCTFEYVSQ |
| 1273) | | PFLMDLEGKQGNFKNLREFVFKNIDGYFKIYSKHTPINLVRDLPQGFSALEPLVDLPIGINITRFQTLLALHRSYLTPG |
| | | DSSSGWTAGAAAYYVGYLQPRTFLLKYNENGTITDAVDCALDPLSETKCTLKSFTVEKGIYQTSNFRVQPTESIVRFPN |
| | | ITNLCPFGEVFNATRFASVYAWNRKRISNCVADYSVLYNSASFSTFKCYGVSPTKLNDLCFTNVYADSFVIRGDEVRQI |
| | | APGQTGKIADYNYKLPDDFTGCVIAWNSNNLDSKVGGNYNYLYRLFRKSNLKPFERDISTEIYQAGSTPCNGVEGENCY |
| | | FPLQSYGFQPTNGVGYQPYRVVVLSFELLHAPATVCGPKKSTNLVKNKCVNFNFNGLTGTGVLTESNKKFLPFQQFGRD |
| | | IADTTDAVRDPQTLEILDITPCSFGGVSVITPGTNTSNQVAVLYQDVNCTEVPVAIHADQLTPTWRVYSTGSNVFQTRA |
| | | GCLIGAEHVNNSYECDIPIGAGICASYQTQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQDKNTQEVFAQVKQIYKTPPIKDFGGFNFSQI |
| | | LPDPSKPSKRSFIEDLLFNKVTLADAGFIKQYGDCLGDIAARDLICAQKFNGLTVLPPLLTDEMIAQYTSALLAGTITS |
| | | GWTFGAGAALQIPFAMQMAYRFNGIGVTQNVLYENQKLIANQFNSAIGKIQDSLSSTASALGKLQDVVNQNAQALNTLV |
| | | KQLSSNFGAISSVLNDILSRLDPPEAEVQIDRLITGRLQSLQTYVTQQLIRAAEIRASANLAATKMSECVLGQSKRVDF |
| | | CGKGYHLMSFPQSAPHGVVFLHVTYVPAQEKNFTTAPAICHDGKAHFPREGVFVSNGTHWFVTQRNFYEPQIITTDNTF |
| | | VSGNCDVVIGIVNNTVYDPLQPELDSFKEELDKYFKNHTSPDVDLGDISGINASVVNIQKEIDRLNEVAKNLNESLIDL |
| | | QELGKYEQYIKWPWYIWLGFIAGLIAIVMVTIMLCCMTSCCSCLKGCCSCGSCCKFDEDDSEPVLKGVKLHYT |
| Murine Ig-Kappa Leader; | 120 | METDTLLLWVLLLWVPGSTGD |
| Protein Sequence | | |
| Murine Ig-Kappa Leader; | 121 | atggaaaccgatacactgctgctgtgggtgctgttgctctgggttccaggatctacaggcgac |
| DNA Sequence | | |
| Human IgKVIII Leader; | 122 | MDMRVPAQLLGLLLLWLRGARC |
| Protein Sequence | | |
| Human IgKVIII Leader; | 123 | atggacatgcgtgtgccggcccaactgcttggtttgctgctcctctggcttcggggcgcaagatgc |
| DNA Sequence | | |
| Human EF-1alpha Core | 124 | gggcagagcgcacatcgcccacagtccccgagaagttggggggaggggtcggcaattgaaccggtgcctagagaaggtg |
| Promoter; DNA Sequence | | gcgcggggtaaactgggaaagtgatgtcgtgtactggctccgcctttttcccgagggtgggggagaaccgtatataagt |
| | | gcagtagtcgccgtgaacgttctttttcgcaacgggtttgccgccagaacacag |
| Human Tetranectin; Protein | 125 | EPPTQKPKKIVNAKKDVVNTKMFEELKSRLDTLAQEVALLKEQQALQTV |
| Sequence | | |
| Human Tetranectin; DNA | 126 | gagccaccaacccagaagcccaagaagattgtaaatgccaagaaagatgttgtgaacacaaagatgtttgaggagctca |
| Sequence | | agagccgtctggacaccctggcccaggaggtggccctgctgaaggagcagcaggccctgcagacggtc |
| Murine Tetranectin; Protein | 127 | ESPTPKAKKAANAKKDLVSSKMFEELKNRMDVLAQEVALLKEKQALQTV |
| Sequence | | |
| Murine Tetranectin; DNA | 128 | gagtcacccactcccaaggccaagaaggctgcaaatgccaagaaagatttggtgagctcaaagatgttcgaggagctca |
| Sequence | | agaacaggatggatgtcctggcccaggaggtggccctgctgaaggagaagcaggccttacagactgtg |
| Murine Pulmonary Surfactant | 129 | MLPFLSMLVLLVQPLGNLGAEMKSLSQRSVPNTCTLVMCSPTENGLPGRDGRDGREGPRGEKGDPGLPGPMGLSGLQGP |
| Associated Protein D (SPD); | | TGPVGPKGENGSAGEPGPKGERGLSGPPGLPGIPGPAGKEGPSGKQGNIGPQGKPGPKGEAGPKGEVGAPGMQGSTGAK |
| Protein Sequence | | GSTGPKGERGAPGVQGAPGNAGAAGPAGPAGPQGAPGSRGPPGLKGDRGVPGDRGIKGESGLPDSAALRQQMEALKGKL |
| | | QRLEVAFSHYQKAALFPDG |
| Murine Pulmonary Surfactant | 130 | atgctgccctttctctccatgcttgtcttgcttgtacagcccctgggaaatctgggagcagaaatgaagagcctctcgc |
| Associated Protein D (SPD); | | agagatcagtacccaacacctgcaccctagtcatgtgtagcccaacagagaatggcctgcctggtcgtgatggacggga |
| DNA Sequence | | tgggagagaaggtccacggggtgagaagggtgatccaggtttgccaggacctatggggctctcagggttgcagggccct |
| | | acaggtccagttggacccaaaggagagaatggctctgctggcgaacctggaccaaagggagaacgtggactaagtggac |
| | | ctccaggacttccaggtattcctggtccagctgggaaagaaggtccctctgggaagcaggggaacataggacctcaagg |
| | | caaaccaggtcctaaaggagaggctgggcccaaaggagaagtaggtgctcctggcatgcaaggatctacaggggcaaaa |
| | | ggctccacaggccccaagggagaaagaggtgcccctggtgtgcaaggagccccagggaatgctggagcagcaggacctg |
| | | ccggacctgccggtccacagggagctccaggttccagggggcccccaggactcaagggggacagaggtgttcctggaga |
| | | cagaggaatcaaaggtgaaagcgggcttccagacagtgctgctctgaggcagcagatggaggccttaaaaggaaaacta |
| | | cagcgtctagaggttgccttctcccactatcagaaagctgcattgttccctgatggc |
| Human Pulmonary | 131 | MLLFLLSALVLLTQPLGYLEAEMKTYSHRIMPSACTLVMCSSVESGLPGRDGRDGREGPRGEKGDPGLPGAAGQAGMPG |
| Surfactant Associated Protein | | QAGPVGPKGDNGSVGEPGPKGDTGPSGPPGPPGVPGPAGREGPLGKQGNIGPQGKPGPKGEAGPKGEVGAPGMQGSAGA |
| D (SPD); Protein Sequence | | RGLAGPKGERGVPGERGVPGNTGAAGSAGAMGPQGSPGARGPPGLKGDKGIPGDKGAKGESGLPDVASLRQQVEALQGQ |
| | | VQHLQAAFSQYKKVELFPNGQSVGEKIFKTAGFVKPFTEAQLLCTQAGGQLASPRSAAENAALQQLVVAKNEAAFLSMT |
| | | DSKTEGKFTYPTGESLVYSNWAPGEPNDDGGSEDCVEIFTNGKWNDRACGEKRLVVCEF |
| Human Pulmonary | 132 | atgctgctcttcctcctctctgcactggtcctgctcacacagcccctgggctacctggaagcagaaatgaagacctact |
| Surfactant Associated Protein | | cccacagaacaatgcccagtgcttgcaccctggtcatgtgtagctcagtggagagtggcctgcctggtcgcgatggacg |
| D (SPD); DNA Sequence | | ggatgggagagagggccctcggggcgagaagggggacccaggtttgccaggagctgcagggcaagcagggatgcctgga |
| | | caagctggcccagttgggcccaaaggggacaatggctctgttggagaacctggaccaaagggagacactgggccaagtg |
| | | gacctccaggacctcccggtgtgcctggtccagctggaagagaaggtcccctggggaagcaggggaacataggacctca |
| | | gggcaagccaggcccaaaaggagaagctgggcccaaaggagaagtaggtgccccaggcatgcagggctcggcaggggca |
| | | agaggcctcgcaggccctaagggagagcgaggtgtccctggtgagcgtggagtccctggaaacacaggggcagcagggt |
| | | ctgctggagccatgggtccccagggaagtccaggtgccaggggacccccgggattgaagggggacaaaggcattcctgg |
| | | agacaaaggagcaaagggagaaagtgggcttccagatgttgcttctctgaggcagcaggttgaggccttacagggacaa |
| | | gtacagcacctccaggctgctttctctcagtataagaaagttgagctcttcccaaatggccaaagtgtcggggagaaga |
| | | ttttcaagacagcaggctttgtaaaaccatttacggaggcacagctgctgtgcacacaggctggtggacagttggcctc |
| | | tccacgctctgccgctgagaatgccgccttgcaacagctggtcgtagctaagaacgaggctgctttcctgagcatgact |
| | | gattccaagacagagggcaagttcacctaccccacaggagagtccctggtctattccaactgggccccaggggagccca |
| | | acgatgatggcgggtcagaggactgtgtggagatcttcaccaatggcaagtggaatgacagggcttgtggagaaaagcg |
| | | tcttgtggtctgcgagttc |
| Human Soluble CD70; | 133 | QRFAQAQQQLPLESLGWDVAELQLNHTGPQQDPRLYWQGGPALGRSFLHGPELDKGQLRIHRDGIYMVHIQVTLAICSS |
| Protein Sequence | | TTASRHHPTTLAVGICSPASRSISLLRLSFHQGCTIASQRLTPLARGDTLCTNLTGTLLPSRNTDETFFGVQWVRP |
| Human Soluble CD70; DNA | 134 | cagcgcttcgcacaggctcagcagcagctgccgctcgagtcacttgggtgggacgtagctgagctgcagctgaatcaca |
| Sequence | | caggacctcagcaggaccccaggctatactggcaggggggcccagcactgggccgctccttcctgcatggaccagagct |
| | | ggacaaggggcagctacgtatccatcgtgatggcatctacatggtacacatccaggtgacgctggccatctgttcctcc |
| | | acgacggcctccaggcaccaccccaccaccctggccgtgggaatctgctctcccgcctcccgtagcatcagcctgctgc |
| | | gtctcagcttccaccaaggttgtaccattgcctcccagcgcctgacgcccctggcccgaggggacacactctgcaccaa |
| | | cctcactgggacacttttgccttcccgaaacactgatgagaccttctttggagtgcagtgggtgcgcccc |
| Murine Soluble CD70; | 135 | SKQQQRLLEHPEPHTAELQLNLTVPRKDPTLRWGAGPALGRSFTHGPELEEGHLRIHQDGLYRLHIQVTLANCSSPGST |
| Protein Sequence | | LQHRATLAVGICSPAAHGISLLRGRFGQDCTVALQRLTYLVHGDVLCTNLTLPLLPSRNADETFFGVQWICP |
| Murine Soluble CD70; DNA | 136 | agtaagcagcaacagaggctgctggagcaccctgagccgcacacagctgagttacagctgaatctcacagttcctcgga |
| Sequence | | aggaccccacactgcgctggggagcaggcccagccttgggaaggtccttcacacacggaccagagctggaggagggcca |
| | | tctgcgtatccatcaagatggcctctacaggctgcatatccaggtgacactggccaactgctcttccccaggcagcacc |
| | | ctgcagcacagggccaccctggctgtgggcatctgctcccccgctgcgcacggcatcagcttgctgcgtgggcgctttg |
| | | gacaggactgtacagtggcattacagcgcctgacatacctggtccacggagatgtcctctgtaccaacctcaccctgcc |
| | | tctgctgccgtcccgcaacgctgatgagaccttctttggagttcagtggatatgccct |
| Murine IL-12p35-P2A-IL- | 137 | MVSVPTASPSASSSSSQCRSSMCQSRYLLFLATLALLNHLSLARVIPVSGPARCLSQSRNLLKTTDDMVKTAREKLKHY |
| 12p40; Protein Sequence | | SCTAEDIDHEDITRDQTSTLKTCLPLELHKNESCLATRETSSTTRGSCLPPQKTSLMMTLCLGSIYEDLKMYQTEFQAI |
| | | NAALQNHNHQQIILDKGMLVAIDELMQSLNHNGETLRQKPPVGEADPYRVKMKLCILLHAFSTRVVTINRVMGYLSSAA |
| | | AAGSGATNFSLLKQAGDVEENPGPGSCPQKLTISWFAIVLLVSPLMAMWELEKDVYVVEVDWTPDAPGETVNLTCDTPE |
| | | EDDITWTSDQRHGVIGSGKTLTITVKEFLDAGQYTCHKGGETLSHSHLLLHKKENGIWSTEILKNFKNKTFLKCEAPNY |
| | | SGRFTCSWLVQRNMDLKFNIKSSSSSPDSRAVTCGMASLSAEKVTLDQRDYEKYSVSCQEDVTCPTAEETLPIELALEA |
| | | RQQNKYENYSTSFFIRDIIKPDPPKNLQMKPLKNSQVEVSWEYPDSWSTPHSYFSLKFFVRIQRKKEKMKETEEGCNQK |
| | | GAFLVEKTSTEVQCKGGNVCVQAQDRYYNSSCSKWACVPCRVRS |
| Murine IL-12p35-P2A-IL- | 138 | atggtcagcgttccaacagcctcaccctcggcatccagcagctcctctcagtgccggtccagcatgtgtcaatcacgct |
| 12p40; DNA Sequence | | acctcctctttttggccacccttgccctcctaaaccacctcagtttggccagggtcattccagtctctggacctgccag |
| | | gtgtcttagccagtcccgaaacctgctgaagaccacagatgacatggtgaagacggccagagaaaaactgaaacattat |
| | | tcctgcactgctgaagacatcgatcatgaagacatcacacgggaccaaaccagcacattgaagacctgtttaccactgg |
| | | aactacacaagaacgagagttgcctggctactagagagacttcttccacaacaagagggagctgcctgcccccacagaa |
| | | gacgtctttgatgatgaccctgtgccttggtagcatctatgaggacttgaagatgtaccagacagagttccaggccatc |
| | | aacgcagcacttcagaatcacaaccatcagcagatcattcttgacaagggcatgctggtggccatcgatgagctgatgc |
| | | agtctctgaatcataatggcgagactctgcgccagaaacctcctgtgggagaagcagacccttacagagtgaaaatgaa |
| | | gctctgcatcctgcttcacgccttcagcacccgcgtcgtgaccatcaacagggtgatgggctatctgagctccgccgcg |
| | | gccgcaggatctggggccaccaacttttcattgctcaagcaggcgggcgatgtggaggaaaaccctggccccggatcct |
| | | gtcctcagaagctaaccatctcctggtttgccatcgttttgctggtgtctccactcatggccatgtgggagctggagaa |
| | | agacgtttatgttgtagaggtggactggactcccgatgcccctggagaaacagtgaacctcacctgtgacacgcctgaa |
| | | gaagatgacatcacctggacctcagaccagagacatggagtcataggctctggaaagaccctgaccatcactgtcaaag |
| | | agtttcttgatgctggccagtacacctgccacaaaggaggcgagactctgagccactcacatctgctgctccacaagaa |
| | | ggaaaatggaatttggtccactgaaattttaaagaatttcaagaacaagactttcctgaagtgtgaagcaccaaattac |
| | | tccggacggttcacgtgctcatggctggtgcaaagaaacatggacttgaagttcaacatcaagagcagtagcagttccc |
| | | ctgactctcgggcagtgacatgtggaatggcgtctctgtctgcagagaaggtcacactggaccaaagggactatgagaa |
| | | gtattcagtgtcctgccaggaggatgtcacctgcccaactgccgaggagaccctgcccattgaactggcgttggaagca |
| | | cggcagcagaataaatatgagaactacagcaccagcttcttcatcagggacatcatcaaaccagacccgcccaagaact |
| | | tgcagatgaagcctttgaagaactcacaggtggaggtcagctgggagtaccctgactcctggagcactccccattccta |
| | | cttctccctcaagttctttgttcgaatccagcgcaagaaagaaaagatgaaggagacagaggaggggtgtaaccagaaa |
| | | ggtgcgttcctcgtagagaagacatctaccgaagtccaatgcaaaggcgggaatgtctgcgtgcaagctcaggatcgct |
| | | attacaattcctcatgcagcaagtgggcatgtgttccctgcagggtccgatcctag |
| Murine IL-12p35 Protein | 139 | MVSVPTASPSASSSSSQCRSSMCQSRYLLFLATLALLNHLSLARVIPVSGPARCLSQSRNLLKTTDDMVKTAREKLKHY |
| Sequence | | SCTAEDIDHEDITRDQTSTLKTCLPLELHKNESCLATRETSSTTRGSCLPPQKTSLMMTLCLGSIYEDLKMYQTEFQAI |
| | | NAALQNHNHQQIILDKGMLVAIDELMQSLNHNGETLRQKPPVGEADPYRVKMKLCILLHAFSTRVVTINRVMGYLSSAA |
| | | AA |
| Murine IL-12p35 DNA | 140 | atggtcagcgttccaacagcctcaccctcggcatccagcagctcctctcagtgccggtccagcatgtgtcaatcacgct |
| Sequence | | acctcctctttttggccacccttgccctcctaaaccacctcagtttggccagggtcattccagtctctggacctgccag |
| | | gtgtcttagccagtcccgaaacctgctgaagaccacagatgacatggtgaagacggccagagaaaaactgaaacattat |
| | | tcctgcactgctgaagacatcgatcatgaagacatcacacgggaccaaaccagcacattgaagacctgtttaccactgg |
| | | aactacacaagaacgagagttgcctggctactagagagacttcttccacaacaagagggagctgcctgcccccacagaa |
| | | gacgtctttgatgatgaccctgtgccttggtagcatctatgaggacttgaagatgtaccagacagagttccaggccatc |
| | | aacgcagcacttcagaatcacaaccatcagcagatcattcttgacaagggcatgctggtggccatcgatgagctgatgc |
| | | agtctctgaatcataatggcgagactctgcgccagaaacctcctgtgggagaagcagacccttacagagtgaaaatgaa |
| | | gctctgcatcctgcttcacgccttcagcacccgcgtcgtgaccatcaacagggtgatgggctatctgagctccgccgcg |
| | | gccgca |
| Exemplary P2A Protein | 141 | GSGATNFSLLKQAGDVEENPGPG |
| Sequence | | |
| Exemplary P2A DNA | 142 | ggatctggggccaccaacttttcattgctcaagcagggggcgatgtggaggaaaaccctggccccgga |
| Sequence | | |
| IL-12p40 Protein Sequence | 143 | SCPQKLTISWFAIVLLVSPLMAMWELEKDVYVVEVDWTPDAPGETVNLTCDTPEEDDITWTSDQRHGVIGSGKTLTITV |
| | | KEFLDAGQYTCHKGGETLSHSHLLLHKKENGIWSTEILKNFKNKTFLKCEAPNYSGRFTCSWLVQRNMDLKFNIKSSSS |
| | | SPDSRAVTCGMASLSAEKVTLDQRDYEKYSVSCQEDVTCPTAEETLPIELALEARQQNKYENYSTSFFIRDIIKPDPPK |
| | | NLQMKPLKNSQVEVSWEYPDSWSTPHSYFSLKFFVRIQRKKEKMKETEEGCNQKGAFLVEKTSTEVQCKGGNVCVQAQD |
| | | RYYNSSCSKWACVPCRVRS |
| IL-12p40 DNA Sequence | 144 | atcctgtcctcagaagctaaccatctcctggtttgccatcgttttgctggtgtctccactcatggccatgtgggagctg |
| | | gagaaagacgtttatgttgtagaggtggactggactcccgatgcccctggagaaacagtgaacctcacctgtgacacgc |
| | | ctgaagaagatgacatcacctggacctcagaccagagacatggagtcataggctctggaaagaccctgaccatcactgt |
| | | caaagagtttcttgatgctggccagtacacctgccacaaaggaggcgagactctgagccactcacatctgctgctccac |
| | | aagaaggaaaatggaatttggtccactgaaattttaaagaatttcaagaacaagactttcctgaagtgtgaagcaccaa |
| | | attactccggacggttcacgtgctcatggctggtgcaaagaaacatggacttgaagttcaacatcaagagcagtagcag |
| | | ttcccctgactctcgggcagtgacatgtggaatggcgtctctgtctgcagagaaggtcacactggaccaaagggactat |
| | | gagaagtattcagtgtcctgccaggaggatgtcacctgcccaactgccgaggagaccctgcccattgaactggcgttgg |
| | | aagcacggcagcagaataaatatgagaactacagcaccagcttcttcatcagggacatcatcaaaccagacccgcccaa |
| | | gaacttgcagatgaagcctttgaagaactcacaggtggaggtcagctgggagtaccctgactcctggagcactccccat |
| | | tcctacttctccctcaagttctttgttcgaatccagcgcaagaaagaaaagatgaaggagacagaggaggggtgtaacc |
| | | agaaaggtgcgttcctcgtagagaagacatctaccgaagtccaatgcaaaggcgggaatgtctgcgtgcaagctcagga |
| | | tcgctattacaattcctcatgcagcaagtgggcatgtgttccctgcagggtccgatcctag |
| Human IL-12p35-P2A-IL- | 145 | MCPARSLLLVATLVLLDHLSLARNLPVATPDPGMFPCLHHSQNLLRAVSNMLQKARQTLEFYPCTSEEIDHEDITKDKT |
| 12p40 Protein Sequence | | STVEACLPLELTKNESCLNSRETSFITNGSCLASRKTSFMMALCLSSIYEDLKMYQVEFKTMNAKLLMDPKRQIFLDQN |
| | | MLAVIDELMQALNFNSETVPQKSSLEEPDFYKTKIKLCILLHAFRIRAVTIDRVMSYLNASATNFSLLKQAGDVEENPG |
| | | PGMCHQQLVISWFSLVFLASPLVAIWELKKDVYVVELDWYPDAPGEMVVLTCDTPEEDGITWTLDQSSEVLGSGKTLTI |
| | | QVKEFGDAGQYTCHKGGEVLSHSLLLLHKKEDGIWSTDILKDQKEPKNKTFLRCEAKNYSGRFTCWWLTTISTDLTFSV |
| | | KSSRGSSDPQGVTCGAATLSAERVRGDNKEYEYSVECQEDSACPAAEESLPIEVMVDAVHKLKYENYTSSFFIRDIIKP |
| | | DPPKNLQLKPLKNSRQVEVSWEYPDTWSTPHSYFSLTFCVQVQGKSKREKKDRVFTDKTSATVICRKNASISVRAQDRY |
| | | YSSSWSEWASVPCS |
| Human IL-12p35-P2A-IL- | 146 | atgtgcccggcgcgcagcctgctgctggtggcgaccctggtgctgctggatcatctgagcctggcgcgcaacctgccgg |
| 12p40 DNA Sequence | | tggcgaccccggatccgggcatgtttccgtgcctgcatcatagccagaacctgctgcgcgcggtgagcaacatgctgca |
| | | gaaagcgcgccagaccctggaattttatccgtgcaccagcgaagaaattgatcatgaagatattaccaaagataaaacc |
| | | agcaccgtggaagcgtgcctgccgctggaactgaccaaaaacgaaagctgcctgaacagccgcgaaaccagctttatta |
| | | ccaacggcagctgcctggcgagccgcaaaaccagctttatgatggcgctgtgcctgagcagcatttatgaagatctgaa |
| | | aatgtatcaggtggaatttaaaaccatgaacgcgaaactgctgatggatccgaaacgccagatttttctggatcagaac |
| | | atgctggcggtgattgatgaactgatgcaggcgctgaactttaacagcgaaaccgtgccgcagaaaagcagcctggaag |
| | | aaccggatttttataaaaccaaaattaaactgtgcattctgctgcatgcgtttcgcattcgcgcggtgaccattgatcg |
| | | cgtgatgagctatctgaacgcgagcgcgaccaactttagcctgctgaaacaggcgggcgatgtggaagaaaacccgggc |
| | | ccgggcatgtgccatcagcagctggtgattagctggtttagcctggtgtttctggcgagcccgctggtggcgatttggg |
| | | aactgaaaaaagatgtgtatgtggtggaactggattggtatccggatgcgccgggcgaaatggtggtgctgacctgcga |
| | | taccccggaagaagatggcattacctggaccctggatcagagcagcgaagtgctgggcagcggcaaaaccctgaccatt |
| | | caggtgaaagaatttggcgatgcgggccagtatacctgccataaaggcggcgaagtgctgagccatagcctgctgctgc |
| | | tgcataaaaaagaagatggcatttggagcaccgatattctgaaagatcagaaagaaccgaaaaacaaaacctttctgcg |
| | | ctgcgaagcgaaaaactatagcggccgctttacctgctggtggctgaccaccattagcaccgatctgacctttagcgtg |
| | | aaaagcagccgcggcagcagcgatccgcagggcgtgacctgcggcgcggcgaccctgagcgcggaacgcgtgcgcggcg |
| | | ataacaaagaatatgaatatagcgtggaatgccaggaagatagcgcgtgcccggcggcggaagaaagcctgccgattga |
| | | agtgatggtggatgcggtgcataaactgaaatatgaaaactataccagcagcttttttattcgcgatattattaaaccg |
| | | gatccgccgaaaaacctgcagctgaaaccgctgaaaaacagccgccaggtggaagtgagctgggaatatccggatacct |
| | | ggagcaccccgcatagctattttagcctgaccttttgcgtgcaggtgcagggcaaaagcaaacgcgaaaaaaaagatcg |
| | | cgtgtttaccgataaaaccagcgcgaccgtgatttgccgcaaaaacgcgagcattagcgtgcgcgcgcaggatcgctat |
| | | tatagcagcagctggagcgaatgggcgagcgtgccgtgcagc |
| Human IL-12p35 Protein | 147 | MCPARSLLLVATLVLLDHLSLARNLPVATPDPGMFPCLHHSQNLLRAVSNMLQKARQTLEFYPCTSEEIDHEDITKDKT |
| Sequence | | STVEACLPLELTKNESCLNSRETSFITNGSCLASRKTSFMMALCLSSIYEDLKMYQVEFKTMNAKLLMDPKRQIFLDQN |
| | | MLAVIDELMQALNFNSETVPQKSSLEEPDFYKTKIKLCILLHAFRIRAVTIDRVMSYLNAS |
| Human IL-12p35 DNA | 148 | atgtgcccggcgcgcagcctgctgctggtggcgaccctggtgctgctggatcatctgagcctggcgcgcaacctgccgg |
| Sequence | | tggcgaccccggatccgggcatgtttccgtgcctgcatcatagccagaacctgctgcgcgcggtgagcaacatgctgca |
| | | gaaagcgcgccagaccctggaattttatccgtgcaccagcgaagaaattgatcatgaagatattaccaaagataaaacc |
| | | agcaccgtggaagcgtgcctgccgctggaactgaccaaaaacgaaagctgcctgaacagccgcgaaaccagctttatta |
| | | ccaacggcagctgcctggcgagccgcaaaaccagctttatgatggcgctgtgcctgagcagcatttatgaagatctgaa |
| | | aatgtatcaggtggaatttaaaaccatgaacgcgaaactgctgatggatccgaaacgccagatttttctggatcagaac |
| | | atgctggcggtgattgatgaactgatgcaggcgctgaactttaacagcgaaaccgtgccgcagaaaagcagcctggaag |
| | | aaccggatttttataaaaccaaaattaaactgtgcattctgctgcatgcgtttcgcattcgcgcggtgaccattgatcg |
| | | cgtgatgagctatctgaacgcgagc |
| Human IL-12p40 Protein | 149 | MCHQQLVISWFSLVFLASPLVAIWELKKDVYVVELDWYPDAPGEMVVLTCDTPEEDGITWTLDQSSEVLGSGKTLTIQV |
| Sequence | | KEFGDAGQYTCHKGGEVLSHSLLLLHKKEDGIWSTDILKDQKEPKNKTFLRCEAKNYSGRFTCWWLTTISTDLTFSVKS |
| | | SRGSSDPQGVTCGAATLSAERVRGDNKEYEYSVECQEDSACPAAEESLPIEVMVDAVHKLKYENYTSSFFIRDIIKPDP |
| | | PKNLQLKPLKNSRQVEVSWEYPDTWSTPHSYFSLTFCVQVQGKSKREKKDRVFTDKTSATVICRKNASISVRAQDRYYS |
| | | SSWSEWASVPCS |
| Human IL-12p40 DNA | 150 | atgtgccatcagcagctggtgattagctggtttagcctggtgtttctggcgagcccgctggtggcgatttgggaactga |
| Sequence | | aaaaagatgtgtatgtggtggaactggattggtatccggatgcgccgggcgaaatggtggtgctgacctgcgatacccc |
| | | ggaagaagatggcattacctggaccctggatcagagcagcgaagtgctgggcagcggcaaaaccctgaccattcaggtg |
| | | aaagaatttggcgatgcgggccagtatacctgccataaaggcggcgaagtgctgagccatagcctgctgctgctgcata |
| | | aaaaagaagatggcatttggagcaccgatattctgaaagatcagaaagaaccgaaaaacaaaacctttctgcgctgcga |
| | | agcgaaaaactatagcggccgctttacctgctggtggctgaccaccattagcaccgatctgacctttagcgtgaaaagc |
| | | agccgcggcagcagcgatccgcagggcgtgacctgcggcgcggcgaccctgagcgcggaacgcgtgcgcggcgataaca |
| | | aagaatatgaatatagcgtggaatgccaggaagatagcgcgtgcccggcggcggaagaaagcctgccgattgaagtgat |
| | | ggtggatgcggtgcataaactgaaatatgaaaactataccagcagcttttttattcgcgatattattaaaccggatccg |
| | | ccgaaaaacctgcagctgaaaccgctgaaaaacagccgccaggtggaagtgagctgggaatatccggatacctggagca |
| | | ccccgcatagctattttagcctgaccttttgcgtgcaggtgcagggcaaaagcaaacgcgaaaaaaaagatcgcgtgtt |
| | | taccgataaaaccagcgcgaccgtgatttgccgcaaaaacgcgagcattagcgtgcgcgcgcaggatcgctattatagc |
| | | agcagctggagcgaatgggcgagcgtgccgtgcagc |
| Human FLT3L Protein | 151 | TQDCSFQHSPISSDFAVKIRELSDYLLQDYPVTVASNLQDEELCGGLWRLVLAQRWMERLKTVAGSKMQGLLERVNTEI |
| Sequence | | HFVTKCAFQPPPSCLRFVQTNISRLLQETSEQLVALKPWITRQNFSRCLELQCQPDSSTLPPPWSPRPLEATAPTAPQP |
| Human FLT3L DNA | 152 | atgacagtgctggcgccagcctggagcccaacaacctatctcctcctgctgctgctgctgagctcgggactcagtggga |
| Sequence | | cccaggactgctccttccaacacagccccatctcctccgacttcgctgtcaaaatccgtgagctgtctgactacctgct |
| | | tcaagattacccagtcaccgtggcctccaacctgcaggacgaggagctctgcgggggcctctggcggctggtcctggca |
| | | cagcgctggatggagcggctcaagactgtcgctgggtccaagatgcaaggcttgctggagcgcgtgaacacggagatac |
| | | actttgtcaccaaatgtgcctttcagccc |
| Murine OX40L Protein | 153 | SSSPAKDPPIQRLRGAVTRCEDGQLFISSYKNEYQTMEVQNNSVVIKCDGLYIIYLKGSFFQEVKIDLHFREDHNPISI |
| Sequence | | PMLNDGRRIVFTVVASLAFKDKVYLTVNAPDTLCEHLQINDGELIVVQLTPGYCAPEGSYHSTVNQVPL |
| Murine OX40L DNA | 154 | agcagcagccctgccaaggaccctcctattcagaggctgaggggcgccgtgacaaggtgcgaggacggacagctgttca |
| Sequence | | tcagcagctacaagaacgagtatcagaccatggaggtgcagaacaacagcgtggtgatcaagtgcgacggcctgtacat |
| | | catctacctgaagggcagcttcttccaagaggtgaagatcgacctgcactttagggaggaccacaaccctatcagcatc |
| | | cctatgctgaacgacggaaggaggatcgtgttcaccgtggtggctagcctggccttcaaggacaaggtgtacctgaccg |
| | | tgaacgcccctgacaccctgtgcgagcacctgcagatcaacgacggcgagctgatcgtggtgcagctgacccctggcta |
| | | ctgcgcccctgagggcagctaccacagcaccgtgaaccaagtgcctctg |
| Human OX40L Protein | 155 | QVSHRYPRIQSIKVQFTEYKKEKGFILTSQKEDEIMKVQNNSVIINCDGFYLISLKGYFSQEVNISLHYQKDEEPLFQL |
| Sequence | | KKVRSVNSLMVASLTYKDKVYLNVTTDNTSLDDFHVNGGELILIHQNPGEFCVL |
| Human OX40L DNA | 156 | caggtatcacatcggtatcctcgaattcaaagtatcaaagtacaatttaccgaatataagaaggagaaaggtttcatcc |
| Sequence | | tcacttcccaaaaggaggatgaaatcatgaaggtgcagaacaactcagtcatcatcaactgtgatgggttttatctcat |
| | | ctccctgaagggctacttctcccaggaagtcaacattagccttcattaccagaaggatgaggagcccctcttccaactg |
| | | aagaaggtcaggtctgtcaactccttgatggtggcctctctgacttacaaagacaaagtctacttgaatgtgaccactg |
| | | acaatacctccctggatgacttccatgtgaatggcggagaactgattcttatccatcaaaatcctggtgaattctgtgt |
| | | cctt |
| Murine CD80 Protein | 157 | VDEQLSKSVKDKVLLPCRYNSPHEDESEDRIYWQKHDKVVLSVIAGKLKVWPEYKNRTLYDNTTYSLIILGLVLSDRGT |
| Sequence | | YSCVVQKKERGTYEVKHLALVKLSIKADFSTPNITESGNPSADTKRITCFASGGFPKPRFSWLENGRELPGINTTISQD |
| | | PESELYTISSQLDFNTTRNHTIKCLIKYGDAHVSEDFTWEKPPEDPPDSKN |
| Murine CD80 DNA | 158 | Gtggacgagcagctgagcaagagcgtgaaggacaaggtgctgctgccttgtaggtacaacagccctcacgaggacgaga |
| Sequence | | gcgaggataggatctactggcagaagcacgacaaggtagtgctgagtgtgatagccggcaagctgaaggtgtggcctga |
| | | gtacaagaataggaccctgtacgacaacaccacctacagcctgatcatcctgggcctggtgctgagcgataggggtacc |
| | | tacagctgcgtggtgcagaagaaggagaggggcacctacgaggtgaagcacctggccctggtgaagctgagcatcaagg |
| | | ccgacttcagcacccctaacatcaccgagagcggcaaccctagcgccgacaccaagaggatcacctgcttcgctagcgg |
| | | cggcttccctaagcctaggttcagctggctggagaacggaagggagctgcctggcataaacacgaccatatctcaagac |
| | | cctgagagcgagctgtacaccatcagctctcagctggacttcaacaccacccgcaatcacaccatcaagtgcctgatca |
| | | agtacggcgacgcccacgtgagcgaggacttcacctgggagaagcctcctgaggaccctcctgacagcaagaac |
| Human CD80 Protein | 159 | VIHVTKEVKEVATLSCGHNVSVEELAQTRIYWQKEKKMVLTMMSGDMNIWPEYKNRTIFDITNNLSIVILALRPSDEGT |
| Sequence | | YECVVLKYEKDAFKREHLAEVTLSVKADFPTPSISDFEIPTSNIRRIICSTSGGFPEPHLSWLENGEELNAINTTVSQD |
| | | PETELYAVSSKLDFNMTTNHSFMCLIKYGHLRVNQTFNWNTTKQEHFPDN |
| Human CD80 DNA | 160 | gttatccacgtgaccaaggaagtgaaagaagtggcaacgctgtcctgtggtcacaatgtttctgttgaagagctggcac |
| Sequence | | aaactcgcatctactggcaaaaggagaagaaaatggtgctgactatgatgtctggggacatgaatatatggcccgagta |
| | | caagaaccggaccatctttgatatcactaataacctctccattgtgatcctggctctgcgcccatctgacgagggcaca |
| | | tacgagtgtgttgttctgaagtatgaaaaagacgctttcaagcgggaacacctggctgaagtgacgttatcagtcaaag |
| | | ctgacttccctacacctagtatatctgactttgaaattccaacttctaatattagaaggataatttgctcaacctctgg |
| | | aggttttccagagcctcacctctcctggttggaaaatggagaagaattaaatgccatcaacacaacagtttcccaagat |
| | | cctgaaactgagctctatgctgttagcagcaaactggatttcaatatgacaaccaaccacagcttcatgtgtctcatca |
| | | agtatggacatttaagagtgaatcagaccttcaactggaatacaaccaagcaagagcattttcctgataac |
| Murine CD86 Protein | 161 | MDPRCTMGLAILIFVTVLLISDAVSVETQAYFNGTAYLPCPFTKAQNISLSELVVFWQDQQKLVLYEHYLGT |
| Sequence | | EKLDSVNAKYLGRTSFDRNNWTLRLHNVQIKDMGSYDCFIQKKPPTGSIILQQTLTELSVIANFSEPEIKLA |
| | | QNVTGNSGINLTCTSKQGHPKPKKMYFLITNSTNEYGDNMQISQDNVTELFSISNSLSLSFPDGVWHMTVVC |
| | | VLETESMKISSKPLNFTQEFPSPQTYWK |
| Murine CD86 DNA | 162 | atggatccgcgctgcaccatgggcctggcgattctgatttttgtgaccgtgctgctgattagcgatgcggtgagcgtgg |
| Sequence | | aaacccaggcgtattttaacggcaccgcgtatctgccgtgcccgtttaccaaagcgcagaacattagcctgagcgaact |
| | | ggtggtgttttggcaggatcagcagaaactggtgctgtatgaacattatctgggcaccgaaaaactggatagcgtgaac |
| | | gcgaaatatctgggccgcaccagctttgatcgcaacaactggaccctgcgcctgcataacgtgcagattaaagatatgg |
| | | gcagctatgattgctttattcagaaaaaaccgccgaccggcagcattattctgcagcagaccctgaccgaactgagcgt |
| | | gattgcgaactttagcgaaccggaaattaaactggcgcagaacgtgaccggcaacagcggcattaacctgacctgcacc |
| | | agcaaacagggccatccgaaaccgaaaaaaatgtattttctgattaccaacagcaccaacgaatatggcgataacatgc |
| | | agattagccaggataacgtgaccgaactgtttagcattagcaacagcctgagcctgagctttccggatggcgtgtggca |
| | | tatgaccgtggtgtgcgtgctggaaaccgaaagcatgaaaattagcagcaaaccgctgaactttacccaggaatttccg |
| | | agcccgcagacctattggaaag |
| Murine IL-18 Protein | 163 | NFGRLHCTTAVIRNINDQVLFVDKRQPVFEDMTDIDQSASEPQTRLIIYMYKDSEVRGLAVTLSVKDSKMSTLSCKNKI |
| Sequence | | ISFEEMDPPENIDDIQSDLIFFQKRVPGHNKMEFESSLYEGHFLACQKEDDAFKLILKKKDENGDKSVMFTLTNLHQS |
| Murine IL-18 DNA Sequence | 164 | atggctgccatgtcagaagactcttgcgtcaacttcaaggaaatgatgtttattgacaacacgctttactttatacctg |
| | | aagaaaatggagacctggaatcagacaactttggccgacttcactgtacaaccgcagtaatacggaatataaatgacca |
| | | agttctcttcgttgacaaaagacagcctgtgttcgaggatatgactgatattgatcaaagtgccagtgaaccccagacc |
| | | agactgataatatacatgtacaaagacagtgaagtaagaggactggctgtgaccctctctgtgaaggatagtaaaatgt |
| | | ctaccctctcctgtaagaacaagatcatttcctttgaggaaatggatccacctgaaaatattgatgatatacaaagtga |
| | | tctcatattctttcagaaacgtgttccaggacacaacaagatggagtttgaatcttcactgtatgaaggacactttctt |
| | | gcttgccaaaaggaagatgatgctttcaaactcattctgaaaaaaaaggatgaaaatggggataaatctgtaatgttca |
| | | ctctcactaacttacatcaaagttag |
| Secreted Human IL-18 | 165 | YFGKLESKLSVIRNLNDQVLFIDQGNRPLFEDMTDSDCRDNAPRTIFIISMYKDSQPRGMAVTISVKCEKISTLSCENK |
| Protein Sequence | | IISFKEMNPPDNIKDTKSDIIFFQRSVPGHDNKMQFESSSYEGYFLACEKERDLFKLILKKEDELGDRSIMFTVQNED |
| Secreted Human IL-18 DNA | 166 | aactttggccgacttcactgtacaaccgcagtaatacggaatataaatgaccaagttctcttcgttgacaaaagacagc |
| Sequence | | ctgtgttcgaggatatgactgatattgatcaaagtgccagtgaaccccagaccagactgataatatacatgtacaaaga |
| | | cagtgaagtaagaggactggctgtgaccctctctgtgaaggatagtaaaatgtctaccctctcctgtaagaacaagatc |
| | | atttcctttgaggaaatggatccacctgaaaatattgatgatatacaaagtgatctcatattctttcagaaacgtgttc |
| | | caggacacaacaagatggagtttgaatcttcactgtatgaaggacactttcttgcttgccaaaaggaagatgatgcttt |
| | | caaactcattctgaaaaaaaaggatgaaaatggggataaatctgtaatgttcactctcactaacttacatcaaagttag |
| Murine IL-15 Protein | 167 | NWIDVRYDLEKIESLIQSIHIDTTLYTDSDFHPSCKVTAMNCFLLELQVILHEYSNMTLNETVRNVLYLANSTLSSNKN |
| Sequence | | VAESGCKECEELEEKTFTEFLQSFIRIVQMFINTS |
| Murine IL-15 DNA Sequence | 168 | aactggatagatgtaagatatgacctggagaaaattgaaagccttattcaatctattcatattgacaccactttataca |
| | | ctgacagtgactttcatcccagttgcaaagttactgcaatgaactgctttctcctggaattgcaggttattttacatga |
| | | gtacagtaacatgactcttaatgaaacagtaagaaacgtgctctaccttgcaaacagcactctgtcttctaacaagaat |
| | | gtagcagaatctggctgcaaggaatgtgaggagctggaggagaaaaccttcacagagtttttgcaaagctttatacgca |
| | | ttgtccaaatgttcatcaacacgtcc |
| Human IL-15 Protein | 169 | NWVNVISDLKKIEDLIQSMHIDATLYTESDVHPSCKVTAMKCFLLELQVISLESGDASIHDTVENLIILANNSLSSNGN |
| Sequence | | VTESGCKECEELEEKNIKEFLQSFVHIVQMFINTS |
| Human IL-15 DNA | 170 | aactgggtgaatgtaataagtgatttgaaaaaaattgaagatcttattcaatctatgcatattgatgctactttatata |
| Sequence | | cggaaagtgatgttcaccccagttgcaaagtaacagcaatgaagtgctttctcttggagttacaagttatttcacttga |
| | | gtccggagatgcaagtattcatgatacagtagaaaatctgatcatcctagcaaacaacagtttgtcttctaatgggaat |
| | | gtaacagaatctggatgcaaagaatgtgaggaactggaggaaaaaaatattaaagaatttttgcagagttttgtacata |
| | | ttgtccaaatgttcatcaacacttct |
| Murine IL-7 Protein | 171 | ECHIKDKEGKAYESVLMISIDELDKMTGTDSNCPNNEPNFFRKHVCDDTKEAAFLNRAARKLKQFLKMNISEEFNVHLL |
| Sequence | | TVSQGTQTLVNCTSKEEKNVKEQKKNDACFLKRLLREIKTCWNKILKGSI |
| Murine IL-7 DNA Sequence | 172 | gagtgccacattaaagacaaagaaggtaaagcatatgagagtgtactgatgatcagcatcgatgaattggacaaaatga |
| | | caggaactgatagtaattgcccgaataatgaaccaaacttttttagaaaacatgtatgtgatgatacaaaggaagctgc |
| | | ttttctaaatcgtgctgctcgcaagttgaagcaatttcttaaaatgaatatcagtgaagaattcaatgtccacttacta |
| | | acagtatcacaaggcacacaaacactggtgaactgcacaagtaaggaagaaaaaaacgtaaaggaacagaaaaagaatg |
| | | atgcatgtttcctaaagagactactgagagaaaaaaaacttgttggaataaaattttgaagggcagtatataa |
| Human IL-7 Protein | 173 | DCDIEGKDGKQYESVLMVSIDQLLDSMKEIGSNCLNNEFNFFKRHICDANKEGMFLFRAARKLRQFLKMNSTGDFDLHL |
| Sequence | | LKVSEGTTILLNCTGQVKGRKPAALGEAQPTKSLEENKSLKEQKKLNDLCFLKRLLQEIKTCWNKILMGTKEH |
| Human IL-7 DNA Sequence | 174 | gattgtgatattgaaggtaaagatggcaaacaatatgagagtgttctaatggtcagcatcgatcaattattggacagca |
| | | tgaaagaaattggtagcaattgcctgaataatgaatttaacttttttaaaagacatatctgtgatgctaataaggaagg |
| | | tatgtttttattccgtgctgctcgcaagttgaggcaatttcttaaaatgaatagcactggtgattttgatctccactta |
| | | ttaaaagtttcagaaggcacaacaatactgttgaactgcactggccaggttaaaggaagaaaaccagctgccctgggtg |
| | | aagcccaaccaacaaagagtttggaagaaaataaatctttaaaggaacagaaaaaactgaatgacttgtgtttcctaaa |
| | | gagactattacaagagataaaaacttgttggaataaaattttgatgggcactaaagaacactga |
| Murine IL-33 Protein | 175 | AASVDTLSIQGTSLLTQSPASLSTYNDQSVSFVLENGCYVINVDDSGKDQEQDQVLLRYYESPCPASQSGDGVDGKKLM |
| Sequence | | VNMSPIKDTDIWLHANDKDYSVELQRGDVSPPEQAFFVLHKKSSDFVSFECKNLPGTYIGVKDNQLALVEEKDESCNNI |
| | | MFKLSKI |
| Murine IL-33 DNA Sequence | 176 | gctgcgtctgttgacacattgagcatccaaggaacttcacttttaacacagtctcctgcctccctgagtacatacaatg |
| | | accaatctgttagttttgttttggagaatggatgttatgtgatcaatgttgacgactctggaaaagaccaagagcaaga |
| | | ccaggtgctactacgctactatgagtctccctgtcctgcaagtcaatcaggcgacggtgtggatgggaagaagctgatg |
| | | gtgaacatgagttccatcaaagacacagacatctggctgcatgccaacgacaaggactactccgtggagcttcaaaggg |
| | | gtgacgtctcgcctccggaacaggccttcttcgtccttcacaaaaagtcctcggactttgtttcatttgaatgcaagaa |
| | | tcttcctggcacttacataggagtgaaggacaaccagctggctctagtggaggaaaaagatgagagctgcaacaatatt |
| | | atgtttaagctctcgaaaatctaa |
| Human IL-33 Protein | 177 | AFGISGVQKYTRALHDSSITGISPITEYLASLSTYNDQSITFALEDESYEIYVEDLKKDEKKDKVLLSYYESQHPSNES |
| Sequence | | GDGVDGKMLMVTLSPTKDFWLHANNKEHSVELHKCEKPLPDQAFFVLHNMHSNCVSFECKTDPGVFIGVKDNHLALIKV |
| | | DSSENLCTENILFKLSET |
| Human IL-33 DNA | 178 | gcctttggtatatcaggggtccagaaatatactagagcacttcatgattcaagtatcacaggaatttcacctattacag |
| Sequence | | agtatcttgcttctctaagcacatacaatgatcaatccattacttttgctttggaggatgaaagttatgagatatatgt |
| | | tgaagacttgaaaaaagatgaaaagaaagataaggtgttactgagttactatgagtctcaacacccctcaaatgaatca |
| | | ggtgacggtgttgatggtaagatgttaatggtaaccctgagtcctacaaaagacttctggttgcatgccaacaacaagg |
| | | aacactctgtggagctccataagtgtgaaaaaccactgccagaccaggccttctttgtccttcataatatgcactccaa |
| | | ctgtgtttcatttgaatgcaagactgatcctggagtgtttataggtgtaaaggataatcatcttgctctgattaaagta |
| | | gactcttctgagaatttgtgtactgaaaatatcttgtttaagctctctgaaact |
| Murine IL-21 Protein | 179 | HKSSPQGPDRLLIRLRHLIDIVEQLKIYENDLDPELLSAPQDVKGHCEHAAFACFQKAKLKPSNPGNNKTFIIDLVAQL |
| Sequence | | RRRLPARRGGKKQKHIAKCPSCDSYEKRTPKEFLERLKWLLQKMIHQHLS |
| Murine IL-21 DNA Sequence | 180 | cataaatcaagcccccaagggccagatcgcctcctgattagacttcgtcaccttattgacattgttgaacagctgaaaa |
| | | tctatgaaaatgacttggatcctgaacttctatcagctccacaagatgtaaaggggcactgtgagcatgcagcttttgc |
| | | ctgttttcagaaggccaaactcaagccatcaaaccctggaaacaataagacattcatcattgacctcgtggcccagctc |
| | | aggaggaggctgcctgccaggaggggaggaaagaaacagaagcacatagctaaatgcccttcctgtgattcgtatgaga |
| | | aaaggacacccaaagaattcctagaaagactaaaatggctccttcaaaagatgattcatcagcatctctcc |
| Human IL-21 Protein | 181 | HKSSSQGQDRHMIRMRQLIDIVDQLKNYVNDLVPEFLPAPEDVETNCEWSAFSCFQKAQLKSANTGNNERIINVSIKKL |
| Sequence | | KRKPPSTNAGRRQKHRLTCPSCDSYEKKPPKEFLERFKSLLQKMIHQHLSSRTHGSEDS |
| Human IL-21 DNA | 182 | cacaaatcaagctcccaaggtcaagatcgccacatgattagaatgcgtcaacttatagatattgttgatcagctgaaaa |
| Sequence | | attatgtgaatgacttggtccctgaatttctgccagctccagaagatgtagagacaaactgtgagtggtcagctttttc |
| | | ctgttttcagaaggcccaactaaagtcagcaaatacaggaaacaatgaaaggataatcaatgtatcaattaaaaagctg |
| | | aagaggaaaccaccttccacaaatgcagggagaagacagaaacacagactaacatgcccttcatgtgattcttatgaga |
| | | aaaaaccacccaaagaattcctagaaagattcaaatcacttctccaaaagatgattcatcagcatctgtcctctagaac |
| | | acacggaagtgaagattcc |
| Murine IL-23a p19-P2A-IL- | 183 | MLDCRAVIMLWLLPWVTQGLAVPRSSSPDWAQCQQLSRNLCMLAWNAHAPAGHMNLLREEEDEETKNNVPRIQCEDGCD |
| 12 p40 Protein Sequence | | PQGLKDNSQFCLQRIRQGLAFYKHLLDSDIFKGEPALLPDSPMEQLHTSLLGLSQLLQPEDHPRETQQMPSLSSSQQWQ |
| | | RPLLRSKILRSLQAFLAIAARVFAHGAATLTEPLVPTAATNFSLLKQAGDVEENPGPCPQKLTISWFAIVLLVSPLMAM |
| | | WELEKDVYVVEVDWTPDAPGETVNLTCDTPEEDDITWTSDQRHGVIGSGKTLTITVKEFLDAGQYTCHKGGETLSHSHL |
| | | LLHKKENGIWSTEILKNFKNKTFLKCEAPNYSGRFTCSWLVQRNMDLKFNIKSSSSSPDSRAVTCGTASLSAEKVTLDQ |
| | | RDYEKYSVSCQEDVTCPTAEETLPIELALEARQQNKYENYSTSFFIRDIIKPDPPKNLQMKPLKNSQVEVSWEYPDSWS |
| | | TPHSYFSLKFFVRIQRKKEKMKETEEGCNQKGALLVEKTSTEVQCKGGNVCVQAQDRYYNSSCSKWACVPCRVRS |
| Murine IL-23a p19-P2A-IL- | 184 | atgctggattgcagagcagtaataatgctatggctgttgccctgggtcactcagggcctggctgtgcctaggagtagca |
| 12 p40 DNA Sequence | | gtcctgactgggctcagtgccagcagctctctcggaatctctgcatgctagcctggaacgcacatgcaccagcgggaca |
| | | tatgaatctactaagagaagaagaggatgaagagactaaaaataatgtgccccgtatccagtgtgaagatggttgtgac |
| | | ccacaaggactcaaggacaacagccagttctgcttgcaaaggatccgccaaggtctggctttttataagcacctgcttg |
| | | actctgacatcttcaaaggggagcctgctctactccctgatagccccatggagcaacttcacacctccctactaggact |
| | | cagccaactcctccagccagaggatcacccccgggagacccaacagatgcccagcctgagttctagtcagcagtggcag |
| | | cgcccccttctccgttccaagatccttcgaagcctccaggcctttttggccatagctgcccgggtctttgcccacggag |
| | | cagcaactctgactgagcccttagtgccaacagctgccaccaacttttcattgctcaagcaggcgggcgatgtggagga |
| | | aaaccctggccccggatcctgtcctcagaagctaaccatctcctggtttgccatcgttttgctggtgtctccactcatg |
| | | gccatgtgggagctggagaaagacgtttatgttgtagaggtggactggactcccgatgcccctggagaaacagtgaacc |
| | | tcacctgtgacacgcctgaagaagatgacatcacctggacctcagaccagagacatggagtcataggctctggaaagac |
| | | cctgaccatcactgtcaaagagtttctagatgctggccagtacacctgccacaaaggaggcgagactctgagccactca |
| | | catctgctgctccacaagaaggaaaatggaatttggtccactgaaattttaaaaaatttcaaaaacaagactttcctga |
| | | agtgtgaagcaccaaattactccggacggttcacgtgctcatggctggtgcaaagaaacatggacttgaagttcaacat |
| | | caagagcagtagcagttcccctgactctcgggcagtgacatgtggaacggcgtctctgtctgcagagaaggtcacactg |
| | | gaccaaagggactatgagaagtattcagtgtcctgccaggaggatgtcacctgcccaactgccgaggagaccctgccca |
| | | ttgaactggcgttggaagcacggcagcagaataaatatgagaactacagcaccagcttcttcatcagggacatcatcaa |
| | | accagacccgcccaagaacttgcagatgaagcctttgaagaactcacaggtggaggtcagctgggagtaccctgactcc |
| | | tggagcactccccattcctacttctccctcaagttctttgttcgaatccagcgcaagaaagaaaagatgaaggagacag |
| | | aggaggggtgtaaccagaaaggtgcgctcctcgtagagaagacatctaccgaagtccaatgcaaaggcgggaatgtctg |
| | | cgtgcaagctcaggatcgctattacaattcctcgtgcagcaagtgggcatgtgttccctgcagggtccgatcctag |
| Murine IL-23a p19 Protein | 185 | MLDCRAVIMLWLLPWVTQGLAVPRSSSPDWAQCQQLSRNLCMLAWNAHAPAGHMNLLREEEDEETKNNVPRIQCEDGCD |
| Sequence | | PQGLKDNSQFCLQRIRQGLAFYKHLLDSDIFKGEPALLPDSPMEQLHTSLLGLSQLLQPEDHPRETQQMPSLSSSQQWQ |
| | | RPLLRSKILRSLQAFLAIAARVFAHGAATLTEPLVPTA |
| Murine IL-23a p19 DNA | 186 | gccaccatgctggggagcagagctgtaatgctgctgttgctgctgccctggacagctcagggcagagctgtgcctgggg |
| Sequence | | gcagcagccctgcctggactcagtgccagcagctttcacagaagctctgcacactggcctggagtgcacatccactagt |
| | | gggacacatggatctaagagaagagggagatgaagagactacaaatgatgttccccatatccagtgtggagatggctgt |
| | | gacccccaaggactcagggacaacagtcagttctgcttgcaaaggatccaccagggtctgattttttatgagaagctgc |
| | | taggatcggatattttcacaggggagccttctctgctccctgatagccctgtgggccagcttcatgcctccctactggg |
| | | cctcagccaactcctgcagcctgagggtcaccactgggagactcagcagattccaagcctcagtcccagccagccatgg |
| | | cagcgtctccttctccgcttcaaaatccttcgcagcctccaggcctttgtggctgtagccgcccgggtctttgcccatg |
| | | gagcagcaaccctgagtccc |
| Murine IL-12 p40 Protein | 187 | CPQKLTISWFAIVLLVSPLMAMWELEKDVYVVEVDWTPDAPGETVNLTCDTPEEDDITWTSDQRHGVIGSGKTLTITVK |
| Sequence | | EFLDAGQYTCHKGGETLSHSHLLLHKKENGIWSTEILKNFKNKTFLKCEAPNYSGRFTCSWLVQRNMDLKFNIKSSSSS |
| | | PDSRAVTCGTASLSAEKVTLDQRDYEKYSVSCQEDVTCPTAEETLPIELALEARQQNKYENYSTSFFIRDIIKPDPPKN |
| | | LQMKPLKNSQVEVSWEYPDSWSTPHSYFSLKFFVRIQRKKEKMKETEEGCNQKGALLVEKTSTEVQCKGGNVCVQAQDR |
| | | YYNSSCSKWACVPCRVRS |
| Murine IL-12 p40 DNA | 188 | ggatccatgtgtcaccagcagttggtcatctcttggttttccctggtttttctggcatctcccctcgtggccatatggg |
| Sequence | | aactgaagaaagatgtttatgtcgtagaattggattggtatccggatgcccctggagaaatggtggtcctcacctgtga |
| | | cacccctgaagaagatggtatcacctggaccttggaccagagcagtgaggtcttaggctctggcaaaaccctgaccatc |
| | | caagtcaaagagtttggagatgctggccagtacacctgtcacaaaggaggcgaggttctaagccattcgctcctgctgc |
| | | ttcacaaaaaggaagatggaatttggtccactgatattttaaaggaccagaaagaacccaaaaataagacctttctaag |
| | | atgcgaggccaagaattattctggacgtttcacctgctggtggctgacgacaatcagtactgatttgacattcagtgtc |
| | | aaaagcagcagaggctcttctgacccccaaggggtgacgtgcggagctgctacactctctgcagagagagtcagagggg |
| | | acaacaaggagtatgagtactcagtggagtgccaggaggacagtgcctgcccagctgctgaggagagtctgcccattga |
| | | ggtcatggtggatgccgttcacaagctcaagtatgaaaactacaccagcagcttcttcatcagggacatcatcaaacct |
| | | gacccacccaagaacttgcagctgaagccattaaagaattctcggcaggtggaggtcagctgggagtaccctgacacct |
| | | ggagtactccacattcctacttctccctgacattctgcgttcaggtccagggcaagagcaagagagaaaagaaagatag |
| | | agtcttcacggacaagacctcagccacggtcatctgccgcaaaaatgccagcattagcgtgcgggcccaggaccgctac |
| | | tatagctcatcttggagcgaatgggcatctgtgccctgcagttag |
| Human IL-23a p19-P2A-IL- | 189 | MLGSRAVMLLLLLPWTAQGRAVPGGSSPAWTQCQQLSQKLCTLAWSAHPLVGHMDLREEGDEETTNDVPHIQCGDGCDP |
| 12 p40 Protein Sequence | | QGLRDNSQFCLQRIHQGLIFYEKLLGSDIFTGEPSLLPDSPVGQLHASLLGLSQLLQPEGHHWETQQIPSLSPSQPWQR |
| | | LLLRFKILRSLQAFVAVAARVFAHGAATLSPATNFSLLKQAGDVEENPGPMCHQQLVISWFSLVFLASPLVAIWELKKD |
| | | VYVVELDWYPDAPGEMVVLTCDTPEEDGITWTLDQSSEVLGSGKTLTIQVKEFGDAGQYTCHKGGEVLSHSLLLLHKKE |
| | | DGIWSTDILKDQKEPKNKTFLRCEAKNYSGRFTCWWLTTISTDLTFSVKSSRGSSDPQGVTCGAATLSAERVRGDNKEY |
| | | EYSVECQEDSACPAAEESLPIEVMVDAVHKLKYENYTSSFFIRDIIKPDPPKNLQLKPLKNSRQVEVSWEYPDTWSTPH |
| | | SYFSLTFCVQVQGKSKREKKDRVFTDKTSATVICRKNASISVRAQDRYYSSSWSEWASVPCS |
| Human IL-23a p19-P2A-IL- | 190 | gccaccatgctggggagcagagctgtaatgctgctgttgctgctgccctggacagctcagggcagagctgtgcctgggg |
| 12 p40 DNA Sequence | | gcagcagccctgcctggactcagtgccagcagctttcacagaagctctgcacactggcctggagtgcacatccactagt |
| | | gggacacatggatctaagagaagagggagatgaagagactacaaatgatgttccccatatccagtgtggagatggctgt |
| | | gacccccaaggactcagggacaacagtcagttctgcttgcaaaggatccaccagggtctgattttttatgagaagctgc |
| | | taggatcggatattttcacaggggagccttctctgctccctgatagccctgtgggccagcttcatgcctccctactggg |
| | | cctcagccaactcctgcagcctgagggtcaccactgggagactcagcagattccaagcctcagtcccagccagccatgg |
| | | cagcgtctccttctccgcttcaaaatccttcgcagcctccaggcctttgtggctgtagccgcccgggtctttgcccatg |
| | | gagcagcaaccctgagtcccgccaccaacttttcattgctcaagcaggcgggcgatgtggaggaaaaccctggccccgg |
| | | atccatgtgtcaccagcagttggtcatctcttggttttccctggtttttctggcatctcccctcgtggccatatgggaa |
| | | ctgaagaaagatgtttatgtcgtagaattggattggtatccggatgcccctggagaaatggtggtcctcacctgtgaca |
| | | cccctgaagaagatggtatcacctggaccttggaccagagcagtgaggtcttaggctctggcaaaaccctgaccatcca |
| | | agtcaaagagtttggagatgctggccagtacacctgtcacaaaggaggcgaggttctaagccattcgctcctgctgctt |
| | | cacaaaaaggaagatggaatttggtccactgatattttaaaggaccagaaagaacccaaaaataagacctttctaagat |
| | | gcgaggccaagaattattctggacgtttcacctgctggtggctgacgacaatcagtactgatttgacattcagtgtcaa |
| | | aagcagcagaggctcttctgacccccaaggggtgacgtgcggagctgctacactctctgcagagagagtcagaggggac |
| | | aacaaggagtatgagtactcagtggagtgccaggaggacagtgcctgcccagctgctgaggagagtctgcccattgagg |
| | | tcatggtggatgccgttcacaagctcaagtatgaaaactacaccagcagcttcttcatcagggacatcatcaaacctga |
| | | cccacccaagaacttgcagctgaagccattaaagaattctcggcaggtggaggtcagctgggagtaccctgacacctgg |
| | | agtactccacattcctacttctccctgacattctgcgttcaggtccagggcaagagcaagagagaaaagaaagatagag |
| | | tcttcacggacaagacctcagccacggtcatctgccgcaaaaatgccagcattagcgtgcgggcccaggaccgctacta |
| | | tagctcatcttggagcgaatgggcatctgtgccctgcagttag |
| Human IL-23a p19 Protein | 191 | MLGSRAVMLLLLLPWTAQGRAVPGGSSPAWTQCQQLSQKLCTLAWSAHPLVGHMDLREEGDEETTNDVPHIQCGDGCDP |
| Sequence | | QGLRDNSQFCLQRIHQGLIFYEKLLGSDIFTGEPSLLPDSPVGQLHASLLGLSQLLQPEGHHWETQQIPSLSPSQPWQR |
| | | LLLRFKILRSLQAFVAVAARVFAHGAATLSP |
| Human IL-23a p19 DNA | 192 | gccaccatgctggggagcagagctgtaatgctgctgttgctgctgccctggacagctcagggcagagctgtgcctgggg |
| Sequence | | gcagcagccctgcctggactcagtgccagcagctttcacagaagctctgcacactggcctggagtgcacatccactagt |
| | | gggacacatggatctaagagaagagggagatgaagagactacaaatgatgttccccatatccagtgtggagatggctgt |
| | | gacccccaaggactcagggacaacagtcagttctgcttgcaaaggatccaccagggtctgattttttatgagaagctgc |
| | | taggatcggatattttcacaggggagccttctctgctccctgatagccctgtgggccagcttcatgcctccctactggg |
| | | cctcagccaactcctgcagcctgagggtcaccactgggagactcagcagattccaagcctcagtcccagccagccatgg |
| | | cagcgtctccttctccgcttcaaaatccttcgcagcctccaggcctttgtggctgtagccgcccgggtctttgcccatg |
| | | gagcagcaaccctgagtccc |
| Human IL-12 p40 Protein | 193 | MCHQQLVISWFSLVFLASPLVAIWELKKDVYVVELDWYPDAPGEMVVLTCDTPEEDGITWTLDQSSEVLGSGKTLTIQV |
| Sequence | | KEFGDAGQYTCHKGGEVLSHSLLLLHKKEDGIWSTDILKDQKEPKNKTFLRCEAKNYSGRFTCWWLTTISTDLTFSVKS |
| | | SRGSSDPQGVTCGAATLSAERVRGDNKEYEYSVECQEDSACPAAEESLPIEVMVDAVHKLKYENYTSSFFIRDIIKPDP |
| | | PKNLQLKPLKNSRQVEVSWEYPDTWSTPHSYFSLTFCVQVQGKSKREKKDRVFTDKTSATVICRKNASISVRAQDRYYS |
| | | SSWSEWASVPCS |
| Human IL-12 p40 DNA | 194 | ggatccatgtgtcaccagcagttggtcatctcttggttttccctggtttttctggcatctcccctcgtggccatatggg |
| Sequence | | aactgaagaaagatgtttatgtcgtagaattggattggtatccggatgcccctggagaaatggtggtcctcacctgtga |
| | | cacccctgaagaagatggtatcacctggaccttggaccagagcagtgaggtcttaggctctggcaaaaccctgaccatc |
| | | caagtcaaagagtttggagatgctggccagtacacctgtcacaaaggaggcgaggttctaagccattcgctcctgctgc |
| | | ttcacaaaaaggaagatggaatttggtccactgatattttaaaggaccagaaagaacccaaaaataagacctttctaag |
| | | atgcgaggccaagaattattctggacgtttcacctgctggtggctgacgacaatcagtactgatttgacattcagtgtc |
| | | aaaagcagcagaggctcttctgacccccaaggggtgacgtgcggagctgctacactctctgcagagagagtcagagggg |
| | | acaacaaggagtatgagtactcagtggagtgccaggaggacagtgcctgcccagctgctgaggagagtctgcccattga |
| | | ggtcatggtggatgccgttcacaagctcaagtatgaaaactacaccagcagcttcttcatcagggacatcatcaaacct |
| | | gacccacccaagaacttgcagctgaagccattaaagaattctcggcaggtggaggtcagctgggagtaccctgacacct |
| | | ggagtactccacattcctacttctccctgacattctgcgttcaggtccagggcaagagcaagagagaaaagaaagatag |
| | | agtcttcacggacaagacctcagccacggtcatctgccgcaaaaatgccagcattagcgtgcgggcccaggaccgctac |
| | | tatagctcatcttggagcgaatgggcatctgtgccctgcagttag |
| HER2-LAMP Protein | 195 | MAPRSARRPLLLLLLLLLLGLMHCASAAMFMVKNGNGTACIMANFSAAFSVNYDTKSGPKNMTLDLPSDATVVLNRSSC |
| Sequence | | GKENTSDPSLVIAFGRGHTLTLNFTRNATRYSVQLMSFVYNLSDTHLFPNASSKEIKTVESITDIRADIDKKYRCVSGT |
| | | QVHMNNVTVTLHDATIQAYLSNSSFSRGETRCEQDLETQVCTGTDMKLRLPASPETHLDMLRHLYQGCQVVQGNLELTY |
| | | LPTNASLSFLQDIQEVQGYVLIAHNQVRQVPLQRLRIVRGTQLFEDNYALAVLDNGDPLNNTTPVTGASPGGLRELQLR |
| | | SLTEILKGGVLIQRNPQLCYQDTILWKDIFHKNNQLALTLIDTNRSRACHPCSPMCKGSRCWGESSEDCQSLTRTVCAG |
| | | GCARCKGPLPTDCCHEQCAAGCTGPKHSDCLACLHFNHSGICELHCPALVTYNTDTFESMPNPEGRYTFGASCVTACPY |
| | | NYLSTDVGSCTLVCPLHNQEVTAEDGTQRCEKCSKPCARVCYGLGMEHLREVRAVTSANIQEFAGCKKIFGSLAFLPES |
| | | FDGDPASNTAPLQPEQLQVFETLEEITGYLYISAWPDSLPDLSVFQNLQVIRGRILHNGAYSLTLQGLGISWLGLRSLR |
| | | ELGSGLALIHHNTHLCFVHTVPWDQLFRNPHQALLHTANRPEDECVGEGLACHQLCARGHCWGPGPTQCVNCSQFLRGQ |
| | | ECVEECRVLQGLPREYVNARHCLPCHPECQPQNGSVTCFGPEADQCVACAHYKDPPFCVARCPSGVKPDLSYMPIWKFP |
| | | DEEGACQPCPINCTHEFTCLLASMGLQLNLTYERKDNTTVTRLLNINPNKTSASGSCGAHLVTLELHSEGTTVLLFQFG |
| | | MNASSSRFFLQGIQLNTILPDARDPAFKAANGSLRALQATVGNSYKCNAEEHVRVTKAFSVNIFKVWVQAFKVEGGQFG |
| | | SVEECLLDENSM |
| mSPD + sCD40L protein | 196 | MLPFLSMLVLLVQPLGNLGAEMKSLSQRSVPNTCTLVMCSPTENGLPGRDGRDGREGPRGEKGDPGLPGPMGLSGLQGP |
| sequence | | TGPVGPKGENGSAGEPGPKGERGLSGPPGLPGIPGPAGKEGPSGKQGNIGPQGKPGPKGEAGPKGEVGAPGMQGSTGAK |
| | | GSTGPKGERGAPGVQGAPGNAGAAGPAGPAGPQGAPGSRGPPGLKGDRGVPGDRGIKGESGLPDSAALRQQMEALKGKL |
| | | QRLEVAFSHYQKAALFPDGHRRLDKVEEEVNLHEDFVFIKKLKRCNKGEGSLSLLNCEEMRRQFEDLVKDITLNKEEKK |
| | | ENSFEMQRGDEDPQIAAHVVSEANSNAASVLQWAKKGYYTMKSNLVMLENGKQLTVKREGLYYVYTQVTFCSNREPSSQ |
| | | RPFIVGLWLKPSSGSERILLKAANTHSSSQLCEQQSVHLGGVFELQAGASVFVNVTEASQVIHRVGFSSFGLLKL |
| HER2-LAMP-sCD40L; | 197 | atggtgaaaaatggcaacgggaccgcgtgcataatggccaacttctctgctgccttctcagtgaactacgacaccaaga |
| HER2 (Extracellular | | gtggccctaagaacatgacccttgacctgccatcagatgccacagtggtgctcaaccgcagctcctgtggaaaagagaa |
| Domain) +LAMP +EF- | | cacttctgaccccagtctcgtgattgcttttggaagaggacatacactcactctcaatttcacgagaaatgcaacacgt |
| 1alpha +SPD +sCD40L; | | tacagcgtccagctcatgagttttgtttataacttgtcagacacacaccttttccccaatgcgagctccaaagaaatca |
| DNA Sequence | | agactgtggaatctataactgacatcagggcagatatagataaaaaatacagatgtgttagtggcacccaggtccacat |
| | | gaacaacgtgaccgtaacgctccatgatgccaccatccaggcgtacctttccaacagcagcttcagccggggagagaca |
| | | cgctgtgaacaagacctcgagacacaagtctgcaccggcaccgacatgaagctgagactgcccgccagccccgaaaccc |
| | | atctggacatgctgcggcatctgtaccaaggctgtcaagtggtgcaaggcaatctggaactgacatatctgcctaccaa |
| | | cgcctctctgagctttctgcaagacatccaagaagtgcaaggctacgtcctcattgcccacaaccaagtccgacaagtg |
| | | ccactgcagcggctgagaatcgtgcggggcacccagctgttcgaggacaactatgctctggccgtgctggacaacggcg |
| | | accctctgaacaacaccacaccagtgactggcgcctctcccggcggactgagagaactgcagctgcgctctctgaccga |
| | | gattctgaagggcggcgtgctgatccagcggaaccctcagctgtgctaccaagacaccattctgtggaaggacatcttc |
| | | cacaagaacaaccagctggctctgaccctcatcgacaccaacagaagccgggcttgccacccttgcagccccatgtgca |
| | | agggcagcagatgttggggcgagagcagcgaggactgccagtctctgaccagaaccgtgtgtgccggcggatgcgccag |
| | | atgcaagggccctctgccaaccgattgctgccacgaacagtgcgccgctggctgtaccggccccaagcacagcgattgt |
| | | ctggcttgtctgcacttcaaccactccggcatctgcgagctgcactgcccagccctcgtgacatacaacaccgacacat |
| | | tcgagagcatgcccaaccccgagggcagatacacattcggcgccagctgtgtgaccgcttgcccctacaactatctgag |
| | | caccgacgtgggcagctgcaccctcgtgtgccctctgcacaatcaagaagtgaccgccgaggacggcacccagagatgc |
| | | gagaagtgctccaagccttgcgccagagtgtgctacggactgggcatggaacatctgcgggaagtgcgggccgtgacca |
| | | gcgccaatatccaagaatttgccggctgcaagaagattttcggcagtctggcctttctgcccgagagcttcgatggcga |
| | | ccccgcctctaacacagcccctctgcagccagagcagctccaagtgttcgagacactggaagagatcaccggctatctg |
| | | tacatcagcgcttggcccgactctctgcccgatctgagcgtgttccagaatctgcaagtcatcagaggccggattctgc |
| | | acaacggcgcctattctctgacactgcaaggactgggaatcagctggctgggactgcggagtctgagagagctgggcag |
| | | cggactggcactgatccaccacaacacccatctgtgcttcgtgcacaccgtgccttgggatcagctgttccggaatccc |
| | | caccaagctctgctgcacaccgccaacagacccgaggatgagtgtgtgggcgaggggctggcttgccatcagctctgtg |
| | | ccagaggacactgttggggacccggccctacccagtgcgtgaactgctcccagtttctgcggggccaagaatgcgtgga |
| | | agagtgcagagtgctccaaggactgccccgcgagtacgtgaacgccagacactgtctgccatgccaccccgagtgccag |
| | | cctcagaatggcagcgtgacatgcttcggccccgaggccgatcagtgtgtggcttgcgctcactacaaggaccctccct |
| | | tctgcgtggcccggtgtccttctggcgtgaaacccgatctgtcctacatgcctatctggaagttccccgacgaggaagg |
| | | cgcttgtcagccttgccccatcaactgcacccacgaattcacctgcctgctggccagcatggggctgcagctgaacctc |
| | | acctatgagaggaaggacaacacgacggtgacaaggcttctcaacatcaaccccaacaagacctcggccagcgggagct |
| | | gcggcgcccacctggtgactctggagctgcacagcgagggcaccaccgtcctgctcttccagttcgggatgaatgcaag |
| | | ttctagccggtttttcctacaaggaatccagttgaatacaattcttcctgacgccagagaccctgcctttaaagctgcc |
| | | aacggctccctgcgagcgctgcaggccacagtcggcaattcctacaagtgcaacgcggaggagcacgtccgtgtcacga |
| | | aggcgttttcagtcaatatattcaaagtgtgggtccaggctttcaaggtggaaggtggccagtttggctctgtggagga |
| | | gtgtctgctggacgagaacagcatgctgatccccatcgctgtgggtggtgccctggcggggctggtcctcatcgtcctc |
| | | atcgcctacctcgtcggcaggaagaggagtcacgcaggctaccagactatctagtaaagatctttttccctctgccaaa |
| | | aattatggggacatcatgaagccccttgagcatctgacttctggctaataaaggaaatttattttcattgcaatagtgt |
| | | gttggaattttttgtgtctctcactcggaaggacataagggcggccgcggtacccgtgaggctccggtgcccgtcagtg |
| | | ggcagagcgcacatcgcccacagtccccgagaagttggggggaggggtcggcaattgaaccggtgcctagagaaggtgg |
| | | cgcggggtaaactgggaaagtgatgtcgtgtactggctccgcctttttcccgagggtgggggagaaccgtatataagtg |
| | | cagtagtcgccgtgaacgttctttttcgcaacgggtttgccgccagaacacagtctagaggatccgccgccaccatgga |
| | | aaccgatacactgctgctgtgggtgctgttgctctgggttccaggatctacaggcgacatgctgccctttctctccatg |
| | | cttgtcttgcttgtacagcccctgggaaatctgggagcagaaatgaagagcctctcgcagagatcagtacccaacacct |
| | | gcaccctagtcatgtgtagcccaacagagaatggcctgcctggtcgtgatggacgggatgggagagaaggtccacgggg |
| | | tgagaagggtgatccaggtttgccaggacctatggggctctcagggttgcagggccctacaggtccagttggacccaaa |
| | | ggagagaatggctctgctggcgaacctggaccaaagggagaacgtggactaagtggacctccaggacttccaggtattc |
| | | ctggtccagctgggaaagaaggtccctctgggaagcaggggaacataggacctcaaggcaaaccaggtcctaaaggaga |
| | | ggctgggcccaaaggagaagtaggtgctcctggcatgcaaggatctacaggggcaaaaggctccacaggccccaaggga |
| | | gaaagaggtgcccctggtgtgcaaggagccccagggaatgctggagcagcaggacctgccggacctgccggtccacagg |
| | | gagctccaggttccagggggcccccaggactcaagggggacagaggtgttcctggagacagaggaatcaaaggtgaaag |
| | | cgggcttccagacagtgctgctctgaggcagcagatggaggccttaaaaggaaaactacagcgtctagaggttgccttc |
| | | tcccactatcagaaagctgcattgttccctgatggccatagaagattggataaggtcgaagaggaagtaaaccttcatg |
| | | aagattttgtattcataaaaaagctaaagagatgcaacaaaggagaaggatctttatccttgctgaactgtgaggagat |
| | | gagaaggcaatttgaagaccttgtcaaggatataacgttaaacaaagaagagaaaaaagaaaacagctttgaaatgcaa |
| | | agaggtgatgaggatcctcaaattgcagcacacgttgtaagcgaagccaacagtaatgcagcatccgttctacagtggg |
| | | ccaagaaaggatattataccatgaaaagcaacttggtaatgcttgaaaatgggaaacagctgacggttaaaagagaagg |
| | | actctattatgtctacactcaagtcaccttctgctctaatcgggagccttcgagtcaacgcccattcatcgtcggcctc |
| | | tggctgaagcccagcagtggatctgagagaatcttactcaaggcggcaaatacccacagttcctcccagctttgcgagc |
| | | agcagtctgttcacttgggcggagtgtttgaattacaagctggtgcttctgtgtttgtcaacgtgactgaagcaagcca |
| | | agtgatccacagagttggcttctcatcttttggcttactcaaactctgaacgcgtttcctgtgccttctagttgccagc |
| | | catctgttgtttgcccctcccccgtgccttccttgaccctggaaggtgccactcccactgtcctttcctaataaaatga |
| | | ggaaattgcatcgcattgtctgagtaggtgtcattctattctggggggtggggtggggcaggacagcaagggggaggat |
| | | tgggaagacaatagcaggcatgctggggatgcggtgggctctatggcccggg |
| LAMP Signal Sequence | 198 | MAPRSARRPLLLLLLLLLLGLMHCASAAMFMVKNGNGTACIMANFSAAFSVNYDTKSGPKNMTLDLPSDATVVLNRSSC |
| (residues 1-28) and Luminal | | GKENTSDPSLVIAFGRGHTLTLNFTRNATRYSVQLMSFVYNLSDTHLFPNASSKEIKTVESITDIRADIDKKYRCVSGT |
| Domain 1 | | QVHMNNVTVTLHDATIQAYLSNSSFSRGETRCEQDLE |
| LAMP Signal Sequence and | 199 | atggcgccccgcagcgcccggcgacccctgctgctgctactgctgttgctgctgctcggcctcatgcattgtgcgtcag |
| Luminal Domain 1 | | cagcaatgtttatggtgaaaaatggcaacgggaccgcgtgcataatggccaacttctctgctgccttctcagtgaacta |
| | | cgacaccaagagtggccctaagaacatgacccttgacctgccatcagatgccacagtggtgctcaaccgcagctcctgt |
| | | ggaaaagagaacacttctgaccccagtctcgtgattgcttttggaagaggacatacactcactctcaatttcacgagaa |
| | | atgcaacacgttacagcgtccagctcatgagttttgtttataacttgtcagacacacaccttttccccaatgcgagctc |
| | | caaagaaatcaagactgtggaatctataactgacatcagggcagatatagataaaaaatacagatgtgttagtggcacc |
| | | caggtccacatgaacaacgtgaccgtaacgctccatgatgccaccatccaggcgtacctttccaacagcagcttcagcc |
| | | ggggagagacacgctgtgaacaagacctcgag |
| HER2 (Extracellular | 200 | TQVCTGTDMKLRLPASPETHLDMLRHLYQGCQVVQGNLELTYLPTNASLSFLQDIQEVQGYVLIAHNQVRQVPLQRLRI |
| Domain) Protein Sequence | | VRGTQLFEDNYALAVLDNGDPLNNTTPVTGASPGGLRELQLRSLTEILKGGVLIQRNPQLCYQDTILWKDIFHKNNQLA |
| | | LTLIDTNRSRACHPCSPMCKGSRCWGESSEDCQSLTRTVCAGGCARCKGPLPTDCCHEQCAAGCTGPKHSDCLACLHFN |
| | | HSGICELHCPALVTYNTDTFESMPNPEGRYTFGASCVTACPYNYLSTDVGSCTLVCPLHNQEVTAEDGTQRCEKCSKPC |
| | | ARVCYGLGMEHLREVRAVTSANIQEFAGCKKIFGSLAFLPESFDGDPASNTAPLQPEQLQVFETLEEITGYLYISAWPD |
| | | SLPDLSVFQNLQVIRGRILHNGAYSLTLQGLGISWLGLRSLRELGSGLALIHHNTHLCFVHTVPWDQLFRNPHQALLHT |
| | | ANRPEDECVGEGLACHQLCARGHCWGPGPTQCVNCSQFLRGQECVEECRVLQGLPREYVNARHCLPCHPECQPQNGSVT |
| | | CFGPEADQCVACAHYKDPPFCVARCPSGVKPDLSYMPIWKFPDEEGACQPCPINCTHEF |
| HER2 (Extracellular | 201 | acccaggtgtgcaccggcaccgatatgaaactgcgcctgccggcgagcccggaaacccatctggatatgctgcgccatc |
| Domain) DNA Sequence | | tgtatcagggctgccaggtggtgcagggcaacctggaactgacctatctgccgaccaacgcgagcctgagctttctgca |
| | | ggatattcaggaagtgcagggctatgtgctgattgcgcataaccaggtgcgccaggtgccgctgcagcgcctgcgcatt |
| | | gtgcgcggcacccagctgtttgaagataactatgcgctggcggtgctggataacggcgatccgctgaacaacaccaccc |
| | | cggtgaccggcgcgagcccgggcggcctgcgcgaactgcagctgcgcagcctgaccgaaattctgaaaggcggcgtgct |
| | | gattcagcgcaacccgcagctgtgctatcaggataccattctgtggaaagatatttttcataaaaacaaccagctggcg |
| | | ctgaccctgattgataccaaccgcagccgcgcgtgccatccgtgcagcccgatgtgcaaaggcagccgctgctggggcg |
| | | aaagcagcgaagattgccagagcctgacccgcaccgtgtgcgcgggcggctgcgcgcgctgcaaaggcccgctgccgac |
| | | cgattgctgccatgaacagtgcgcggcgggctgcaccggcccgaaacatagcgattgcctggcgtgcctgcattttaac |
| | | catagcggcatttgcgaactgcattgcccggcgctggtgacctataacaccgatacctttgaaagcatgccgaacccgg |
| | | aaggccgctatacctttggcgcgagctgcgtgaccgcgtgcccgtataactatctgagcaccgatgtgggcagctgcac |
| | | cctggtgtgcccgctgcataaccaggaagtgaccgcggaagatggcacccagcgctgcgaaaaatgcagcaaaccgtgc |
| | | gcgcgcgtgtgctatggcctgggcatggaacatctgcgcgaagtgcgcgcggtgaccagcgcgaacattcaggaatttg |
| | | cgggctgcaaaaaaatttttggcagcctggcgtttctgccggaaagctttgatggcgatccggcgagcaacaccgcgcc |
| | | gctgcagccggaacagctgcaggtgtttgaaaccctggaagaaattaccggctatctgtatattagcgcgtggccggat |
| | | agcctgccggatctgagcgtgtttcagaacctgcaggtgattcgcggccgcattctgcataacggcgcgtatagcctga |
| | | ccctgcagggcctgggcattagctggctgggcctgcgcagcctgcgcgaactgggcagcggcctggcgctgattcatca |
| | | taacacccatctgtgctttgtgcataccgtgccgtgggatcagctgtttcgcaacccgcatcaggcgctgctgcatacc |
| | | gcgaaccgcccggaagatgaatgcgtgggcgaaggcctggcgtgccatcagctgtgcgcgcgcggccattgctggggcc |
| | | cgggcccgacccagtgcgtgaactgcagccagtttctgcgcggccaggaatgcgtggaagaatgccgcgtgctgcaggg |
| | | cctgccgcgcgaatatgtgaacgcgcgccattgcctgccgtgccatccggaatgccagccgcagaacggcagcgtgacc |
| | | tgctttggcccggaagcggatcagtgcgtggcgtgcgcgcattataaagatccgccgttttgcgtggcgcgctgcccga |
| | | gcggcgtgaaaccggatctgagctatatgccgatttggaaatttccggatgaagaaggcgcgtgccagccgtgcccgat |
| | | taactgcacccatgaattt |
| Luminal Domain 2 of LAMP | 202 | TCLLASMGLQLNLTYERKDNTTVTRLLNINPNKTSASGSCGAHLVTLELHSEGTTVLLFQFGMNASSSRFFLQGIQLNT |
| Protein Sequence | | ILPDARDPAFKAANGSLRALQATVGNSYKCNAEEHVRVTKAFSVNIFKVWVQAFKVEGGQFGSVEECLLDENSM |
| Luminal Domain 2 of LAMP | 203 | acctgcctgctggccagcatggggctgcagctgaacctcacctatgagaggaaggacaacacgacggtgacaaggcttc |
| DNA Sequence | | tcaacatcaaccccaacaagacctcggccagcgggagctgcggcgcccacctggtgactctggagctgcacagcgaggg |
| | | caccaccgtcctgctcttccagttcgggatgaatgcaagttctagccggtttttcctacaaggaatccagttgaataca |
| | | attcttcctgacgccagagaccctgcctttaaagctgccaacggctccctgcgagcgctgcaggccacagtcggcaatt |
| | | cctacaagtgcaacgcggaggagcacgtccgtgtcacgaaggcgttttcagtcaatatattcaaagtgtgggtccaggc |
| | | tttcaaggtggaaggtggccagtttggctctgtggaggagtgtctgctggacgagaacagcatg |
| sCD40L Protein Sequence | 204 | HRRLDKVEEEVNLHEDFVFIKKLKRCNKGEGSLSLLNCEEMRRQFEDLVKDITLNKEEKKENSFEMQRGDEDPQIAAHV |
| | | VSEANSNAASVLQWAKKGYYTMKSNLVMLENGKQLTVKREGLYYVYTQVTFCSNREPSSQRPFIVGLWLKPSSGSERIL |
| | | LKAANTHSSSQLCEQQSVHLGGVFELQAGASVFVNVTEASQVIHRVGFSSFGLLKL |
| sCD40L DNA Sequence | 205 | catcgccgcctggataaagtggaagaagaagtgaacctgcatgaagattttgtgtttattaaaaaactgaaacgctgca |
| | | acaaaggcgaaggcagcctgagcctgctgaactgcgaagaaatgcgccgccagtttgaagatctggtgaaagatattac |
| | | cctgaacaaagaagaaaaaaaagaaaacagctttgaaatgcagcgcggcgatgaagatccgcagattgcggcgcatgtg |
| | | gtgagcgaagcgaacagcaacgcggcgagcgtgctgcagtgggcgaaaaaaggctattataccatgaaaagcaacctgg |
| | | tgatgctggaaaacggcaaacagctgaccgtgaaacgcgaaggcctgtattatgtgtatacccaggtgaccttttgcag |
| | | caaccgcgaaccgagcagccagcgcccgtttattgtgggcctgtggctgaaaccgagcagcggcagcgaacgcattctg |
| | | ctgaaagcggcgaacacccatagcagcagccagctgtgcgaacagcagagcgtgcatctgggcggcgtgtttgaactgc |
| | | aggcgggcgcgagcgtgtttgtgaacgtgaccgaagcgagccaggtgattcatcgcgtgggctttagcagctttggcct |
| | | gctgaaactgtaa |
| | 206 | |
| SPD + mFLT3L protein | 207 | MLPFLSMLVLLVQPLGNLGAEMKSLSQRSVPNTCTLVMCSPTENGLPGRDGRDGREGPRGEKGDPGLPGPMGLSGLQGP |
| sequence | | TGPVGPKGENGSAGEPGPKGERGLSGPPGLPGIPGPAGKEGPSGKQGNIGPQGKPGPKGEAGPKGEVGAPGMQGSTGAK |
| | | GSTGPKGERGAPGVQGAPGNAGAAGPAGPAGPQGAPGSRGPPGLKGDRGVPGDRGIKGESGLPDSAALRQQMEALKGKL |
| | | QRLEVAFSHYQKAALFPDGPCLRGTPDCYFSHSPISSNFKVKFRELTDHLLKDYPVTVAVNLQDEKHCKALWSLFLAQR |
| | | WIEQLKTVAGSKMQTLLEDVNTEIHFVTSCTFQPLPECLRFVQTNISHLLKDTCTQLLALKPCIGKACQNFSRCLEVQC |
| | | QPDSSTLLPPRSPIALEATELPEPRPRQ |
| HER2-LAMP-mFLT3L; | 208 | atggcgccccgcagcgcccggcgacccctgctgctgctactgctgttgctgctgctcggcctcatgcattgtgcgtcag |
| HER2 (Extracellular | | cagcaatgtttatggtgaaaaatggcaacgggaccgcgtgcataatggccaacttctctgctgccttctcagtgaacta |
| Domain) +LAMP +EF- | | cgacaccaagagtggccctaagaacatgacccttgacctgccatcagatgccacagtggtgctcaaccgcagctcctgt |
| 1 alpha +SPD +mFLT3L; | | ggaaaagagaacacttctgaccccagtctcgtgattgcttttggaagaggacatacactcactctcaatttcacgagaa |
| DNA Sequence | | atgcaacacgttacagcgtccagctcatgagttttgtttataacttgtcagacacacaccttttccccaatgcgagctc |
| | | caaagaaatcaagactgtggaatctataactgacatcagggcagatatagataaaaaatacagatgtgttagtggcacc |
| | | caggtccacatgaacaacgtgaccgtaacgctccatgatgccaccatccaggcgtacctttccaacagcagcttcagcc |
| | | ggggagagacacgctgtgaacaagacctcgagacacaagtctgcaccggcaccgacatgaagctgagactgcccgccag |
| | | ccccgaaacccatctggacatgctgcggcatctgtaccaaggctgtcaagtggtgcaaggcaatctggaactgacatat |
| | | ctgcctaccaacgcctctctgagctttctgcaagacatccaagaagtgcaaggctacgtcctcattgcccacaaccaag |
| | | tccgacaagtgccactgcagcggctgagaatcgtgcggggcacccagctgttcgaggacaactatgctctggccgtgct |
| | | ggacaacggcgaccctctgaacaacaccacaccagtgactggcgcctctcccggcggactgagagaactgcagctgcgc |
| | | tctctgaccgagattctgaagggcggcgtgctgatccagcggaaccctcagctgtgctaccaagacaccattctgtgga |
| | | aggacatcttccacaagaacaaccagctggctctgaccctcatcgacaccaacagaagccgggcttgccacccttgcag |
| | | ccccatgtgcaagggcagcagatgttggggcgagagcagcgaggactgccagtctctgaccagaaccgtgtgtgccggc |
| | | ggatgcgccagatgcaagggccctctgccaaccgattgctgccacgaacagtgcgccgctggctgtaccggccccaagc |
| | | acagcgattgtctggcttgtctgcacttcaaccactccggcatctgcgagctgcactgcccagccctcgtgacatacaa |
| | | caccgacacattcgagagcatgcccaaccccgagggcagatacacattcggcgccagctgtgtgaccgcttgcccctac |
| | | aactatctgagcaccgacgtgggcagctgcaccctcgtgtgccctctgcacaatcaagaagtgaccgccgaggacggca |
| | | cccagagatgcgagaagtgctccaagccttgcgccagagtgtgctacggactgggcatggaacatctgcgggaagtgcg |
| | | ggccgtgaccagcgccaatatccaagaatttgccggctgcaagaagattttcggcagtctggcctttctgcccgagagc |
| | | ttcgatggcgaccccgcctctaacacagcccctctgcagccagagcagctccaagtgttcgagacactggaagagatca |
| | | ccggctatctgtacatcagcgcttggcccgactctctgcccgatctgagcgtgttccagaatctgcaagtcatcagagg |
| | | ccggattctgcacaacggcgcctattctctgacactgcaaggactgggaatcagctggctgggactgcggagtctgaga |
| | | gagctgggcagcggactggcactgatccaccacaacacccatctgtgcttcgtgcacaccgtgccttgggatcagctgt |
| | | tccggaatccccaccaagctctgctgcacaccgccaacagacccgaggatgagtgtgtgggcgaggggctggcttgcca |
| | | tcagctctgtgccagaggacactgttggggacccggccctacccagtgcgtgaactgctcccagtttctgcggggccaa |
| | | gaatgcgtggaagagtgcagagtgctccaaggactgccccgcgagtacgtgaacgccagacactgtctgccatgccacc |
| | | ccgagtgccagcctcagaatggcagcgtgacatgcttcggccccgaggccgatcagtgtgtggcttgcgctcactacaa |
| | | ggaccctcccttctgcgtggcccggtgtccttctggcgtgaaacccgatctgtcctacatgcctatctggaagttcccc |
| | | gacgaggaaggcgcttgtcagccttgccccatcaactgcacccacgaattcacctgcctgctggccagcatggggctgc |
| | | agctgaacctcacctatgagaggaaggacaacacgacggtgacaaggcttctcaacatcaaccccaacaagacctcggc |
| | | cagcgggagctgcggcgcccacctggtgactctggagctgcacagcgagggcaccaccgtcctgctcttccagttcggg |
| | | atgaatgcaagttctagccggtttttcctacaaggaatccagttgaatacaattcttcctgacgccagagaccctgcct |
| | | ttaaagctgccaacggctccctgcgagcgctgcaggccacagtcggcaattcctacaagtgcaacgcggaggagcacgt |
| | | ccgtgtcacgaaggcgttttcagtcaatatattcaaagtgtgggtccaggctttcaaggtggaaggtggccagtttggc |
| | | tctgtggaggagtgtctgctggacgagaacagcatgctgatccccatcgctgtgggtggtgccctggcggggctggtcc |
| | | tcatcgtcctcatcgcctacctcgtcggcaggaagaggagtcacgcaggctaccagactatctagtaaagatctttttc |
| | | cctctgccaaaaattatggggacatcatgaagccccttgagcatctgacttctggctaataaaggaaatttattttcat |
| | | tgcaatagtgtgttggaattttttgtgtctctcactcggaaggacataagggcggccgcggtacccgtgaggctccggt |
| | | gcccgtcagtgggcagagcgcacatcgcccacagtccccgagaagttggggggaggggtcggcaattgaaccggtgcct |
| | | agagaaggtggcgcggggtaaactgggaaagtgatgtcgtgtactggctccgcctttttcccgagggtgggggagaacc |
| | | gtatataagtgcagtagtcgccgtgaacgttctttttcgcaacgggtttgccgccagaacacagtctagaggatccgcc |
| | | gccaccatggaaaccgatacactgctgctgtgggtgctgttgctctgggttccaggatctacaggcgacatgctgccct |
| | | ttctctccatgcttgtcttgcttgtacagcccctgggaaatctgggagcagaaatgaagagcctctcgcagagatcagt |
| | | acccaacacctgcaccctagtcatgtgtagcccaacagagaatggcctgcctggtcgtgatggacgggatgggagagaa |
| | | ggtccacggggtgagaagggtgatccaggtttgccaggacctatggggctctcagggttgcagggccctacaggtccag |
| | | ttggacccaaaggagagaatggctctgctggcgaacctggaccaaagggagaacgtggactaagtggacctccaggact |
| | | tccaggtattcctggtccagctgggaaagaaggtccctctgggaagcaggggaacataggacctcaaggcaaaccaggt |
| | | cctaaaggagaggctgggcccaaaggagaagtaggtgctcctggcatgcaaggatctacaggggcaaaaggctccacag |
| | | gccccaagggagaaagaggtgcccctggtgtgcaaggagccccagggaatgctggagcagcaggacctgccggacctgc |
| | | cggtccacagggagctccaggttccagggggcccccaggactcaagggggacagaggtgttcctggagacagaggaatc |
| | | aaaggtgaaagcgggcttccagacagtgctgctctgaggcagcagatggaggccttaaaaggaaaactacagcgtctag |
| | | aggttgccttctcccactatcagaaagctgcattgttccctgatggcccctgcctgagaggcacccctgattgctactt |
| | | ctctcacagccccatcagcagcaacttcaaagtgaagttcagagagctgaccgaccatctgctgaaggactaccctgtg |
| | | accgtggccgtgaacctgcaggatgagaagcactgcaaagccctgtggtccctgtttctggcccagagatggatcgagc |
| | | agctgaaaacagtggccggcagcaagatgcagaccctgctggaagatgtgaacaccgagatccacttcgtgaccagctg |
| | | caccttccagcctctgcctgagtgcctgagattcgtgcagaccaacatcagccatctgctcaaagacacatgcacccag |
| | | ctgctggccctgaagccttgtatcggcaaagcctgccagaacttcagccggtgcctggaagttcagtgccagcctgata |
| | | gcagcacactgctgcctccaagaagccctatcgctctggaagccacagagctgcctgagcctagacctcggcag |
| mFLT3L; Protein Sequence | 209 | PCLRGTPDCYFSHSPISSNFKVKFRELTDHLLKDYPVTVAVNLQDEKHCKALWSLFLAQRWIEQLKTVAGSKMQTLLED |
| | | VNTEIHFVTSCTFQPLPECLRFVQTNISHLLKDTCTQLLALKPCIGKACQNFSRCLEVQCQPDSSTLLPPRSPIALEAT |
| | | ELPEPRPRQ |
| mFLT3L; DNA Sequence | 210 | ccgtgcctgcgcggcaccccggattgctattttagccatagcccgattagcagcaactttaaagtgaaatttcgcgaac |
| | | tgaccgatcatctgctgaaagattatccggtgaccgtggcggtgaacctgcaggatgaaaaacattgcaaagcgctgtg |
| | | gagcctgtttctggcgcagcgctggattgaacagctgaaaaccgtggcgggcagcaaaatgcagaccctgctggaagat |
| | | gtgaacaccgaaattcattttgtgaccagctgcacctttcagccgctgccggaatgcctgcgctttgtgcagaccaaca |
| | | ttagccatctgctgaaagatacctgcacccagctgctggcgctgaaaccgtgcattggcaaagcgtgccagaactttag |
| | | ccgctgcctggaagtgcagtgccagccggatagcagcaccctgctgccgccgcgcagcccgattgcgctggaagcgacc |
| | | gaactgccggaaccgcgcccgcgccag |
| | 211 | |
| HER2-LAMP-IL-12; HER2 | 212 | atggcgccccgcagcgcccggcgacccctgctgctgctactgctgttgctgctgctcggcctcatgcattgtgcgtcag |
| (Extracellular domain) | | cagcaatgtttatggtgaaaaatggcaacgggaccgcgtgcataatggccaacttctctgctgccttctcagtgaacta |
| +LAMP +EF-1alpha +IL-12; | | cgacaccaagagtggccctaagaacatgacccttgacctgccatcagatgccacagtggtgctcaaccgcagctcctgt |
| DNA Sequence | | ggaaaagagaacacttctgaccccagtctcgtgattgcttttggaagaggacatacactcactctcaatttcacgagaa |
| | | atgcaacacgttacagcgtccagctcatgagttttgtttataacttgtcagacacacaccttttccccaatgcgagctc |
| | | caaagaaatcaagactgtggaatctataactgacatcagggcagatatagataaaaaatacagatgtgttagtggcacc |
| | | caggtccacatgaacaacgtgaccgtaacgctccatgatgccaccatccaggcgtacctttccaacagcagcttcagcc |
| | | ggggagagacacgctgtgaacaagacctcgagacacaagtctgcaccggcaccgacatgaagctgagactgcccgccag |
| | | ccccgaaacccatctggacatgctgcggcatctgtaccaaggctgtcaagtggtgcaaggcaatctggaactgacatat |
| | | ctgcctaccaacgcctctctgagctttctgcaagacatccaagaagtgcaaggctacgtcctcattgcccacaaccaag |
| | | tccgacaagtgccactgcagcggctgagaatcgtgcggggcacccagctgttcgaggacaactatgctctggccgtgct |
| | | ggacaacggcgaccctctgaacaacaccacaccagtgactggcgcctctcccggcggactgagagaactgcagctgcgc |
| | | tctctgaccgagattctgaagggcggcgtgctgatccagcggaaccctcagctgtgctaccaagacaccattctgtgga |
| | | aggacatcttccacaagaacaaccagctggctctgaccctcatcgacaccaacagaagccgggcttgccacccttgcag |
| | | ccccatgtgcaagggcagcagatgttggggcgagagcagcgaggactgccagtctctgaccagaaccgtgtgtgccggc |
| | | ggatgcgccagatgcaagggccctctgccaaccgattgctgccacgaacagtgcgccgctggctgtaccggccccaagc |
| | | acagcgattgtctggcttgtctgcacttcaaccactccggcatctgcgagctgcactgcccagccctcgtgacatacaa |
| | | caccgacacattcgagagcatgcccaaccccgagggcagatacacattcggcgccagctgtgtgaccgcttgcccctac |
| | | aactatctgagcaccgacgtgggcagctgcaccctcgtgtgccctctgcacaatcaagaagtgaccgccgaggacggca |
| | | cccagagatgcgagaagtgctccaagccttgcgccagagtgtgctacggactgggcatggaacatctgcgggaagtgcg |
| | | ggccgtgaccagcgccaatatccaagaatttgccggctgcaagaagattttcggcagtctggcctttctgcccgagagc |
| | | ttcgatggcgaccccgcctctaacacagcccctctgcagccagagcagctccaagtgttcgagacactggaagagatca |
| | | ccggctatctgtacatcagcgcttggcccgactctctgcccgatctgagcgtgttccagaatctgcaagtcatcagagg |
| | | ccggattctgcacaacggcgcctattctctgacactgcaaggactgggaatcagctggctgggactgcggagtctgaga |
| | | gagctgggcagcggactggcactgatccaccacaacacccatctgtgcttcgtgcacaccgtgccttgggatcagctgt |
| | | tccggaatccccaccaagctctgctgcacaccgccaacagacccgaggatgagtgtgtgggcgaggggctggcttgcca |
| | | tcagctctgtgccagaggacactgttggggacccggccctacccagtgcgtgaactgctcccagtttctgcggggccaa |
| | | gaatgcgtggaagagtgcagagtgctccaaggactgccccgcgagtacgtgaacgccagacactgtctgccatgccacc |
| | | ccgagtgccagcctcagaatggcagcgtgacatgcttcggccccgaggccgatcagtgtgtggcttgcgctcactacaa |
| | | ggaccctcccttctgcgtggcccggtgtccttctggcgtgaaacccgatctgtcctacatgcctatctggaagttcccc |
| | | gacgaggaaggcgcttgtcagccttgccccatcaactgcacccacgaattcacctgcctgctggccagcatggggctgc |
| | | agctgaacctcacctatgagaggaaggacaacacgacggtgacaaggcttctcaacatcaaccccaacaagacctcggc |
| | | cagcgggagctgcggcgcccacctggtgactctggagctgcacagcgagggcaccaccgtcctgctcttccagttcggg |
| | | atgaatgcaagttctagccggtttttcctacaaggaatccagttgaatacaattcttcctgacgccagagaccctgcct |
| | | ttaaagctgccaacggctccctgcgagcgctgcaggccacagtcggcaattcctacaagtgcaacgcggaggagcacgt |
| | | ccgtgtcacgaaggcgttttcagtcaatatattcaaagtgtgggtccaggctttcaaggtggaaggtggccagtttggc |
| | | tctgtggaggagtgtctgctggacgagaacagcatgctgatccccatcgctgtgggtggtgccctggcggggctggtcc |
| | | tcatcgtcctcatcgcctacctcgtcggcaggaagaggagtcacgcaggctaccagactatctagtaaagatctttttc |
| | | cctctgccaaaaattatggggacatcatgaagccccttgagcatctgacttctggctaataaaggaaatttattttcat |
| | | tgcaatagtgtgttggaattttttgtgtctctcactcggaaggacataagggcggccgcggtacccgtgaggctccggt |
| | | gcccgtcagtgggcagagcgcacatcgcccacagtccccgagaagttggggggaggggtcggcaattgaaccggtgcct |
| | | agagaaggtggcgcggggtaaactgggaaagtgatgtcgtgtactggctccgcctttttcccgagggtgggggagaacc |
| | | gtatataagtgcagtagtcgccgtgaacgttctttttcgcaacgggtttgccgccagaacacagtctagaggatccgcc |
| | | gccaccatggagaggaccctggtgtgcctggtggtgatcttcctgggcaccgtggcccacaagagcagccctcaaggcc |
| | | ctgataggctgctgattaggctgaggcacctgatcgacatcgtggagcagctgaagatctacgagaacgacctggaccc |
| | | tgagctgctgagcgcccctcaagacgtgaagggccactgcgagcacgccgccttcgcctgctttcagaaggccaagctg |
| | | aagcctagcaaccctggcaacaacaagaccttcatcatcgacctggtggctcagctgaggaggaggctgcctgctagga |
| | | ggggcggcaagaagcagaagcacatcgccaagtgccctagctgcgacagctacgagaagaggacccctaaggagttcct |
| | | ggagaggctgaagtggctgctgcagaagatgatccatcagcacctgagctgaac |
| Murine IL-12 (IL-12 p36- | 213 | MVSVPTASPSASSSSSQCRSSMCQSRYLLFLATLALLNHLSLARVIPVSGPARCLSQSRNLLKTTDDMVKTAREKLKHY |
| P2A-IL-12p40) Protein | | SCTAEDIDHEDITRDQTSTLKTCLPLELHKNESCLATRETSSTTRGSCLPPQKTSLMMTLCLGSIYEDLKMYQTEFQAI |
| Sequence | | NAALQNHNHQQIILDKGMLVAIDELMQSLNHNGETLRQKPPVGEADPYRVKMKLCILLHAFSTRVVTINRVMGYLSSAA |
| | | AAGSGATNFSLLKQAGDVEENPGPGSCPQKLTISWFAIVLLVSPLMAMWELEKDVYVVEVDWTPDAPGETVNLTCDTPE |
| | | EDDITWTSDQRHGVIGSGKTLTITVKEFLDAGQYTCHKGGETLSHSHLLLHKKENGIWSTEILKNFKNKTFLKCEAPNY |
| | | SGRFTCSWLVQRNMDLKFNIKSSSSSPDSRAVTCGMASLSAEKVTLDQRDYEKYSVSCQEDVTCPTAEETLPIELALEA |
| | | RQQNKYENYSTSFFIRDIIKPDPPKNLQMKPLKNSQVEVSWEYPDSWSTPHSYFSLKFFVRIQRKKEKMKETEEGCNQK |
| | | GAFLVEKTSTEVQCKGGNVCVQAQDRYYNSSCSKWACVPCRVRS |
| Murine IL-12 (IL-12 p36- | 214 | atggtcagcgttccaacagcctcaccctcggcatccagcagctcctctcagtgccggtccagcatgtgtcaatcacgct |
| P2A-IL-12p40) DNA | | acctcctctttttggccacccttgccctcctaaaccacctcagtttggccagggtcattccagtctctggacctgccag |
| Sequence | | gtgtcttagccagtcccgaaacctgctgaagaccacagatgacatggtgaagacggccagagaaaaactgaaacattat |
| | | tcctgcactgctgaagacatcgatcatgaagacatcacacgggaccaaaccagcacattgaagacctgtttaccactgg |
| | | aactacacaagaacgagagttgcctggctactagagagacttcttccacaacaagagggagctgcctgcccccacagaa |
| | | gacgtctttgatgatgaccctgtgccttggtagcatctatgaggacttgaagatgtaccagacagagttccaggccatc |
| | | aacgcagcacttcagaatcacaaccatcagcagatcattcttgacaagggcatgctggtggccatcgatgagctgatgc |
| | | agtctctgaatcataatggcgagactctgcgccagaaacctcctgtgggagaagcagacccttacagagtgaaaatgaa |
| | | gctctgcatcctgcttcacgccttcagcacccgcgtcgtgaccatcaacagggtgatgggctatctgagctccgccgcg |
| | | gccgcaggatctggggccaccaacttttcattgctcaagcaggcgggcgatgtggaggaaaaccctggccccggatcct |
| | | gtcctcagaagctaaccatctcctggtttgccatcgttttgctggtgtctccactcatggccatgtgggagctggagaa |
| | | agacgtttatgttgtagaggtggactggactcccgatgcccctggagaaacagtgaacctcacctgtgacacgcctgaa |
| | | gaagatgacatcacctggacctcagaccagagacatggagtcataggctctggaaagaccctgaccatcactgtcaaag |
| | | agtttcttgatgctggccagtacacctgccacaaaggaggcgagactctgagccactcacatctgctgctccacaagaa |
| | | ggaaaatggaatttggtccactgaaattttaaagaatttcaagaacaagactttcctgaagtgtgaagcaccaaattac |
| | | tccggacggttcacgtgctcatggctggtgcaaagaaacatggacttgaagttcaacatcaagagcagtagcagttccc |
| | | ctgactctcgggcagtgacatgtggaatggcgtctctgtctgcagagaaggtcacactggaccaaagggactatgagaa |
| | | gtattcagtgtcctgccaggaggatgtcacctgcccaactgccgaggagaccctgcccattgaactggcgttggaagca |
| | | cggcagcagaataaatatgagaactacagcaccagcttcttcatcagggacatcatcaaaccagacccgcccaagaact |
| | | tgcagatgaagcctttgaagaactcacaggtggaggtcagctgggagtaccctgactcctggagcactccccattccta |
| | | cttctccctcaagttctttgttcgaatccagcgcaagaaagaaaagatgaaggagacagaggaggggtgtaaccagaaa |
| | | ggtgcgttcctcgtagagaagacatctaccgaagtccaatgcaaaggcgggaatgtctgcgtgcaagctcaggatcgct |
| | | attacaattcctcatgcagcaagtgggcatgtgttccctgcagggtccgatcctag |
| | 215 | |
| HER2-LAMP-IL-21; HER2 | 216 | atggcgccccgcagcgcccggcgacccctgctgctgctactgctgttgctgctgctcggcctcatgcattgtgcgtcag |
| (Extracellular domain) | | cagcaatgtttatggtgaaaaatggcaacgggaccgcgtgcataatggccaacttctctgctgccttctcagtgaacta |
| +LAMP +EF-1alpha +IL-21; | | cgacaccaagagtggccctaagaacatgacccttgacctgccatcagatgccacagtggtgctcaaccgcagctcctgt |
| DNA Sequence | | ggaaaagagaacacttctgaccccagtctcgtgattgcttttggaagaggacatacactcactctcaatttcacgagaa |
| | | atgcaacacgttacagcgtccagctcatgagttttgtttataacttgtcagacacacaccttttccccaatgcgagctc |
| | | caaagaaatcaagactgtggaatctataactgacatcagggcagatatagataaaaaatacagatgtgttagtggcacc |
| | | caggtccacatgaacaacgtgaccgtaacgctccatgatgccaccatccaggcgtacctttccaacagcagcttcagcc |
| | | ggggagagacacgctgtgaacaagacctcgagacacaagtctgcaccggcaccgacatgaagctgagactgcccgccag |
| | | ccccgaaacccatctggacatgctgcggcatctgtaccaaggctgtcaagtggtgcaaggcaatctggaactgacatat |
| | | ctgcctaccaacgcctctctgagctttctgcaagacatccaagaagtgcaaggctacgtcctcattgcccacaaccaag |
| | | tccgacaagtgccactgcagcggctgagaatcgtgcggggcacccagctgttcgaggacaactatgctctggccgtgct |
| | | ggacaacggcgaccctctgaacaacaccacaccagtgactggcgcctctcccggcggactgagagaactgcagctgcgc |
| | | tctctgaccgagattctgaagggcggcgtgctgatccagcggaaccctcagctgtgctaccaagacaccattctgtgga |
| | | aggacatcttccacaagaacaaccagctggctctgaccctcatcgacaccaacagaagccgggcttgccacccttgcag |
| | | ccccatgtgcaagggcagcagatgttggggcgagagcagcgaggactgccagtctctgaccagaaccgtgtgtgccggc |
| | | ggatgcgccagatgcaagggccctctgccaaccgattgctgccacgaacagtgcgccgctggctgtaccggccccaagc |
| | | acagcgattgtctggcttgtctgcacttcaaccactccggcatctgcgagctgcactgcccagccctcgtgacatacaa |
| | | caccgacacattcgagagcatgcccaaccccgagggcagatacacattcggcgccagctgtgtgaccgcttgcccctac |
| | | aactatctgagcaccgacgtgggcagctgcaccctcgtgtgccctctgcacaatcaagaagtgaccgccgaggacggca |
| | | cccagagatgcgagaagtgctccaagccttgcgccagagtgtgctacggactgggcatggaacatctgcgggaagtgcg |
| | | ggccgtgaccagcgccaatatccaagaatttgccggctgcaagaagattttcggcagtctggcctttctgcccgagagc |
| | | ttcgatggcgaccccgcctctaacacagcccctctgcagccagagcagctccaagtgttcgagacactggaagagatca |
| | | ccggctatctgtacatcagcgcttggcccgactctctgcccgatctgagcgtgttccagaatctgcaagtcatcagagg |
| | | ccggattctgcacaacggcgcctattctctgacactgcaaggactgggaatcagctggctgggactgcggagtctgaga |
| | | gagctgggcagcggactggcactgatccaccacaacacccatctgtgcttcgtgcacaccgtgccttgggatcagctgt |
| | | tccggaatccccaccaagctctgctgcacaccgccaacagacccgaggatgagtgtgtgggcgaggggctggcttgcca |
| | | tcagctctgtgccagaggacactgttggggacccggccctacccagtgcgtgaactgctcccagtttctgcggggccaa |
| | | gaatgcgtggaagagtgcagagtgctccaaggactgccccgcgagtacgtgaacgccagacactgtctgccatgccacc |
| | | ccgagtgccagcctcagaatggcagcgtgacatgcttcggccccgaggccgatcagtgtgtggcttgcgctcactacaa |
| | | ggaccctcccttctgcgtggcccggtgtccttctggcgtgaaacccgatctgtcctacatgcctatctggaagttcccc |
| | | gacgaggaaggcgcttgtcagccttgccccatcaactgcacccacgaattcacctgcctgctggccagcatggggctgc |
| | | agctgaacctcacctatgagaggaaggacaacacgacggtgacaaggcttctcaacatcaaccccaacaagacctcggc |
| | | cagcgggagctgcggcgcccacctggtgactctggagctgcacagcgagggcaccaccgtcctgctcttccagttcggg |
| | | atgaatgcaagttctagccggtttttcctacaaggaatccagttgaatacaattcttcctgacgccagagaccctgcct |
| | | ttaaagctgccaacggctccctgcgagcgctgcaggccacagtcggcaattcctacaagtgcaacgcggaggagcacgt |
| | | ccgtgtcacgaaggcgttttcagtcaatatattcaaagtgtgggtccaggctttcaaggtggaaggtggccagtttggc |
| | | tctgtggaggagtgtctgctggacgagaacagcatgctgatccccatcgctgtgggtggtgccctggcggggctggtcc |
| | | tcatcgtcctcatcgcctacctcgtcggcaggaagaggagtcacgcaggctaccagactatctagtaaagatctttttc |
| | | cctctgccaaaaattatggggacatcatgaagccccttgagcatctgacttctggctaataaaggaaatttattttcat |
| | | tgcaatagtgtgttggaattttttgtgtctctcactcggaaggacataagggcggccgcggtacccgtgaggctccggt |
| | | gcccgtcagtgggcagagcgcacatcgcccacagtccccgagaagttggggggaggggtcggcaattgaaccggtgcct |
| | | agagaaggtggcgcggggtaaactgggaaagtgatgtcgtgtactggctccgcctttttcccgagggtgggggagaacc |
| | | gtatataagtgcagtagtcgccgtgaacgttctttttcgcaacgggtttgccgccagaacacagtctagaggatccgcc |
| | | gccaccatggagaggaccctggtgtgcctggtggtgatcttcctgggcaccgtggcccacaagagcagccctcaaggcc |
| | | ctgataggctgctgattaggctgaggcacctgatcgacatcgtggagcagctgaagatctacgagaacgacctggaccc |
| | | tgagctgctgagcgcccctcaagacgtgaagggccactgcgagcacgccgccttcgcctgctttcagaaggccaagctg |
| | | aagcctagcaaccctggcaacaacaagaccttcatcatcgacctggtggctcagctgaggaggaggctgcctgctagga |
| | | ggggcggcaagaagcagaagcacatcgccaagtgccctagctgcgacagctacgagaagaggacccctaaggagttcct |
| | | ggagaggctgaagtggctgctgcagaagatgatccatcagcacctgagctgaac |
| Exemplary Murine IL-21 | 217 | MERTLVCLVVIFLGTVAHKSSPQGPDRLLIRLRHLIDIVEQLKIYENDLDPELLSAPQDVKGHCEHAAFACFQKAKLKP |
| Protein Sequence | | SNPGNNKTFIIDLVAQLRRRLPARRGGKKQKHIAKCPSCDSYEKRTPKEFLERLKWLLQKMIHQHLS |
| Exemplary Murine IL-21 | 218 | atggagaggaccctggtgtgcctggtggtgatcttcctgggcaccgtggcccacaagagcagccctcaaggccctgata |
| DNA Sequence | | ggctgctgattaggctgaggcacctgatcgacatcgtggagcagctgaagatctacgagaacgacctggaccctgagct |
| | | gctgagcgcccctcaagacgtgaagggccactgcgagcacgccgccttcgcctgctttcagaaggccaagctgaagcct |
| | | agcaaccctggcaacaacaagaccttcatcatcgacctggtggctcagctgaggaggaggctgcctgctaggaggggcg |
| | | gcaagaagcagaagcacatcgccaagtgccctagctgcgacagctacgagaagaggacccctaaggagttcctggagag |
| | | gctgaagtggctgctgcagaagatgatccatcagcacctgagctga |
| CD80 (Extracellular Domain | 219 | VDEQLSKSVKDKVLLPCRYNSPHEDESEDRIYWQKHDKVVLSVIAGKLKVWPEYKNRTLYDNTTYSLIILGLVLSDRGT |
| +Fc Domain) Protein | | YSCVVQKKERGTYEVKHLALVKLSIKADFSTPNITESGNPSADTKRITCFASGGFPKPRFSWLENGRELPGINTTISQD |
| Sequence | | PESELYTISSQLDFNTTRNHTIKCLIKYGDAHVSEDFTWEKPPEDPPDSKNDIGGGGSGGGGSPRGPTIKPCPPCKCPA |
| | | PNLEGGPSVFIFPPKIKDVLMISLSPIVTCVVVDVSEDDPDVQISWFVNNVEVHTAQTQTHREDYNSTLRVVSALPIQH |
| | | QDWMSGKAFACAVNNKDLPAPIERTISKPKGSVRAPQVYVLPPPEEEMTKKQVTLTCMVTDFMPEDIYVEWTNNGKTEL |
| | | NYKNTEPVLDSDGSYFMYSKLRVEKKNWVERNSYSCSVVHEGLHNHHTTKSFSRTPGK |
| CD80 (Extracellular Domain | 220 | gtggacgagcagctgagcaagagcgtgaaggacaaggtgctgctgccttgtaggtacaacagccctcacgaggacgaga |
| +Fc Domain) DNA | | gcgaggataggatctactggcagaagcacgacaaggtagtgctgagtgtgatagccggcaagctgaaggtgtggcctga |
| Sequence | | gtacaagaataggaccctgtacgacaacaccacctacagcctgatcatcctgggcctggtgctgagcgataggggtacc |
| | | tacagctgcgtggtgcagaagaaggagaggggcacctacgaggtgaagcacctggccctggtgaagctgagcatcaagg |
| | | ccgacttcagcacccctaacatcaccgagagcggcaaccctagcgccgacaccaagaggatcacctgcttcgctagcgg |
| | | cggcttccctaagcctaggttcagctggctggagaacggaagggagctgcctggcataaacacgaccatatctcaagac |
| | | cctgagagcgagctgtacaccatcagctctcagctggacttcaacaccacccgcaatcacaccatcaagtgcctgatca |
| | | agtacggcgacgcccacgtgagcgaggacttcacctgggagaagcctcctgaggaccctcctgacagcaagaacgatat |
| | | cggaggcggaggaagcggaggcggaggaagccccagagggcccacaatcaagccctgtcctccatgcaaatgcccagca |
| | | cctaacctcgagggtggaccatccgtcttcatcttccctccaaagatcaaggatgtactcatgatctccctgagcccca |
| | | tagtcacatgtgtggtggtggatgtgagcgaggatgacccagatgtccagatcagctggtttgtgaacaacgtggaagt |
| | | acacacagctcagacacaaacccatagagaggattacaacagtactctccgggtggtcagtgccctccccatccagcac |
| | | caggactggatgagtggcaaggcgttcgcatgcgcggtcaacaacaaagacctcccagcgcccatcgagagaaccatct |
| | | caaaacccaaagggtcagtaagagctccacaggtatatgtcttgcctccaccagaagaagagatgactaagaaacaggt |
| | | cactctgacctgcatggtcacagacttcatgcctgaagacatttacgtggagtggaccaacaacgggaaaacagagcta |
| | | aactacaagaacactgaaccagtcctggactctgatggttcttacttcatgtacagcaagctgagagtggaaaagaaga |
| | | actgggtggaaagaaatagctactcctgttcagtggtccacgagggtctgcacaatcaccacacgactaagagcttctc |
| | | ccggactccgggtaaa |
| NY-ESO1-LAMP Protein | 221 | MAPRSARRPLLLLLLLLLLGLMHCASAAMFMVKNGNGTACIMANFSAAFSVNYDTKSGPKNMTLDLPSDATVVLNRSSC |
| Sequence | | GKENTSDPSLVIAFGRGHTLTLNFTRNATRYSVQLMSFVYNLSDTHLFPNASSKEIKTVESITDIRADIDKKYRCVSGT |
| | | QVHMNNVTVTLHDATIQAYLSNSSFSRGETRCEQDLEMQAEGRGTGGSTGDADGPGGPGIPDGPGGNAGGPGEAGATGG |
| | | RGPRGAGAARASGPGGGAPRGPHGGAASGLNGCCRCGARGPESRLLEFYLAMPFATPMEAELARRSLAQDAPPLPVPGV |
| | | LLKEFTVSGNILTIRLTAADHRQLQLSISSCLQQQRRTCLLASMGLQLNLTYERKDNTTVTRLLNINPNKTSASGSCGA |
| | | HLVTLELHSEGTTVLLFQFGMNASSSRFFLQGIQLNTILPDARDPAFKAANGSLRALQATVGNSYKCNAEEHVRVTKAF |
| | | SVNIFKVWVQAFKVEGGQFGSVEECLLDENSM |
| NY-ESO1-LAMP-IL-15; | 222 | atggcgccccgcagcgcccggcgacccctgctgctgctactgctgttgctgctgctcggcctcatgcattgtgcgtcag |
| NY-ESO1 +LAMP +EF- | | cagcaatgtttatggtgaaaaatggcaacgggaccgcgtgcataatggccaacttctctgctgccttctcagtgaacta |
| 1alpha +Ig-Kappa +IL-15; | | cgacaccaagagtggccctaagaacatgacccttgacctgccatcagatgccacagtggtgctcaaccgcagctcctgt |
| DNA Sequence | | ggaaaagagaacacttctgaccccagtctcgtgattgcttttggaagaggacatacactcactctcaatttcacgagaa |
| | | atgcaacacgttacagcgtccagctcatgagttttgtttataacttgtcagacacacaccttttccccaatgcgagctc |
| | | caaagaaatcaagactgtggaatctataactgacatcagggcagatatagataaaaaatacagatgtgttagtggcacc |
| | | caggtccacatgaacaacgtgaccgtaacgctccatgatgccaccatccaggcgtacctttccaacagcagcttcagcc |
| | | ggggagagacacgctgtgaacaagacctcgagatgcaagccgaaggcagaggaaccggtggatctactggggatgctga |
| | | tggtcccggtggacccggtattccagatggacccggcggaaatgctggcggtcccggcgaagctggtgctactggtgga |
| | | agaggaccaagaggcgctggtgccgctagagcttctggccccggtggcggagcccctagaggaccacatggcggagctg |
| | | catctggactgaatggctgctgtagatgcggcgccagaggaccagaaagccggctgcttgagttctatctggccatgcc |
| | | tttcgccacaccaatggaagccgagctggctagacgcagtctggcccaagatgctcctccacttccagttccgggcgtg |
| | | ctgctgaaagagtttaccgtcagcggcaacattctgaccatccggctgacagccgccgaccatagacagctgcaactga |
| | | gcatctccagctgcctccagcagcagagaagagaattcacctgcctgctggccagcatggggctgcagctgaacctcac |
| | | ctatgagaggaaggacaacacgacggtgacaaggcttctcaacatcaaccccaacaagacctcggccagcgggagctgc |
| | | ggcgcccacctggtgactctggagctgcacagcgagggcaccaccgtcctgctcttccagttcgggatgaatgcaagtt |
| | | ctagccggtttttcctacaaggaatccagttgaatacaattcttcctgacgccagagaccctgcctttaaagctgccaa |
| | | cggctccctgcgagcgctgcaggccacagtcggcaattcctacaagtgcaacgcggaggagcacgtccgtgtcacgaag |
| | | gcgttttcagtcaatatattcaaagtgtgggtccaggctttcaaggtggaaggtggccagtttggctctgtggaggagt |
| | | gtctgctggacgagaacagcatgctgatccccatcgctgtgggtggtgccctggcggggctggtcctcatcgtcctcat |
| | | cgcctacctcgtcggcaggaagaggagtcacgcaggctaccagactatctagtaaagatctttttccctctgccaaaaa |
| | | ttatggggacatcatgaagccccttgagcatctgacttctggctaataaaggaaatttattttcattgcaatagtgtgt |
| | | tggaattttttgtgtctctcactcggaaggacataagggcggccgcggtacccgtgaggctccggtgcccgtcagtggg |
| | | cagagcgcacatcgcccacagtccccgagaagttggggggaggggtcggcaattgaaccggtgcctagagaaggtggcg |
| | | cggggtaaactgggaaagtgatgtcgtgtactggctccgcctttttcccgagggtgggggagaaccgtatataagtgca |
| | | gtagtcgccgtgaacgttctttttcgcaacgggtttgccgccagaacacagtctagaggatccgccgccaccatggaaa |
| | | ccgatacactgctgctgtgggtgctgttgctctgggttccaggatctacaggcgacaactggatcgacgtccgctacga |
| | | cctggaaaagatcgagagcctgatccagagcatccacatcgacaccacactgtacaccgacagcgactttcaccccagc |
| | | tgcaaagtgaccgccatgaactgctttctgctggaactgcaagtgatcctgcacgagtacagcaacatgaccctgaacg |
| | | agacagtgcggaacgtgctgtacctggccaatagcaccctgagcagcaacaagaacgtggccgagagcggctgcaaaga |
| | | gtgcgaggaactggaagagaaaaccttcaccgagtttctgcagagcttcatccggatcgtgcagatgttcatcaacacc |
| | | agc |
| NY-ESO1 Protein Sequence | 223 | MQAEGRGTGGSTGDADGPGGPGIPDGPGGNAGGPGEAGATGGRGPRGAGAARASGPGGGAPRGPHGGAASGLNGCCRCG |
| | | ARGPESRLLEFYLAMPFATPMEAELARRSLAQDAPPLPVPGVLLKEFTVSGNILTIRLTAADHRQLQLSISSCLQQQRR |
| NY-ESO1 (-tm) DNA | 224 | atgcaagccgaaggcagaggaaccggtggatctactggggatgctgatggtcccggtggacccggtattccagatggac |
| Sequence | | ccggcggaaatgctggcggtcccggcgaagctggtgctactggtggaagaggaccaagaggcgctggtgccgctagagc |
| | | ttctggccccggtggcggagcccctagaggaccacatggcggagctgcatctggactgaatggctgctgtagatgcggc |
| | | gccagaggaccagaaagccggctgcttgagttctatctggccatgcctttcgccacaccaatggaagccgagctggcta |
| | | gacgcagtctggcccaagatgctcctccacttccagttccgggcgtgctgctgaaagagtttaccgtcagcggcaacat |
| | | tctgaccatccggctgacagccgccgaccatagacagctgcaactgagcatctccagctgcctccagcagcagagaaga |
| Ig-kappa and IL-15 protein | 225 | METDTLLLWVLLLWVPGSTGDNWIDVRYDLEKIESLIQSIHIDTTLYTDSDFHPSCKVTAMNCFLLELQVILHEYSNMT |
| sequence | | LNETVRNVLYLANSTLSSNKNVAESGCKECEELEEKTFTEFLQSFIRIVQMFINTS |
| Ig-kappa and IL-15 DNA | 226 | |
| sequence | | |
| ITI-COVID-19 Bicistronic | 227 | SccFIG.28C |
| Vaccine (ITI-Bicistronic-S1- | | |
| LAMP-RBG pA-EF2-S2P | | |
| BHG pA; First Generation | | |
| COVID-19 Vaccine); Protein | | |
| Sequence | | |
| ITI-COVID-19 Bicistronic | 228 | SccFIG.28C |
| Vaccine (ITI-Bicistronic-S1- | | |
| LAMP-RBG pA-EF2-S2P | | |
| BHG pA; First Generation | | |
| COVID-19 Vaccine); DNA | | |
| Sequence | | |
| Spike-LAMP polypeptide | 229 | MAPRSARRPLLLLLLLLLLGLMHCASAAMFMVKNGNGTACIMANFSAAFSVNYDTKSGPKNMTLDLPSDATVVLNRSSC |
| sequence used for Spike- | | GKENTSDPSLVIAFGRGHTLTLNFTRNATRYSVQLMSFVYNLSDTHLFPNASSKEIKTVESITDIRADIDKKYRCVSGT |
| LAMP-sCD40L; SARS-CoV- | | QVHMNNVTVTLHDATIQAYLSNSSFSRGETRCEQDLEVNFTTRTQLPPAYTNSFTRGVYYPDKVFRSSVLHSTQDLFLP |
| 2 B.1.351 Spike (South Africa | | FFSNVTWFHAIHVSGTNGTKRFANPVLPFNDGVYFASTEKSNIIRGWIFGTTLDSKTQSLLIVNNATNVVIKVCEFQFC |
| Variant, without SP and TM) | | NDPFLGVYYHKNNKSWMESEFRVYSSANNCTFEYVSQPFLMDLEGKQGNFKNLREFVFKNIDGYFKIYSKHTPINLVRG |
| +LAMP +EF-1alpha +SPD | | LPQGFSALEPLVDLPIGINITRFQTLHISYLTPGDSSSGWTAGAAAYYVGYLQPRTFLLKYNENGTITDAVDCALDPLS |
| +sCD40L | | ETKCTLKSFTVEKGIYQTSNFRVQPTESIVRFPNITNLCPFGEVFNATRFASVYAWNRKRISNCVADYSVLYNSASFST |
| | | FKCYGVSPTKLNDLCFTNVYADSFVIRGDEVRQIAPGQTGNIADYNYKLPDDFTGCVIAWNSNNLDSKVGGNYNYLYRL |
| | | FRKSNLKPFERDISTEIYQAGSTPCNGVKGFNCYFPLQSYGFQPTYGVGYQPYRVVVLSFELLHAPATVCGPKKSTNLV |
| | | KNKCVNFNFNGLTGTGVLTESNKKFLPFQQFGRDIADTTDAVRDPQTLEILDITPCSFGGVSVITPGTNTSNQVAVLYQ |
| | | GVNCTEVPVAIHADQLTPTWRVYSTGSNVFQTRAGCLIGAEHVNNSYECDIPIGAGICASYQTQTNSPRRARSVASQSI |
| | | IAYTMSLGVENSVAYSNNSIAIPTNFTISVTTEILPVSMTKTSVDCTMYICGDSTECSNLLLQYGSFCTQLNRALTGIA |
| | | VEQDKNTQEVFAQVKQIYKTPPIKDFGGFNFSQILPDPSKPSKRSFIEDLLFNKVTLADAGFIKQYGDCLGDIAARDLI |
| | | CAQKFNGLTVLPPLLTDEMIAQYTSALLAGTITSGWTFGAGAALQIPFAMQMAYRFNGIGVTQNVLYENQKLIANQFNS |
| | | AIGKIQDSLSSTASALGKLQDVVNQNAQALNTLVKQLSSNFGAISSVLNDILSRLDKVEAEVQIDRLITGRLQSLQTYV |
| | | TQQLIRAAEIRASANLAATKMSECVLGQSKRVDFCGKGYHLMSFPQSAPHGVVFLHVTYVPAQEKNFTTAPAICHDGKA |
| | | HFPREGVFVSNGTHWFVTQRNFYEPQIITTDNTFVSGNCDVVIGIVNNTVYDPLQPELDSFKEELDKYFKNHTSPDVDL |
| | | GDISGINASVVNIQKEIDRLNEVAKNLNESLIDLQELGKYEQYIKWPWYCMTSCCSCLKGCCSCGSCCKFDEDDSEPVL |
| | | KGVKLHYTTCLLASMGLQLNLTYERKDNTTVTRLLNINPNKTSASGSCGAHLVTLELHSEGTTVLLFQFGMNASSSRFF |
| | | LQGIQLNTILPDARDPAFKAANGSLRALQATVGNSYKCNAEEHVRVTKAFSVNIFKVWVQAFKVEGGQFGSVEECLLDE |
| | | NSM |
| Spike-LAMP-sCD40L; | 230 | atggcgccccgcagcgcccggcgacccctgctgctgctactgctgttgctgctgctcggcctcatgcattgtgcgtcag |
| SARS-CoV-2 B.1.351 Spike | | cagcaatgtttatggtgaaaaatggcaacgggaccgcgtgcataatggccaacttctctgctgccttctcagtgaacta |
| (South Africa Variant, | | cgacaccaagagtggccctaagaacatgacccttgacctgccatcagatgccacagtggtgctcaaccgcagctcctgt |
| Without SP and TM) | | ggaaaagagaacacttctgaccccagtctcgtgattgcttttggaagaggacatacactcactctcaatttcacgagaa |
| +LAMP +EF-1alpha +SPD | | atgcaacacgttacagcgtccagctcatgagttttgtttataacttgtcagacacacaccttttccccaatgcgagctc |
| +sCD40L; DNA Sequence | | caaagaaatcaagactgtggaatctataactgacatcagggcagatatagataaaaaatacagatgtgttagtggcacc |
| | | caggtccacatgaacaacgtgaccgtaacgctccatgatgccaccatccaggcgtacctttccaacagcagcttcagcc |
| | | ggggagagacacgctgtgaacaagacctcgaggtcaacttcaccaccagaacacagctgcctccagcctacaccaacag |
| | | cttcaccagaggcgtgtactaccccgacaaggtgttcagatccagcgtgctgcactctacccaggacctgttcctgcct |
| | | ttcttcagcaacgtgacctggttccacgccatccacgtgtccggcaccaatggcaccaagagattcgccaatcctgtgc |
| | | tgcccttcaacgacggggtgtactttgccagcaccgagaagtccaacatcatcagaggctggatcttcggcaccacact |
| | | ggacagcaagacccagagcctgctgatcgtgaacaacgccaccaacgtggtcatcaaagtgtgcgagttccagttctgc |
| | | aacgaccccttcctgggcgtctactaccacaagaacaacaagagctggatggaaagcgagttccgggtgtacagcagcg |
| | | ccaacaactgcaccttcgagtacgtgtcccagcctttcctgatggacctggaaggcaagcagggcaacttcaagaacct |
| | | gcgcgagttcgtgttcaagaacatcgacggctacttcaaaatctacagcaagcacacccctatcaacctcgtgcgggga |
| | | ctgcctcagggcttttctgctctggaacccctggtggatctgcccatcggcatcaacatcacccggtttcagaccctgc |
| | | acatcagctacctgacacctggcgatagcagctctggatggacagctggcgccgctgcctactatgtgggatacctgca |
| | | gcctcggaccttcctgctgaagtacaacgagaacggcaccatcaccgacgccgtggattgtgctctggatcctctgagc |
| | | gagacaaagtgcaccctgaagtccttcaccgtggaaaagggcatctaccagaccagcaacttccgggtgcagcccaccg |
| | | aatccatcgtgcggttccccaatatcaccaatctgtgccccttcggcgaggtgttcaatgccaccagattcgcctctgt |
| | | gtacgcctggaaccggaagcggatcagcaattgcgtggccgactactccgtgctgtacaactccgccagcttcagcacc |
| | | ttcaagtgctacggcgtgtcccctaccaagctgaacgacctgtgcttcacaaacgtgtacgccgacagcttcgtgatcc |
| | | ggggagatgaagtgcggcagattgcccctggacagaccggcaatatcgccgactacaactacaagctgcccgacgactt |
| | | caccggctgtgtgattgcctggaacagcaacaacctggactccaaagtcggcggcaactacaattacctgtaccggctg |
| | | ttccggaagtccaatctgaagcccttcgagcgggacatcagcaccgaaatctatcaggccggcagcaccccttgcaatg |
| | | gcgtgaagggctttaactgctacttcccactgcagtcctacggcttccagccaacatacggcgtgggctaccagcctta |
| | | cagagtggtggtgctgagcttcgagctgctgcatgctcctgccacagtgtgcggccctaagaaaagcaccaatctcgtg |
| | | aagaacaaatgcgtgaacttcaacttcaacggcctgaccggcaccggcgtgctgacagagagcaacaagaagttcctgc |
| | | cattccagcagttcggccgggacattgccgataccacagatgctgtcagagatccccagacactggaaatcctggacat |
| | | caccccatgcagcttcggcggagtgtctgtgatcacccctggcaccaacaccagcaatcaggtggcagtgctgtaccag |
| | | ggcgtgaactgtacagaggtgccagtggccattcacgccgatcagctgacccctacttggcgggtgtactccacaggca |
| | | gcaatgtgttccagaccagagccggctgtctgatcggagccgagcacgtgaacaatagctacgagtgcgacatccccat |
| | | cggcgctggcatctgtgccagctatcagacccagacaaacagccccagacgggctagaagtgtggccagccagagcatc |
| | | attgcctacacaatgtctctgggcgtcgagaacagcgtggcctactccaacaactctatcgctatccccaccaatttca |
| | | ccatcagcgtgaccaccgagatcctgcctgtgtccatgaccaagaccagcgtggactgcaccatgtacatctgcggcga |
| | | ttccaccgagtgctccaacctgctgctgcagtacggcagcttctgcacccagctgaatagagccctgacagggatcgcc |
| | | gtggaacaggacaagaacacccaagaggtgttcgcccaagtgaagcaaatctacaagacccctcctatcaaggacttcg |
| | | gcggcttcaatttcagccagattctgcccgatcctagcaagcccagcaagcggagcttcatcgaggacctgctgttcaa |
| | | caaagtgacactggccgacgccggcttcatcaagcagtatggcgattgtctgggcgacattgcagcccgggatctgatt |
| | | tgcgcccagaagtttaacggactgaccgtgctgcctcctctgctgaccgatgagatgatcgcccagtacacatctgccc |
| | | tgctggccggcacaatcacaagcggctggacatttggagctggcgctgccctgcagatcccctttgctatgcagatggc |
| | | ctaccggttcaacggcatcggagtgacccagaatgtgctgtacgagaaccagaagctgatcgccaaccagttcaacagc |
| | | gccatcggcaagatccaggacagcctgagcagcacagccagcgctctgggaaagctgcaggacgtggtcaaccagaatg |
| | | cccaggcactgaacaccctggtcaagcagctgtctagcaacttcggcgccatcagctctgtgctgaacgatatcctgag |
| | | cagactggacaaggtggaagccgaggtgcagatcgacagactgatcaccggaaggctgcagtccctgcagacctacgtt |
| | | acccagcagctgatcagagccgccgagattagagcctctgccaatctggccgccaccaagatgtctgagtgtgtgctgg |
| | | gccagagcaagagagtggacttttgcggcaagggctaccacctgatgagcttccctcagtctgcaccacacggcgtggt |
| | | gtttctgcacgtgacatacgtgcccgctcaagagaagaacttcacaacagcccctgccatctgccacgacggcaaagcc |
| | | cactttcctagagaaggcgtgttcgtgtccaacggcacccattggttcgtgacccagcggaacttctacgagccccaga |
| | | tcatcaccaccgacaacaccttcgtgtctggcaactgcgacgtcgtgatcggcattgtgaacaataccgtgtacgaccc |
| | | tctgcagcccgagctggacagcttcaaagaggaactggataagtactttaagaaccacacaagccccgacgtggacctg |
| | | ggcgatatcagcggaatcaatgccagcgtcgtgaacatccagaaagagatcgaccggctgaacgaggtggccaagaatc |
| | | tgaacgagagcctgatcgacctgcaagaactggggaagtacgagcagtacatcaagtggccttggtactgcatgaccag |
| | | ctgctgtagctgcctgaagggctgttgcagctgtggctcctgctgcaagttcgacgaggacgattctgagcccgtgctg |
| | | aaaggcgtgaagctgcactacacagaattcacctgcctgctggccagcatggggctgcagctgaacctcacctatgaga |
| | | ggaaggacaacacgacggtgacaaggcttctcaacatcaaccccaacaagacctcggccagcgggagctgcggcgccca |
| | | cctggtgactctggagctgcacagcgagggcaccaccgtcctgctcttccagttcgggatgaatgcaagttctagccgg |
| | | tttttcctacaaggaatccagttgaatacaattcttcctgacgccagagaccctgcctttaaagctgccaacggctccc |
| | | tgcgagcgctgcaggccacagtcggcaattcctacaagtgcaacgcggaggagcacgtccgtgtcacgaaggcgttttc |
| | | agtcaatatattcaaagtgtgggtccaggctttcaaggtggaaggtggccagtttggctctgtggaggagtgtctgctg |
| | | gacgagaacagcatgctgatccccatcgctgtgggtggtgccctggcggggctggtcctcatcgtcctcatcgcctacc |
| | | tcgtcggcaggaagaggagtcacgcaggctaccagactatctagtaaagatctttttccctctgccaaaaattatgggg |
| | | acatcatgaagccccttgagcatctgacttctggctaataaaggaaatttattttcattgcaatagtgtgttggaattt |
| | | tttgtgtctctcactcggaaggacataagggcggccgcggtacccgtgaggctccggtgcccgtcagtgggcagagcgc |
| | | acatcgcccacagtccccgagaagttggggggaggggtcggcaattgaaccggtgcctagagaaggtggcgcggggtaa |
| | | actgggaaagtgatgtcgtgtactggctccgcctttttcccgagggtgggggagaaccgtatataagtgcagtagtcgc |
| | | cgtgaacgttctttttcgcaacgggtttgccgccagaacacagtctagaggatccgccgccaccatgctgccctttctc |
| | | tccatgcttgtcttgcttgtacagcccctgggaaatctgggagcagaaatgaagagcctctcgcagagatcagtaccca |
| | | acacctgcaccctagtcatgtgtagcccaacagagaatggcctgcctggtcgtgatggacgggatgggagagaaggtcc |
| | | acggggtgagaagggtgatccaggtttgccaggacctatggggctctcagggttgcagggccctacaggtccagttgga |
| | | cccaaaggagagaatggctctgctggcgaacctggaccaaagggagaacgtggactaagtggacctccaggacttccag |
| | | gtattcctggtccagctgggaaagaaggtccctctgggaagcaggggaacataggacctcaaggcaaaccaggtcctaa |
| | | aggagaggctgggcccaaaggagaagtaggtgctcctggcatgcaaggatctacaggggcaaaaggctccacaggcccc |
| | | aagggagaaagaggtgcccctggtgtgcaaggagccccagggaatgctggagcagcaggacctgccggacctgccggtc |
| | | cacagggagctccaggttccagggggcccccaggactcaagggggacagaggtgttcctggagacagaggaatcaaagg |
| | | tgaaagcgggcttccagacagtgctgctctgaggcagcagatggaggccttaaaaggaaaactacagcgtctagaggtt |
| | | gccttctcccactatcagaaagctgcattgttccctgatggccatagaagattggataaggtcgaagaggaagtaaacc |
| | | ttcatgaagattttgtattcataaaaaagctaaagagatgcaacaaaggagaaggatctttatccttgctgaactgtga |
| | | ggagatgagaaggcaatttgaagaccttgtcaaggatataacgttaaacaaagaagagaaaaaagaaaacagctttgaa |
| | | atgcaaagaggtgatgaggatcctcaaattgcagcacacgttgtaagcgaagccaacagtaatgcagcatccgttctac |
| | | agtgggccaagaaaggatattataccatgaaaagcaacttggtaatgcttgaaaatgggaaacagctgacggttaaaag |
| | | agaaggactctattatgtctacactcaagtcaccttctgctctaatcgggagccttcgagtcaacgcccattcatcgtc |
| | | ggcctctggctgaagcccagcagtggatctgagagaatcttactcaaggggcaaatacccacagttcctcccagcttt |
| | | gcgagcagcagtctgttcacttgggcggagtgtttgaattacaagctggtgcttctgtgtttgtcaacgtgactgaagc |
| | | aagccaagtgatccacagagttggcttctcatcttttggcttactcaaactc |
| SARS-CoV-2 B.1.351 | 231 | VNFTTRTQLPPAYTNSFTRGVYYPDKVFRSSVLHSTQDLFLPFFSNVTWFHAIHVSGTNGTKRFANPVLPFNDGVYFAS |
| SPIKE (South African | | TEKSNIIRGWIFGTTLDSKTQSLLIVNNATNVVIKVCEFQFCNDPFLGVYYHKNNKSWMESEFRVYSSANNCTFEYVSQ |
| Variant (Without SP and | | PFLMDLEGKQGNFKNLREFVFKNIDGYFKIYSKHTPINLVRGLPQGFSALEPLVDLPIGINITRFQTLHISYLTPGDSS |
| TM); Protein Sequence | | SGWTAGAAAYYVGYLQPRTFLLKYNENGTITDAVDCALDPLSETKCTLKSFTVEKGIYQTSNFRVQPTESIVRFPNITN |
| | | LCPFGEVFNATRFASVYAWNRKRISNCVADYSVLYNSASFSTFKCYGVSPTKLNDLCFTNVYADSFVIRGDEVRQIAPG |
| | | QTGNIADYNYKLPDDFTGCVIAWNSNNLDSKVGGNYNYLYRLFRKSNLKPFERDISTEIYQAGSTPCNGVKGFNCYFPL |
| | | QSYGFQPTYGVGYQPYRVVVLSFELLHAPATVCGPKKSTNLVKNKCVNFNFNGLTGTGVLTESNKKFLPFQQFGRDIAD |
| | | TTDAVRDPQTLEILDITPCSFGGVSVITPGTNTSNQVAVLYQGVNCTEVPVAIHADQLTPTWRVYSTGSNVFQTRAGCL |
| | | IGAEHVNNSYECDIPIGAGICASYQTQTNSPRRARSVASQSIIAYTMSLGVENSVAYSNNSIAIPTNFTISVTTEILPV |
| | | SMTKTSVDCTMYICGDSTECSNLLLQYGSFCTQLNRALTGIAVEQDKNTQEVFAQVKQIYKTPPIKDFGGFNFSQILPD |
| | | PSKPSKRSFIEDLLFNKVTLADAGFIKQYGDCLGDIAARDLICAQKFNGLTVLPPLLTDEMIAQYTSALLAGTITSGWT |
| | | FGAGAALQIPFAMQMAYRFNGIGVTQNVLYENQKLIANQFNSAIGKIQDSLSSTASALGKLQDVVNQNAQALNTLVKQL |
| | | SSNFGAISSVLNDILSRLDKVEAEVQIDRLITGRLQSLQTYVTQQLIRAAEIRASANLAATKMSECVLGQSKRVDFCGK |
| | | GYHLMSFPQSAPHGVVFLHVTYVPAQEKNFTTAPAICHDGKAHFPREGVFVSNGTHWFVTQRNFYEPQIITTDNTFVSG |
| | | NCDVVIGIVNNTVYDPLQPELDSFKEELDKYFKNHTSPDVDLGDISGINASVVNIQKEIDRLNEVAKNLNESLIDLQEL |
| | | GKYEQYIKWPWYCMTSCCSCLKGCCSCGSCCKFDEDDSEPVLKGVKLHYT |
| SARS-CoV-2 B.1.351 | 232 | gtgaactttaccacccgcacccagctgccgccggcgtataccaacagctttacccgcggcgtgtattatccggataaag |
| SPIKE (South African | | tgtttcgcagcagcgtgctgcatagcacccaggatctgtttctgccgttttttagcaacgtgacctggtttcatgcgat |
| Variant (Without SP and | | tcatgtgagcggcaccaacggcaccaaacgctttgcgaacccggtgctgccgtttaacgatggcgtgtattttgcgagc |
| TM); DNA Sequence | | accgaaaaaagcaacattattcgcggctggatttttggcaccaccctggatagcaaaacccagagcctgctgattgtga |
| | | acaacgcgaccaacgtggtgattaaagtgtgcgaatttcagttttgcaacgatccgtttctgggcgtgtattatcataa |
| | | aaacaacaaaagctggatggaaagcgaatttcgcgtgtatagcagcgcgaacaactgcacctttgaatatgtgagccag |
| | | ccgtttctgatggatctggaaggcaaacagggcaactttaaaaacctgcgcgaatttgtgtttaaaaacattgatggct |
| | | attttaaaatttatagcaaacataccccgattaacctggtgcgcggcctgccgcagggctttagcgcgctggaaccgct |
| | | ggtggatctgccgattggcattaacattacccgctttcagaccctgcatattagctatctgaccccgggcgatagcagc |
| | | agcggctggaccgcgggcgcggcggcgtattatgtgggctatctgcagccgcgcacctttctgctgaaatataacgaaa |
| | | acggcaccattaccgatgcggtggattgcgcgctggatccgctgagcgaaaccaaatgcaccctgaaaagctttaccgt |
| | | ggaaaaaggcatttatcagaccagcaactttcgcgtgcagccgaccgaaagcattgtgcgctttccgaacattaccaac |
| | | ctgtgcccgtttggcgaagtgtttaacgcgacccgctttgcgagcgtgtatgcgtggaaccgcaaacgcattagcaact |
| | | gcgtggcggattatagcgtgctgtataacagcgcgagctttagcacctttaaatgctatggcgtgagcccgaccaaact |
| | | gaacgatctgtgctttaccaacgtgtatgcggatagctttgtgattcgcggcgatgaagtgcgccagattgcgccgggc |
| | | cagaccggcaacattgcggattataactataaactgccggatgattttaccggctgcgtgattgcgtggaacagcaaca |
| | | acctggatagcaaagtgggcggcaactataactatctgtatcgcctgtttcgcaaaagcaacctgaaaccgtttgaacg |
| | | cgatattagcaccgaaatttatcaggcgggcagcaccccgtgcaacggcgtgaaaggctttaactgctattttccgctg |
| | | cagagctatggctttcagccgacctatggcgtgggctatcagccgtatcgcgtggtggtgctgagctttgaactgctgc |
| | | atgcgccggcgaccgtgtgcggcccgaaaaaaagcaccaacctggtgaaaaacaaatgcgtgaactttaactttaacgg |
| | | cctgaccggcaccggcgtgctgaccgaaagcaacaaaaaatttctgccgtttcagcagtttggccgcgatattgcggat |
| | | accaccgatgcggtgcgcgatccgcagaccctggaaattctggatattaccccgtgcagctttggcggcgtgagcgtga |
| | | ttaccccgggcaccaacaccagcaaccaggtggcggtgctgtatcagggcgtgaactgcaccgaagtgccggtggcgat |
| | | tcatgcggatcagctgaccccgacctggcgcgtgtatagcaccggcagcaacgtgtttcagacccgcgcgggctgcctg |
| | | attggcgcggaacatgtgaacaacagctatgaatgcgatattccgattggcgcgggcatttgcgcgagctatcagaccc |
| | | agaccaacagcccgcgccgcgcgcgcagcgtggcgagccagagcattattgcgtataccatgagcctgggcgtggaaaa |
| | | cagcgtggcgtatagcaacaacagcattgcgattccgaccaactttaccattagcgtgaccaccgaaattctgccggtg |
| | | agcatgaccaaaaccagcgtggattgcaccatgtatatttgcggcgatagcaccgaatgcagcaacctgctgctgcagt |
| | | atggcagcttttgcacccagctgaaccgcgcgctgaccggcattgcggtggaacaggataaaaacacccaggaagtgtt |
| | | tgcgcaggtgaaacagatttataaaaccccgccgattaaagattttggcggctttaactttagccagattctgccggat |
| | | ccgagcaaaccgagcaaacgcagctttattgaagatctgctgtttaacaaagtgaccctggcggatgcgggctttatta |
| | | aacagtatggcgattgcctgggcgatattgcggcgcgcgatctgatttgcgcgcagaaatttaacggcctgaccgtgct |
| | | gccgccgctgctgaccgatgaaatgattgcgcagtataccagcgcgctgctggcgggcaccattaccagcggctggacc |
| | | tttggcgcgggcgcggcgctgcagattccgtttgcgatgcagatggcgtatcgctttaacggcattggcgtgacccaga |
| | | acgtgctgtatgaaaaccagaaactgattgcgaaccagtttaacagcgcgattggcaaaattcaggatagcctgagcag |
| | | caccgcgagcgcgctgggcaaactgcaggatgtggtgaaccagaacgcgcaggcgctgaacaccctggtgaaacagctg |
| | | agcagcaactttggcgcgattagcagcgtgctgaacgatattctgagccgcctggataaagtggaagcggaagtgcaga |
| | | ttgatcgcctgattaccggccgcctgcagagcctgcagacctatgtgacccagcagctgattcgcgcggcggaaattcg |
| | | cgcgagcgcgaacctggcggcgaccaaaatgagcgaatgcgtgctgggccagagcaaacgcgtggatttttgcggcaaa |
| | | ggctatcatctgatgagctttccgcagagcgcgccgcatggcgtggtgtttctgcatgtgacctatgtgccggcgcagg |
| | | aaaaaaactttaccaccgcgccggcgatttgccatgatggcaaagcgcattttccgcgcgaaggcgtgtttgtgagcaa |
| | | cggcacccattggtttgtgacccagcgcaacttttatgaaccgcagattattaccaccgataacacctttgtgagcggc |
| | | aactgcgatgtggtgattggcattgtgaacaacaccgtgtatgatccgctgcagccggaactggatagctttaaagaag |
| | | aactggataaatattttaaaaaccataccagcccggatgtggatctgggcgatattagcggcattaacgcgagcgtggt |
| | | gaacattcagaaagaaattgatcgcctgaacgaagtggcgaaaaacctgaacgaaagcctgattgatctgcaggaactg |
| | | ggcaaatatgaacagtatattaaatggccgtggtattgcatgaccagctgctgcagctgcctgaaaggctgctgcagct |
| | | gcggcagctgctgcaaatttgatgaagatgatagcgaaccggtgctgaaaggcgtgaaactgcattatacc |
| SPD-xCD40L protein | 233 | MLPFLSMLVLLVQPLGNLGAEMKSLSQRSVPNTCTLVMCSPTENGLPGRDGRDGREGPRGEKGDPGLPGPMGLSGLQGP |
| sequence | | TGPVGPKGENGSAGEPGPKGERGLSGPPGLPGIPGPAGKEGPSGKQGNIGPQGKPGPKGEAGPKGEVGAPGMQGSTGAK |
| | | GSTGPKGERGAPGVQGAPGNAGAAGPAGPAGPQGAPGSRGPPGLKGDRGVPGDRGIKGESGLPDSAALRQQMEALKGKL |
| | | QRLEVAFSHYQKAALFPDGHRRLDKVEEEVNLHEDFVFIKKLKRCNKGEGSLSLLNCEEMRRQFEDLVKDITLNKEEKK |
| | | ENSFEMQRGDEDPQIAAHVVSEANSNAASVLQWAKKGYYTMKSNLVMLENGKQLTVKREGLYYVYTQVTFCSNREPSSQ |
| | | RPFIVGLWLKPSSGSERILLKAANTHSSSQLCEQQSVHLGGVFELQAGASVFVNVTEASQVIHRVGFSSFGLLKL |
| CD161-LAMP Protein | 234 | MAPRSARRPLLLLLLLLLLGLMHCASAAMFMVKNGNGTACIMANFSAAFSVNYDTKSGPKNMTLDLPSDATVVLNRSSC |
| Sequence | | GKENTSDPSLVIAFGRGHTLTLNFTRNATRYSVQLMSFVYNLSDTHLFPNASSKEIKTVESITDIRADIDKKYRCVSGT |
| | | QVHMNNVTVTLHDATIQAYLSNSSFSRGETRCEQDLEQKSSIEKCSVDIQQSRNKTTERPGLLNCPIYWQQLREKCLLF |
| | | SHTVNPWNNSLADCSTKESSLLLIRDKDELIHTQNLIRDKAILFWIGLNFSLSEKNWKWINGSFLNSNDLEIRGDAKEN |
| | | SCISISQTSVYSEYCSTEIRWICQKELTPVRNKVYPDSTCLLASMGLQLNLTYERKDNTTVTRLLNINPNKTSASGSCG |
| | | AHLVTLELHSEGTTVLLFQFGMNASSSRFFLQGIQLNTILPDARDPAFKAANGSLRALQATVGNSYKCNAEEHVRVTKA |
| | | FSVNIFKVWVQAFKVEGGQFGSVEECLLDENSM |
| CD161-LAMP-sCD40L; | 235 | atggcgccccgcagcgcccggcgacccctgctgctgctactgctgttgctgctgctcggcctcatgcattgtgcgtcag |
| CD161 (Extracellular | | cagcaatgtttatggtgaaaaatggcaacgggaccgcgtgcataatggccaacttctctgctgccttctcagtgaacta |
| Domain) +LAMP +EF- | | cgacaccaagagtggccctaagaacatgacccttgacctgccatcagatgccacagtggtgctcaaccgcagctcctgt |
| 1 alpha +SPD +sCD40L; | | ggaaaagagaacacttctgaccccagtctcgtgattgcttttggaagaggacatacactcactctcaatttcacgagaa |
| DNA Sequence | | atgcaacacgttacagcgtccagctcatgagttttgtttataacttgtcagacacacaccttttccccaatgcgagctc |
| | | caaagaaatcaagactgtggaatctataactgacatcagggcagatatagataaaaaatacagatgtgttagtggcacc |
| | | caggtccacatgaacaacgtgaccgtaacgctccatgatgccaccatccaggcgtacctttccaacagcagcttcagcc |
| | | ggggagagacacgctgtgaacaagacctcgagcagaagtccagcatcgagaagtgcagcgtggacatccagcagagccg |
| | | gaacaagaccaccgaaagacccggcctgctgaactgccctatctactggcagcagctgagagagaagtgcctgctgttc |
| | | agccacaccgtgaatccctggaacaacagcctggccgactgcagcacaaaagagagcagcctgctgctgatcagagaca |
| | | aggacgagctgatccacacacagaacctgatccgcgacaaggccatcctgttctggatcggcctgaacttcagcctgag |
| | | cgagaagaactggaagtggatcaacggcagcttcctgaactccaacgacctggaaatccggggcgacgccaaagagaac |
| | | agctgcatcagcatcagccagaccagcgtgtacagcgagtactgctccaccgagatcagatggatctgccagaaagaac |
| | | tgacccctgtgcggaacaaggtgtaccccgattctgaattcacctgcctgctggccagcatggggctgcagctgaacct |
| | | cacctatgagaggaaggacaacacgacggtgacaaggcttctcaacatcaaccccaacaagacctcggccagcgggagc |
| | | tgcggcgcccacctggtgactctggagctgcacagcgagggcaccaccgtcctgctcttccagttcgggatgaatgcaa |
| | | gttctagccggtttttcctacaaggaatccagttgaatacaattcttcctgacgccagagaccctgcctttaaagctgc |
| | | caacggctccctgcgagcgctgcaggccacagtcggcaattcctacaagtgcaacgcggaggagcacgtccgtgtcacg |
| | | aaggcgttttcagtcaatatattcaaagtgtgggtccaggctttcaaggtggaaggtggccagtttggctctgtggagg |
| | | agtgtctgctggacgagaacagcatgctgatccccatcgctgtgggtggtgccctggcggggctggtcctcatcgtcct |
| | | catcgcctacctcgtcggcaggaagaggagtcacgcaggctaccagactatctagtaaagatctttttccctctgccaa |
| | | aaattatggggacatcatgaagccccttgagcatctgacttctggctaataaaggaaatttattttcattgcaatagtg |
| | | tgttggaattttttgtgtctctcactcggaaggacataagggcggccgcggtacccgtgaggctccggtgcccgtcagt |
| | | gggcagagcgcacatcgcccacagtccccgagaagttggggggaggggtcggcaattgaaccggtgcctagagaaggtg |
| | | gcgcggggtaaactgggaaagtgatgtcgtgtactggctccgcctttttcccgagggtgggggagaaccgtatataagt |
| | | gcagtagtcgccgtgaacgttctttttcgcaacgggtttgccgccagaacacagtctagaggatccgccgccaccatgg |
| | | aaaccgatacactgctgctgtgggtgctgttgctctgggttccaggatctacaggcgacatgctgccctttctctccat |
| | | gcttgtcttgcttgtacagcccctgggaaatctgggagcagaaatgaagagcctctcgcagagatcagtacccaacacc |
| | | tgcaccctagtcatgtgtagcccaacagagaatggcctgcctggtcgtgatggacgggatgggagagaaggtccacggg |
| | | gtgagaagggtgatccaggtttgccaggacctatggggctctcagggttgcagggccctacaggtccagttggacccaa |
| | | aggagagaatggctctgctggcgaacctggaccaaagggagaacgtggactaagtggacctccaggacttccaggtatt |
| | | cctggtccagctgggaaagaaggtccctctgggaagcaggggaacataggacctcaaggcaaaccaggtcctaaaggag |
| | | aggctgggcccaaaggagaagtaggtgctcctggcatgcaaggatctacaggggcaaaaggctccacaggccccaaggg |
| | | agaaagaggtgcccctggtgtgcaaggagccccagggaatgctggagcagcaggacctgccggacctgccggtccacag |
| | | ggagctccaggttccagggggcccccaggactcaagggggacagaggtgttcctggagacagaggaatcaaaggtgaaa |
| | | gcgggcttccagacagtgctgctctgaggcagcagatggaggccttaaaaggaaaactacagcgtctagaggttgcctt |
| | | ctcccactatcagaaagctgcattgttccctgatggccatagaagattggataaggtcgaagaggaagtaaaccttcat |
| | | gaagattttgtattcataaaaaagctaaagagatgcaacaaaggagaaggatctttatccttgctgaactgtgaggaga |
| | | tgagaaggcaatttgaagaccttgtcaaggatataacgttaaacaaagaagagaaaaaagaaaacagctttgaaatgca |
| | | aagaggtgatgaggatcctcaaattgcagcacacgttgtaagcgaagccaacagtaatgcagcatccgttctacagtgg |
| | | gccaagaaaggatattataccatgaaaagcaacttggtaatgcttgaaaatgggaaacagctgacggttaaaagagaag |
| | | gactctattatgtctacactcaagtcaccttctgctctaatcgggagccttcgagtcaacgcccattcatcgtcggcct |
| | | ctggctgaagcccagcagtggatctgagagaatcttactcaaggcggcaaatacccacagttcctcccagctttgcgag |
| | | cagcagtctgttcacttgggcggagtgtttgaattacaagctggtgcttctgtgtttgtcaacgtgactgaagcaagcc |
| | | aagtgatccacagagttggcttctcatcttttggcttactcaaactc |
| CD161 (Extracellular | 236 | QKSSIEKCSVDIQQSRNKTTERPGLLNCPIYWQQLREKCLLFSHTVNPWNNSLADCSTKESSLLLIRDKDELIHTQNLI |
| Domain) Protein Sequence | | RDKAILFWIGLNFSLSEKNWKWINGSFLNSNDLEIRGDAKENSCISISQTSVYSEYCSTEIRWICQKELTPVRNKVYPD |
| | | S |
| CD161 (Extracellular | 237 | cagaagtccagcatcgagaagtgcagcgtggacatccagcagagccggaacaagaccaccgaaagacccggcctgctga |
| Domain) DNA Sequence | | actgccctatctactggcagcagctgagagagaagtgcctgctgttcagccacaccgtgaatccctggaacaacagcct |
| | | ggccgactgcagcacaaaagagagcagcctgctgctgatcagagacaaggacgagctgatccacacacagaacctgatc |
| | | cgcgacaaggccatcctgttctggatcggcctgaacttcagcctgagcgagaagaactggaagtggatcaacggcagct |
| | | tcctgaactccaacgacctggaaatccggggcgacgccaaagagaacagctgcatcagcatcagccagaccagcgtgta |
| | | cagcgagtactgctccaccgagatcagatggatctgccagaaagaactgacccctgtgcggaacaaggtgtaccccgat |
| | | tct |
| SPD-sCD40L protein | 238 | MLPFLSMLVLLVQPLGNLGAEMKSLSQRSVPNTCTLVMCSPTENGLPGRDGRDGREGPRGEKGDPGLPGPMGLSGLQGP |
| sequence | | TGPVGPKGENGSAGEPGPKGERGLSGPPGLPGIPGPAGKEGPSGKQGNIGPQGKPGPKGEAGPKGEVGAPGMQGSTGAK |
| | | GSTGPKGERGAPGVQGAPGNAGAAGPAGPAGPQGAPGSRGPPGLKGDRGVPGDRGIKGESGLPDSAALRQQMEALKGKL |
| | | QRLEVAFSHYQKAALFPDGHRRLDKVEEEVNLHEDFVFIKKLKRCNKGEGSLSLLNCEEMRRQFEDLVKDITLNKEEKK |
| | | ENSFEMQRGDEDPQIAAHVVSEANSNAASVLQWAKKGYYTMKSNLVMLENGKQLTVKREGLYYVYTQVTFCSNREPSSQ |
| | | RPFIVGLWLKPSSGSERILLKAANTHSSSQLCEQQSVHLGGVFELQAGASVFVNVTEASQVIHRVGFSSFGLLKL |
| SPD-sCD40L DNA | 239 | atgctgccctttctctccatgcttgtcttgcttgtacagcccctgggaaatctgggagcagaaatgaagagcctctcgc |
| sequence | | agagatcagtacccaacacctgcaccctagtcatgtgtagcccaacagagaatggcctgcctggtcgtgatggacggga |
| | | tgggagagaaggtccacggggtgagaagggtgatccaggtttgccaggacctatggggctctcagggttgcagggccct |
| | | acaggtccagttggacccaaaggagagaatggctctgctggcgaacctggaccaaagggagaacgtggactaagtggac |
| | | ctccaggacttccaggtattcctggtccagctgggaaagaaggtccctctgggaagcaggggaacataggacctcaagg |
| | | caaaccaggtcctaaaggagaggctgggcccaaaggagaagtaggtgctcctggcatgcaaggatctacaggggcaaaa |
| | | ggctccacaggccccaagggagaaagaggtgcccctggtgtgcaaggagccccagggaatgctggagcagcaggacctg |
| | | ccggacctgccggtccacagggagctccaggttccagggggcccccaggactcaagggggacagaggtgttcctggaga |
| | | cagaggaatcaaaggtgaaagcgggcttccagacagtgctgctctgaggcagcagatggaggccttaaaaggaaaacta |
| | | cagcgtctagaggttgccttctcccactatcagaaagctgcattgttccctgatggccatagaagattggataaggtcg |
| | | aagaggaagtaaaccttcatgaagattttgtattcataaaaaagctaaagagatgcaacaaaggagaaggatctttatc |
| | | cttgctgaactgtgaggagatgagaaggcaatttgaagaccttgtcaaggatataacgttaaacaaagaagagaaaaaa |
| | | gaaaacagctttgaaatgcaaagaggtgatgaggatcctcaaattgcagcacacgttgtaagcgaagccaacagtaatg |
| | | cagcatccgttctacagtgggccaagaaaggatattataccatgaaaagcaacttggtaatgcttgaaaatgggaaaca |
| | | gctgacggttaaaagagaaggactctattatgtctacactcaagtcaccttctgctctaatcgggagccttcgagtcaa |
| | | cgcccattcatcgtcggcctctggctgaagcccagcagtggatctgagagaatcttactcaaggcggcaaatacccaca |
| | | gttcctcccagctttgcgagcagcagtctgttcacttgggcggagtgtttgaattacaagctggtgcttctgtgtttgt |
| | | caacgtgactgaagcaagccaagtgatccacagagttggcttctcatcttttggcttactcaaactctga |
| | 240 | |
| HER2-LAMP-OX40L; | 241 | atggcgccccgcagcgcccggcgacccctgctgctgctactgctgttgctgctgctcggcctcatgcattgtgcgtcag |
| HER2 (Extracellular | | cagcaatgtttatggtgaaaaatggcaacgggaccgcgtgcataatggccaacttctctgctgccttctcagtgaacta |
| Domain) +LAMP +EF- | | cgacaccaagagtggccctaagaacatgacccttgacctgccatcagatgccacagtggtgctcaaccgcagctcctgt |
| 1alpha +IL-2 SP +OX40L | | ggaaaagagaacacttctgaccccagtctcgtgattgcttttggaagaggacatacactcactctcaatttcacgagaa |
| (Extracellular Domain +Fc | | atgcaacacgttacagcgtccagctcatgagttttgtttataacttgtcagacacacaccttttccccaatgcgagctc |
| Domain); DNA Sequence | | caaagaaatcaagactgtggaatctataactgacatcagggcagatatagataaaaaatacagatgtgttagtggcacc |
| | | caggtccacatgaacaacgtgaccgtaacgctccatgatgccaccatccaggcgtacctttccaacagcagcttcagcc |
| | | ggggagagacacgctgtgaacaagacctcgagacacaagtctgcaccggcaccgacatgaagctgagactgcccgccag |
| | | ccccgaaacccatctggacatgctgcggcatctgtaccaaggctgtcaagtggtgcaaggcaatctggaactgacatat |
| | | ctgcctaccaacgcctctctgagctttctgcaagacatccaagaagtgcaaggctacgtcctcattgcccacaaccaag |
| | | tccgacaagtgccactgcagcggctgagaatcgtgcggggcacccagctgttcgaggacaactatgctctggccgtgct |
| | | ggacaacggcgaccctctgaacaacaccacaccagtgactggcgcctctcccggcggactgagagaactgcagctgcgc |
| | | tctctgaccgagattctgaagggcggcgtgctgatccagcggaaccctcagctgtgctaccaagacaccattctgtgga |
| | | aggacatcttccacaagaacaaccagctggctctgaccctcatcgacaccaacagaagccgggcttgccacccttgcag |
| | | ccccatgtgcaagggcagcagatgttggggcgagagcagcgaggactgccagtctctgaccagaaccgtgtgtgccggc |
| | | ggatgcgccagatgcaagggccctctgccaaccgattgctgccacgaacagtgcgccgctggctgtaccggccccaagc |
| | | acagcgattgtctggcttgtctgcacttcaaccactccggcatctgcgagctgcactgcccagccctcgtgacatacaa |
| | | caccgacacattcgagagcatgcccaaccccgagggcagatacacattcggcgccagctgtgtgaccgcttgcccctac |
| | | aactatctgagcaccgacgtgggcagctgcaccctcgtgtgccctctgcacaatcaagaagtgaccgccgaggacggca |
| | | cccagagatgcgagaagtgctccaagccttgcgccagagtgtgctacggactgggcatggaacatctgcgggaagtgcg |
| | | ggccgtgaccagcgccaatatccaagaatttgccggctgcaagaagattttcggcagtctggcctttctgcccgagagc |
| | | ttcgatggcgaccccgcctctaacacagcccctctgcagccagagcagctccaagtgttcgagacactggaagagatca |
| | | ccggctatctgtacatcagcgcttggcccgactctctgcccgatctgagcgtgttccagaatctgcaagtcatcagagg |
| | | ccggattctgcacaacggcgcctattctctgacactgcaaggactgggaatcagctggctgggactgcggagtctgaga |
| | | gagctgggcagcggactggcactgatccaccacaacacccatctgtgcttcgtgcacaccgtgccttgggatcagctgt |
| | | tccggaatccccaccaagctctgctgcacaccgccaacagacccgaggatgagtgtgtgggcgaggggctggcttgcca |
| | | tcagctctgtgccagaggacactgttggggacccggccctacccagtgcgtgaactgctcccagtttctgcggggccaa |
| | | gaatgcgtggaagagtgcagagtgctccaaggactgccccgcgagtacgtgaacgccagacactgtctgccatgccacc |
| | | ccgagtgccagcctcagaatggcagcgtgacatgcttcggccccgaggccgatcagtgtgtggcttgcgctcactacaa |
| | | ggaccctcccttctgcgtggcccggtgtccttctggcgtgaaacccgatctgtcctacatgcctatctggaagttcccc |
| | | gacgaggaaggcgcttgtcagccttgccccatcaactgcacccacgaattcacctgcctgctggccagcatggggctgc |
| | | agctgaacctcacctatgagaggaaggacaacacgacggtgacaaggcttctcaacatcaaccccaacaagacctcggc |
| | | cagcgggagctgcggcgcccacctggtgactctggagctgcacagcgagggcaccaccgtcctgctcttccagttcggg |
| | | atgaatgcaagttctagccggtttttcctacaaggaatccagttgaatacaattcttcctgacgccagagaccctgcct |
| | | ttaaagctgccaacggctccctgcgagcgctgcaggccacagtcggcaattcctacaagtgcaacgcggaggagcacgt |
| | | ccgtgtcacgaaggcgttttcagtcaatatattcaaagtgtgggtccaggctttcaaggtggaaggtggccagtttggc |
| | | tctgtggaggagtgtctgctggacgagaacagcatgctgatccccatcgctgtgggtggtgccctggcggggctggtcc |
| | | tcatcgtcctcatcgcctacctcgtcggcaggaagaggagtcacgcaggctaccagactatctagtaaagatctttttc |
| | | cctctgccaaaaattatggggacatcatgaagccccttgagcatctgacttctggctaataaaggaaatttattttcat |
| | | tgcaatagtgtgttggaattttttgtgtctctcactcggaaggacataagggcggccgcggtacccgtgaggctccggt |
| | | gcccgtcagtgggcagagcgcacatcgcccacagtccccgagaagttggggggaggggtcggcaattgaaccggtgcct |
| | | agagaaggtggcgcggggtaaactgggaaagtgatgtcgtgtactggctccgcctttttcccgagggtgggggagaacc |
| | | gtatataagtgcagtagtcgccgtgaacgttctttttcgcaacgggtttgccgccagaacacagtctagaggatccgcc |
| | | gccaccatgtacaggatgcaactcctgtcttgcattgcactaagtcttgcacttgtcacgaattcgataagcagcagcc |
| | | ctgccaaggaccctcctattcagaggctgaggggcgccgtgacaaggtgcgaggacggacagctgttcatcagcagcta |
| | | caagaacgagtatcagaccatggaggtgcagaacaacagcgtggtgatcaagtgcgacggcctgtacatcatctacctg |
| | | aagggcagcttcttccaagaggtgaagatcgacctgcactttagggaggaccacaaccctatcagcatccctatgctga |
| | | acgacggaaggaggatcgtgttcaccgtggtggctagcctggccttcaaggacaaggtgtacctgaccgtgaacgcccc |
| | | tgacaccctgtgcgagcacctgcagatcaacgacggcgagctgatcgtggtgcagctgacccctggctactgcgcccct |
| | | gagggcagctaccacagcaccgtgaaccaagtgcctctggatatcggaggcggaggaagcggaggcggaggaagcccca |
| | | gagggcccacaatcaagccctgtcctccatgcaaatgcccagcacctaacctcgagggtggaccatccgtcttcatctt |
| | | ccctccaaagatcaaggatgtactcatgatctccctgagccccatagtcacatgtgtggtggtggatgtgagcgaggat |
| | | gacccagatgtccagatcagctggtttgtgaacaacgtggaagtacacacagctcagacacaaacccatagagaggatt |
| | | acaacagtactctccgggtggtcagtgccctccccatccagcaccaggactggatgagtggcaaggcgttcgcatgcgc |
| | | ggtcaacaacaaagacctcccagcgcccatcgagagaaccatctcaaaacccaaagggtcagtaagagctccacaggta |
| | | tatgtcttgcctccaccagaagaagagatgactaagaaacaggtcactctgacctgcatggtcacagacttcatgcctg |
| | | aagacatttacgtggagtggaccaacaacgggaaaacagagctaaactacaagaacactgaaccagtcctggactctga |
| | | tggttcttacttcatgtacagcaagctgagagtggaaaagaagaactgggtggaaagaaatagctactcctgttcagtg |
| | | gtccacgagggtctgcacaatcaccacacgactaagagcttctcccggactccgggtaaa |
| IL-2 SP-OX40L extracellular | 242 | MYRMQLLSCIALSLALVINSSSSPAKDPPIQRLRGAVTRCEDGQLFISSYKNEYQTMEVQNNSVVIKCDGLYIIYLKGS |
| domain-Fc protein | | FFQEVKIDLHFREDHNPISIPMLNDGRRIVFTVVASLAFKDKVYLTVNAPDTLCEHLQINDGELIVVQLTPGYCAPEGS |
| sequence | | YHSTVNQVPLDIGGGGSGGGGSPRGPTIKPCPPCKCPAPNLEGGPSVFIFPPKIKDVLMISLSPIVTCVVVDVSEDDPD |
| | | VQISWFVNNVEVHTAQTQTHREDYNSTLRVVSALPIQHQDWMSGKAFACAVNNKDLPAPIERTISKPKGSVRAPQVYVL |
| | | PPPEEEMTKKQVTLTCMVTDFMPEDIYVEWTNNGKTELNYKNTEPVLDSDGSYFMYSKLRVEKKNWVERNSYSCSVVHE |
| | | GLHNHHTTKSFSRTPGK |
| OX40L (Extracellular | 243 | SSSPAKDPPIQRLRGAVTRCEDGQLFISSYKNEYQTMEVQNNSVVIKCDGLYIIYLKGSFFQEVKIDLHFREDHNPISI |
| Domain + Fc Domain) | | PMLNDGRRIVFTVVASLAFKDKVYLTVNAPDTLCEHLQINDGELIVVQLTPGYCAPEGSYHSTVNQVPLDIGGGGSGGG |
| Protein Sequence | | GSPRGPTIKPCPPCKCPAPNLEGGPSVFIFPPKIKDVLMISLSPIVTCVVVDVSEDDPDVQISWFVNNVEVHTAQTQTH |
| | | REDYNSTLRVVSALPIQHQDWMSGKAFACAVNNKDLPAPIERTISKPKGSVRAPQVYVLPPPEEEMTKKQVTLTCMVTD |
| | | FMPEDIYVEWTNNGKTELNYKNTEPVLDSDGSYFMYSKLRVEKKNWVERNSYSCSVVHEGLHNHHTTKSFSRTPGK |
| OX40L (Extracellular | 244 | agcagcagccctgccaaggaccctcctattcagaggctgaggggcgccgtgacaaggtgcgaggacggacagctgttca |
| Domain + Fc Domain) DNA | | tcagcagctacaagaacgagtatcagaccatggaggtgcagaacaacagcgtggtgatcaagtgcgacggcctgtacat |
| Sequence | | catctacctgaagggcagcttcttccaagaggtgaagatcgacctgcactttagggaggaccacaaccctatcagcatc |
| | | cctatgctgaacgacggaaggaggatcgtgttcaccgtggtggctagcctggccttcaaggacaaggtgtacctgaccg |
| | | tgaacgcccctgacaccctgtgcgagcacctgcagatcaacgacggcgagctgatcgtggtgcagctgacccctggcta |
| | | ctgcgcccctgagggcagctaccacagcaccgtgaaccaagtgcctctggatatcggaggcggaggaagcggaggcgga |
| | | ggaagccccagagggcccacaatcaagccctgtcctccatgcaaatgcccagcacctaacctcgagggtggaccatccg |
| | | tcttcatcttccctccaaagatcaaggatgtactcatgatctccctgagccccatagtcacatgtgtggtggtggatgt |
| | | gagcgaggatgacccagatgtccagatcagctggtttgtgaacaacgtggaagtacacacagctcagacacaaacccat |
| | | agagaggattacaacagtactctccgggtggtcagtgccctccccatccagcaccaggactggatgagtggcaaggcgt |
| | | tcgcatgcgcggtcaacaacaaagacctcccagcgcccatcgagagaaccatctcaaaacccaaagggtcagtaagagc |
| | | tccacaggtatatgtcttgcctccaccagaagaagagatgactaagaaacaggtcactctgacctgcatggtcacagac |
| | | ttcatgcctgaagacatttacgtggagtggaccaacaacgggaaaacagagctaaactacaagaacactgaaccagtcc |
| | | tggactctgatggttcttacttcatgtacagcaagctgagagtggaaaagaagaactgggtggaaagaaatagctactc |
| | | ctgttcagtggtccacgagggtctgcacaatcaccacacgactaagagcttctcccggactccgggtaaa |
| EF-1alpha core promoter | 245 | gggcagagcgcacatcgcccacagtccccgagaagttggggggaggggtcggcaattgaaccggtgcctagagaaggtg |
| | | gcgcggggtaaactgggaaagtgatgtcgtgtactggctccgcctttttcccgagggtgggggagaaccgtatataagt |
| | | gcagtagtcgccgtgaacgttctttttcgcaacgggtttgccgccagaacacag |
| Murine IL-2 signal peptide | 246 | MYRMQLLSCIALSLALVINS |
| (SP) amino acid sequence | | |
| Murine IL-2 SP nucleotide | 247 | atgtacaggatgcaactcctgtcttgcattgcactaagtcttgcacttgtcacgaattcg |
| sequence | | |
| Human IL-2 SP amino acid | 248 | MYRMQLLSCIALSLALVINS |
| sequence | | |
| Human IL-2 SP nucleotide | 249 | atgtacaggatgcaactcctgtcttgcattgcactaagtcttgcacttgtcacaaacagt |
| sequence | | |
| | 250 | |
| HER2-LAMP-CD80; HER2 | 251 | atggcgccccgcagcgcccggcgacccctgctgctgctactgctgttgctgctgctcggcctcatgcattgtgcgtcag |
| (Extracellular Domain) | | cagcaatgtttatggtgaaaaatggcaacgggaccgcgtgcataatggccaacttctctgctgccttctcagtgaacta |
| +LAMP +EF-1alpha +IL-2 | | cgacaccaagagtggccctaagaacatgacccttgacctgccatcagatgccacagtggtgctcaaccgcagctcctgt |
| SP +CD80 (Extracellular | | ggaaaagagaacacttctgaccccagtctcgtgattgcttttggaagaggacatacactcactctcaatttcacgagaa |
| Domain +Fc Domain); DNA | | atgcaacacgttacagcgtccagctcatgagttttgtttataacttgtcagacacacaccttttccccaatgcgagctc |
| Sequence | | caaagaaatcaagactgtggaatctataactgacatcagggcagatatagataaaaaatacagatgtgttagtggcacc |
| | | caggtccacatgaacaacgtgaccgtaacgctccatgatgccaccatccaggcgtacctttccaacagcagcttcagcc |
| | | ggggagagacacgctgtgaacaagacctcgagacacaagtctgcaccggcaccgacatgaagctgagactgcccgccag |
| | | ccccgaaacccatctggacatgctgcggcatctgtaccaaggctgtcaagtggtgcaaggcaatctggaactgacatat |
| | | ctgcctaccaacgcctctctgagctttctgcaagacatccaagaagtgcaaggctacgtcctcattgcccacaaccaag |
| | | tccgacaagtgccactgcagcggctgagaatcgtgcggggcacccagctgttcgaggacaactatgctctggccgtgct |
| | | ggacaacggcgaccctctgaacaacaccacaccagtgactggcgcctctcccggcggactgagagaactgcagctgcgc |
| | | tctctgaccgagattctgaagggcggcgtgctgatccagcggaaccctcagctgtgctaccaagacaccattctgtgga |
| | | aggacatcttccacaagaacaaccagctggctctgaccctcatcgacaccaacagaagccgggcttgccacccttgcag |
| | | ccccatgtgcaagggcagcagatgttggggcgagagcagcgaggactgccagtctctgaccagaaccgtgtgtgccggc |
| | | ggatgcgccagatgcaagggccctctgccaaccgattgctgccacgaacagtgcgccgctggctgtaccggccccaagc |
| | | acagcgattgtctggcttgtctgcacttcaaccactccggcatctgcgagctgcactgcccagccctcgtgacatacaa |
| | | caccgacacattcgagagcatgcccaaccccgagggcagatacacattcggcgccagctgtgtgaccgcttgcccctac |
| | | aactatctgagcaccgacgtgggcagctgcaccctcgtgtgccctctgcacaatcaagaagtgaccgccgaggacggca |
| | | cccagagatgcgagaagtgctccaagccttgcgccagagtgtgctacggactgggcatggaacatctgcgggaagtgcg |
| | | ggccgtgaccagcgccaatatccaagaatttgccggctgcaagaagattttcggcagtctggcctttctgcccgagagc |
| | | ttcgatggcgaccccgcctctaacacagcccctctgcagccagagcagctccaagtgttcgagacactggaagagatca |
| | | ccggctatctgtacatcagcgcttggcccgactctctgcccgatctgagcgtgttccagaatctgcaagtcatcagagg |
| | | ccggattctgcacaacggcgcctattctctgacactgcaaggactgggaatcagctggctgggactgcggagtctgaga |
| | | gagctgggcagcggactggcactgatccaccacaacacccatctgtgcttcgtgcacaccgtgccttgggatcagctgt |
| | | tccggaatccccaccaagctctgctgcacaccgccaacagacccgaggatgagtgtgtgggcgaggggctggcttgcca |
| | | tcagctctgtgccagaggacactgttggggacccggccctacccagtgcgtgaactgctcccagtttctgcggggccaa |
| | | gaatgcgtggaagagtgcagagtgctccaaggactgccccgcgagtacgtgaacgccagacactgtctgccatgccacc |
| | | ccgagtgccagcctcagaatggcagcgtgacatgcttcggccccgaggccgatcagtgtgtggcttgcgctcactacaa |
| | | ggaccctcccttctgcgtggcccggtgtccttctggcgtgaaacccgatctgtcctacatgcctatctggaagttcccc |
| | | gacgaggaaggcgcttgtcagccttgccccatcaactgcacccacgaattcacctgcctgctggccagcatggggctgc |
| | | agctgaacctcacctatgagaggaaggacaacacgacggtgacaaggcttctcaacatcaaccccaacaagacctcggc |
| | | cagcgggagctgcggcgcccacctggtgactctggagctgcacagcgagggcaccaccgtcctgctcttccagttcggg |
| | | atgaatgcaagttctagccggtttttcctacaaggaatccagttgaatacaattcttcctgacgccagagaccctgcct |
| | | ttaaagctgccaacggctccctgcgagcgctgcaggccacagtcggcaattcctacaagtgcaacgcggaggagcacgt |
| | | ccgtgtcacgaaggcgttttcagtcaatatattcaaagtgtgggtccaggctttcaaggtggaaggtggccagtttggc |
| | | tctgtggaggagtgtctgctggacgagaacagcatgctgatccccatcgctgtgggtggtgccctggcggggctggtcc |
| | | tcatcgtcctcatcgcctacctcgtcggcaggaagaggagtcacgcaggctaccagactatctagtaaagatctttttc |
| | | cctctgccaaaaattatggggacatcatgaagccccttgagcatctgacttctggctaataaaggaaatttattttcat |
| | | tgcaatagtgtgttggaattttttgtgtctctcactcggaaggacataagggcggccgcggtacccgtgaggctccggt |
| | | gcccgtcagtgggcagagcgcacatcgcccacagtccccgagaagttggggggaggggtcggcaattgaaccggtgcct |
| | | agagaaggtggcgcggggtaaactgggaaagtgatgtcgtgtactggctccgcctttttcccgagggtgggggagaacc |
| | | gtatataagtgcagtagtcgccgtgaacgttctttttcgcaacgggtttgccgccagaacacagtctagaggatccgcc |
| | | gccaccatgtacaggatgcaactcctgtcttgcattgcactaagtcttgcacttgtcacgaattcgatagtggacgagc |
| | | agctgagcaagagcgtgaaggacaaggtgctgctgccttgtaggtacaacagccctcacgaggacgagagcgaggatag |
| | | gatctactggcagaagcacgacaaggtagtgctgagtgtgatagccggcaagctgaaggtgtggcctgagtacaagaat |
| | | aggaccctgtacgacaacaccacctacagcctgatcatcctgggcctggtgctgagcgataggggtacctacagctgcg |
| | | tggtgcagaagaaggagaggggcacctacgaggtgaagcacctggccctggtgaagctgagcatcaaggccgacttcag |
| | | cacccctaacatcaccgagagcggcaaccctagcgccgacaccaagaggatcacctgcttcgctagcggcggcttccct |
| | | aagcctaggttcagctggctggagaacggaagggagctgcctggcataaacacgaccatatctcaagaccctgagagcg |
| | | agctgtacaccatcagctctcagctggacttcaacaccacccgcaatcacaccatcaagtgcctgatcaagtacggcga |
| | | cgcccacgtgagcgaggacttcacctgggagaagcctcctgaggaccctcctgacagcaagaacgatatcggaggcgga |
| | | ggaagcggaggcggaggaagccccagagggcccacaatcaagccctgtcctccatgcaaatgcccagcacctaacctcg |
| | | agggtggaccatccgtcttcatcttccctccaaagatcaaggatgtactcatgatctccctgagccccatagtcacatg |
| | | tgtggtggtggatgtgagcgaggatgacccagatgtccagatcagctggtttgtgaacaacgtggaagtacacacagct |
| | | cagacacaaacccatagagaggattacaacagtactctccgggtggtcagtgccctccccatccagcaccaggactgga |
| | | tgagtggcaaggcgttcgcatgcgcggtcaacaacaaagacctcccagcgcccatcgagagaaccatctcaaaacccaa |
| | | agggtcagtaagagctccacaggtatatgtcttgcctccaccagaagaagagatgactaagaaacaggtcactctgacc |
| | | tgcatggtcacagacttcatgcctgaagacatttacgtggagtggaccaacaacgggaaaacagagctaaactacaaga |
| | | acactgaaccagtcctggactctgatggttcttacttcatgtacagcaagctgagagtggaaaagaagaactgggtgga |
| | | aagaaatagctactcctgttcagtggtccacgagggtctgcacaatcaccacacgactaagagcttctcccggactccg |
| | | ggtaaa |
| IL-2 SP + CD80 extracellular | 252 | MYRMQLLSCIALSLALVTNSVDEQLSKSVKDKVLLPCRYNSPHEDESEDRIYWQKHDKVVLSVIAGKLKVWPEYKNRTL |
| domain + FC | | YDNTTYSLIILGLVLSDRGTYSCVVQKKERGTYEVKHLALVKLSIKADFSTPNITESGNPSADTKRITCFASGGFPKPR |
| | | FSWLENGRELPGINTTISQDPESELYTISSQLDFNTTRNHTIKCLIKYGDAHVSEDFTWEKPPEDPPDSKNDIGGGGSG |
| | | GGGSPRGPTIKPCPPCKCPAPNLEGGPSVFIFPPKIKDVLMISLSPIVTCVVVDVSEDDPDVQISWFVNNVEVHTAQTQ |
| | | THREDYNSTLRVVSALPIQHQDWMSGKAFACAVNNKDLPAPIERTISKPKGSVRAPQVYVLPPPEEEMTKKQVTLTCMV |
| | | TDFMPEDIYVEWTNNGKTELNYKNTEPVLDSDGSYFMYSKLRVEKKNWVERNSYSCSVVHEGLHNHHTTKSFSRTPGK |
| CD80 (Extracellular Domain | 253 | VDEQLSKSVKDKVLLPCRYNSPHEDESEDRIYWQKHDKVVLSVIAGKLKVWPEYKNRTLYDNTTYSLIILGLVLSDRGT |
| + Fc Domain) Protein | | YSCVVQKKERGTYEVKHLALVKLSIKADFSTPNITESGNPSADTKRITCFASGGFPKPRFSWLENGRELPGINTTISQD |
| Sequence | | PESELYTISSQLDFNTTRNHTIKCLIKYGDAHVSEDFTWEKPPEDPPDSKNDIGGGGSGGGGSPRGPTIKPCPPCKCPA |
| | | PNLEGGPSVFIFPPKIKDVLMISLSPIVTCVVVDVSEDDPDVQISWFVNNVEVHTAQTQTHREDYNSTLRVVSALPIQH |
| | | QDWMSGKAFACAVNNKDLPAPIERTISKPKGSVRAPQVYVLPPPEEEMTKKQVTLTCMVTDFMPEDIYVEWTNNGKTEL |
| | | NYKNTEPVLDSDGSYFMYSKLRVEKKNWVERNSYSCSVVHEGLHNHHTTKSFSRTPGK |
| CD80 (Extracellular Domain | 881 | gtggacgagcagctgagcaagagcgtgaaggacaaggtgctgctgccttgtaggtacaacagccctcacgaggacgaga |
| + Fc Domain) DNA | | gcgaggataggatctactggcagaagcacgacaaggtagtgctgagtgtgatagccggcaagctgaaggtgtggcctga |
| Sequence | | gtacaagaataggaccctgtacgacaacaccacctacagcctgatcatcctgggcctggtgctgagcgataggggtacc |
| | | tacagctgcgtggtgcagaagaaggagaggggcacctacgaggtgaagcacctggccctggtgaagctgagcatcaagg |
| | | ccgacttcagcacccctaacatcaccgagagcggcaaccctagcgccgacaccaagaggatcacctgcttcgctagcgg |
| | | cggcttccctaagcctaggttcagctggctggagaacggaagggagctgcctggcataaacacgaccatatctcaagac |
| | | cctgagagcgagctgtacaccatcagctctcagctggacttcaacaccacccgcaatcacaccatcaagtgcctgatca |
| | | agtacggcgacgcccacgtgagcgaggacttcacctgggagaagcctcctgaggaccctcctgacagcaagaacgatat |
| | | cggaggcggaggaagcggaggcggaggaagccccagagggcccacaatcaagccctgtcctccatgcaaatgcccagca |
| | | cctaacctcgagggtggaccatccgtcttcatcttccctccaaagatcaaggatgtactcatgatctccctgagcccca |
| | | tagtcacatgtgtggtggtggatgtgagcgaggatgacccagatgtccagatcagctggtttgtgaacaacgtggaagt |
| | | acacacagctcagacacaaacccatagagaggattacaacagtactctccgggtggtcagtgccctccccatccagcac |
| | | caggactggatgagtggcaaggcgttcgcatgcgcggtcaacaacaaagacctcccagcgcccatcgagagaaccatct |
| | | caaaacccaaagggtcagtaagagctccacaggtatatgtcttgcctccaccagaagaagagatgactaagaaacaggt |
| | | cactctgacctgcatggtcacagacttcatgcctgaagacatttacgtggagtggaccaacaacgggaaaacagagcta |
| | | aactacaagaacactgaaccagtcctggactctgatggttcttacttcatgtacagcaagctgagagtggaaaagaaga |
| | | actgggtggaaagaaatagctactcctgttcagtggtccacgagggtctgcacaatcaccacacgactaagagcttctc |
| | | ccggactccgggtaaa |
| Exemplary human LAMP1- | 879 | |
| Large T antigen fragment | | |
| sequence including LAMP1 | | |
| transmembrane and | | |
| cytoplasmic regions and | | |
| LAMP1 signal sequence | | |
| Exemplary human LAMP1- | 880 | |
| Large T antigen fragment | | |
| sequence including LAMP1 | | |
| signal sequence, and | | |
| excluding LAMP1 | | |
| transmembrane and | | |
| cytoplasmic regions | | |
| Complimentary sequence to | 882 | See FIG. 28C |
| ITI-COVID-19 Bicistronic | | |
| Vaccine (ITI-Bicistronic-S1- | | |
| LAMP-RBG pA-EF2-S2P | | |
| BHG pA; First Generation | | |
| COVID-19 Vaccine); DNA | | |
| Sequence (SEQ ID NO: 228) | | |

### Example 2. A Second Generation Bicistronic Covid Vaccine (2-V-Covid) Elicits T Cell and Antibody Responses in Mice

A major challenge for generating sufficient antigen-specific T cell and antibody responses is the low immunogenicity of DNA vaccines. To increase T cell response to the DNA vaccine, a new bicistronic DNA vaccine was designed. This Example discusses a second generation COVID-19 DNA vaccine, Spike-LAMP-sCD40L (2-V-Covid vaccine; SARS-COV-2 B.1.351 Spike (South Africa Variant, without SP and TM)+LAMP+EF-1alpha+SPD+sCD40L; SEQ ID NOS: 242-243), comprising (1) full-length SARS-COV-2 spike protein fused with LAMP, and (2) soluble CD40L (sCD40L), both of which were expressed separately. This bicistronic DNA vaccine was designed to induce local expression of sCD40L at relatively low levels, thereby providing a safe approach to using systemic recombinant CD40L or agonistic anti-CD40 antibodies (van Mierlo et al., 2002).

CD40 ligand (CD40L; CD154) enhances the adaptive immune responses by stimulating dendritic cells and B cells. DNA vaccines comprising the CD40L gene have been shown to enhance T-cell and antibody responses in vivo. (Not shown.) To produce a COVID vaccine with improved immunogenicity, a second generation vaccine was designed to express SARS-COV-2 full-length spike protein and soluble CD40L in two separate cassettes. Immunogenicity of this 2-V COVID vaccine was evaluated in BALB/c mice. After two immunizations, the new vaccine induced stronger spike-specific T-cell responses and higher levels of spike-specific antibody responses compared to the first generation COVID-19 DNA vaccine described in Example 1. The data discussed in the following Examples suggest that the new vaccine is more immunogenic and has the potential to improve protection against COVID 19 and future emerging infectious disease.

CD40L is a transmembrane protein expressed on the surface of activated T cells, particularly CD4 T cells. CD40L stimulates CD40-dependent activation of antigen-presenting cells (APCs), such as dendritic cells (DCs) and macrophages, as well as B cells for enhancing T cell and antibody responses (Grewal & Flavell, 1998; Schoenberger et al., 1998). Recombinant soluble CD40L or agonistic antibodies have been used in clinical and demonstrated promising results in various cancers (Beatty et al., 2011, 2017; Vonderheide et al., 2001). Consequently, these immunostimulatory functions of CD40L have made it a promising vaccine adjuvant against infectious disease and cancers. As discussed herein and in the Examples that follow, sCD40L enhanced spike-specific T cell and antibody responses. The data discussed herein support the use of bicistronic DNA vaccines against infectious disease.

### A. Materials and Methods

### 1. Vaccine Constructs

The ITI-COVID-19 bicistronic vaccine (ITI-Bicistronic-S1-LAMP-RBG pA-EF2-S2P BHG pA; first generation COVID-19 DNA vaccine, “ITI-bicistronic vaccine,”; shown in FIG. 28C, SEQ ID NO: 228) was previously constructed as described in Example 1 above.

The second generation Spike-LAMP-sCD40L construct “2-V COVID vaccine,” SEQ ID NO: 228) was also constructed according to methods described in Example 1 above. The 2-V COVID vaccine had two separate cassettes driven independently by CMV promoter and EF1 promoter. The first cassette expressed full-length spike gene of the SARS-COV-2 South Africa variant (B.1.351) infectious clone (SEQ ID NO: 227). The second cassette expressed soluble murine CD40L extracellular domain fused with mouse pulmonary surfactant associated protein D (SPD) protein (SEQ ID NO: 233).

The control vector (CV) used is the vector without any gene insertions.

### 2. Reagents

Antibodies for flow cytometry and enzyme-linked immunospot (ELISPOT) were purchased from Biolegend, SA-HRP and AEC kit were purchased from BD. Antibody to spike proteins, and recombinant spike (S1) (Cat #40591-V08H) and RBD (Cat #40592-V08B) proteins were purchased from Sino Biologics. Rabbit polyclonal anti-S1 antibody was purchased from Sino Biologics. Antibody titers were evaluated using HRP anti-mouse antibodies from Southern Biotech. Renilla Luciferase Reporter Assay was purchased from Promega (Cat #E2710). Epivax peptides were synthesized by GenScript.

The pre-make lentiviral particles for overexpression of human ACE2 were ordered from Gen Target Inc (Cat #LvP1310)

JPT PepMix™ SARS-COV-2 overlapping peptide pool was purchased from JPT (Cat #PM-WCPV-S). The peptide pool contained a total of 315 peptides (delivered in two subpools of 158 and 157 peptides) derived from a peptide scan (15mer segments with an 11 amino acid overlap) through Spike (UniProt: PODTC2).

The pseudotyped luciferase rSARS-Cov-2 Spike virus purchased from Creative Diagnostics (No: Cov-PS01, Lot. No.: CL-114A). it is based on SARS-Cov-2 Wuhan-Hu-1 with luciferase as a reporter.

### 3. Transient Transfections of Constructs

293T cells were transiently transfected with control vector (CV) or bicistronic vaccine constructs using lipofectamine 2000 (Invitrogen). 48-hours post-transfection, the supernatant was collected, centrifuged, and filtered. The supernatant was analyzed for expression of soluble CD40L.

In some embodiments, 293T cells may be transduced with human ACE2. 293T-ACE2 cell line was made by transducing 293T cell with a lentivirus expressing human ACE2 (angiotensin I converting enzyme 2, NM_021804), the lentivirus was made by GeneTarget Inc, it contains a RFP and Blasticidin dual selection markers. After infection, 15 ug/ml of Blasticidin are used to isolate the single cell clone that expresses both RFP and ACEs. As shown in the expression of human ACE2 was detected by staining with anti-human ACE2 antibody (Sino Biological, rabbit Pab, Cat #: 10108-T60). The 293T-ACE3 clone 2 and clone 5 have higher expression of hACE2, the clone 5 was used for the neutralization test in this study.

### 4. Western Blot and Immunoprecipitation Methods

Biotinylated anti-CD40L antibody (Clone MR1 BioLegend Cat #106503) was bound to streptavidin-coated magnetic beads and used to isolate CD40L. The beads were loaded onto gel for Western blot imaging. The following antibodies were also used for Western blot: (1) primary anti-CD40L/CD154 antibody (Invitrogen PA5-78983) and secondary antibody of goat anti-rabbit IgG-HRP (Southern Biotech 4030-05).

### 5. Immunization and Serum Collection

Six-to eight-week old female BALB/c mice were bred and maintained at a licensed animal facility.

The immunization schedule for ITI-bicistronic vaccine and 2-V COVID vaccine is shown in Table 2. Mice were immunized with DNA vaccines by ID injection to the ear followed by electroporation. Blood samples were collected before immunization and 14 days after the 2nd immunization. At day 28, mice were sacrificed as scheduled and spleens and sera were collected for the measurement of T cells and antibodies responses.

TABLE 2
Immunization Schedule.

| | | Concentration | Dose | | # | Volume | Mice | | | | |
|---|---|---|---|---|---|---|---|---|---|---|---|
| Group | Treatment | (mg/mL) | (μg) | Route | Mice | (μL) | ID | Day 0 | Day 14 | Day 14 | Day 28 |
| A | Control | 3.3 | 20 | ID | 10 | 20 | 001-010 | 1^{st} | Terminated | 2^{nd} | Termination |
| | Vector | | | (ear) | | | | immunization | 5 mice in | immunization | for ELISA |
| | (CV) | | | | | | | | each group | | and |
| B | ITI- | 2.0 | 20 | ID | 10 | 20 | 011-020 | | for ELISA | | ELISPOT |
| | Bicistronic | | | (ear) | | | | | and | | |
| | Vaccine | | | | | | | | | | |
| C | Spike- | 2.0 | 20 | IM | 10 | 20 | 021-030 | | | | |
| | LAMP- | | | (ear) | | | | | | | |
| | sCD40L | | | | | | | | | | |
| | (2-V | | | | | | | | | | |
| | COVID | | | | | | | | | | |
| | vaccine) | | | | | | | | | | |

### 6. Evaluation of Antigen-Specific T Cell Response by ELISPOT

To assess antigen-specific T cell response in the vaccinated mice, splenocytes from vaccinated mice were evaluated for antigen-specific IFNγ by Enzyme-linked immunospot (ELISPOT). ELISPOT assays were performed as described herein. Briefly, splenocytes were plated at 3×10⁵ cells/well and co-cultured with 1 μg/ml of JPT overlapping spike peptides, 0.25 μg/mL of concavalin A, or medium alone in a total volume of 200 μl/well T cell media for 48 h at 37° C. in 5% CO₂. The plates were developed with 50 μl/well AEC development solution for up to 30 min. Color development was stopped by washing under running tap water. After air dried, colored spots were counted using an AID ELISPOT High-Resolution Reader System and AID ELISPOT Software version 3.5 (Autoimmun Diagnostika GmbH). Student T test was used to determine the significant difference between the cells transfected with ITI-bicistronic vaccine and 2-V COVID vaccine.

### 7. ELISA

The murine antibody response to vaccine was assessed by indirect ELISA. ELISA plates (MaxiSorp) were coated with 1 μg/ml recombinant SARS-Cov-2 spike S1, or RBD protein overnight and then blocked with 2% BSA in PBS. Serum samples were diluted (1:2) in PBS-T. Samples were detected with 1:6000 goat anti-mouse IgG-HRP (Southern Biotech, Birmingham, Al). Reaction was developed with SureBlue TMB Substrate and stopped with TMB Stop Solution from KPL (Gaithersburg, MD). Plates were read (OD450) by using Epoch ELISA reader (BioTek, Winooski, VT).

8. Sars-Cov-2 Pseudovirus Neutralization Test (pVNT)

In some embodiments, a SARS-COV-2 pseudovirus neutralization test was used. Pseudotyped Luciferase rSARS-COV-2 Spike was purchased from Creative Diagnostics Inc., this lentivirus-based SARS-COV-2 S pseudotyped virus is a replication-restricted, recombinant pseudotyped lentiviral particles containing SARS-COV-2 spike protein (based on Wuhan-Hu-1 Isolate). Because the infectivity of Pseudotyped Luciferase rSARS-COV-2 is restricted to a single round of replication, it encodes Renilla luciferase in their lentiviral vector genome. When its genome integrates after entry into cells, luciferase expression and activity is proportional to the number of cells that were transduced.

To determine pVNT, 20 μL of SARS-COV-2 spike pseudotyped virus (105 RLU) may be pre-incubated with twofold serial-diluted test serum samples (starting dilution of 1:10) in a final volume of 50 μL for 1 hour at 37° C., followed by adding 5×10⁴ HEK293T-ACE2 cells in a volume of 50 μL. At 48-72 hours post-infection, an equal volume of Renilla Luciferase substrate (Promega, Cat #2710) is added and the luminescence signal was measured using microplate reader (BioTek) with Gen5 software. Measurements may be done in duplicate for CV pooled serum samples and triplicate for group B pooled serum samples. The % of neutralization and the IC50 of pVNT are calculated as previously described (Le Bert et al., 2020).

### 9. Flow Cytometry and Intracellular Cytokine Staining (ICS)

Splenocytes were stimulated with spike peptides at concentration of 2 μg/mL for 6 hours. After 6 hours incubations, the cells were stained with Zombie aqua, followed by surface staining, fixation with Perm/fixation solution (BD Biosciences), and stained with intracellular antibody in 1× perm/wash buffer. Samples were analyzed on a CytoFlex flow cytometer (Beckman Coulter) and analyzed using Kaluza software (Beckman Coulter).

Cells were stained for intracellular cytokine for IFNγ, TNFα, and IL-2. CD4 T cells (effector memory CD4) were gated on CD3+CD4+CD8-CD44+CD62L− lymphocytes. CD8 T cells (effector memory CD4) were gated on CD3+CD4-CD8+CD44+CD62L− lymphocytes.

### B. Results

### 1. Soluble CD40 Ligand (SCD40L) was Expressed by the 2-V Covid Vaccine

293T cells were transfected with 2-V COVID vaccine and analyzed for expression of sCD40L. Using immunoprecipitation and Western blot methods, expression of sCD40L from the 2-V COVID vaccine was confirmed (FIG. 12; boxed bands indicate sCD40L).

### 2. 2-V Covid Vaccine Elicited a T Cell Response

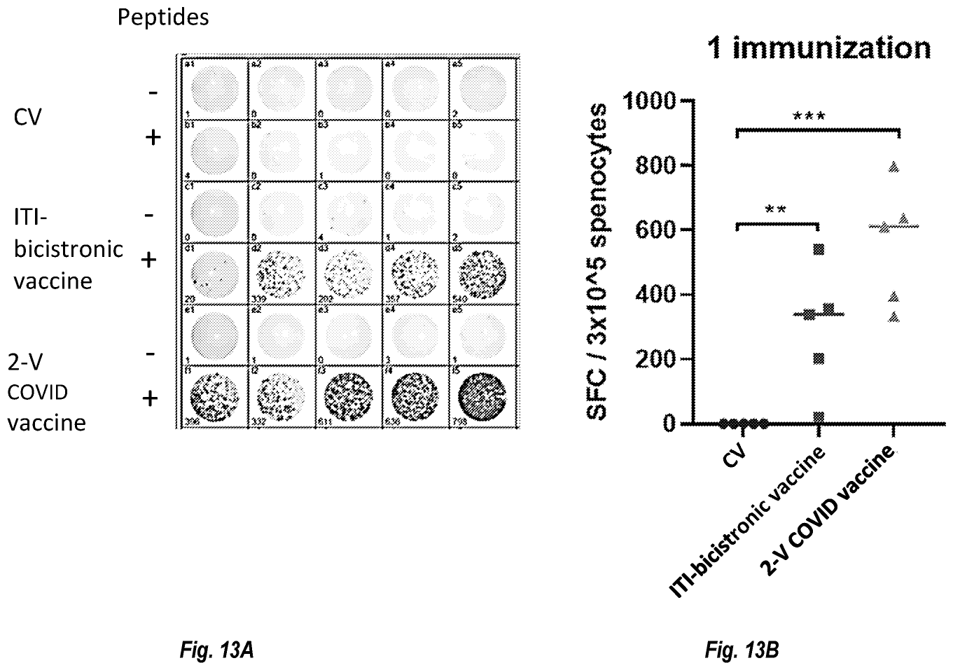

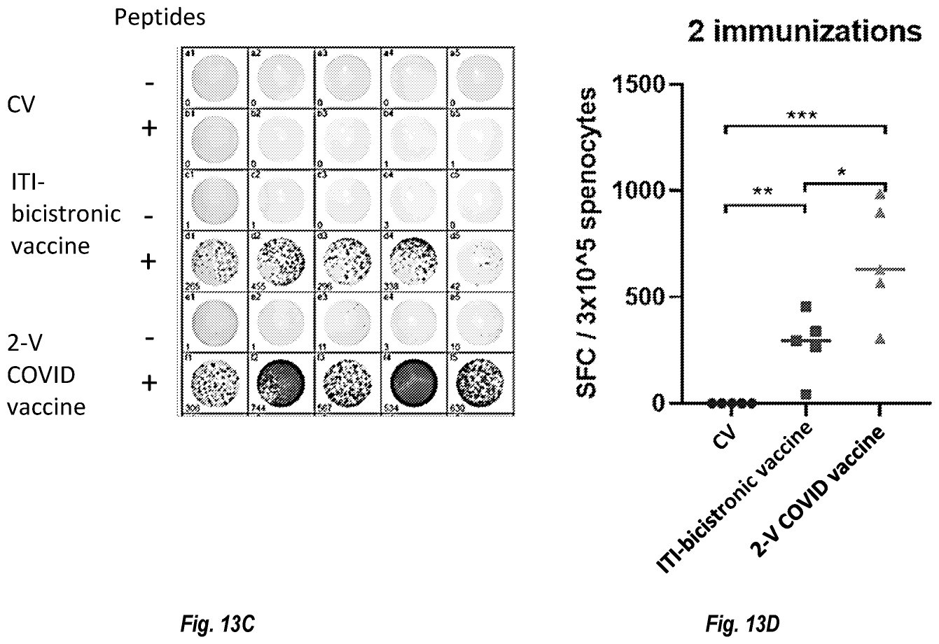

Immunogenicity of the 2-V COVID vaccine was determined at 14 days after the first immunization and at 14 days after the second immunization (Table 2). The data show that the 2-V COVID vaccine induced a significantly enhanced T cell response compared to the first generation COVID-19 vaccine (FIGS. 13A-D). The data indicate that the presence of sCD40L improved the 2-V COVID vaccine by eliciting greater T-cell response.

### 3. 2-V Covid Vaccine Stimulated Spike-Specific CD4 and CD8 T Cells In Vivo

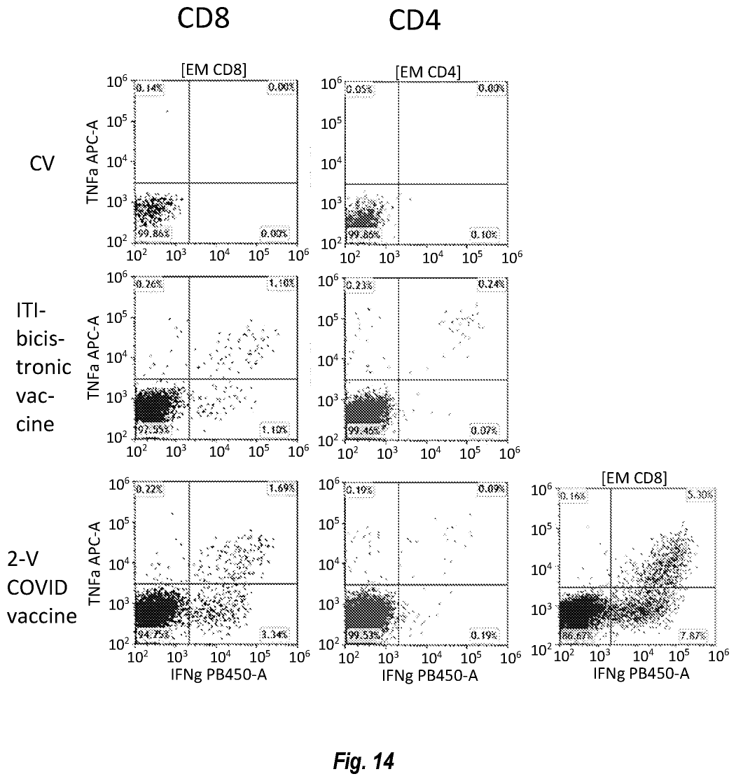

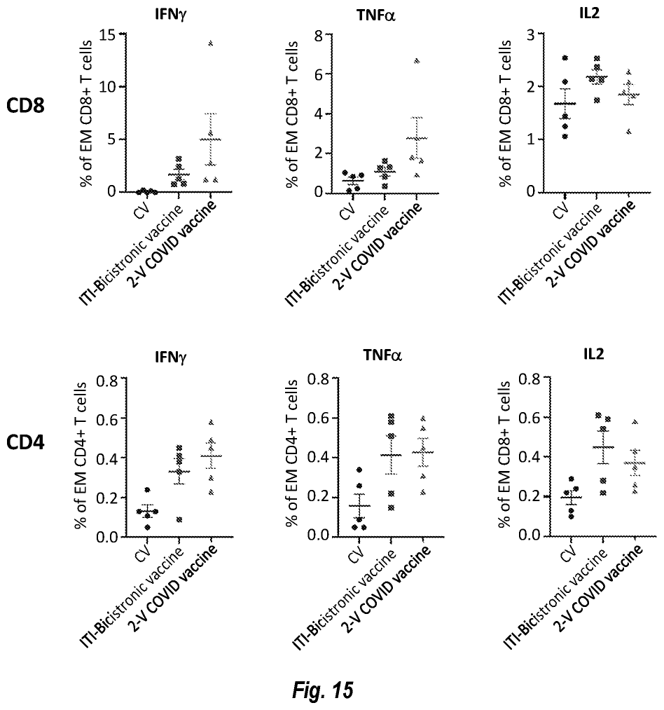

Greater numbers of CD4+ and CD8+ T cells were observed in splenocytes from mice vaccinated with the 2-V vaccine compared to the ITI-bicistronic vaccine (FIG. 14). The data indicate that the presence of sCD40L provided the enhancement. The CD4+ and CD8+ T cells were also stained for intracellular cytokines, and greater percentages of IFNγ, TNFα, and IL-2 in mice vaccinated with the 2-V vaccine compared to the ITI-bicistronic vaccine (FIG. 15).

### 4. 2-V Covid Vaccine Elicited a Spike-Specific Antibody Response In Vivo

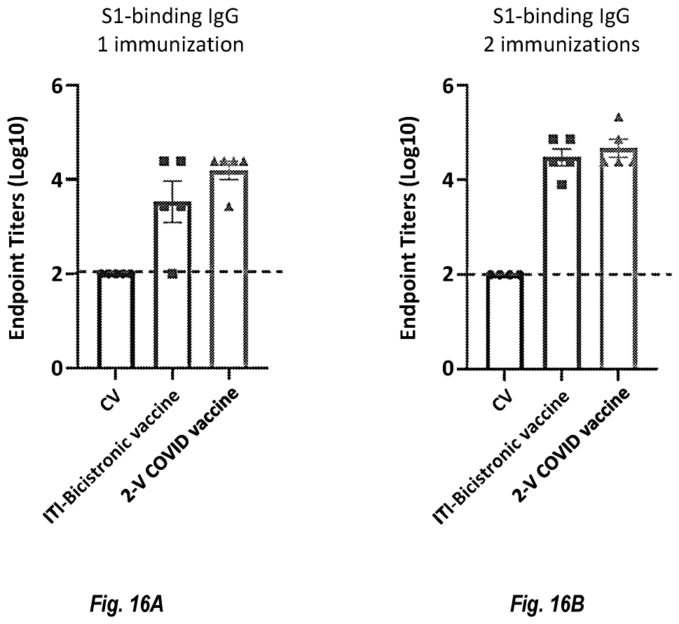

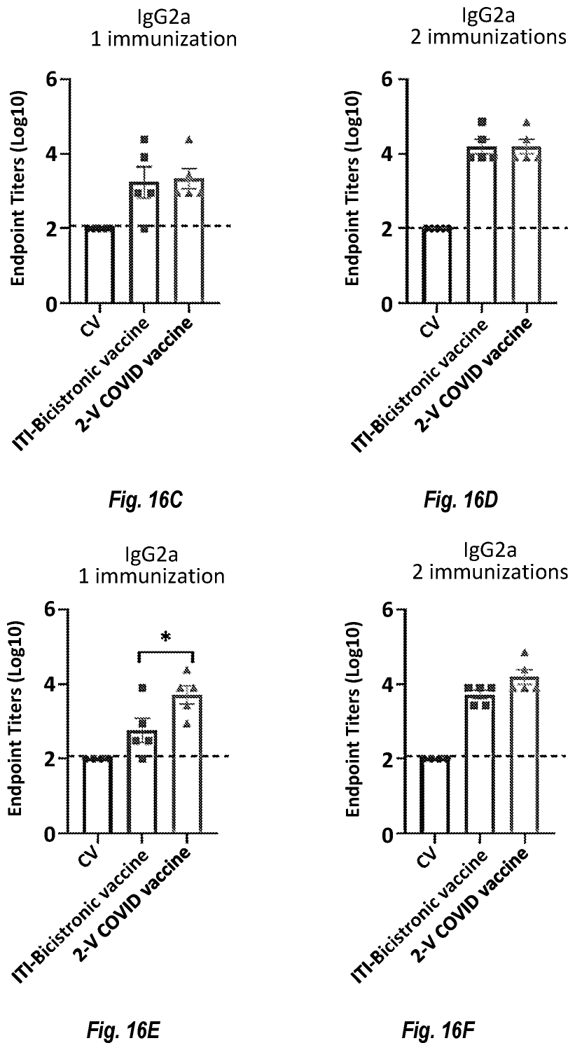

Serum from immunized mice was analyzed by ELISA for $1-specific antibodies after one or two immunizations. FIGS. 16A-B show total IgG of S1-binding antibodies. FIGS. 16C-D show IgG2a antibodies. FIGS. 16E-F show IgG1 antibodies. The data demonstrate that 2-V COVID vaccine elicited a superior S1-specific antibody response compared to ITI-bicistronic vaccine. The IgG1 response was particularly pronounced after a single dose of the 2-V COVID vaccine (FIG. 16E).

### 5. Summary

The 2-V vaccine significantly enhanced spike-specific T cell responses, including both CD4+ and CD8+ T cell responses, compared to the first generation vaccine. The 2-V vaccine also enhanced S1-specific antibody responses, particularly IgG1 levels after a single immunization dose.

### Example 3. Soluble CD40L (SCD40L) is Expressed from the Her2-LAMP-SCD40L Bicistronic Vaccine

This Example discusses HER2-LAMP-sCD40L (FIG. 11; SEQ ID NO: 197), a bicistronic DNA vaccine encoding HER2-LAMP and a 4-trimer version of sCD40L. The data discussed herein support the use of bicistronic DNA vaccines against cancer.

CD40 ligand (CD40L) is a transmembrane protein expressed on the surface of activated T cells, particularly on CD4 T cells, that stimulates CD40-dependent activation of antigen-presenting cells (APCs), resulting in enhancement of T cell and antibody responses. Soluble multimeric forms of CD40L (sCD40L) may act as adjuvant to enhance vaccine immunogenicity. As described in this Example, the HER2-LAMP-sCD40L bicistronic construct was tested against a construct that expresses HER2-LAMP but without expressing any second polypeptide such as sCD40L. The HER2-LAMP-sCD40L elicited significantly enhanced HER2-specific T cell and antibody responses in mice compared with mice immunized with the control HER2-LAMP DNA. Intracellular staining revealed that inclusion of sCD40L in the vaccine induced potent antigen specific T cell (IFNgamma) production, primarily in CD4 T cells. Furthermore, in a murine TSA breast cancer mocel, HER2-LAMP-sCD40L significantly inhibited tumor growth and prolonged survival in a therapeutic vaccine setting, suggesting that the HER2-LAMP-sCD40L vaccine is an effective strategy to promote anti-tumor efficacy in vivo.

### A. Materials and Methods

In general, materials and methods used in this Example were as described in Examples 1-2 above, with the following modifications.

### 1. Vaccine Constructs

The HER2-LAMP-sCD40L bicistronic construct (FIG. 11; SEQ ID NO: 197) discussed in these Examples comprises two expression cassettes. The first cassette is driven by a CMV promoter to express a LAMP-HER2/ErBB2 fusion protein (SEQ ID NO: 195; or SEQ ID NO: 198 followed by SEQ ID NO: 200 followed by SEQ ID NO: 202). The second cassette is driven by an EF1 promoter to express soluble murine CD40 ligand (sCD40L; GenBank accession no. X65453.2), which encodes a 4-trimer soluble CD40L (Gómez et al., 2009; Stone et al., 2006) fused to the body of surfactant protein D (SPD) (SEQ ID NO: 196). Three amino acids, HRR, are present between SPD and sCD40L. The construct was designed so as to deliver the HER2 antigen to the MHC II compartment, which may enhance both antibody generation and CD4 T cell response, while the sCD40L polypeptide construct is secreted.

A HER2-LAMP construct without sCD40L was used as a control vaccine.

A bicistronic DNA vaccine encoding spike (Spike-LAMP) was used as a control vector (i.e., negative control).

### 2. Immunization and Serum Collection

Six-to eight-week old female C57BL/6 mice were bred and maintained at a licensed animal facility. The immunization schedule for HER2-LAMP-sCD40L vaccine is shown in FIG. 13A and Table 3. Mice were immunized with 20 μg of control vector or vaccine by intradermal (ID) injection to the ear. The experiment was terminated one week after the second dose, i.e., on day 22. Splenocytes were treated with 1 μg/mL HER2 pooled peptides for 48 hours.

TABLE 3
Immunization Schedule.

| | | Concentration | Dose | | # | Volume | Mice | | | |
|---|---|---|---|---|---|---|---|---|---|---|
| Group | Treatment | (mg/mL) | (μg) | Route | Mice | (μL) | ID | Day 0 | Day 14 | Day 22 |
| A | Control | 3.1 | 20 | ID | 5 | 20 | 001-005 | 1^{st} | 2^{nd} | Termination |
| | Vector | | | (ear) | | | | immunization | immunization | to determine |
| | Spike- | | | | | | | | | the T cell |
| | LAMP | | | | | | | | | and antibody |
| B | LAMP- | 3.0 | 20 | ID | 7 | 20 | 006-012 | | | |
| | Hinge- | | | (ear) | | | | | | |
| | HER2 | | | | | | | | | |
| C | LAMP- | 2.0 | 20 | IM | 7 | 20 | 013-019 | | | |
| | HER2- | | | (ear) | | | | | | |
| | sCD40L | | | | | | | | | |

### 3. Transient Transfections of Constructs and ELISA

293T cells transfected with HER2-LAMP-sCD40L were analyzed for expression of sCD40L. A bicistronic DNA vaccine encoding spike (Spike-LAMP) as a negative control. Five days after transfection, the supernatant from these cells were collected and sCD40L was detected using ELISA.

### 4. Evaluation of Antigen-Specific T Cell Response by ELISPOT

Materials and methods were as described in Example 2 above, except overlapping HER2 peptides (purchased from Genscript or JPT) were used instead of overlapping spike peptides.

### 5. Flow Cytometry and Intracellular Cytokine Staining (ICS)

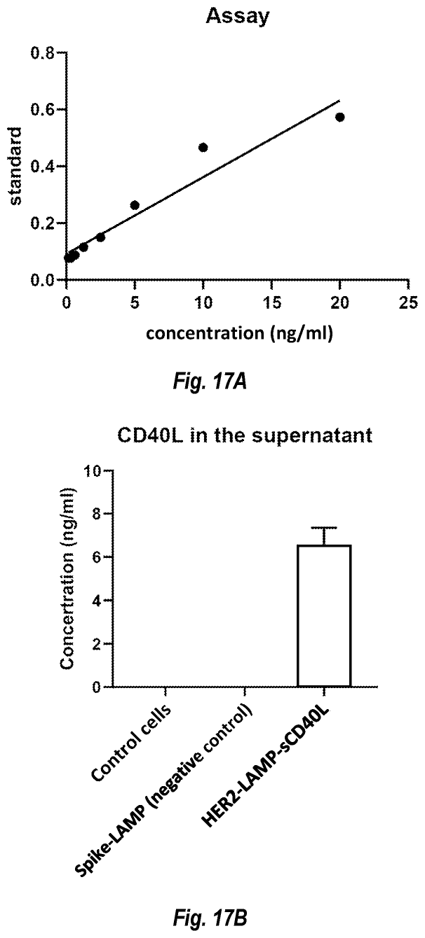

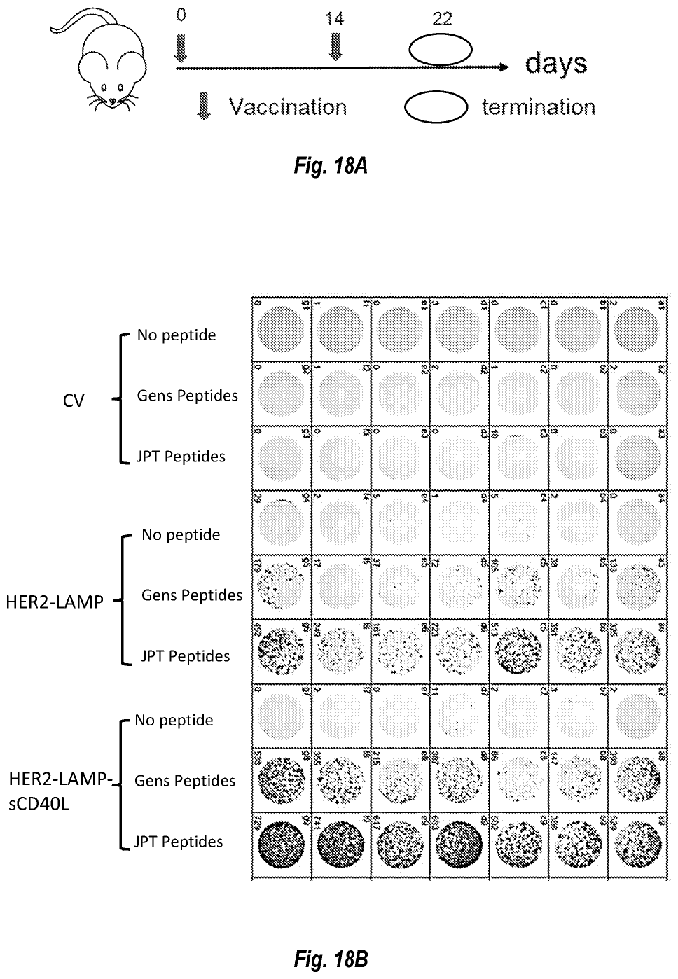

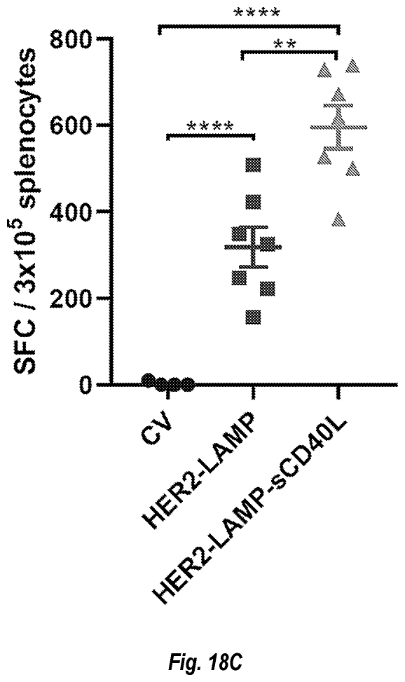

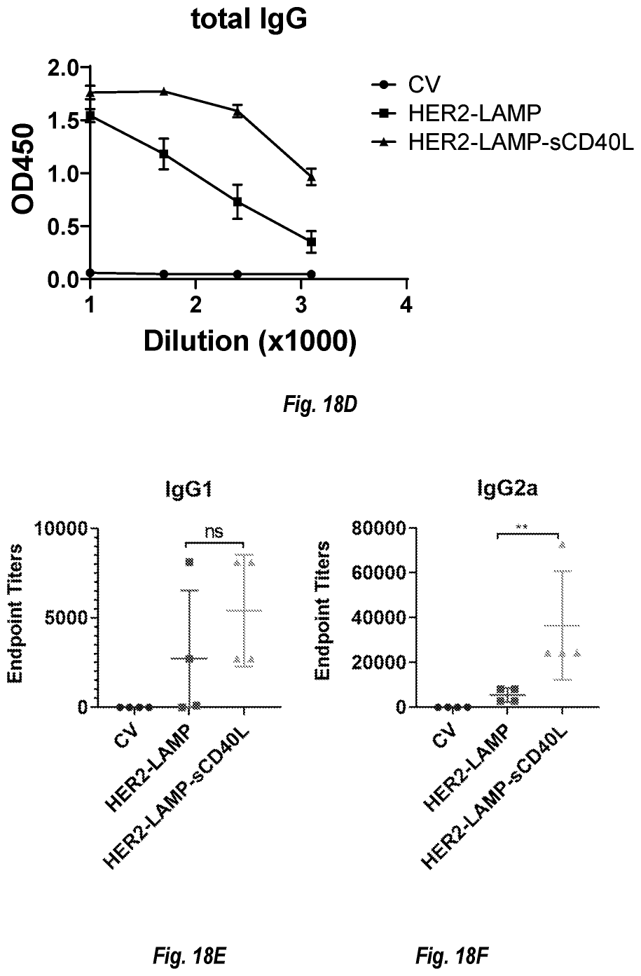

Splenocytes from immunized mice (FIG. 18A) were incubated for five hours with brefeldin A and monensin, with or without the HER2 peptide pool.

### 6. ELISA

The murine antibody response to HER2 was assessed by indirect ELISA. ELISA plates (MaxiSorp) were coated with 1 μg/ml HER2 protein overnight.

### 7. Statistics

Statistical analysis was performed using Prism software. One-way ANOVA was used to compare each group.

### 8. Murine Breast Tumor Model

Mice were immunized with two doses of HER2-LAMP-sCD40L or HER2-LAMP, followed by an injection of 2×10⁵ HER2-expressing TSA (murine mammary cancer) cells. Blood cells were stimulated with 1 μg/mL of HER2 peptide pool and analyzed by ELISPOT. Tumors were measured using a caliper.

### B. Results

### 1. Soluble CD40 Ligand (SCD40L) was Expressed by Her2-LAMP-SCD40L

293T cells were transfected with HER2-LAMP-sCD40L and analyzed for expression of sCD40L. Using ELISA, expression of sCD40L from HER2-LAMP-sCD40L was confirmed (Fig. FIGS. 17A-B). For example, sCD40L was detected in the supernatant of the 293T cells.

### 2. Her2-Lamp-SCD40L Elicited Her2-Specific Cellular and Humoral Response

Immunogenicity of HER2-LAMP-sCD40L was determined day 22, which is one week after the second immunization dose (FIG. 18A and Table 3). The data show that mice vaccinated with HER2-LAMP-sCD40L had significant T cell response compared to mice vaccinated with HER2-LAMP, i.e., a vaccine constructed without sCD40L (FIGS. 18B-C). The data also show that mice vaccinated with HER2-LAMP-sCD40L had increased total IgG (FIG. 18D), IgG1 (FIG. 18D), and IgG2a (FIG. 18D) antibodies compared to mice vaccinated with HER2-LAMP control. Together, the data indicate that the presence of sCD40L facilitated improved T-cell and antibody responses in vivo.

3. Her2-LAMP-SCD40L Stimulated CD4 T Cells More Efficiently than CD8 T Cells

Splenocytes from immunized mice were incubated for five hours in the presence of brefeldin A and monensin (see methods part 5) with or without the HER2 peptide pool. Cells were then intracellularly stained and analyzed by flow cytometry. The effector memory CD4 or CD8 T cells were gated on CD3+CD44+CD62L−CD4+ or CD8% lymphocytes. Data in FIGS. 19A-B is indicated as mean+/−SEM in FIG. 19A and representative FACS plots in FIG. 19B. As shown in FIGS. 19A-B, HER2-LAMP-sCD40L stimulated CD4 T cells more efficiently than CD8 T cells. The data also indicate that the presence of sCD40L provided the enhanced stimulation of both CD4 and CD8 T cells (FIGS. 19A-B).

### 4. Her2-LAMP-SCD40L Elicited a Her2-Specific Antibody Response In Vivo

Serum from immunized mice was analyzed by ELISA for HER2-specific IgG antibodies after two immunizations of either HER2-LAMP-sCD40L or HER2-LAMP or a vector control (CV). FIG. 20 shows total IgG of HER2-binding antibodies. The data demonstrate that the presence of sCD40L elicited an improved antibody response compared to HER2-LAMP.

### 5. Summary

The HER2-LAMP-sCD40L vaccine elicited robust HER2-specific T cell and antibody responses in vivo. The vaccine also enhanced CD4 T cells compared to CD8 T cells, suggesting that sCD40L preferentially acts on CD4 T cells in vivo. Example 4 below discusses the effect of HER2-LAMP-sCD40L in a murine breast tumor model.

In separate work (not shown), a HER2-LAMP-sCD40L self amplifying RNA construct was also prepared and used to transfect BHK-21 cells, and it was confirmed by Western blot that the transfected cells secreted sCD40L.

### Example 4. Anti-Tumor Effect of Bicistronic Her2-Lamp IREG Expressing DNA Vaccines in a TSA Breast Cancer Model

Vaccine-induced immune responses can be enhanced by expression of immune response-enhancing-genes (IREGs), which can amplify the immune response, alter quality of the immune response, and/or create a tumor microenvironment conducive to immune cell infiltration. The preceding Example discusses that the addition of CD40L to DNA vaccines elicited enhanced T-cell and antibody responses in vivo. This Example discusses HER2-LAMP vaccines with the IREG Flt3L, IL-21, IL-21, or OX40L. The data demonstrate that HER2-LAMP-IREG vaccines enhance immunogenicity and have anti-tumor properties.

### A. Materials and Methods

In general, materials and methods used in this Example were as described in Examples 1-3 above, with the following modifications.

### 1. Vaccine Constructs

HER2-LAMP-sCD40L (SEQ ID NO: 197) and HER2-LAMP control were as discussed in Examples 1 and 3 above.

HER2-LAMP-mFLT3L (SEQ ID NO: 208), HER2-LAMP-IL-12 (SEQ ID NO: 212), HER2-LAMP-IL-21 (SEQ ID NO: 216), and HER2-LAMP-OX40L (SEQ ID NO: 241) were constructed as described in Example 1.

The control vector (CV) used the vector without any gene insertions.

### 2. Immunization

Six-to eight-week old female BALB/c mice were purchased from the Jackson Laboratory (Maine, USA. Two days after subcutaneous (sc) injection with 2×10⁵ TSA-HER tumor cells, the mice were immunized with DNA vaccines in the ear by ID. Mice were immunized three more times as shown in Table 4.

TABLE 4
Immunization Schedule.

| | | Dose | # | Vol | Mouse | | | | | | |
|---|---|---|---|---|---|---|---|---|---|---|---|
| Group | Vaccine | (μg) | Mice | (μL) | ID | Day 0 | Day 2 | Day 9 | Day 16 | Day 23 | Day 45 |
| A | Control | 20 | 10 | 20 | 41-50 | sc | First | Second | Third | Fourth | Last day |
| | Vector | | | | | injection | immunization | immunization | immunization | immunization | for |
| | (CV) | | | | | of | (ID ear) | (ID ear) | (ID ear) | (ID ear) | monitoring |
| B | HER2- | 20 | 10 | 20 | 51-60 | 2 × 10⁵ | | | | | tumor |
| | LAMP- | | | | | TSA- | | | | | growth |
| | CD40L | | | | | HER2 | | | | | and |
| C | HER2- | 20 | 10 | 20 | 61-70 | cells | | | | | mouse |
| | LAMP- | | | | | | | | | | survival |
| | Flt3L | | | | | | | | | | |
| D | HER2- | 20 | 10 | 20 | 71-80 | | | | | | |
| | LAMP- | | | | | | | | | | |
| | IL-21 | | | | | | | | | | |
| E | HER2- | 20 | 10 | 20 | 81-90 | | | | | | |
| | LAMP- | | | | | | | | | | |
| | IL-12 | | | | | | | | | | |
| F | HER2- | 20 | 10 | 20 | 91-100 | | | | | | |
| | LAMP- | | | | | | | | | | |
| | OX40L | | | | | | | | | | |

### 3. Tumor Monitoring

2×10⁵ TSA-HER2 tumor cells were injected into mammary fat pad 2 days before the first immunization. Tumors were measured twice or once every week. Tumor diameter was calculated as the square root of length×width, and tumor volume was calculated using the formula 4/3πr³. Mice were euthanized when the tumor volume reached 2,000 mm³ or moribund.

### 4. Statistics

Results are shown as the mean±standard deviation. Statistical analyses were performed by using the GraphPad Prism software, version 9.0.2. Two-tailed student's T test was used. * p<0.05; ** p<0.01; *** p<0.001; **** p<0.0001.

### B. Results

### 1. Her2-LAMP-SCD40L Demonstrated Anti-Tumor Effect Against Her2 In Vivo

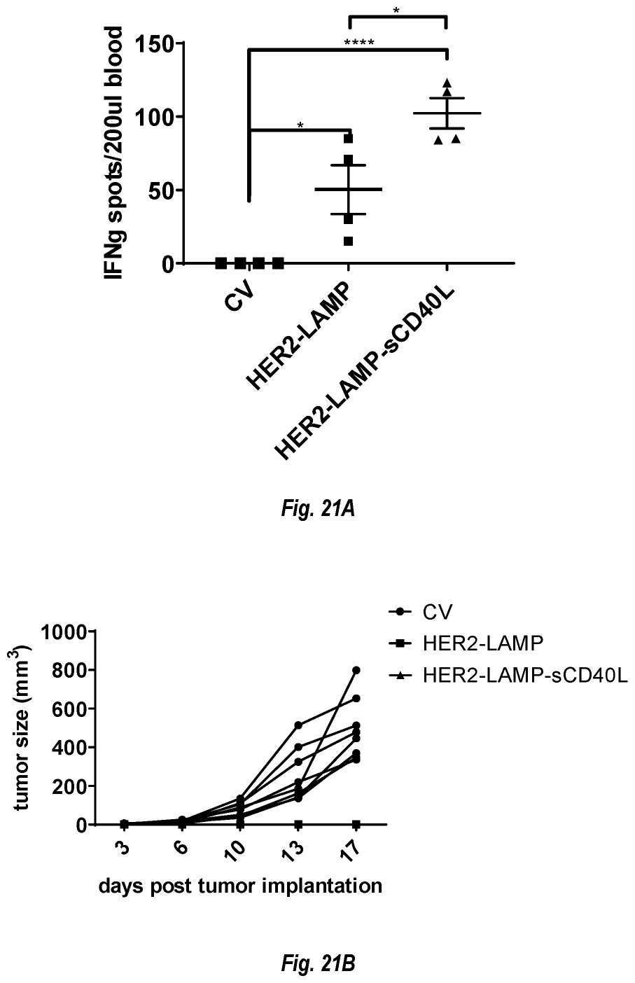

The effect of HER2-LAMP-sCD40L in a murine breast tumor model was tested. Mice were immunized with two doses of HER2-LAMP-sCD40L or HER2-LAMP before being challenged with HER2-expressing TSA cells. Blood cells from mice treated with HER2-LAMP-sCD40L had more IFNγ spots (FIG. 21A). Both HER2-LAMP-sCD40L or HER2-LAMP had an anti-tumor effect as mice treated with a either vaccine did not have tumor growth for 17 days (FIG. 21B). The mice were also observed for 45 days to determine if the vaccines had an impact on mouse survival. Probability of survival for were treated mice are shown in FIG. 22B. While both HER2-LAMP-sCD40L and HER2-LAMP enhanced mouse survival, HER2-LAMP-sCD40L appeared to be more protective against the HER2-expressing cells.

2. Bicistronic Vaccines with Immune Response-Enhancing Genes (IREGS) Demonstrated Anti-Tumor Effect In Vivo

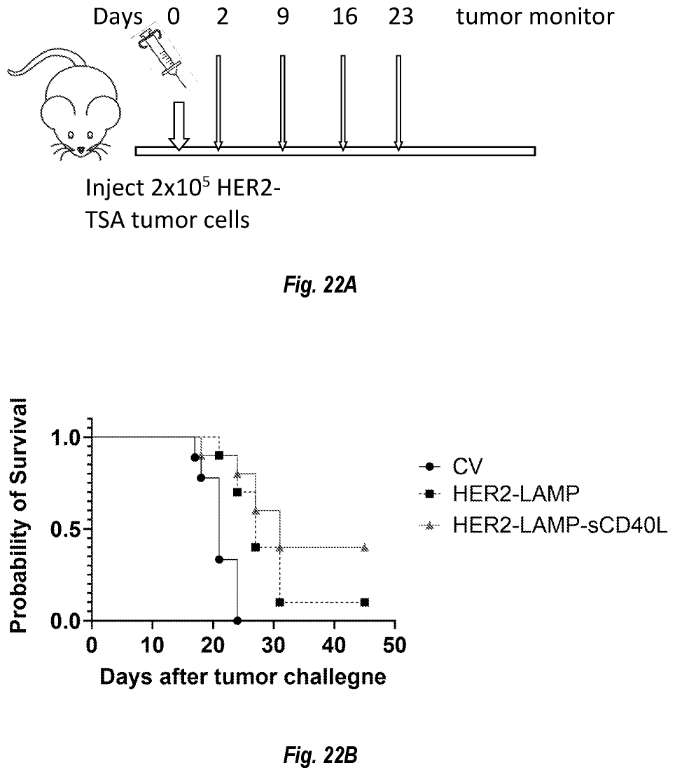

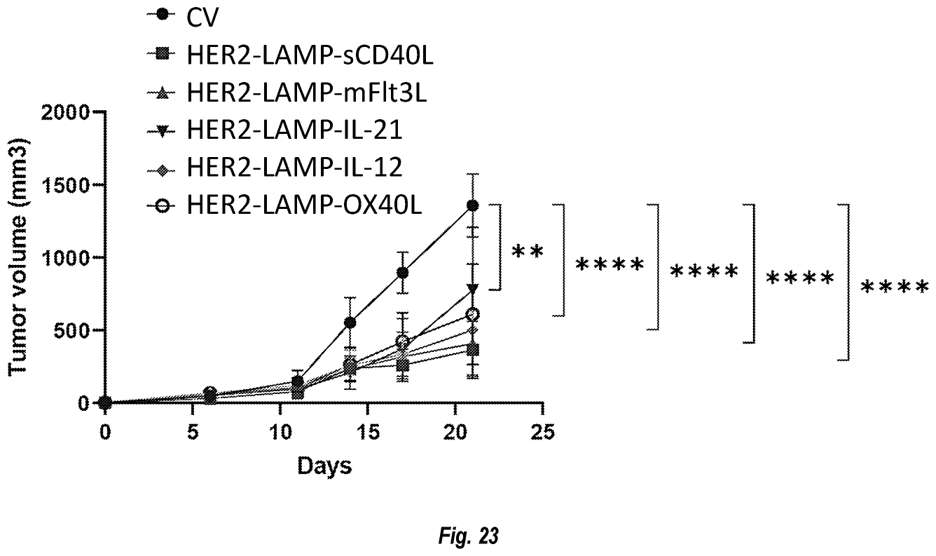

The effect of HER2-LAMP in combination with other IREGs was tested. In addition to HER2-LAMP-CD40L, the following for vaccines were tested: (1) HER2-LAMP-Flt3L (SEQ ID NO: 208), (2) HER2-LAMP-IL-21 (SEQ ID NO: 216), (3) HER2-LAMP-IL-12 (SEQ ID NO: 212), and (4) HER2-LAMP-OX40L (SEQ ID NO: 241). In all instances, tumors in vaccinated mice showed slower growth rate than the tumors in CV-treated mice (FIG. 23). Of the five HER2-LAMP vaccines tested, HER2-LAMP-IL-21 showed the lowest levels of tumor suppression whereas HER2-LAMP-CD40L and HER2-LAMP-Flt3L showed the highest levels of tumor suppression (FIG. 23).

3. Bicistronic Vaccines with Immune Response-Enhancing Genes (IREGS) Improve Mouse Survival

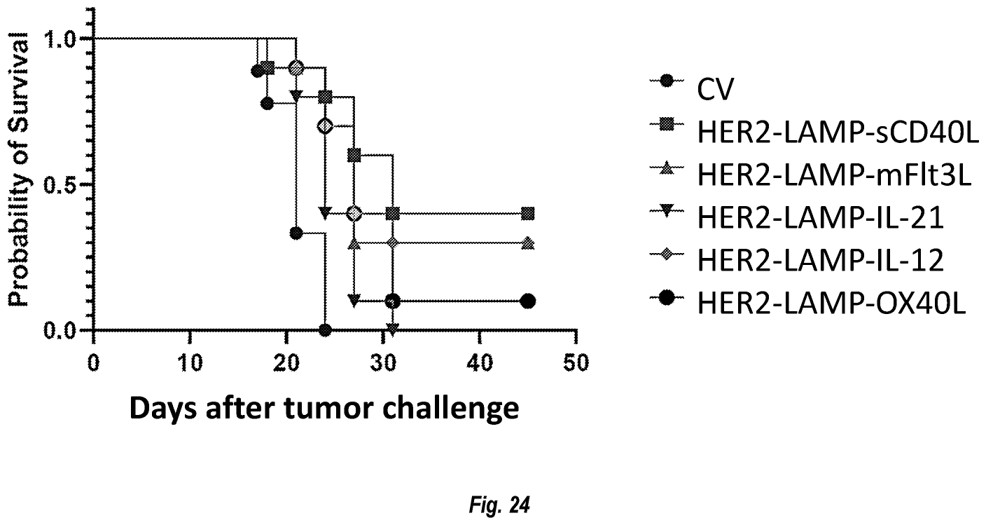

All five HER2-LAMP-IREG vaccines-(1) HER2-LAMP-CD40L, (2) HER2-LAMP-Flt3L, (3) HER2-LAMP-IL-21, (4) HER2-LAMP-IL-12, and (5) HER2-LAMP-OX40L, were tested for their influence on mouse survival. The mice were first injected subcutaneously with TSA-HER2 cells, then administered four separate immunizations at 2, 9, 16, and 23 days after injection of the TSA-HER2 cells. Over the course of 45 days, mouse survival was monitored. The data show that all immunized mice had improved survival compared to mice treated with a control vector (CV) (FIG. 24). The mice that received HER2-LAMP-CD40L showed the highest probability of survival (FIG. 24).

### 4. Summary

Tumor growth was suppressed by all tested HER2-LAMP-IREG vaccines. CD40L provided the most anti-tumor protection, followed Flt3L and IL-12. Also, all HER2-LAMP-IREG vaccines improved mouse survival. Improvement in mouse survival correspond with vaccine suppression of tumor growth.

### Example 5. The Bicistronic Her2-LAMP-IL-15 Vaccine Elicits Antigen-Specific T-Cell Response in Mice

IL-15 is a T helper type 1 cytokine (Chen et al., 2014) that has been demonstrated to have a marked antitumor immune response and may reverse host tolerance of tumor antigens in certain preclinical trials. IL-15 is a 14-15 kDa 4 alpha-helix-bundle family cytokine family member that stimulates the generation of natural killer (NK) cells, natural killer T (NKT) cells, gamma delta (γδ) T cells, ILC1 cells, intraepithelial lymphocytes (IELs), innate cells expressing CD103+ CD56+ CD44+ and memory CD8 T cells (Motegi et al., 2008; Wu & Xu, 2010). IL-15, like IL-2, stimulates proliferation of T cells, induces generation of cytotoxic lymphocytes and memory phenotype CD8 T cells, and stimulates proliferation and maintenance of NK cells. in contrast to IL-2, IL-15 does not mediate activation-induced cell death (AICD), does not consistently activate Tregs, and may cause less capillary leak syndrome. IL-15 efficacy was observed in multiple murine immunotherapy trials, including trials with syngeneic transgene adenocarcinoma mouse prostate cancer cells (TRAMP-C2), Pmel-1 mice, B16 melanoma cells, Mc38 cells, and CT26 colon carcinoma cells. These studies suggest that IL-15 may be more effective than IL-2 in cancer therapy (Guo et al., 2021; Heon et al., 2015; Klebanoff et al., 2004; Morris et al., 2014; Rauch et al., 2014).

This Example discusses the effect of IL-15 on the mouse immune response induced by a bicistronic HER2 DNA vaccine). The immune response induced by HER2-LAMP, HER2-LAMP-CD40L and HER2-LAMP-IL-15 were compared.

### A. Materials and Methods

In general, materials and methods used in this Example were as described in Examples 1-4 above, with the following modifications.

### 1. Vaccine Constructs

The vaccines discussed in this Example are LAMP-Hinge-HER2 (expressing HER2-LAMP alone without an IREG polypeptide), a HER2-LAMP-IL-15 construct (expressing polypeptides HER2-LAMP, see, e.g., SEQ ID NO: 195, and secreted IL-15, e.g., IL-15 with an Ig-kappa signal sequence SEQ ID NO: 225), LAMP-HER2-CD40L (SEQ ID NO: 197), and control vector (CV, i.e., the vector without any gene insertions). These vaccines were prepared as described in the preceding Examples.

### 2. Immunization

Six-to eight-week old female BALB/c mice were bred and maintained at a licensed animal facility. Mice were treated with vaccines as shown in Table 5 below by intradermal (ID) injection in the ear. Mice were immunized with vaccine on days 0 and 16. Mice were bled on day 36. Serum was collected and stored at −30° C. Spleens were collected on days 14 and 36 and processed for ELISPOT to evaluate HER2-specific T cell responses.

TABLE 5
Immunization Schedule.

| | | Concentration | Dose | # | Vol | Mouse | | | | Day | | |
|---|---|---|---|---|---|---|---|---|---|---|---|---|
| Group | Vaccine | (mg/mL) | (μg) | Mice | (μL) | ID | Day −1 | Day 0 | Day 14 | 15-17 | Day 29 | Day 36 |
| A | Control | 3.3 | 20 | 10 | 20 | 2095- | Prebleed | First | Terminated | Second | Bled | Terminated |
| | Vector | | | | | 2104 | | [symbol] | [symbol] | [symbol] | serum | [symbol] |
| | (CV) | | | | | | | | | | | |
| B | LAMP- | 3.0 | 20 | 10 | 20 | 2105- | | | | | | |
| | Hinge- | | | | | 2114 | | | | | | |
| | HER2 | | | | | | | | | | | |
| C | HER2- | 2.0 | 20 | 10 | 20 | 2115- | | | | | | |
| | LAMP | | | | | 2124 | | | | | | |
| | CD40L | | | | | | | | | | | |
| D | HER2- | 1 | 20 | 10 | 50 | 2125- | | | | | | |
| | LAMP | | | | | 2134 | | | | | | |
| | IL-15 | | | | | | | | | | | |

[symbol]

indicates data missing or illegible when filed

### 3. Evaluation of Antigen-Specific T Cell Response

Splenocytes were depleted of red blood cells (RBCs) and co-cultured in U-bottom 96-well plates in 200 μl/well T cell media (RPMI-1640 with L-Glutamine and HEPES (ATCC), 1% penicillin, 1% streptomycin, and 5×10⁵M betamercaptoethanol (3ME)) at 1×10⁶ cells/well and 1 μg/mL HER2 peptide mix or medium alone under Brefeldin A for 5 hours at 37° C. in 5% CO₂. The plates were centrifuged at 1200 rpm for 6 minutes and cell pellets were collected for intracellular staining.

### 4. ELISPOT

While general materials and methods that relate to ELISPOT analysis for T cell response were as described in Examples 2-3 above, modifications and particular details that relate to this Example are provided herein. 96-well nitrocellulose plates (Millipore), were coated overnight at 4° C. with 100 μL/well of capture monoclonal antibody in PBS. The plates were washed three times in 200 μL/well T cell media and blocked with 200 μL/well T cell media for at least 2 hours at room temperature. splenocytes were plated at 3×10⁵ cells/well and co-cultured with 1 μg/well T cell media (RPMI-1640 with L-Glutamine and HEPES (ATCC), 1% penicillin, 1% streptomycin, and 5×10⁵M betamercaptoethanol (BME)) for 48 hours at 37° C. in 5% CO₂. The plates were washed two times with 200 μL/well PBS and two times with 200 μL/well PBS-T (0.05% Tween in PBS). Diluted detection antibodies (50 μL/well in PBS-T/0.5% BSA) were added and plates were incubated for 2 hours with shaking at room temperature. Plates were washed four times with PBS four times. Streptavidin-alkaline phosphates diluted in PBS (50 μL/well) were added and incubated for 2 hours. Plates were washed with PBS four times and developed with 50 μL/well of 3-amino-9-ethylcarbazole (AEC, BD Bioscience) substrate for 10 min. Color development was stopped by washing under running tap water. After drying 72 hours at room temperature in the dark, colored spots were counted using an AID ELISPOT High-Resolution Reader System and AID ELISPOT Software version 3.5 (Autoimmun Diagnostika GmbH).

### 5. Flow Cytometry

While general materials and methods that relate to ELISPOT analysis for T cell response were as described in the preceding Examples, modifications and particular details that relate to this Example are provided herein. Cells were first labeled with Zombie aqua fixable viability dye in PBS (1:500 dilution) followed by surface antibodies (1:100 dilution) in staining buffer (4% FBS, 2% rat serum, 2% mouse serum in PBS). For intracellular staining cells were stained with Zombie aqua, followed by surface staining, fixation with 4% paraformaldehyde, and stained with intracellular antibody in permeabilization buffer (PBS with 1% FCS, 0.1% saponin).

### 6. Statistics

T-test was performed using GraphPad Prism 6.0 software to evaluate the statistical significance. Each mouse's RPMI result was deducted from the results of the antigen activation.

### B. Results

### 1. Her2-LAMP-IL-5 Elicited Strong T-Cell Response

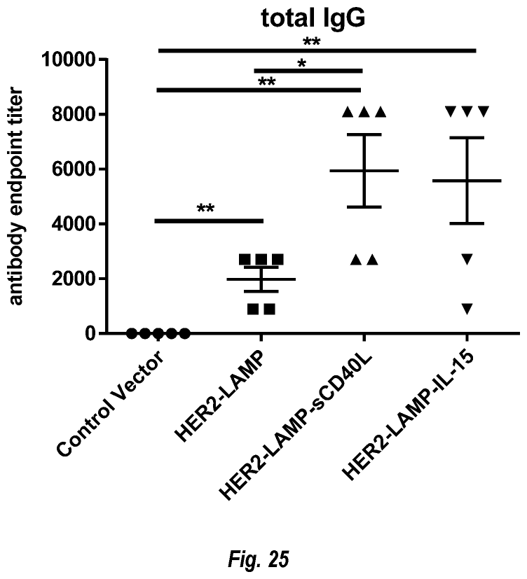

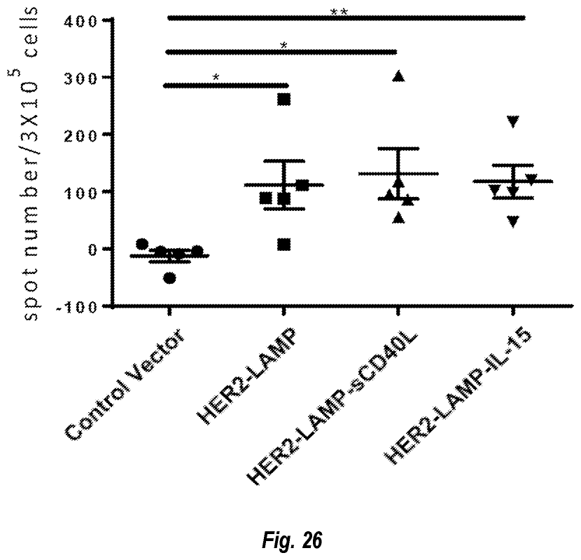

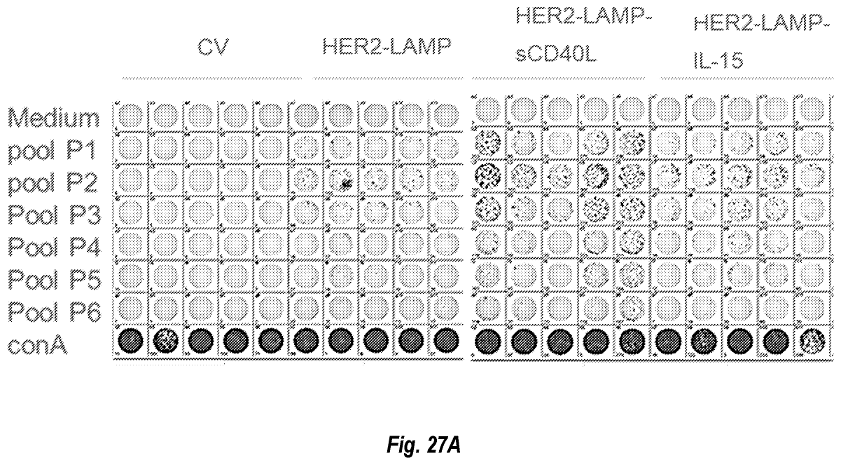

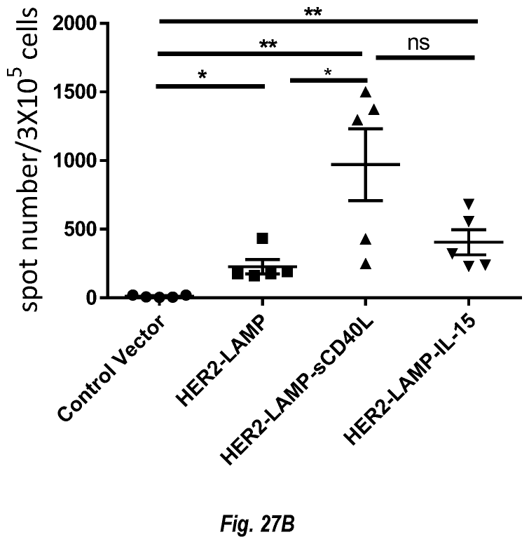

When compared to a HER2 vaccine without an IREG (LAMP-Hinge-HER2), two bicistronic vaccines with IREG, HER2-LAMP-IL-15, and HER2-LAMP-CD40L, induced stronger antigen-specific antibody response as measured by ELISA (FIG. 25). The bicistronic vaccines also elicited strong T-cell response. While one dose either bicistronic vaccine was sufficient to elicit said response (FIG. 26), two doses of HER2-LAMP-IL-15 elicited a stronger response than two doses of either LAMP-Hinge-HER2 or HER2-LAMP-CD40L (FIGS. 27A-B).

### 2. Summary

The immune response induced by Hinge-LAMP-HER2, HER2-LAMP-IL-15, and HER2-LAMP-CD40L were compared. Both bicistronic vaccines, HER2-LAMP-IL-15, and HER2-LAMP-CD40L, induced robust T cell response compared to Hinge-LAMP-HER2, especially when two doses of each vaccine were administered. Both bicistronic vaccines enhanced CD4 T cells to produce IFNγ compared with CD8 T cells. However, there was no significant difference between HER2-CD40L and HER2-IL-15 treatments. A study is ongoing to determine the anti-tumor activity of HER2-LAMP-IL-15.

Variations, modifications, and other implementations of what is described herein will occur to those of ordinary skill in the art without departing from the spirit and scope of the disclosure and the claims. All of the patents, patent applications, international applications, and references identified are expressly incorporated herein by reference in their entireties.

### Example 6. Anti-Tumor Activity of Bicistronic Her2-LAMP-Soluble-CD40L In Vivo

### A. Introduction

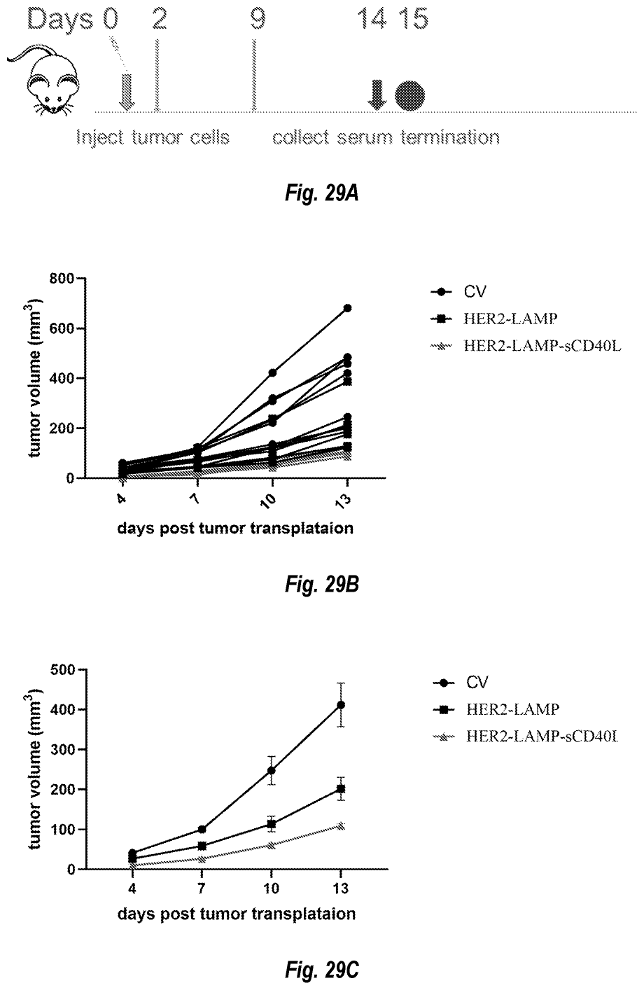

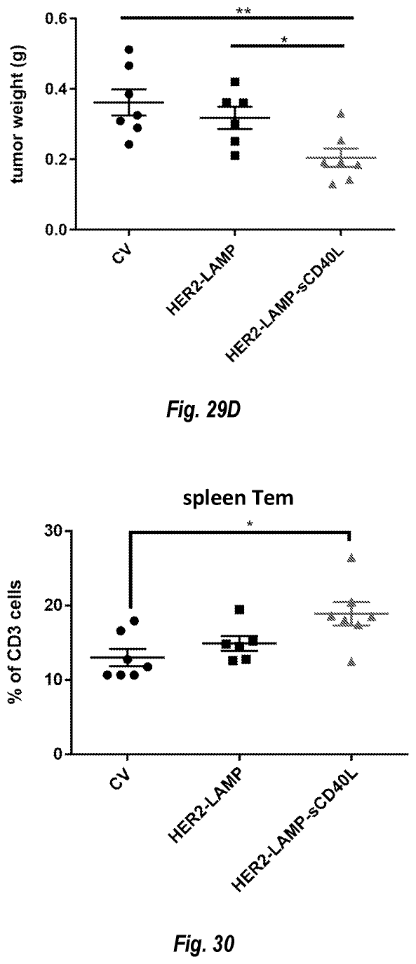

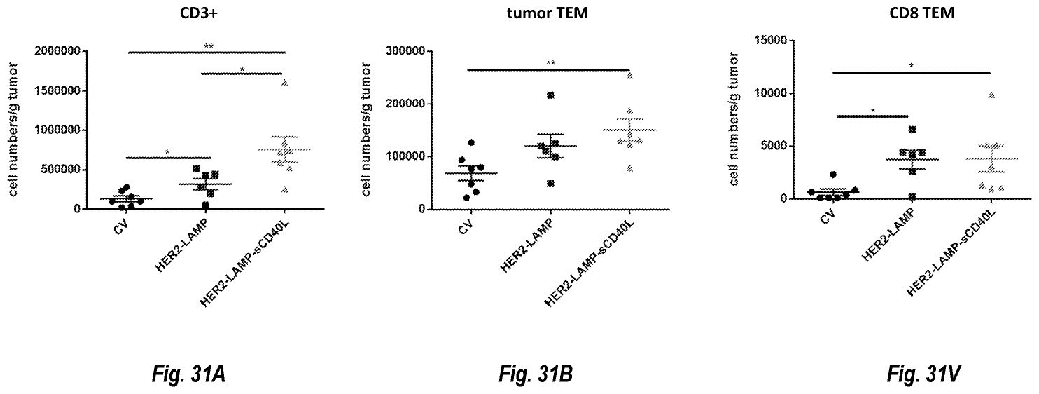

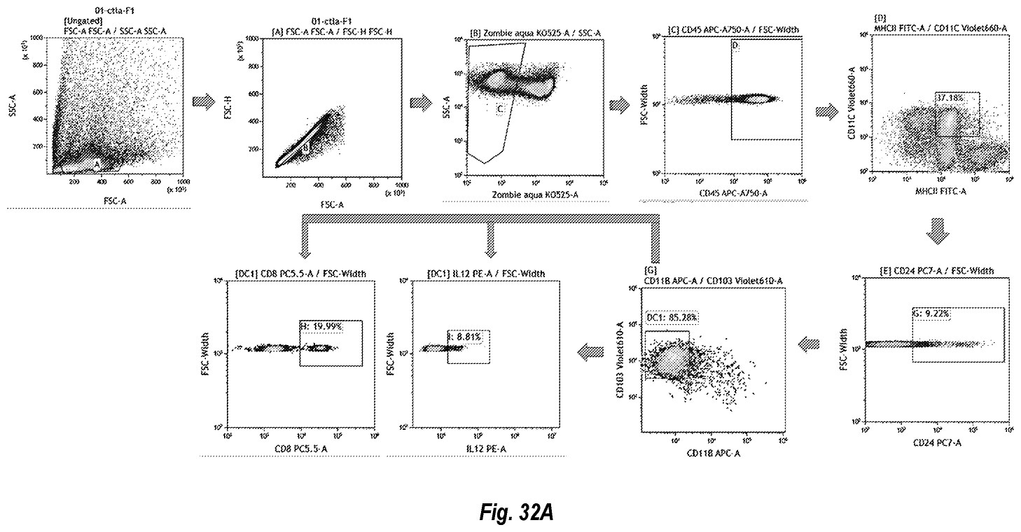

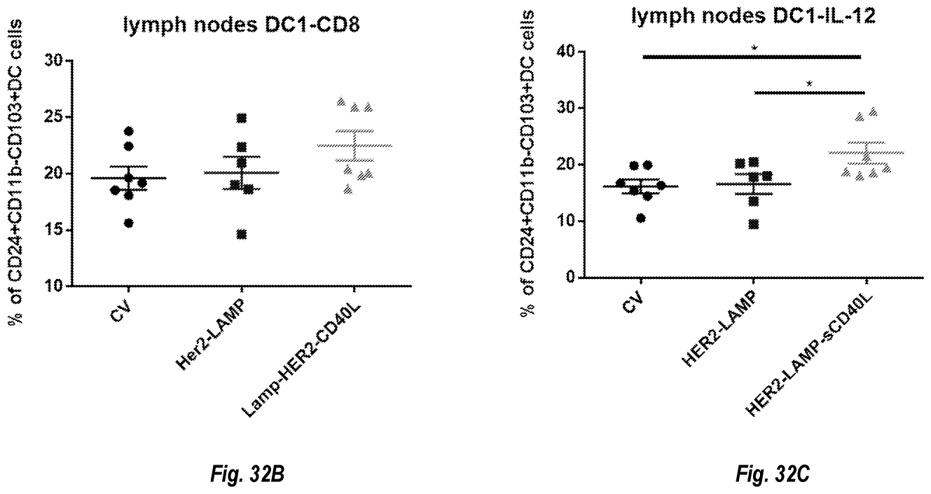

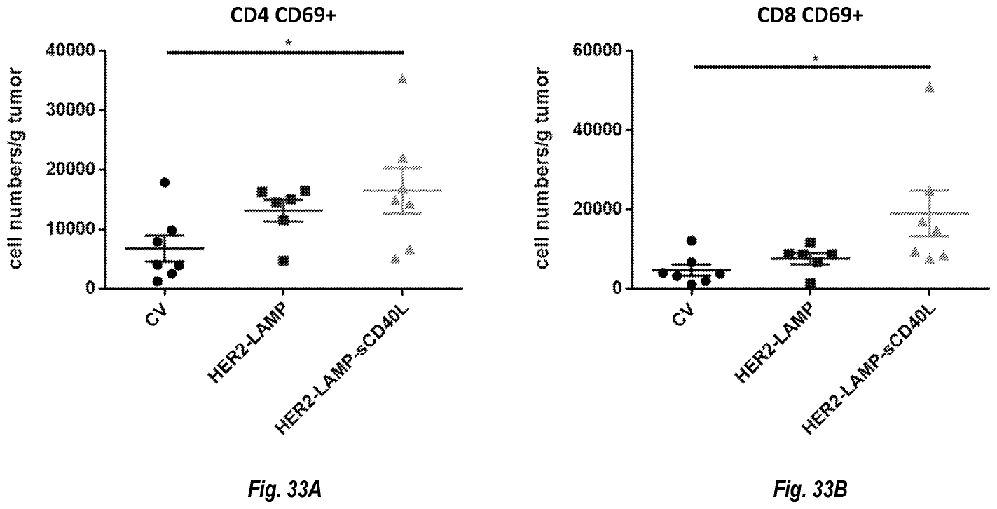

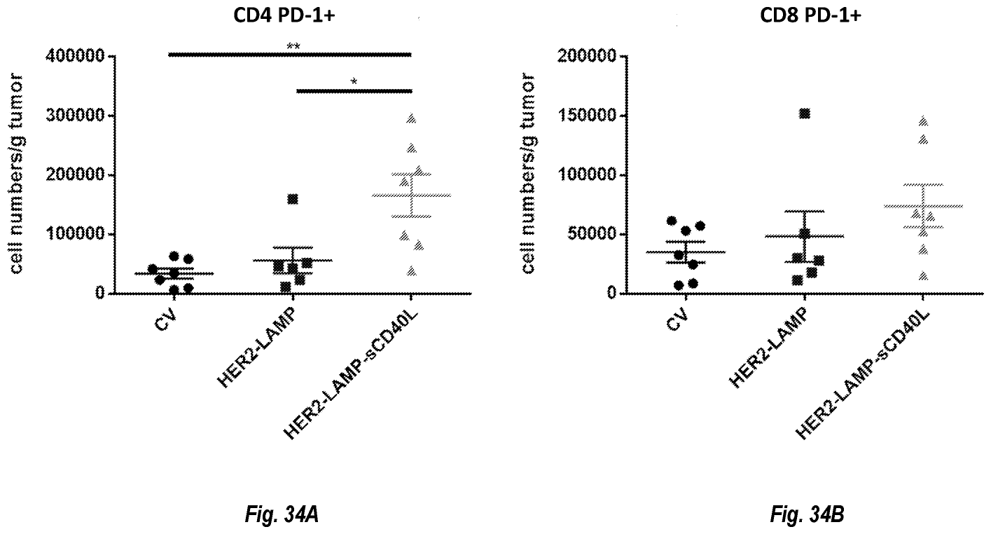

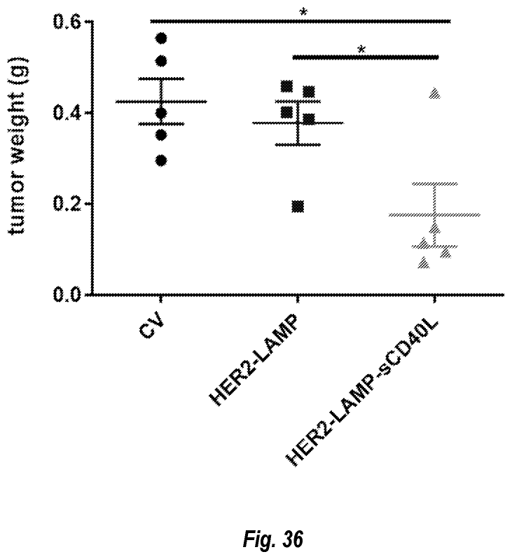

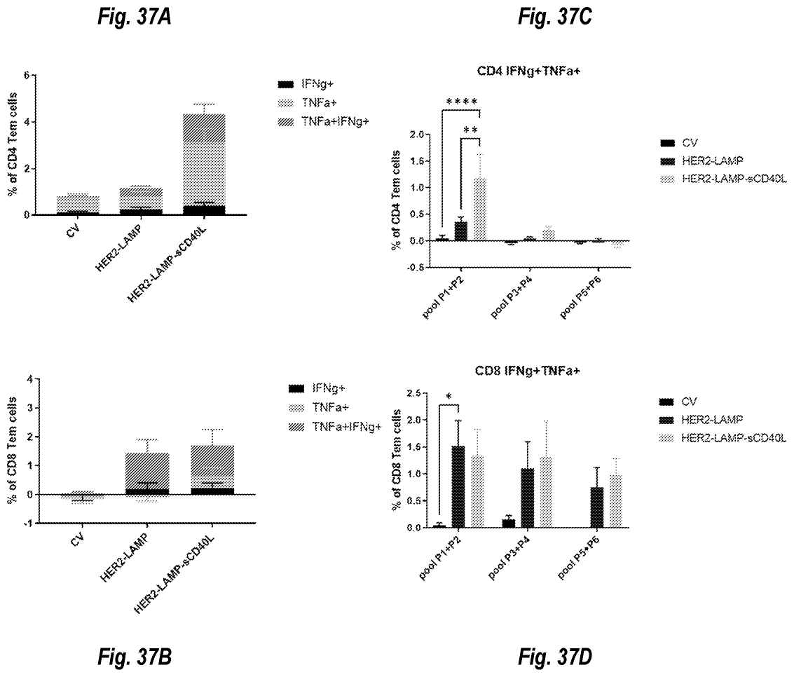

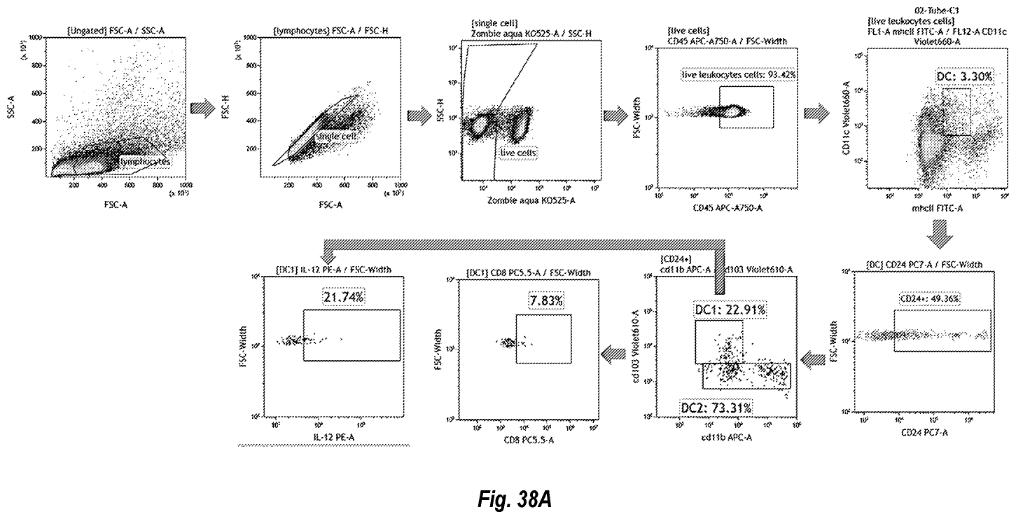

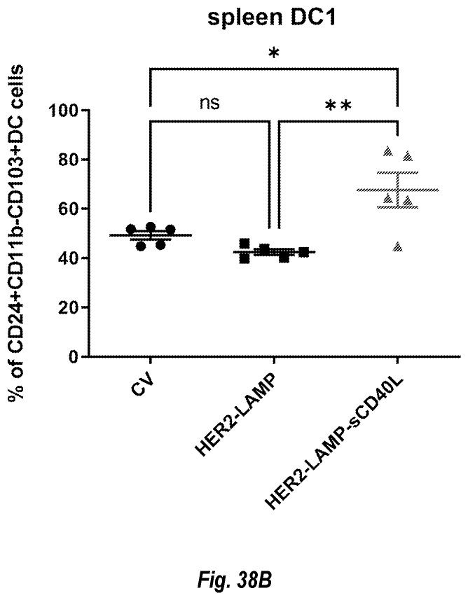

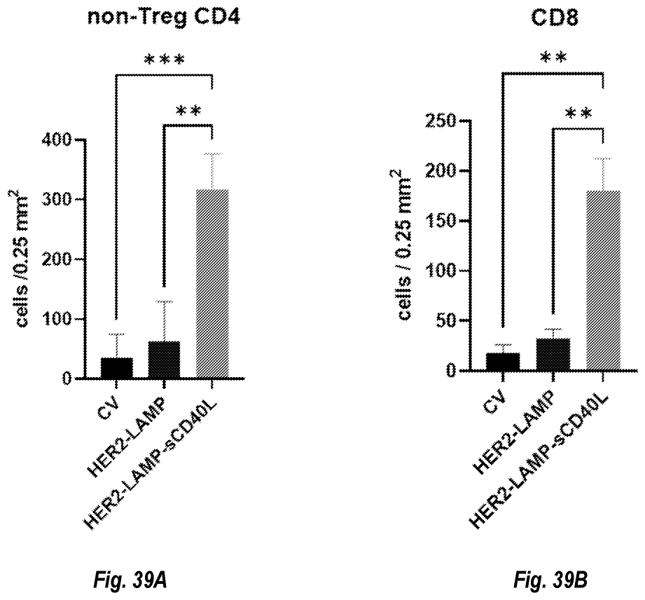

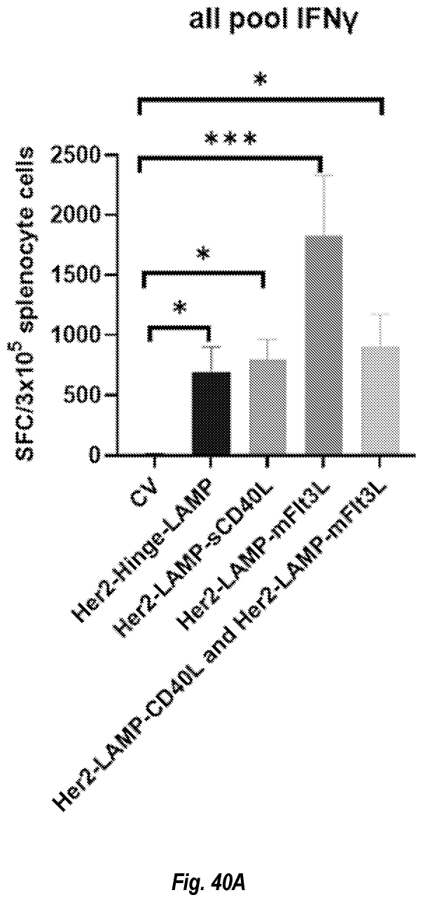

The anti-tumor efficacy of non-bicistronic HER2-LAMP and bicistronic HER2-LAMP-sCD40L, delivered by DNA vectors, was compared in a breast tumor model and the cytokine profile in the tumor microenvironment was explored. The administration and analysis schedule is shown in FIG. 29A. The overall results, collectively shown in FIGS. 29-39, showed that bicistronic HER2-LAMP-sCD40L enhances anti-tumor effect by significantly suppression of tumor growth (FIGS. 29 and 36); bicistronic HER2-LAMP-sCD40L systematically affects memory T cells and infiltration of T cells into the tumor micro-environment (TME) (FIGS. 30-31); bicistronic HER2-LAMP-sCD40L induces antigen-presenting cells to produce IL-12 by activation of DC1 dendritic cells in the draining lymph nodes (FIG. 32); bicistronic HER2-LAMP-sCD40L activates inflammatory response in the TME, turning the tumor from “cold” to “hot” (FIG. 33); bicistronic HER2-LAMP-sCD40L promotes CD4 T cells to produce PD-1 in the TME (FIG. 34); bicistronic Her2-LAMP-sCD40L elicits stronger IFNg production compared to Her2-LAMP DNA vaccine in the spleens (FIG. 37); soluble CD40L expressed by the constructs induces antigen-presenting cells to enhance DC1 activation in the spleen (FIG. 38); and bicistronic Her2-LAMP-sCD40L increases CD4 and CD8 T cells in the peritumor and intratumor tissues (FIG. 39).

### B. Summary and Results

To test the ability of non-bicistronic HER2-LAMP and bicistronic HER2-LAMP-sCD40L to suppress tumor growth in vivo, in a breast tumor model, female BALB/c mice were inoculated with 1×10⁵ TSA cells expressing Her2 (injected into mammary fat pad) on day zero, followed by immunization with 20 μg of control vector, Her2-LAMP, or Bicistronic-Her2-LAMP-sCD40L in 20 μl PBS on day 2, and 9. There were seven mice in each treatment group. Tumor size was monitored by caliper. Tumor diameter was calculated as the square root of length×width, and tumor volume was calculated using the formula 4/3πr³. Serum was collected 5 days after the last dose (or on day 14). The experiment was terminated 6 days after the last dose (or on day 15), and tumors were weighed at the termination. FIG. 29A shows a schematic of the study design. FIG. 29B-C show results from a run of this experiment with seven mice in each group. These figures show the change in tumor size (in mm³) from 4 to 13 days post tumor transplantation, for the individual animals and for each animal group. As can be seen in FIGS. 29B-C, the bicistronic constructs suppressed tumor growth more effectively than either the HER2-LAMP or control. FIG. 29D shows the change in tumor weight (in g). As can be seen in the figure, there was a statistically significant reduction in tumor weight comparing the bicistronic construct to the control (** p<0.01) as well as a statistically significant difference in tumor weight between the HER2-LAMP and bicistronic construct groups (* p<0.05).

For subsequent experiments, tumors were minced with scissors into 2- to 4-mm pieces placed into a gentleMACS™ C tube containing 2.5 ml dissociation medium (2.35 ml RPMI with 100 ul of enzyme D, 50 ul of enzyme R and 12.5 μl of enzyme A). Tumors were processed on the gentleMACS™ dissociator, followed by incubation at 37° C. The cells were filtered through a 70-mm mesh filter and washed twice with 10 ml RPMI (300 g for 7 mins), then resuspended in PBS/1% FBS staining buffer and incubated with fluorescent antibodies, and subjected to flow cytometry as described below. Fluorescently coupled CD3, CD45, CD11c, CD11b, CD103, PD-1, PD-L1, NK1.1, CD25, FoxP3, CD24, IL-12, IL-2, CD4, CD8, CD44, CD62L, IFNγ, TNFα monoclonal antibodies and Zombie aqua fixable viability kit were purchased from BioLegend (San Diego, CA). One-Way ANOVA was performed using GraphPad Prism® 6.0 software to evaluate the statistical significance, as described below. Each mouse's RPMI result was deducted from the results of the antigen activation.

FIG. 30 shows the percentage of CD3+ memory T cells in splenocytes at termination in each of the treatment groups. Splenocytes were stained by Zombie, a cell surface marker, and intracellular staining according to an in-house staining protocol. Cells were gated on Zombie−CD44+CD62L−CD3+ memory T cells. Data in FIG. 30 is representative of mean±SEM (7 mice per group). Based on this experiment, there was a statistically significant increase in CD3+ memory T cells in the bicistronic HER2-LAMP-sCD40L treatment group compared to the control, as shown in the figure (* p<0.05).

FIGS. 31A-C show that the bicistronic HER2-LAMP-sCD40L construct promoted infiltration of T cells into the tumor microenvironment (TME). One day after termination, tumors were cleaned, weighed, digested using Miltenyi® tumor dissociation kit and the gentleMACS™ dissociator, and stained with fluorescent antibodies. Cells were gated on CD45+CD3+Zombie− population for CD4 and CD8, and CD45+CD3+Zombie− CD44+CD62L− for effector memory T cells (TEM cells). Results are shown in FIGS. 31A-C, and the data is representative of mean±SEM. As the data shows, there is statistically higher infiltration of T cells into the tumor microenvironment in mice administered the bicistronic construct compared to the control or HER2-LAMP construct.

FIGS. 32A-C show that the soluble CD40L expressed from the bicistronic constructs in the mice and that it enhanced activation of type 1 dendritic cells (DC1 cells) producing IL-12 in draining lymph nodes. After termination, lymphocyte cells were prepared and stained by the Zombie surface marker, and intracellular staining was conducted according to an in-house staining protocol. DC1 cells were gated on Zombie−CD45+MHCII+CD11c+CD24+CD103+ cells, as shown in FIG. 32A. DC cells were defined as MHCII+CD11C+, while DC1 cells were defined as MHCII+CD11C+CD24+CD103+CD11b−, and DC2 cells were defined as MHCII+CD11C+CD24+CD11b+. FIG. 32B shows the percentage of DC1 cells expressing CD8, while FIG. 32C shows the percentage of DC1 cells expressing IL-12. One-way ANOVA statistical analysis shows that the bicistronic constructs led to a statistically significant increase in such IL-12-expressing DC1 cells compared to both the control and the HER2-LAMP experiments.

FIGS. 33A-B show that the bicistronic HER2-LAMP-sCD40L construct activates an inflammatory signal in the tumor microenvironment. One day after termination of the experiments, tumors were cleaned, weighed, digested using Miltenyi® tumor dissociation kit and the gentleMACS™ dissociator, and stained with fluorescent antibodies. Cells were gated on Zombie−CD45+CD69CD4+/CD8+ cells. Data is representative of mean±SEM. As shown in the figures, there was a statistically significant increase in CD4+CD69+ and CD8+CD69+ cells in the tumor after treatment with the bicistronic construct compared to the control (* indicates p<0.5).

Further data, shown in FIGS. 34A-B, indicates that the bicistronic construct also promoted T cells to produce PD-1 in the tumor microenvironment. One day after termination of the experiments, tumors were cleaned, weighed, digested using Miltenyi® tumor dissociation kit and the gentleMACS™ dissociator, and stained with fluorescent antibodies. Cells were gated on CD45+CD3+PD1+CD4+Zombie− population for CD4 and CD45+CD3+PD1+CD8+Zombie− for CD8. As shown in the figures, the bicistronic construct showed a statistically significant increase in the number of CD4 PD-1+ cells compared with both the control and the HER2-LAMP construct.

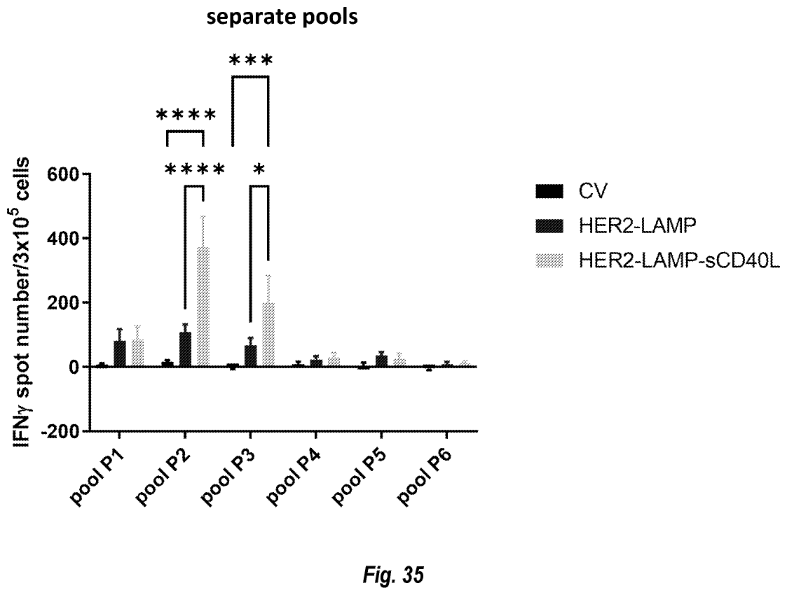

Further data showing that bicistronic HER2-LAMP-sCD40L suppressed tumor growth in a statistically significant fashion in comparison to HER2-LAMP and control, following the protocol set forth in FIG. 29A, are provided in FIG. 36, in which the experiment was repeated with 5 mice per group. Following this run of the experiment with 5 mice per group, additional data were collected. FIG. 35 shows that the bicistronic construct also induced a stronger T cell response against particular pooled peptides of HER2 extracellular domain (ECD). At the termination of the experiment, splenocytes from the mice were incubated with 1 μg/ml of Her2 pooled peptides (individual peptides were synthesized from GenScript and pooled in six different pools called P1-P6) for 48 hrs. Data represent original spots (top) and mean IFNγ spot forming cells ±SEM (bottom, n=5). Two-way ANOVA was used for statistical analysis. * p<0.05, *** p<0.001, **** p<0.0001. Specifically, splenocytes from vaccinated mice were evaluated for antigen-specific IFNγ production by Enzyme-linked immunospot (ELISPOT), and colored spots following addition of detection antibodies were counted using an AID ELISPOT High-Resolution Reader System and AID ELISPOT Software version 3.5 (Autoimmun Diagnostika GmbH). For flow cytometry, cells were first labelled with Zombie aqua fixable viability dye in PBS (1:500 dilution), followed by surface antibodies (1:100 dilution) in staining buffer (4% FBS, 2% rat serum, 2% mouse serum in PBS). For intracellular staining cells were stained with Zombie aqua, followed by surface staining, fixation with 4% paraformaldehyde, and stained with intracellular antibody in permeabilization buffer (PBS with 1% FCS 0.1% saponin). Samples were analyzed on a CytoFlex® flow cytometer (Beckman Coulter) and analyzed using Kaluza® software (Beckman Coulter). Bar graphs from the FACS analysis provided in FIG. 37A-D show that the bicistronic HER2-LAMP-sCD40L construct induced polyfunctional CD4 effector memory T cells in the spleen. For this analysis, splenocytes were incubated with Her2 pooled peptides (individual peptide was synthesized from GenScript) in the presence of Brefeldin A and Monesin for 6 hrs. Cells were harvested and stained by Zombie, surface marker, and were intracellularly stained and were gated on memory T cells. FIG. 37A-D show bar graphs from the FACS. Data is representative of mean plus/minus SEM. Two-way ANOVA was used for statistical analysis, where *P<0.05, ** p<0.01, **** p<0.0001.

Dendritic cells from this second run of the experiment with 5 mice per group were also analyzed. As shown in FIGS. 38A-B, splenocytes were harvested and stained by Zombie, and gated on Zombie−CD45+MHCII+CD11c+CD24+CD103+, and the percentage of such CD24+CD11b−CD103+ dendritic cells was determined. As shown in FIG. 38B, there were statistically more such cells in the spleen of mice receiving the bicistronic construct compared to the control and compared to the HER2-LAMP construct (* p<0.05, ** p<0.01; n=5 per group).

Finally, FIG. 39 further shows the results of cell staining data indicating that the sCD40L expressed from the bicistronic construct increased CD4+ and CD8+ T cells in the tumor in a statistically significant fashion compared to the control and the HER2-LAMP constructs. Specifically, after termination of the in vivo experiments, tumors were collected and fixed in 10% formalin. The tissues were cut and mounted on slides and stained with DAPI, anti-CD4 or anti-CD8, and anti-FoxP3 by Ultivue (Cambridge, MA, USA). FIGS. 39A-B show bar graphs analyzed from the original imaging data. Data is representative of mean±SEM. n=3 for CV and HER2-LAMP and n=4 for Her2-LAMP-sCD40L. One-way ANOVA was used for statistical analysis. ** p<0.01, *** p<0.001.

### Example 7. In Vivo Studies with Bicistronic Her2-LAMP-mFlt3L and Her2-LAMP-SCD40L

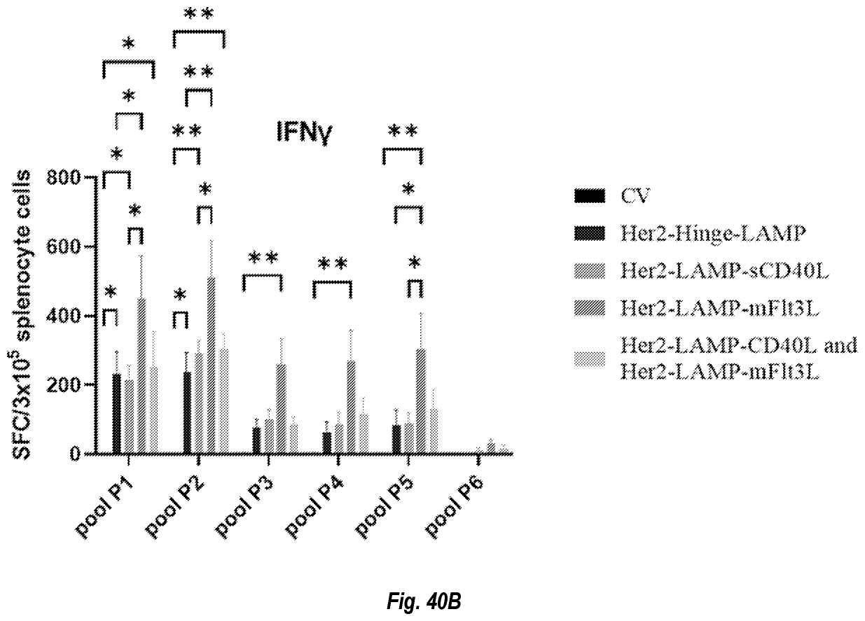

The objective of this study was to determine if the immune response would be enhanced by immunization with both bicistronic HER2-LAMP-sCD40L and HER2-LAMP-mFlt3L constructs provided as DNA vectors. Six to eight-week old Balb/c mice were purchased from the Jackson Laboratory (Maine, USA). 20 μg of Control vector, HER2-LAMP (also called HER2-hinge-LAMP), Bicistronic-Her2-LAMP-sCD40L, Bicistronic-HER2-LAMP-mFlt3L, or both Her2-LAMP-sCD40L and HER2-LAMP-mFlt3L vaccines were used in a total volume of 20 μl per mouse per dose for intradermal injection. Seven mice were included in each group. Mice were immunized on days 0, and 14, and terminated 10 days after the second dose. Her2-specific T cell responses were evaluated by ELISPOT and FACS assays. The experiment was terminated ten days after the last dose. Splenocytes were incubated with Her2 pooled peptides (GenScript) for 48 hrs. Data are shown in FIGS. 40A-B and represent original spots (top) and mean IFNγ spot forming cells ±SEM. One-Way ANOVA was used for statistical analysis (* p<0.05, ** p<0.01, *** p<0.001). As can be seen in FIGS. 40A-B, Her2-LAMP-mFlt3L induced significantly stronger T cells response recalled by Her2 pooled peptides 1, 2, 3, 4 and 5, while Her2-LAMP and Her2-LAMP-sCD40L only elicited T cell response against pooled peptide 1 and 2. Furthermore, each of the bicistronic constructs, when administered individually led to splenocyte recognition of particular pooled Her2 ECD peptides, but no synergistic effect was seen with respect to such recognition.

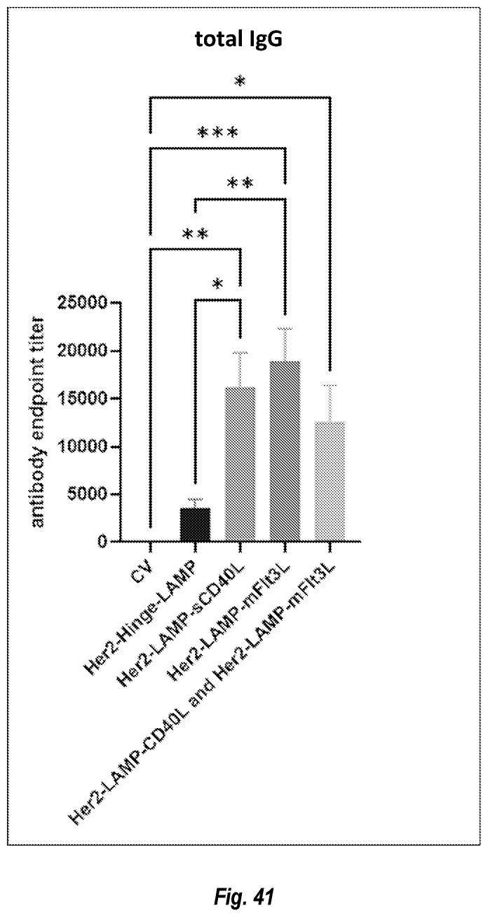

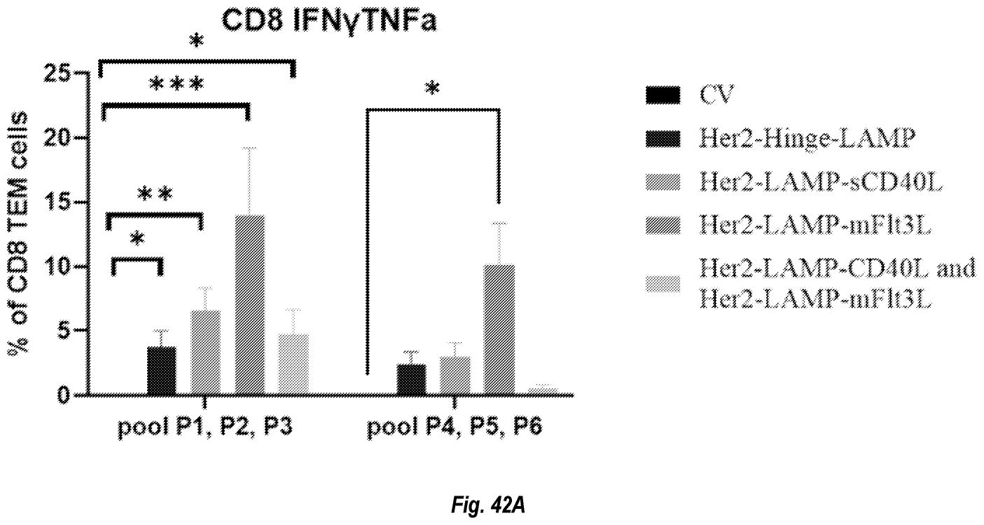

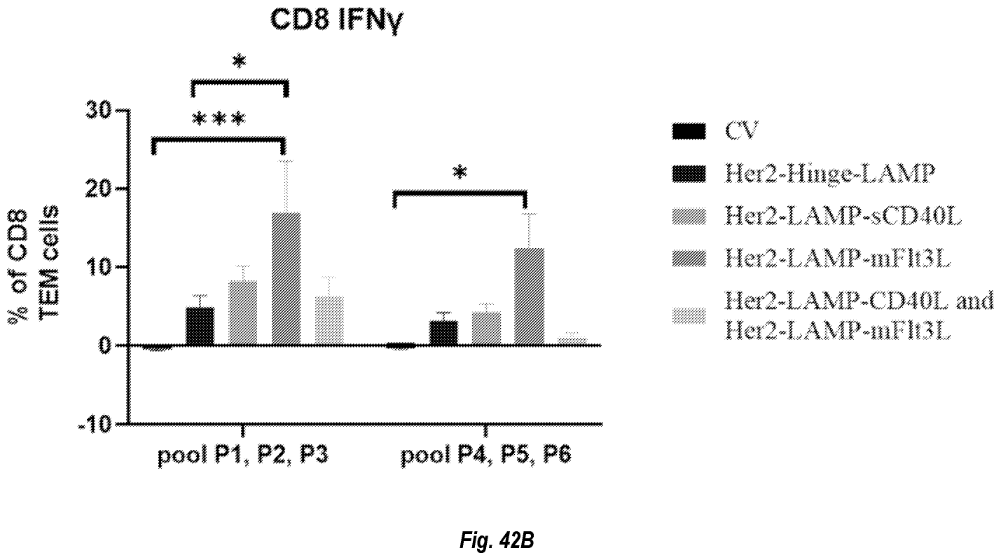

As shown in FIG. 41, bicistronic Her2-LAMP-sCD40L, Her2-LAMP-mFlt3L, and the combined vaccines also induced higher antibody response compared to the monocistronic DNA, with the Her2-LAMP-mFlt3L vaccine showing the highest antibody response. Specifically, at the termination of the experiment, serum samples were collected, and HER2-specific IgG were measured by indirect ELISA. The data are shown in FIG. 41, and represent mean of antibody titers ±SEM. n=6 per group. T-test was used for statistical analysis. * p<0.05, ** p<0.01, *** p<0.001.

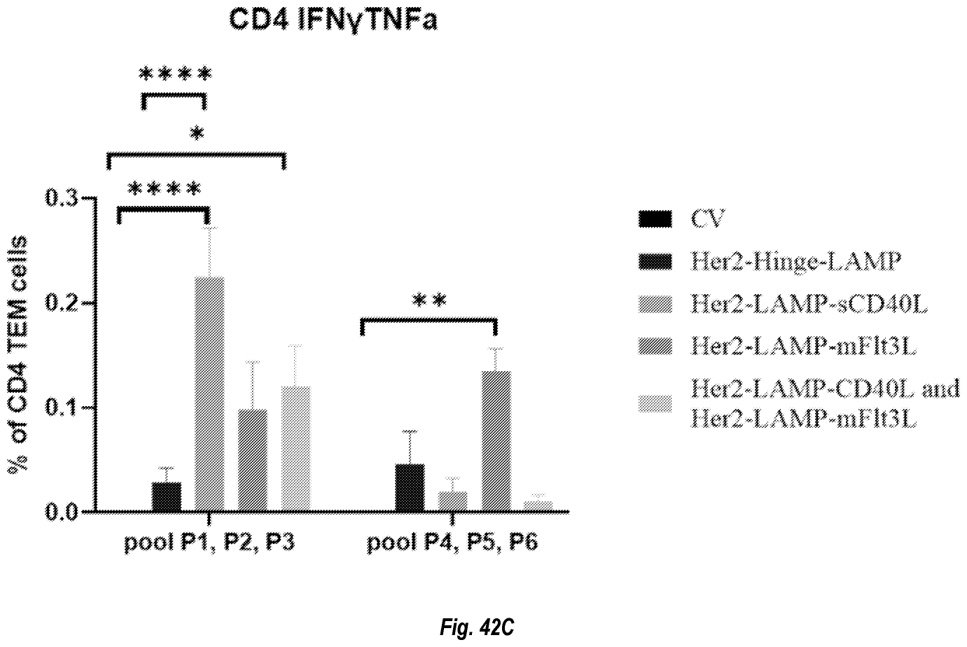

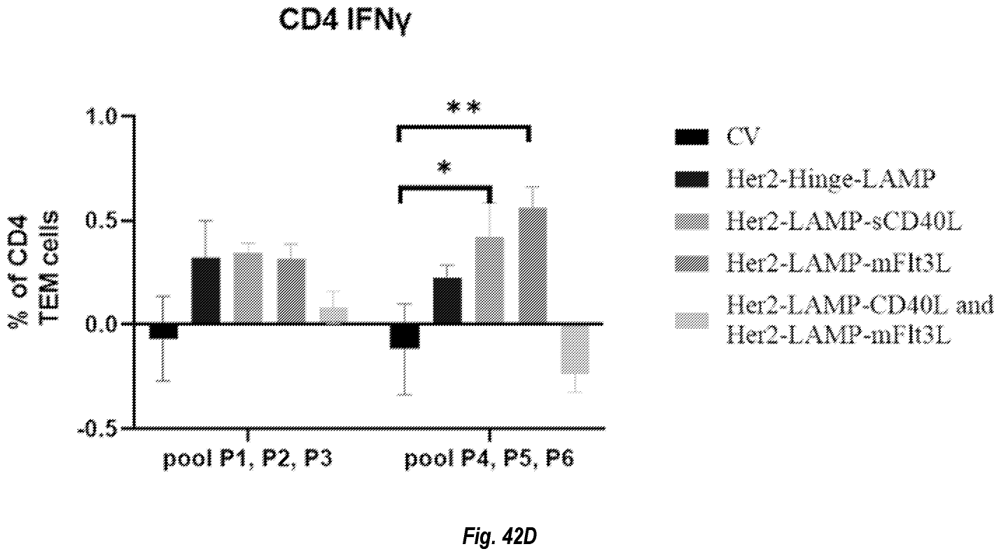

The studies further demonstrated that Her2-LAMP-sCD40L increases polyfunctional CD4 T cells while Her2-LAMP-mFlt3L enhances polyfunctional CD8 T cells in comparison of the Her2-LAMP DNA vaccine, as shown in FIG. 42A-D. For this analysis, splenocytes (1×10⁶/well) were stimulated with Her2 pooled peptides (1 μg/ml, GenScript) in T cell media (RPMI with 10% heat inactivated FBS, 1% penicillin/streptomycin, and 1X β-ME) with monensin and brefeldin A for 5 hours. Cells were harvested and stained by Zombie, surface marker, and intracellular staining according to an in-house staining protocol. Cells were gated for FACS on memory T cells. FIGS. 42A-D show bar graphs analyzed from the original FACS. Data is representative of mean±SEM. One-Way ANOVA was used for statistical analysis. * p<0.05, ** p<0.01, *** p<0.001, **** p<0.0001.

### Example 8. In Vivo Studies with Bicistronic Her2-LAMP-mFlt3L and Her2-LAMP-IL-12

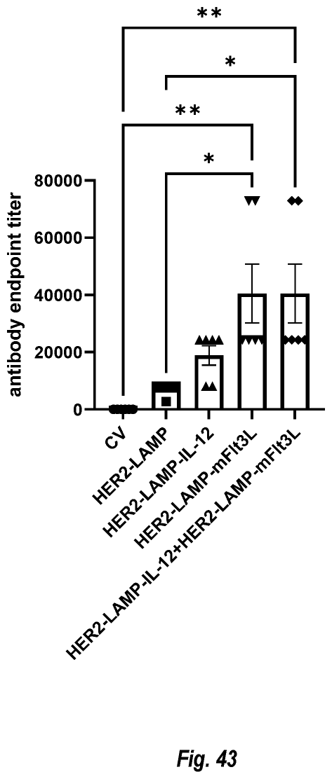

In further experiments, female Balb/c mice were injected with 20 μg of a DNA vector encoding Her2-LAMP or Her2-LAMP-IL-12 or Her2-LAMP-mFlt3L or 20 μg of control vector or mixture of Her2-LAMP-IL-12 and Her2-LAMP-mFlt3L intradermally on day 0, and 14. Serum samples were collected on day 22. Her 2 specific IgG were measured by indirect ELISA. Results are shown in FIG. 43, indicating that Her2-LAMP-mFlt3L constructs provided a higher antibody response than Her2-LAMP-IL-12 constructs.

### REFERENCES

Beatty, G. L., Chiorean, E. G., Fishman, M. P., Saboury, B., Teitelbaum, U. R., Sun, W., Huhn, R. D., Song, W., Li, D., Sharp, L. L., Torigian, D. A., O'Dwyer, P. J., & Vonderheide, R. H. (2011). CD40 agonists alter tumor stroma and show efficacy against pancreatic carcinoma in mice and humans. Science (New York, N.Y.), 331 (6024), 1612-1616. https://doi.org/10.1126/science.1198443
Beatty, G. L., Li, Y., & Long, K. B. (2017). Cancer immunotherapy: Activating innate and adaptive immunity through CD40 agonists. Expert Review of Anticancer Therapy, 17 (2), 175-186. https://doi.org/10.1080/14737140.2017.1270208
Chen, X., Ni, J., Meng, H., Li, D., Wei, Y., Luo, Y., & Wu, Y. (2014). Interleukin-15: A potent adjuvant enhancing the efficacy of an autologous whole-cell tumor vaccine against Lewis lung carcinoma. Molecular Medicine Reports, 10 (4), 1828-1834. https://doi.org/10.3892/mmr.2014.2474
Gómez, C. E., Nájera, J. L., Sánchez, R., Jiménez, V., & Esteban, M. (2009). Multimeric soluble CD40 ligand (sCD40L) efficiently enhances HIV specific cellular immune responses during DNA prime and boost with attenuated poxvirus vectors MVA and NYVAC expressing HIV antigens. Vaccine, 27 (24), 3165-3174. https://doi.org/10.1016/j.vaccine.2009.03.049
Grewal, I. S., & Flavell, R. A. (1998). CD40 and CD154 in cell-mediated immunity. Annual Review of Immunology, 16, 111-135. https://doi.org/10.1146/annurev.immunol.16.1.111
Guo, J., Liang, Y., Xue, D., Shen, J., Cai, Y., Zhu, J., Fu, Y.-Y., & Peng, H. (2021). Tumor-conditional IL-15 pro-cytokine reactivates anti-tumor immunity with limited toxicity. Cell Research, 31 (11), 1190-1198. https://doi.org/10.1038/s41422-021-00543-4
Heon, E. K., Wulan, H., Macdonald, L. P., Malek, A. O., Braunstein, G. H., Eaves, C. G., Schattner, M. D., Allen, P. M., Alexander, M. O., Hawkins, C. A., McGovern, D. W., Freeman, R. L., Amir, E. P., Huse, J. D., Zaltzman, J. S., Kauff, N. P., Meyers, P. G., Gleason, M. H., Overholtzer, M. G., . . . . Press, O. A. (2015). IL-15 induces strong but short-lived tumor-infiltrating CD8 T cell responses through the regulation of Tim-3 in breast cancer. Biochemical and Biophysical Research Communications, 464 (1), 360-366. https://doi.org/10.1016/j.bbrc.2015.06.162
Klebanoff, C. A., Finkelstein, S. E., Surman, D. R., Lichtman, M. K., Gattinoni, L., Theoret, M. R., Grewal, N., Spiess, P. J., Antony, P. A., Palmer, D. C., Tagaya, Y., Rosenberg, S. A., Waldmann, T. A., & Restifo, N. P. (2004). IL-15 enhances the in vivo antitumor activity of tumor-reactive CD8+ T cells. Proceedings of the National Academy of Sciences of the United States of America, 101 (7), 1969-1974. https://doi.org/10.1073/pnas.0307298101
Le Bert, N., Tan, A. T., Kunasegaran, K., Tham, C. Y. L., Hafezi, M., Chia, A., Chng, M. H. Y., Lin, M., Tan, N., Linster, M., Chia, W. N., Chen, M. I.-C., Wang, L.-F., Ooi, E. E., Kalimuddin, S., Tambyah, P. A., Low, J. G.-H., Tan, Y.-J., & Bertoletti, A. (2020). SARS-COV-2-specific T cell immunity in cases of COVID-19 and SARS, and uninfected controls. Nature, 584 (7821), 457-462. https://doi.org/10.1038/s41586-020-2550-z
Morris, J. C., Ramlogan-Steel, C. A., Yu, P., Black, B. A., Mannan, P., Allison, J. P., Waldmann, T. A., & Steel, J. C. (2014). Vaccination with tumor cells expressing IL-15 and IL-15Rα inhibits murine breast and prostate cancer. Gene Therapy, 21 (4), 393-401. https://doi.org/10.1038/gt.2014.10
Motegi, A., Kinoshita, M., Inatsu, A., Habu, Y., Saitoh, D., & Seki, S. (2008). IL-15-induced CD8+CD122+ T cells increase antibacterial and anti-tumor immune responses: Implications for immune function in aged mice. Journal of Leukocyte Biology, 84 (4), 1047-1056. https://doi.org/10.1189/jlb.0807530
Rauch, D. A., Harding, J. C., & Ratner, L. (2014). IL-15 deficient tax mice reveal a role for IL-1a in tumor immunity. PloS One, 9 (1), e85028. https://doi.org/10.1371/journal.pone.0085028
Schoenberger, S. P., Toes, R. E., van der Voort, E. I., Offringa, R., & Melief, C. J. (1998). T-cell help for cytotoxic T lymphocytes is mediated by CD40-CD40L interactions. Nature, 393 (6684), 480-483. https://doi.org/10.1038/31002
Stone, G. W., Barzee, S., Snarsky, V., Kee, K., Spina, C. A., Yu, X.-F., & Kornbluth, R. S. (2006). Multimeric soluble CD40 ligand and GITR ligand as adjuvants for human immunodeficiency virus DNA vaccines. Journal of Virology, 80 (4), 1762-1772. https://doi.org/10.1128/JVI.80.4.1762-1772.2006
van Mierlo, G. J. D., den Boer, A. T., Medema, J. P., van der Voort, E. I. H., Fransen, M. F., Offringa, R., Melief, C. J. M., & Toes, R. E. M. (2002). CD40 stimulation leads to effective therapy of CD40 (−) tumors through induction of strong systemic cytotoxic T lymphocyte immunity. Proceedings of the National Academy of Sciences of the United States of America, 99 (8), 5561-5566. https://doi.org/10.1073/pnas.082107699
Vonderheide, R. H., Dutcher, J. P., Anderson, J. E., Eckhardt, S. G., Stephans, K. F., Razvillas, B., Garl, S., Butine, M. D., Perry, V. P., Armitage, R. J., Ghalie, R., Caron, D. A., & Gribben, J. G. (2001). Phase I study of recombinant human CD40 ligand in cancer patients. Journal of Clinical Oncology: Official Journal of the American Society of Clinical Oncology, 19 (13), 3280-3287. https://doi.org/10.1200/JCO.2001.19.13.3280
Wu, Z., & Xu, Y. (2010). IL-15R alpha-IgG1-Fc enhances IL-2 and IL-15 anti-tumor action through NK and CD8+ T cells proliferation and activation. Journal of Molecular Cell Biology, 2 (4), 217-222. https://doi.org/10.1093/jmcb/mjq012

### EQUIVALENTS

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. An isolated nucleic acid molecule comprising
a. a first polynucleotide sequence encoding a polypeptide comprising two homology domains of a luminal domain of a LAMP protein, and an antigenic domain heterologous to the LAMP protein (collectively a “LAMP-antigen Construct”), wherein the antigenic domain is placed between the two homology domains; and
b. a second polynucleotide sequence encoding at least one second polypeptide comprising an immune response enhancing gene polypeptide (IREG) or an extracellular domain of an IREG comprising a secretion signal sequence.

2. The isolated nucleic acid molecule of claim 1, wherein the LAMP protein is selected from LAMP-1, LAMP2, LAMP-3, lysosomal integral membrane protein-2 (“LIMP 2”), Macrosailin, Endolyn, LAMP5 or limbic system-associated membrane protein (“LIMBIC”).

3. The isolated nucleic acid molecule of claim 2, wherein the LAMP protein is comprises an amino acid sequence selected from any one of SEQ ID NO: 1-113, or comprises an amino acid sequence at least 70%, at least 75%, at least 80%, at least 85%, at least 90%, at least 95%, at least 96%, at least 97%, at least 98%, or at least 99% identical to SEQ ID NO: 1-113.

4. (canceled)

5. The isolated nucleic acid molecule of claim 2, wherein the LAMP protein is LAMP-1 and wherein the two homology domains of the LAMP-antigen Construct comprise LAMP-1 Homology Domain 1 and LAMP-1 Homology Domain 2.

6. The isolated nucleic acid molecule of claim 5, wherein the human LAMP-1 Homology Domain 1 comprises (a) the amino acid sequence of residues 29-194 of SEQ ID NO: 1 or comprises the amino acid sequence of residues 29-195 of SEQ ID NO: 198, or (b) a variant of (a) wherein said variant comprises an amino acid sequence at least 95% or at least 95% identical to the amino acid sequence of (a); and/or the human LAMP-1 Homology Domain 2 comprises the amino acid sequence of residues 228-381 of SEQ ID NO: 1.

7. (canceled)

8. The isolated nucleic acid molecule of claim 1, wherein the LAMP-antigen Construct comprises a linker between at least one of the two homology domains and the antigenic domain.

9. The isolated nucleic acid molecule of claim 8, wherein the linker comprises an amino acid sequence of GPGPG or PMGLP.

10. The isolated nucleic acid molecule of claim 1, wherein the LAMP-antigen Construct further comprises a transmembrane domain and/or cytoplasmic domain of a LAMP Protein.

11. The isolated nucleic acid molecule of claim 10, wherein the transmembrane domain comprises residues 383 to 405 of SEQ ID NO: 1 and/or wherein the cytoplasmic domain comprises residues 406-417 of SEQ ID NO: 1.

12. The isolated nucleic acid molecule of claim 1, wherein the LAMP-antigen Construct further comprises a signal sequence.

13. The isolated nucleic acid molecule of claim 12, wherein the signal sequence is derived from a LAMP Protein, such as a signal sequence comprising residues 1-28 of SEQ ID NO: 1 or residues 1-28 of SEQ ID NO: 198.

14. (canceled)

15. (canceled)

16. The isolated nucleic acid molecule of claim 1, wherein the IREG comprises one or more of CD40L, CD80, OX40, Flt3L, GM-CSF, IL-12, IL-21, IL-23, IL-15, CD70, CD86, IL-7, IL-18, or IL-33, or an extracellular domain thereof, optionally wherein the CD40L, CD80, OX40, Flt3L, GM-CSF, IL-12, IL-21, IL-23, or IL-15 is fused to an Fc domain of an immunoglobulin.

17. The isolated nucleic acid molecule of claim 1, wherein the secretion signal sequence is heterologous to the IREG and/or derived from IgK VIII, Ig-kappa, tetranectin, or IL-2, and/or wherein the second polypeptide further comprises pulmonary surfactant associated protein D (SPD).

18. (canceled)

19. The isolated nucleic acid molecule of claim 17, wherein the second polypeptide is expressed under the control of an EF-1alpha core promoter, such as that of SEQ ID NO: 124.

20. A composition comprising the isolated nucleic acid molecule of claim 1.

21. A host cell comprising the isolated nucleic acid molecule of claim 1.

22. A composition comprising the host cell of claim 21.

23. A method of treating a subject having a disease or a disorder or of inducing an immune response in a subject with a disease or disorder or at risk of developing a disease or disorder, wherein the method comprises administering to the subject the isolated nucleic acid molecule of claim 1, the composition comprising the isolated nucleic acid molecule, or the host cell comprising the isolated nucleic acid molecule, in an amount sufficient to treat the disease or disorder or to induce an immune response in the subject,
optionally wherein the method further comprises administering at least one second therapeutic to the subject.

24. (canceled)

25. The isolated nucleic acid molecule of claim 1, wherein
the first polynucleotide sequence encodes a polypeptide comprising two homology domains of a luminal domain of a LAMP protein, and an antigenic domain comprising HER2 extracellular domain (collectively “HER2-LAMP”), wherein the antigenic domain is placed between the two LAMP homology domains.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. The isolated nucleic acid of claim 1, wherein the isolated nucleic acid comprises DNA, mRNA, or self-amplifying RNA.
